(19) United States
(12) Patent Application Publication
(10) Pub. No.: US 2023/0372337 A1
(43) Pub. Date: Nov. 23, 2023

(54) Combination Therapy Comprising an AXL Inhibitor

(71) Applicant: BERGENBIO ASA, Bergen (NO)

(72) Inventor: James LORENS, Bergen (NO)

(73) Assignee: BERGENBIO ASA, Bergen (NO)

(21) Appl. No.: 17/913,686

(22) PCT Filed: Mar. 23, 2021
(86) PCT No.: PCT/EP2021/057406

§ 371 Date: Sep. 22, 2022

(30) Foreign Application Priority Data
Mar. 23, 2020 (GB) 2004189.3

(51) Int. Cl. A61K 31/502; A61P 35/00; A61K 31/704; A61K 39/395; C07K 16/28; C07K 16/40; A61K 31/282; A61K 31/519

(52) U.S. Cl. CPC A61K 31/502; A61P 35/00; A61K 31/704; A61K 39/3955; C07K 16/2818; C07K 16/40; A61K 31/282; A61K 31/519; C07K 2317/565; C07K 2317/56; C07K 2317/24

(57) ABSTRACT

This disclosure relates to a combination therapy for treating patients suffering from a proliferative disease. More particularly, the disclosure relates to combination therapies comprising an AXL inhibitor, an immune checkpoint modulator (ICM), and a chemotherapeutic agent and/or radiotherapy for treating patients suffering from cancer, as well as compositions and methods for treating patients with said combination therapy.

### CROSS-REFERENCE TO RELATED APPLICATION

This application is a national stage entry pursuant to 35 U.S.C. § 371 of International Application PCT/EP2021/057406 filed Mar. 23, 2021, which claims priority to Great Britain Application No. 2004189.3 filed Mar. 23, 2020, each of which is incorporated by reference herein in its entirety.

### FIELD

This disclosure relates to a combination therapy for treating patients suffering from a proliferative disease. More particularly, the disclosure relates to combination therapies comprising an AXL inhibitor, an immune checkpoint modulator (ICM), and a chemotherapeutic agent for treating patients suffering from cancer, as well as compositions and methods for treating patients with said combination therapy.

### BACKGROUND

AXL

All of the protein kinases that have been identified to date in the human genome share a highly conserved catalytic domain of around 300 amino acids. This domain folds into a bi-lobed structure in which resides ATP-binding and catalytic sites. The complexity of protein kinase regulation allows many potential mechanisms of inhibition including competition with activating ligands, modulation of positive and negative regulators, interference with protein dimerization, and allosteric or competitive inhibition at the substrate or ATP binding sites.

AXL (also known as UFO, ARK, and Tyro7; nucleotide accession numbers NM_021913 and NM_001699; protein accession numbers NP_068713 and NP_001690) is a receptor protein tyrosine kinase (RTK) that comprises a C-terminal extracellular ligand binding domain and N-terminal cytoplasmic region containing the catalytic domain. The extracellular domain of AXL has a unique structure that juxtaposes immunoglobulin and fibronectin Type Ill repeats and is reminiscent of the structure of neural cell adhesion molecules. AXL and its two close relatives, Mer/Nyk and Sky (Tyro3/Rse/Dtk), collectively known as the Tyro3 family of RTK's, all bind and are stimulated to varying degrees by the same ligand, GAS6 (growth arrest specific-6), a ˜76 kDa secreted protein with significant homology to the coagulation cascade regulator, Protein S. In addition to binding to ligands, the AXL extracellular domain has been shown to undergo homophilic interactions that mediate cell aggregation, suggesting that one important function of AXL may be to mediate cell-cell adhesion.

AXL is predominantly expressed in the vasculature in both endothelial cells (EC's) and vascular smooth muscle cells (VSMC's) and in cells of the myeloid lineage and is also detected in breast epithelial cells, chondrocytes, Sertoli cells and neurons. Several functions including protection from apoptosis induced by serum starvation, TNF-α or the viral protein E1A, as well as migration and cell differentiation have been ascribed to AXL signalling in cell culture. AXL has been found to serve as a key checkpoint for interferon (IFN) signaling (Rothlin et al, 2007; Huang et al, 2015); in the context of viral responses, the Zika virus has been found to antagonize the IFN action by interacting with AXL (Chen et al, 2018). However, Axl−/− mice exhibit no overt developmental phenotype and the physiological function of AXL in vivo is not clearly established in the literature.

AXL Pathology

The overexpression of AXL and/or its ligand has also been reported in a wide variety of solid tumor types including, but not limited to, breast, renal, endometrial, ovarian, thyroid, non-small cell lung carcinoma, and uveal melanoma as well as in myeloid leukemias. Furthermore, it possesses transforming activity in NIH3T3 and 32D cells. It has been demonstrated that loss of Axl expression in tumor cells blocks the growth of solid human neoplasms in an in vivo MDA-MB-231 breast carcinoma xenograft model. Taken together, these data suggest AXL signalling can independently regulate EC angiogenesis and tumor growth and thus represents a novel target class for tumor therapeutic development.

The expression of AXL and GAS6 proteins is upregulated in a variety of other disease states including endometriosis, vascular injury and kidney disease and AXL signalling is functionally implicated in the latter two indications. AXL-GAS6 signalling amplifies platelet responses and is implicated in thrombus formation. AXL may thus potentially represent a therapeutic target for a number of diverse pathological conditions including solid tumors, including, but not limited to, breast, renal, endometrial, ovarian, thyroid, non-small cell lung carcinoma and uveal melanoma; liquid tumors, including but not limited to, leukemias (particularly myeloid leukemias) and lymphomas; endometriosis, vascular disease/injury (including but not limited to restenosis, atherosclerosis and thrombosis), psoriasis; visual impairment due to macular degeneration; diabetic retinopathy and retinopathy of prematurity; kidney disease (including but not limited to glomerulonephritis, diabetic nephropathy and renal transplant rejection), rheumatoid arthritis; osteoporosis, osteoarthritis and cataracts.

AXL Inhibitors

In view of the role played by AXL in numerous pathological conditions, the development of safe and effective AXL inhibitors has been a topic of interest in recent years. Different groups of AXL inhibitors are discussed in, inter alia, US20070213375, US 20080153815, US20080188454, US20080176847, US20080188455, US20080182862, US20080188474, US20080117789, US20090111816, WO2007/0030680, WO2008/045978, WO2008/083353, WO2008/0083357, WO2008/083354, WO2008/083356, WO2008/080134, WO2009/054864, and WO2008/083367.

Combination Therapies Using AXL Inhibitors

The combination of one or more of the above cited AXL inhibitors with one or more other agents is discussed in, for example, WO2010/083465 and WO2016/193680, with WO2016/193680 focussing on combinations of AXL inhibitors with agents having immune-regulatory or modulatory activity. For example, inhibition of AXL with the small molecule Bemcentinib (BGB324/R428) was found to enhance the efficacy of immune checkpoint inhibitor treatment with anti PD1 and/or anti CTLA4.

Combination Therapies Using Immune Checkpoint Modulators

It is increasingly recognized that the effectiveness of conventional cytotoxic chemotherapeutic treatments is at least partially mediated through its interplay with the tumor and host immune response. The different classes of cytotoxic drugs have specific effects on the immune contexture, with varying ability to induce immunogenic cell death and influence suppressive and effector immune cells (Galluzzi et al, 2015; Yan et al, 2018).

The combination of inhibition of the PD1/PDL1-axis with cytotoxic chemotherapy is currently being explored in several clinical trials, including a phase 3 clinical trial in triple negative breast cancer (TNBC) (Emens et al, 2016), with a reported increased median overall survival as compared to chemotherapy alone in a phase 3 clinical trial in non-small cell lung cancer (NSCLC) (Gandhi et al, 2018).

The complexity of tumour biology and its interaction with the immune system, along with the potential for serious side-effects inherent in such powerful therapies, means that research is ongoing to identify efficacious combination therapies, and specific disorders and/or subjects that will benefit most from such treatments.

### SUMMARY

The present authors sought to investigate the mechanistic interaction of combination therapies including AXL inhibition (with bemcentinib), immune checkpoint blockade (with anti-CTLA4/anti-PD1) and cytotoxic chemotherapy (with the anthracycline doxorubicin). Chemotherapeutic agents cause cell death of cancer cells (e.g. localised tumor cell death), the release of tumour antigens, and a subsequent immune response which may include upregulation of release of type I IFNs. Type I IFNs can in turn activate AXL—active AXL downregulates the IFN response and inhibits the immune response. AXL inhibition is also known to potentiate chemotherapy, independently of the immune system. Immune checkpoint inhibitors modulate the body's immune system, thereby facilitating immune the immune response to disease.

By investigating the efficacy of a triple combination therapy including AXL inhibition (with bemcentinib), immune checkpoint blockade (with anti-CTLA4/anti-PD1) and cytotoxic chemotherapy (with the anthracycline doxorubicin) in the 4T1 syngeneic mammary carcinoma model and Yumm 1.7 syngeneic melanoma model, the present authors have discovered that such combination therapies are able to significantly delay tumor growth, increase mouse survival, and increase the number of long term responder animals as compared to individual and sub-combination treatments.

Accordingly, in a first aspect the present disclosure provides a method of treating an AXL-related disease, the method comprising administering to a subject in need thereof a therapeutically effective amount of an AXL inhibitor, wherein the AXL inhibitor is administered in combination with: one or more immune checkpoint modulator (ICM); and, one or more chemotherapeutic agent.

The AXL inhibitor may be a compound of formula (I) as described in more detail elsewhere herein:

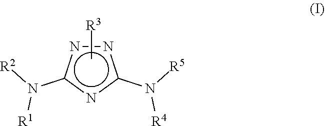

The AXL inhibitor may be bemcentinib. The AXL inhibitor may also be an antibody; for example, an antibody comprising the 6 CDRs having the sequences of SEQ ID Nos. 1 to 6, or the 6 CDRs having the sequences of SEQ ID Nos. 7 to 12.

The immune checkpoint modulator (ICM) may be an immune checkpoint inhibitor (ICI), or a T cell co-stimulatory agonist. For example, the ICM may be an immune checkpoint modulating antibody selected from the group consisting of: anti-CTLA-4 antibodies, anti-PD-1 antibodies, anti-PD-L1 antibodies, anti-4-1BB antibodies, anti-OX-40 antibodies, anti-GITR antibodies, anti-CD27 antibodies, anti-CD28 antibodies, anti-CD40 antibodies, anti-LAG3 antibodies, anti-ICOS antibodies, anti-TWEAKR antibodies, anti-HVEM antibodies, anti-TIM-1 antibodies, anti-TIM-3 antibodies, anti-VISTA antibodies, and anti-TIGIT antibodies. The immune checkpoint modulator (ICM) may be selected from the group consisting of: anti-CTLA-4 antibodies, anti-PD-1 antibodies, and anti-PD-L1 antibodies.

The chemotherapeutic agent may be a chemotherapeutic agent which induces immunogenic cell death of cancer cells and/or which induces an immune response in the subject. The chemotherapeutic agent may be a chemotherapeutic agent which induces a type I interferon response in the subject. The chemotherapeutic agent may be an anthracycline, for example, doxorubicin, daunorubicin, epirubicin, idarubicin, mitoxantrone, or valrubicin. The chemotherapeutic agent may be doxorubicin. The chemotherapeutic agent may be a taxane, for example, docetaxel, paclitaxel, or abraxane. The chemotherapeutic agent may be docetaxel.

The AXL-related disease may be a proliferative disease, a solid tumour, or cancer. The cancer may be selected from the group consisting of: breast cancer, lung cancer, non-small-cell lung cancer, melanoma, mesothelioma, acute myeloid leukemia (AML), myelodysplastic syndrome (MDS), pancreas cancer, kidney cancer, urothelial carcinoma, and glioblastoma. The cancer may be breast cancer. The cancer may be melanoma. The cancer may be lung cancer.

The AXL-related disease may be a cancer or tumor having or expected to have low tumor mutation burden (TMB) and/or low numbers of oncogenic driver mutations. Such cancers/tumours are associated with poor response to immunotherapies. Thus, the AXL-related disease may be a cancer or tumor that is, or is expected to be, refractory, non-responsive, or otherwise not benefit from treatment with one or more immune checkpoint modulator (ICM).

In a second aspect the present disclosure provides an AXL inhibitor, an immune checkpoint modulator (ICM), and a chemotherapeutic agent, for use in a method of treating an AXL-related disease according to the first aspect.

Also included in the second aspect are: an AXL inhibitor for use in a method of treating an AXL-related disease according to the first aspect; an immune checkpoint modulator (ICM) for use in a method of treating an AXL-related disease according to the first aspect; and, a chemotherapeutic agent for use in a method of treating an AXL-related disease according to the first aspect.

In addition, included in the second aspect are: an AXL inhibitor and an immune checkpoint modulator (ICM) for use in a method of treating an AXL-related disease according to the first aspect; an AXL inhibitor and a chemotherapeutic agent for use in a method of treating an AXL-related disease according to the first aspect; and, an immune checkpoint modulator (ICM) and a chemotherapeutic agent for use in a method of treating an AXL-related disease according to the first aspect.

In a third aspect the present disclosure provides use of an AXL inhibitor, an immune checkpoint modulator (ICM), and a chemotherapeutic agent in the manufacture of a medicament for treating a disorder in a subject, wherein the treatment comprises a method of treating an AXL-related disease according to the first aspect.

Also included in the third aspect are: use of an AXL inhibitor in the manufacture of a medicament for treating a disorder in a subject, wherein the treatment comprises a method of treating an AXL-related disease according to the first aspect; use of an immune checkpoint modulator (ICM) in the manufacture of a medicament for treating a disorder in a subject, wherein the treatment comprises a method of treating an AXL-related disease according to the first aspect; and, use of a chemotherapeutic agent in the manufacture of a medicament for treating a disorder in a subject, wherein the treatment comprises a method of treating an AXL-related disease according to the first aspect.

In addition, included in the third aspect are: use of an AXL inhibitor and an immune checkpoint modulator (ICM) in the manufacture of a medicament for treating a disorder in a subject, wherein the treatment comprises a method of treating an AXL-related disease according to the first aspect; use of an AXL inhibitor and a chemotherapeutic agent in the manufacture of a medicament for treating a disorder in a subject, wherein the treatment comprises a method of treating an AXL-related disease according to the first aspect; and, use of an immune checkpoint modulator (ICM) and a chemotherapeutic agent in the manufacture of a medicament for treating a disorder in a subject, wherein the treatment comprises a method of treating an AXL-related disease according to the first aspect.

In a fourth aspect, the present disclosure provides: a kit comprising an AXL inhibitor, an immune checkpoint modulator (ICM), and a chemotherapeutic agent, for use in a method of treating an Axl-related disease according to the first aspect; a kit comprising an AXL inhibitor and an immune checkpoint modulator (ICM), for use in a method of treating an Axl-related disease according to the first aspect; a kit comprising an AXL inhibitor and a chemotherapeutic agent, for use in a method of treating an Axl-related disease according to the first aspect; and, a kit comprising an immune checkpoint modulator (ICM) and a chemotherapeutic agent, for use in a method of treating an Axl-related disease according to the first aspect.

In a fifth aspect, the present disclosure provides pharmaceutical composition comprising: an AXL inhibitor, an immune checkpoint modulator (ICM), and/or a chemotherapeutic agent; and, a pharmaceutically acceptable excipient, as well as such compositions for use in a method of treating an Axl-related disease according to the first aspect.

In a sixth aspect, the present disclosure provides methods of selecting a subject to be treated in a method of treating an Axl-related disease according to the first aspect. These include:

A method of selecting a subject for treatment with an AXL inhibitor, wherein a subject is selected for treatment if the subject has been, will be, or is being treated with one or more chemotherapeutic agent and/or one or more immune checkpoint modulator (ICM).

A method of selecting a subject for treatment with one or more immune checkpoint modulator (ICM), wherein a subject is selected for treatment if the subject has been, will be, or is being treated with an AXL inhibitor and/or one or more chemotherapeutic agent.

A method of selecting a subject for treatment with one or more chemotherapeutic agent, wherein a subject is selected for treatment if the subject has been, will be, or is being treated with an AXL inhibitor and/or one or more immune checkpoint modulator (ICM).

A method of selecting a subject for treatment with an AXL inhibitor and one or more immune checkpoint modulator (ICM), wherein a subject is selected for treatment if the subject has been, will be, or is being treated with one or more chemotherapeutic agent.

A method of selecting a subject for treatment with an AXL inhibitor and one or more chemotherapeutic agent, wherein a subject is selected for treatment if the subject has been, will be, or is being treated with one or more immune checkpoint modulator (ICM).

A method of selecting a subject for treatment with one or more immune checkpoint modulator (ICM) and one or more chemotherapeutic agent, wherein a subject is selected for treatment if the subject has been, will be, or is being treated with an AXL inhibitor.

In these methods of the sixth aspect, a subject may be selected for treatment if the subject is refractory, non-responsive, or otherwise does not benefit from the recited treatments. For example, a subject may be selected for treatment if the subject is refractory, non-responsive, or otherwise does not benefit from treatment with one or more immune checkpoint modulator (ICM).

These methods of the sixth aspect of the disclosure may further comprise administering to the subject a therapeutically effective amount of an AXL inhibitor, an immune checkpoint modulator (ICM), and/or a chemotherapeutic agent as appropriate.

The disclosure includes the combination of the aspects and preferred features described except where such a combination is clearly impermissible or expressly avoided.

### SUMMARY OF THE FIGURES

FIG. 1. Timeline of the Example 1 combination therapy study. 2 randomizations were performed: the first on day 11 and the second on day 16. The mechanistic study was stopped on day 19, the same day of immune checkpoint modulator (anti-CTLA4/anti-PD1 checkpoint inhibitor; CPI) treatment initiation. AXL inhibitor treatment (bemcentinib) was stopped after 105 post implantation. Chemotherapeutic agent treatment is with doxorubicin.

FIG. 2. Weight of mice in different groups in the Example 1 combination therapy study. The mice were weighed at the same time as tumor volume was measured. 141 animals from all groups are represented in FIG. 2A. The 4 control groups (41 animals) are represented in different dark blue (FIG. 2A, 2B). All CPI treated groups are represented in FIGS. 2C, 2D, 2E, 2F, 2G. 20 animals for CPI (FIG. 2D, light blue), 22 animals for CPI+doxorubicin (FIG. 2E, red) 20 animals for CPI+Bemcentinib (FIG. 2F, green) and 22 animals for the triple combination treatment (FIG. 2G, brown). FIG. 2A represents all groups during the whole study. FIGS. 2B and 2C represent the weight during the 50 first days of the study for mice not treated with CPI (B) and mice treated with CPI (C). FIGS. 2D-2G represent the weight of animals treated with CPI alone (D), CPI+Dox (E), CPI+Bern (F) and CPI+Bern+Dox (G) during the whole study.

FIG. 3. Tumor growth curves for the Example 1 combination therapy study. The 4 control groups (41 animals) are represented in different dark blue (FIG. 3A, 3B). All CPI treated groups are represented in FIGS. 3C, 3D, 3E, 3F, 3G. 20 animals for CPI (FIG. 3D, light blue), 22 animals for CPI+doxorubicin (FIG. 3E, red) 20 animals for CPI+Bemcentinib (FIG. 3F, green) and 22 animals for the triple combination treatment (FIG. 3G, brown). Tumor volume was measured with a digital caliper.

FIG. 4. Tumor growth curves for the Example 1 combination therapy study, focused on the early events, tumors with tumor volume inferior to 300 mm³ during the 25 first days. CPI treated groups are presented in FIG. 4A, Animals not treated with CPI are presented in FIG. 4B. CPI treated groups are represented in FIG. 4C, 4D, 4E, 4F. 20 animals for CPI (FIG. 4C, light blue), 22 animals for CPI+doxorubicin (FIG. 4D, red) 20 animals for CPI+Bemcentinib (FIG. 4E green) and 22 animals for the triple combination treatment (FIG. 4F, brown). Tumor volume was measured with a digital caliper.

FIG. 5. Kaplan Meier Survival curves (500 mm³) for the Example 1 combination therapy study. Age of mice (days) when the tumor display a volume of 500 mm³ or when the mice are sacrificed due to severe symptoms. FIG. 5 shows data for 11 control animals (dark blue), 10 animals treated with Bemcentinib only (purple), 10 animals treated with Dox only (pink) animals treated with Bern and Dox (light purple) animals treated with CPI alone (light blue), 22 animals for CPI+doxorubicin (red), 20 animals for CPI+Bemcentinib (green) and 22 animals for the triple combination treatment (brown). Median survival (in days) when tumors display 500 mm³ volumes is indicated.

FIG. 6. Kaplan Meier Survival curves (1000 mm³) for the Example 1 combination therapy study. Age of mice (days) when the tumor display a volume of 1000 mm³ or when the mice are sacrificed due to severe symptoms. FIG. 6 shows data for 11 control animals (dark blue), 10 animals treated with Bemcentinib only (purple), 10 animals treated with Dox only (pink) animals treated with Bern and Dox (light purple) animals treated with CPI alone (light blue), 22 animals for CPI+doxorubicin (red), 20 animals for CPI+Bemcentinib (green) and 22 animals for the triple combination treatment (brown). Median survival (in days) when tumors display 1000 mm³ volume is indicated.

FIG. 7. 7A: Table with animals displaying an initial response to the treatment in the Example 1 combination study. The initial response is defined as a decrease of 80% of the tumor volume. The numbers of animals displaying the response as well as the percentage of initial responders are presented. 7B: Growth curves of the initial responders shown in 7A. Only CPI treated animals are showed because no responders were found in animals without CPI treatments. 1 initial responder was found in CPI alone group (light blue), 6 initial responders in the Dox+CPI group (red), 5 in the CPI+Bern group (green), and 11 in the CPI+Bern+Dox group (brown).

FIG. 8. 8A: Table presenting Long term responders, no relapse after 150 days in the Example 1 combination study. 7B: Only CPI treated animals are showed because no responders were found in animals without CPI treatments. 1 responder in the Dox+CPI group (red), 2 in the CPI+Bern group (green), and 4 in the CPI+Bern+Dox group (brown).

FIG. 9. Heatmaps showing gene expression changes of 84 Type I IFN related genes in the Example 1 mechanistic study. Scale bar shows range of regulation with upregulated genes colored red and downregulated genes colored green, no changes in black. 9A: Average of all animals from each group. 9B: Fold change of one animal per group compared to one of the 4 controls

FIG. 10. Timeline of the Example 2 mechanistic study. 11 days post implantations, cages were split in 2 groups—vehicle or bemcentinib treated. In each group of cages, a randomization was performed on day 15 post implantation. Doxorubicin (1, 3, or 6 mg/kg) or DXMAA (18 mg/kg) was given i.t. on day 15. The mechanistic study was stopped on day 16, 17, and 18 (for 24, 48, and 72 hour timepoints respectively).

FIG. 11. Timeline of the Example 3 combination study in a mouse model of oncogene-driven, low tumor burden cancer (Yumm1.7 melanoma).

FIG. 12. Body weight changes of C57Bl6 mice carrying Yumm1.7 tumors in the Example 3 combination study and treated as indicated in Table 12. A,B) Body weight changes (%) post implantation for each individual mouse. during the 45 first days of the experiment. C-F) Body weight changes (%) for each individual mouse post ICB treatment. A) All groups, before treatment, 0-20 days post implantation. B) All groups for the whole experiment, 0-55 days post implantation. C. Data for all groups, post ICB treatment until end of experiment 35 days post implantation. D. All groups except Dox bern and Dox bern ICB, 0-13 days (corresponding to the whole experiment for these groups) post ICB treatment. E) All groups post ICB treatment 0-20 days. F) Dox bern and dox bern ICB groups for the whole experiment post ICB treatment, 0-35 days.

FIG. 13. Tumor volume growth in the Example 3 combination study, for each individual tumor in the indicated treatment groups treated as outlined in Table 12 over the course of 55 days. N=5-7. A. All groups. B. All groups except Dox bern and Dox bern ICB. C. ICB, Dox bern, and dox bern ICB groups. D. Dox bern and Dox bern ICB groups.

FIG. 14. Tumor growth changes following administration of immune check point inhibitors in the Example 3 combination study. Data are given for each individual tumor as percentage tumor reduction (negative value) or growth (positive value) compared to tumor size at time of treatment initiation with CPI.

FIG. 15. A. Transformed survival curves of mice from all groups in the Example 3 combination study. Endpoints for survival were set to days of reaching tumor volume of 500 mm³. B. Median survival from transformed survival curve based on days until tumors display 500 mm³ volumes.

FIG. 16. Transformed survival curves of mice from all groups in the Example 3 combination study. A. Endpoints for survival were set to days of reaching tumor volume of 1000 mm³. B. Median survival from transformed survival curve based on days until tumors display 1000 mm³ volumes.

FIG. 17. Average weight of tumors and spleens of the treated groups in the Example 3 combination study, on the day of sacrifice. A. tumor weight. B. Spleen weight.

### DETAILED DESCRIPTION

Aspects and embodiments of the present disclosure will now be discussed with reference to the accompanying figures. Further aspects and embodiments will be apparent to those skilled in the art. All documents mentioned in this text are incorporated herein by reference.

The present disclosure pertains to a combination therapy for treating patients suffering from a proliferative disease, and more particularly to combination therapies comprising an AXL inhibitor, an immune checkpoint modulator (ICM), and a chemotherapeutic agent for treating patients suffering from cancer, as well as methods of treating patients with said combination therapy.

The combination therapies disclosed herein include: combination therapies comprising an AXL inhibitor, an immune checkpoint modulator (ICM), and a chemotherapeutic agent; combination therapies comprising an AXL inhibitor, an immune checkpoint modulator (ICM), and radiotherapy; and, combination therapies comprising an AXL inhibitor, an immune checkpoint modulator (ICM), a chemotherapeutic agent, and radiotherapy.

Because AXL is frequently overexpressed in many tumor types and is known to serve as a key checkpoint for interferon (IFN) signalling, the present authors sought to determine whether suppressing AXL function in these tumors might stimulate IFN signaling and thus produce an enhanced anticancer T cell response in the cells. By investigating the efficacy of a triple combination therapy including AXL inhibition (with bemcentinib), immune checkpoint blockade (with anti-CTLA4/anti-PD1) and cytotoxic chemotherapy (with the anthracycline doxorubicin) in the 4T1 syngeneic mammary carcinoma model and Yumm 1.7 syngeneic melanoma model, the present authors have discovered that such combination therapies are able to significantly delay tumor growth, increase mouse survival, and increase the number of long term responder animals as compared to individual and sub-combination treatments. Without wishing to be bound by theory, the authors believe that the mechanistic contribution of the chemotherapeutic agent is to induce cell death (apoptosis) and release of tumour antigens, upregulating IFN release and leading to a release of type I IFNs, which in turn activate AXL. Accordingly, it is expected that radiotherapy—either in place of, or in combination with, the chemotherapeutic agent—will have the same mechanistic contribution to efficacy of the disclosed combination therapies.

Accordingly, the present disclosure provides a method of treating an AXL-related disease, the method comprising administering to a subject in need thereof a therapeutically effective amount of an AXL inhibitor. In the disclosed methods of treating an AXL-related disease, the AXL inhibitor is administered in combination with: an immune checkpoint modulator (ICM); and, a chemotherapeutic agent and/or radiotherapy. As used herein, “administration in combination” may mean concurrent administration or may mean separate and/or sequential administration in any order.

The present disclosure also provides an AXL inhibitor, an immune checkpoint modulator (ICM), and/or a chemotherapeutic agent for use in a method of treating an AXL-related disease, as well as the use of an AXL inhibitor, an immune checkpoint modulator (ICM), and/or a chemotherapeutic agent in the manufacture of a medicament for treating a disorder in a subject, wherein the treatment comprises a method of treating an AXL-related disease. The present disclosure also provides an AXL inhibitor, an immune checkpoint modulator (ICM), a chemotherapeutic agent, and/or radiotherapy, for use in a method of treating an AXL-related disease, as well as the use of an AXL inhibitor, an immune checkpoint modulator (ICM), a chemotherapeutic agent, and/or radiotherapy in the manufacture of a medicament for treating a disorder in a subject, wherein the treatment comprises a method of treating an AXL-related disease as disclosed herein.

The present disclosure also provides methods of selecting a subject for treatment with one or more of an AXL inhibitor, an immune checkpoint modulator (ICM), and a chemotherapeutic agent, and pharmaceutical compositions comprising an AXL inhibitor, an immune checkpoint modulator (ICM), and/or a chemotherapeutic agent, and, a pharmaceutically acceptable excipient.

AXL Inhibitors (AXLi)

Small Molecule AXL Inhibitors

General Formula

In some embodiments the AXL inhibitor is a compound of formula (I):

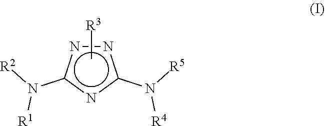

wherein:
R¹, R⁴ and R⁵ are each independently selected from the group consisting of hydrogen, alkyl, alkenyl, aryl, aralkyl, —C(O)R⁸, —C(O)N(R⁶)R⁷, and —C(═NR⁶)N(R⁶)R⁷;
R² and R³ are each independently a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)O R⁸, —R⁹—O—R¹⁰—C(O)N(R⁵)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹) N(R¹¹)H, —R⁹—OC(O)—R⁸, —R⁹—N(R⁶)R⁷, —R⁹—C(O)R³, —R⁹—C(O)OR⁸, —R⁹—C(O)N(R⁶)R⁷, —R⁹—N(R⁶)C(O)OR⁸, —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2);
or R² is a polycyclic heteroaryl containing more than 14 ring atoms as described above and R³ is selected from the group consisting of aryl and heteroaryl, where the aryl and the heteroaryl are each independently optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkenyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N (R¹²)C(O)R¹², —R¹³—N(R¹²)S(O)ₜR¹² (where t is 1 or 2), —R¹³—S(O)OR¹² (where t is 1 or 2), —R¹³—S(O)ₚR¹² (where p is 0, 1 or 2), and —R¹³—S(O)ₜN(R¹²)₂ (where t is 1 or 2);
or R³ is a polycyclic heteroaryl containing more than 14 ring atoms as described above, and R² is selected from the group consisting of aryl and heteroaryl, where the aryl and the heteroaryl are each independently optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkenyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N (R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2);
each R⁶ and R⁷ is independently selected from the group consisting of hydrogen, alkyl, alkenyl, alkynyl, haloalkyl, haloalkenyl, haloalkynyl, hydroxyalkyl, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkenyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, alkenyl, alkynyl, haloalkyl, haloalkenyl, haloalkynyl, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkenyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, and optionally substituted heteroarylalkynyl;
each R⁹ is independently selected from the group consisting of a direct bond, an optionally substituted straight or branched alkylene chain, an optionally substituted straight or branched alkenylene chain and an optionally substituted straight or branched alkynylene chain;
each R¹⁰ is independently selected from the group consisting of an optionally substituted straight or branched alkylene chain, an optionally substituted straight or branched alkenylene chain and an optionally substituted straight or branched alkynylene chain;
each R¹¹ is independently selected from the group consisting of hydrogen, alkyl, cyano, nitro and —OR⁸;
each R¹² is independently selected from the group consisting of hydrogen, alkyl, alkenyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl, optionally substituted heteroarylalkyl, —R¹⁰—OR⁸, —R¹⁰—C N, —R¹⁰—NO₂, —R¹⁰—N(R⁸)₂, —R¹⁰—C(O)OR⁸ and —R¹⁰—C(O)N(R⁸)₂, or two R^{12's}, together with the common nitrogen to which they are both attached, form an optionally substituted N-heterocyclyl or an optionally substituted N-heteroaryl;
each R¹³ is independently selected from the group consisting of a direct bond, an optionally substituted straight or branched alkylene chain and an optionally substituted straight or branched alkenylene chain; and
each R¹⁴ is independently selected from the group consisting of an optionally substituted straight or branched alkylene chain and an optionally substituted straight or branched alkenylene chain;
as an isolated stereoisomer or mixture thereof or as a tautomer or mixture thereof, or a pharmaceutically acceptable salt or N-oxide thereof.

### Some Embodiments

In some embodiments, the compound of formula (I) is a compound of formula (Ia):

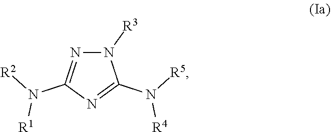

wherein R¹, R², R³, R⁴ and R⁵ are as described above for compounds of formula (I), as an isolated stereoisomer or mixture thereof or as a tautomer or mixture thereof, or a pharmaceutically acceptable salt or N-oxide thereof.

In some embodiments in the compound of formula (Ia) as set forth above, R² and R³ are each independently a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N (R⁶)R⁷, —R⁹—C(O)R⁸, —R⁹—C(O)OR⁸, —R⁹—C(O)N(R⁶)R⁷, —R⁹—N(R⁸)C(O)OR¹², —R⁹—N(R⁶)C(O)R⁸, —R⁹—N(R⁶)S(O)R⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2); and R¹, R⁴, R⁵, each R⁶, each R⁷, each R⁸, each R⁹, each R¹⁰, each R¹¹ and R¹² are as described above for compounds of formula (Ia).

In other embodiments, in the compound of formula (Ia) as set forth above:
R¹, R⁴ and R⁵ are each hydrogen;
each R⁶ and R⁷ is independently selected from the group consisting of hydrogen, alkyl, alkenyl, alkynyl, haloalkyl, hydroxyalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl;
each R⁹ is independently selected from the group consisting of a direct bond and an optionally substituted straight or branched alkylene chain;
each R¹⁰ is an optionally substituted straight or branched alkylene chain; and
each R¹¹ is independently selected from the group consisting of hydrogen, alkyl, cyano, nitro and —OR⁸.

In some embodiments in the compound of formula (Ia) as set forth above:
R² and R³ are each independently a polycyclic heteroaryl containing more than 14 ring atoms selected from the group consisting of 6,7-dihydro-5H-benzo[6,7]cyclohepta[1,2-c]pyridazin-3-yl, 6,7-dihydro-5H-pyrido[2′,3′:6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C (O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR³ (where t is 1 or 2), —R⁹—S(O)ₚR⁸ (where p is 0, 1 or 2), and —R⁹—S(O)ₜN(R⁶)R⁷ (where t is 1 or 2).

In some embodiments in the compound of formula (Ia) is 1-(6,7-dihydro-5H-benzo[6,7]cyclohepta[1,2-c]pyridazin-3-yl)-N³-(5′,5′-dimethyl-6,8,9,10-tetrahydro-5H-spiro[cycloocta[b]pyridine-7,2′-[1,3]dioxane]-3-yl)-1H-1,2,4-triazole-3,5-diamine.

In some embodiments in the compound of formula (Ia) as set forth above, R² is a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜO R⁸ (where t is 1 or 2), —R⁹—S(O)ₚR⁸ (where p is 0, 1 or 2), and —R⁹—S(O)ₜN(R⁶)R⁷ (where t is 1 or 2); R³ is selected from the group consisting of aryl and heteroaryl, where the aryl and the heteroaryl are each independently optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkenyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O) N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹, —R¹³—N(R¹²) S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and R¹, R⁴, R⁵, each R⁶, each R⁷, each R⁸, each R⁹, each R¹⁰, each R¹¹, each R¹², each R¹³ and each R¹⁴ are as described above for compounds of formula (Ia).

In some embodiments in the compound of formula (Ia) as set forth above:
R¹, R⁴ and R⁵ are each hydrogen;
each R⁶ and R⁷ is independently selected from the group consisting of hydrogen, alkyl, alkenyl, alkynyl, haloalkyl, hydroxyalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —R¹⁰—OR⁸, —R¹⁰—CN, —R¹⁰—NO₂, —R¹⁰—N(R′)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl;
each R⁹ is independently selected from the group consisting of a direct bond and an optionally substituted straight or branched alkylene chain;
each R¹⁰ is an optionally substituted straight or branched alkylene chain;
each R¹¹ is independently selected from the group consisting of hydrogen, alkyl, cyano, nitro and —OR⁸;
each R¹² is independently selected from the group consisting of hydrogen, alkyl, alkenyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R^{12's}, together with the common nitrogen to which they are both attached, form an optionally substituted N-heterocyclyl or an optionally substituted N-heteroaryl;
each R¹³ is independently selected from the group consisting of a direct bond and an optionally substituted straight or branched alkylene chain; and
each R¹⁴ is an optionally substituted straight or branched alkylene chain.

In other embodiments, in the compound of formula (Ia) as set forth above:
R¹, R⁴ and R⁵ are each hydrogen;
R² is a polycyclic heteroaryl containing more than 14 ring atoms selected from the group consisting of 6,7-dihydro-5H-benzo[6,7]cyclohepta[1,2-c]pyridazin-3-yl, 6,7-dihydro-5H-pyrido[2′,3′:6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR³, —R⁹—OC(O)—R⁸, —R⁹—N(R⁶)R⁷, —R⁹—C(O)R⁸, —R⁹—C(O)OR⁸, —R⁹—C (O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2);
each R⁶ and R⁷ is independently selected from the group consisting of hydrogen, alkyl, alkenyl, alkynyl, haloalkyl, hydroxyalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl;
each R⁹ is independently selected from the group consisting of a direct bond and an optionally substituted straight or branched alkylene chain;
each R¹⁰ is an optionally substituted straight or branched alkylene chain;
each R¹² is independently selected from the group consisting of hydrogen, alkyl, alkenyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R^{12's}, together with the common nitrogen to which they are both attached, form an optionally substituted N-heterocyclyl or an optionally substituted N-heteroaryl;
each R¹³ is independently selected from the group consisting of a direct bond and an optionally substituted straight or branched alkylene chain; and each R¹⁴ is an optionally substituted straight or branched alkylene chain.

In some embodiments in the compound of formula (Ia) as set forth above:
R² is a polycyclic heteroaryl containing more than 14 ring atoms selected from the group consisting of 6,7-dihydro-5H-benzo[6,7]cyclohepta[1,2-c]pyridazin-3-yl, 6,7-dihydro-5H-pyrido[2′,3′:6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C (O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2); and
R³ is heteroaryl selected from the group consisting of pyridinyl, pyrimidinyl, 4,5-dihydro-1H-benzo[b]azepin-2(3H)-on-8-yl, benzo[d]imidazolyl, 6,7,8,9-tetrahydro-5H-pyrido[3,2-d]azepin-3-yl, 6,7,8,9-tetrahydro-5H-pyrido[3,2-c]azepin-3-yl, 5,6,7,8-tetrahydro-1,6-naphthyridin-3-yl, 5,6,7,8-tetrahydroquinolin-3-yl, 1,2,3,4-tetrahydroisoquinolin-7-yl, 2,3,4,5-tetrahydrobenzo[b]oxepin-7-yl, 3,4-dihydro-2H-benzo[b][1,4]dioxepin-7-yl, benzo[d]oxazol-5-yl, 3,4-dihydro-2H-benzo[b][1,4]oxazin-7-yl, benzo[b]thiophenyl, thieno[3,2-d]pyrimidinyl and 6,7,8,9-tetrahydro-5H-cyclohepta[b]pyridin-3-yl, each optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl, optionally substituted heteroarylalkenyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²) —R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜ N(R¹²)₂ (where t is 1 or 2).

In some embodiments, the compound of formula (Ia), as set forth above, is selected from the group consisting of:
1-(6,7-dimethoxy-quinazolin-4-yl)-N³-(5,7,8,9-tetrahydrospiro[cyclohepta[b]pyridine-6,2′-[1,3]dioxolane]-3-yl)-1H-1,2,4-triazole-3,5-diamine;
1-(2-chloro-7-methylthieno[3,2-d]pyrimidin-4-yl)-N³-(5,7,8,9-tetrahydrospiro[cyclohepta[b]pyridine-6,2′-[1,3]dioxolane]-3-yl)-1H-1,2,4-triazole-3,5-diamine;
1-(2-chloro-7-methylthieno[3,2-d]pyrimidin-4-yl)-N³-(5,6,8,9-tetrahydrospiro[cyclohepta[b]pyridine-7,2′-[1,3]dioxolane]-3-yl)-1H-1,2,4-triazole-3,5-diamine; and
1-(2-chloro-7-methylthieno[3,2-d]pyrimidin-4-yl)-N³-(5′,5′-dimethyl-6,8,9,10-9tetrahydro-5H-spiro[cycloocta[b]pyridine-7,2′-[1,3]dioxane]-3-yl)-1H-1,2,4-triazole-3,5-diamine.

In some embodiments in the compound of formula (Ia) as set forth above, R² is selected from the group consisting of aryl and heteroaryl, where the aryl and the heteroaryl are each independently optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkenyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²) —R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜO R¹² (where t is 1 or 2), —R¹³—S(O)ₚR¹² (where p is 0, 1 or 2), and —R¹³—S(O)ₜN(R¹²)₂ (where t is 1 or 2); R³ is a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N (R⁶)R⁷, —R⁹—C(O)R⁸, —R⁹—C(O)OR⁸, —R⁹—C(O)N(R⁶)R⁷, —R⁹—N(R⁵)C(O)OR¹², —R⁹—N(R⁶)C(O)R⁸, —R⁹—N(R⁶)S(O)ₜR⁸ (where t is 1 or 2), —R⁹—S(O)ₜOR¹² (where t is 1 or 2), —R⁹—S(O)ₚR⁸ (where p is 0, 1 or 2), and —R⁹—S(O)ₜN (R⁶)R⁷ (where t is 1 or 2); and R¹, R⁴, R⁵, each R⁶, each R⁷, each R⁸, each R⁹, each R¹⁰, each R¹¹, each R¹², each R¹³ and each R¹⁴ are as described above for compounds of formula (I).

In some embodiments in the compound of formula (Ia) as set forth above:
R¹, R⁴ and R⁵ are each independently hydrogen;
each R⁶ and R⁷ is independently selected from the group consisting of hydrogen, alkyl, alkenyl, alkynyl, haloalkyl, hydroxyalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, alkenyl, alkynyl, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl;
each R⁹ is independently selected from the group consisting of a direct bond and an optionally substituted straight or branched alkylene chain;
each R¹⁰ is an optionally substituted straight or branched alkylene chain;
each R¹¹ is independently selected from the group consisting of hydrogen, alkyl, cyano, nitro and —OR⁸;
each R¹² is independently selected from the group consisting of hydrogen, alkyl, alkenyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl, optionally substituted heteroarylalkyl, —R¹⁰—OR⁸, —R¹⁰—C N, —R¹⁰—NO₂, —R¹⁰—N(R³)₂, —R¹⁰—C(O)OR⁸ and —R¹⁰—C(O)N(R⁸)₂, or two R^{12's}, together with the common nitrogen to which they are both attached, form an optionally substituted N-heterocyclyl or an optionally substituted N-heteroaryl;
each R¹³ is independently selected from the group consisting of a direct bond and an optionally substituted straight or branched alkylene chain; and
each R¹⁴ is an optionally substituted straight or branched alkylene chain.

In some embodiments in the compound of formula (Ia) as set forth above:
R² is aryl optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkenyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O) N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²) S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2).

In some embodiments in the compound of formula (Ia) as set forth above:
R² is aryl selected from the group consisting of phenyl and 6,7,8,9-tetrahydro-5H-benzo[7]annulene-2-yl, each optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkenyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O) N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²) S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹)₂ (where t is 1 or 2); and
R³ is a polycyclic heteroaryl containing more than 14 ring atoms selected from the group consisting of 6,7-dihydro-5H-benzo[6,7]cyclohepta[1,2-c]pyridazin-3-yl, 6,7-dihydro-5H-pyrido[2′,3′:6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C (O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments in the compound of formula (Ia) as set forth above:
R² is phenyl optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl, optionally substituted heteroarylalkenyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²) —R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜO R¹² (where t is 1 or 2), —R¹³—S(O)ₚR¹² (where p is 0, 1 or 2), and —R¹³—S(O)ₜN(R¹²)₂ (where t is 1 or 2).

In some embodiments in the compound of formula (Ia) as set forth above:
R² is phenyl optionally substituted by one or more substitutents selected from the group consisting of alkyl, halo, haloalkyl, cyano, and optionally substituted heterocyclyl where the optionally substituted heterocyclyl is selected from the group consisting of piperidinyl, piperazinyl, pyrrolidinyl, azepanyl, decahydropyrazino[1,2-a]azepinyl, octahydropyrrolo[3,4-c]pyrrolyl, azabicyclo[3.2.1]octyl, octahydropyrrolo[3,4-b]pyrrolyl, octahydropyrrolo[3,2-c]pyridinyl, 2,7-diazaspiro[4.4]nonanyl and azetidinyl; each independently optionally substituted by one or two substituents selected from the group consisting of —R⁹—OR⁸, —R⁹—N(R⁶)R⁷, —R⁹—C(O)OR⁶, —R⁹—C(O)N(R⁶)R⁷, —R⁹—N(R⁶)C(O)R⁷, —R⁹—N(R⁶)C(O)OR⁷, alkyl, halo, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl;
R³ is selected from the group consisting of 6,7-dihydro-5H-benzo[6,7]cyclohepta[1,2-c]pyridazin-3-yl, 6,7-dihydro-5H-pyrido[2′,3′:6,7]cyclohepta[1,2-c]pyridazin-3-yl, 6,7-dihydro-5H-benzo[6,7]cyclohepta[1,2-b]pyridin-2-yl, 6,7-dihydro-5H-benzo[2,3]thiepino[4,5-c]pyridazin-3-yl, spiro[chromeno[4,3-c]pyridazine-5,1′-cyclopentane]-3-yl and 6,7-dihydro-5H-benzo[6,7]cyclohepta[4,5-c]pyridazin-3-yl, each optionally substituted by one or more substituents selected from the group consisting of alkyl, aryl, halo and —R⁹—OR⁸.

In some embodiments the compound of formula (Ia), as set forth above, is selected from the group consisting of:
N³-(4-(4-cyclohexanylpiperazin-1-yl)phenyl)-1-(6,7-dihydro-5H-benzo[6,7]cyclohepta[1,2-c]pyridazin-3-yl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(pyrrolidin-1-yl)piperidin-1-yl)-1H-1,2,4-triazole-3,5-diamine;
1-(6,7-dihydro-5H-benzo[6,7]cyclohepta[1,2-c]pyridazin-3-yl)-N³-(3-fluoro-4-(4-methyl-3-phenylpiperazin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-(4-(4-piperidin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(indolin-2-on-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(morpholin-4-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(4-(4-cyclopentyl-2-methylpiperazin-1-yl)phenyl)-1H-1,2,4-triazole-3,5-diamine;
1-(6,7-dihydro-5H-benzo[6,7]cyclohepta[1,2-c]pyridazin-3-yl)-N³-(4-(3,5-dimethylpiperazin-1-yl)phenyl)-1H-1,2,4-triazole-3,5-diamine;
1-(6,7-dihydro-5H-benzo[6,7]cyclohepta[1,2-c]pyridazin-3-yl)-N³-(4-(4-(pyrrolidin-1-yl)piperidin-1-yl)-3-cyanophenyl)-1H-1,2,4-triazole-3,5-diamine;
1-(6,7-dihydro-5H-benzo[6,7]cyclohepta[1,2-c]pyridazin-3-yl)-N³-(3-fluoro-4-(3-(diethylamino)pyrrolidin-1-yl)phenyl)-1H-1,2,4-triazole-3,5-diamine;
1-(6,7-dihydro-5H-benzo[6,7]cyclohepta[1,2-c]pyridazin-3-yl)-N³-(4-(4-(bicyclo[2.2.1]heptan-2-yl)piperazin-1-yl)phenyl)-1H-1,2,4-triazole-3,5-diamine;
1-(6,7-dihydro-5H-benzo[6,7]cyclohepta[1,2-c]pyridazin-3-yl)-N³-(4-(4-methylpiperazin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(diethylamino)piperidin-1-yl)phenyl)-1H-1,2,4-triazole-3,5-diamine;
1-(6,7-dihydro-5H-9-methoxybenzo[6,7]cyclohepta[1,2-c]pyridazin-3-yl)-N³-(3-fluoro-4-(4-(pyrrolidin-1-yl)piperdin-1-yl)phenyl)-1H-1,2,4-triazole-3,5-diamine;
1-(6,7-dihydro-5H-10-fluorobenzo[6,7]cyclohepta[1,2-c]pyridazin-3-yl)-N³-(3-fluoro-4-(4-(pyrrolidin-1-yl)piperdin-1-yl)phenyl)-1H-1,2,4-triazole-3,5-diamine;
1-(6,7-dihydro-5H-10-fluorobenzo[6,7]cyclohepta[1,2-c]pyridazin-3-yl)-N³-(3-fluoro-4-(4-(cyclohexyl)piperazin-1-yl)phenyl)-1H-1,2,4-triazole-3,5-diamine;
1-(6,7-dihydro-5H-9-methoxybenzo[6,7]cyclohepta[1,2-c]pyridazin-3-yl)-N³-(3-fluoro-4-(4-(cyclohexyl)piperazin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(4-methylpiperazin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(4-methylpiperidin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-dimethylaminopiperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-chloro-4-(4-pyrrolidin-1-yl-piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-trifluoromethyl-4-(4-pyrrolidin-1-yl-piperidin-1-yl)phenyl)-1H-1,2,4-triazole-3,5-diamine;
1-(6,7-dihydro-5H-9,10-dimethoxybenzo[6,7]cyclohepta[1,2-c]pyridazin-3-yl)-N³-(3-fluoro-4-(4-pyrrolidin-1-yl-piperidin-1-yl)phenyl)-1H-1,2,4-triazole-3,5-diamine;
1-(6,7-dihydro-5H-9,10,11-trimethoxybenzo[6,7]cyclohepta[1,2-c]pyridazin-3-yl)-N³-(3-fluoro-4-(4-pyrrolidin-1-yl-piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(5-methyloctahydropyrrolo[3,4-c]pyrrolyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(3-pyrrolidin-1-yl-piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(3-pyrrolidin-1-yl-azepan-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-N-methylpiperidin-4-yl-piperidin-1-yl)phenyl)-1H-1,2,4-triazole-3,5-diamine;
1-(6,7-dihydro-5H-benzo[6,7]cyclohepta[4,5-c]pyridazin-2-yl)-N³-(3-fluoro-4-(4-(pyrrolidinyl)piperidinyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(5-propyloctahydropyrrolo[3,4-c]pyrrolyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(decahydropyrazino[1,2-a]azepin-2-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(5-cyclopentyloctahydropyrrolo[3,4-c]pyrrolyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(3-(pyrrolidin-1-yl)-8-azabicyclo[3.2.1]oct-8-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-pyrrolidin-1-yl-azepan-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(4-methylpiperazin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(4-isopropylpiperazin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(1-methyloctahydropyrrolo[3,4-b]pyrrol-5-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(N-methylcyclopentylamino)piperidinyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(dipropylamino)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(1-propyloctahydro-1H-pyrrolo[3,2-c]pyridine-5-yl)phenyl)-1H-1,2,4-triazole-3,5-diamine;
1-(6,7-dihydro-5H-benzo[6,7]cyclohepta[4,5-c]pyridazin-2-yl)-N³-(3-fluoro-4-(4-(N-methylpiperazin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(tert-butyloxycarbonylamino)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-aminopiperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(5-cyclohexyloctahydropyrrolo[3,4-c]pyrrolyl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(4-(methylpiperidin-4-yl)phenyl)-1H-1,2,4-triazole-3,5-diamine;
1-(6,7-dihydro-5H-benzo[6,7]cyclohepta[1,2-c]pyridazin-3-yl)-N³-(4-(4-pyrrolidin-1-ylpiperidinyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-pyrrolidin-1-ylpiperidinyl)phenyl)-1H-1,2,4-triazole-3,5-diamine;
1-(6,7-dihydro-5H-benzo[6,7]cyclohepta[1,2-c]pyridazin-3-yl)-N³-(3-methyl-4-(4-pyrrolidin-1-ylpiperidinyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-cyclopentylpiperazinyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-N-methylpiperidin-4-ylpiperazinyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(7-methyl-2,7-diazaspiro[4.4]nonan-2-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(N-isopropylpiperazin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(3-pyrrolidin-1-ylazetidinyl)phenyl)-1H-1,2,4-triazole-3,5-diamine;
1-(6,7-dihydro-5H-benzo[6,7]cyclohepta[1,2-c]pyridazin-3-yl)-N³-(3-methyl-4-(4-(N-methylpiperazin-4-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-((S)-3-(pyrrolidin-1-ylmethyl)pyrrolidinyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(pyrrolidinylmethyl)piperidinyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-((4aR,8aS)-decahydroisoquinolin-2-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(octahydro-1H-pyrido[1,2-a]pyrazin-2-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-(4-(3-pyrrolidin-1-yl)pyrrolidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(5-methyloctahydropyrrolo[3,4-c]pyrrolyl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(octahydropyrrolo[3,4-c]pyrrolyl)phenyl)-1H-1,2,4-triazole-3,5-diamine;
1-(6,7-dihydro-9-chloro-5H-benzo[6,7]cyclohepta[1,2-c]pyridazin-3-yl)-N³-(3-fluoro-4-(4-pyrrolidin-1-ylpiperidin-1-yl)phenyl)-1H-1,2,4-triazole-3,5-diamine;
1-(6,7-dihydro-9-chloro-5H-benzo[6,7]cyclohepta[1,2-c]pyridazin-3-yl)-N³-(3-fluoro-4-(4-(N-methylpiperazin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(4-iodophenyl)-1H-1,2,4-triazole-3,5-diamine;
1-(spiro[chromeno[4,3-c]pyridazine-5,1′-cyclopentane]-3-yl)-N³-(3-fluoro-4-(4-(4-methylpiperazin-1-yl)piperdin-1-yl)phenyl)-1H-1,2,4-triazole-3,5-diamine;
1-(spiro[chromeno[4,3-c]pyridazine-5,1′-cyclopentane]-3-yl)-N³-(3-fluoro-4-(4-(pyrrolidin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-pyrrolidin-1-ylpiperidin-1-yl)phenyl)-1H-1,2,4-triazole-3,5-diamine;
1-(6,7-dihydro-5H-benzo[6,7]cyclohepta[4,5-c]pyridazin-2-yl)-N³-(3-fluoro-4-(4-pyrrolidin-1-ylpiperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(4-methylpiperazin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(3-(3R)-dimethylaminopyrrolidin-1-yl)phenyl)-1H-1,2,4-triazole-3,5-diamine;
1-(6,7-dihydro-5H-benzo[6,7]cyclohepta[4,5-c]pyridazin-2-yl)-N³-(3-methyl-4-(4-pyrrolidin-1-ylpiperidin-1-yl)phenyl)-1H-1,2,4-triazole-3,5-diamine;
1-(6,7-dihydro-5H-benzo[6,7]cyclohepta[4,5-c]pyridazin-2-yl)-N³-(3-fluoro-4-(4-pyrrolidin-1-ylpiperidin-1-yl)phenyl)-1H-1,2,4-triazole-3,5-diamine;
1-(4-phenyl-6,7-dihydro-5H-benzo[6,7]cyclohepta[1,2-b]pyridin-2-yl)-N³-(3-fluoro-4-(4-cyclohexylpiperazin-1-yl)phenyl)-1H-1,2,4-triazole-3,5-diamine;
1-(4-phenyl-6,7-dihydro-5H-benzo[6,7]cyclohepta[1,2-b]pyridin-2-yl)-N³-(4-(4-methylpiperazin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-methylpiperazin-1-yl)phenyl)-1H-1,2,4-triazole-3,5-diamine;
1-(6,7-dihydro-5H-benzo[6,7]cyclohepta[1,2-c]pyridazin-3-yl)-N³-(4-(1-bicyclo[2.2.1]heptan-2-yl)-piperidin-4-ylphenyl)-1H-1,2,4-triazole-3,5-diamine;
1-(6,7-dihydro-5H-benzo[6,7]cyclohepta[1,2-c]pyridazin-3-yl)-N³-(4-(1-cyclopropylmethylpiperidin-4-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-cyclopropylmethylpiperazin-1-yl)phenyl)-1H-1,2,4-triazole-3,5-diamine;
1-(6,7-dihydro-5H-benzo[2,3]thiepino[4,5-c]pyridazin-3-yl)-N³-(4-(1-bicyclo[2.2.1]heptan-2-yl)-piperidin-4-ylphenyl)-1H-1,2,4-triazole-3,5-diamine;
1-(4-phenyl-6,7-dihydro-5H-benzo[6,7]cyclohepta[1,2-b]pyridin-2-yl)-N³-(3-fluoro-4-(4-pyrrolidin-1-ylpiperidin-1-yl)phenyl)-1H-1,2,4-triazole-3,5-diamine, and
1-(6,7-dihydro-5H-pyrido[2′,3′:6,7]cyclohepta[1,2-c]pyridazin-3-yl)-N³-(3-fluoro-4-(4-(pyrrolidin-1-yl)piperidin-1-yl)phenyl)-1H-1,2,4-triazole-3,5-diamine.

In some embodiments in the compound of formula (Ia) as set forth above:
R² is phenyl optionally substituted by one or more substitutents selected from the group consisting of halo, alkyl, heterocyclylalkenyl, —R¹³—OR¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)N(R¹²)₂, and —R¹³—N(R¹²)C(O)R¹²;
R³ is selected from the group consisting of 6,7-dihydro-5H-benzo[6,7]cyclohepta[1,2-c]pyridazin-3-yl and 6,7-dihydro-5H-benzo[6,7]cyclohepta[4,5-c]pyridazin-3-yl, each optionally substituted by one or more substituents selected from the group consisting of alkyl, aryl, halo and —R⁹—OR⁸.

In some embodiments the compound of formula (Ia), as set forth above, is selected from the group consisting of:
1-(6,7-dihydro-5H-benzo[6,7]cyclohepta[1,2-c]pyridazin-3-yl)-N³-(4-(2-(pyrrolidin-1-yl)ethoxy)phenyl)-1H-1,2,4-triazole-3,5-diamine;
1-(6,7-dihydro-5H-benzo[6,7]cyclohepta[1,2-c]pyridazin-3-yl)-N³-(4-(4-(cyclopentyl)piperazin-1-ylcarbonyl)phenyl)-1H-1,2,4-triazole-3,5-diamine;
1-(6,7-dihydro-5H-benzo[6,7]cyclohepta[1,2-c]pyridazin-3-yl)-N³-(4-((2-pyrrolidin-1-ylethyl)aminocarbonyl)phenyl)-1H-1,2,4-triazole-3,5-diamine;
1-(6,7-dihydro-5H-benzo[6,7]cyclohepta[1,2-c]pyridazin-3-yl)-N³-(4-(2,2,6,6-tetramethylpiperidin-1-yl)ethoxyphenyl)-1H-1,2,4-triazole-3,5-diamine;
1-(6,7-dihydro-5H-benzo[6,7]cyclohepta[1,2-c]pyridazin-3-yl)-N³-(4-((2-(dimethylamino)ethyl)aminocarbonyl)phenyl)-1H-1,2,4-triazole-3,5-diamine;
1-(6,7-dihydro-5H-benzo[6,7]cyclohepta[1,2-c]pyridazin-3-yl)-N³-(4-((2-(methoxy)ethyl)aminocarbonyl)phenyl)-1H-1,2,4-triazole-3,5-diamine;
1-(6,7-dihydro-5H-benzo[6,7]cyclohepta[1,2-c]pyridazin-3-yl)-N³-(4-((2-(pyrrolidin-1-yl)ethyl)aminocarbonyl)phenyl)-1H-1,2,4-triazole-3,5-diamine;
1-(6,7-dihydro-5H-benzo[6,7]cyclohepta[1,2-c]pyridazin-3-yl)-N³-(4-((4-(pyrrolidin-1-yl)piperidin-1-yl)carbonyl)phenyl)-1H-1,2,4-triazole-3,5-diamine;
1-(6,7-dihydro-5H-benzo[6,7]cyclohepta[1,2-c]pyridazin-3-yl)-N³-(3-chloro-4-(2-(pyrrolidin-1-yl)ethoxy)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(2-(pyrrolidin-1-yl)ethoxy)phenyl)-1H-1,2,4-triazole-3,5-diamine;
1-(6,7-dihydro-5H-10-fluorobenzo[6,7]cyclohepta[1,2-c]pyridazin-3-yl)-N³-(3-fluoro-4-(2-(pyrrolidin-1-yl)ethoxy)phenyl)-1H-1,2,4-triazole-3,5-diamine;
1-(6,7-dihydro-5H-9-methoxybenzo[6,7]cyclohepta[1,2-c]pyrdazin-3-yl)-N³-(3-fluoro-4-(2-(pyrrolidin-1-yl)ethoxy)phenyl)-1H-1,2,4-triazole-3,5-diamine;
1-(6,7-dihydro-5H-benzo[6,7]cyclohepta[1,2-c]pyridazin-3-yl)-N³-(4-(2-(N-methylcyclopentylamino)ethoxy)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(N-methylpiperidin-4-yl-N-methylamino)phenyl)-1H-1,2,4-triazole-3,5-diamine;
1-(6,7-dihydro-5H-benzo[6,7]cyclohepta[1,2-c]pyridazin-3-yl)-N³-(4-((N-butyl-N-acetoamino)methyl)phenyl)-1H-1,2,4-triazole-3,5-diamine;
1-(6,7-dihydro-5H-benzo[6,7]cyclohepta[1,2-c]pyridazin-3-yl)-N³-(4-(4-(4-methylpiperazin-1-yl)piperidin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(4-(piperidin-1-yl)piperidin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(piperidin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(pyrrolidin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(3-dimethylaminopyrrolidin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(3-diethylaminopyrrolidin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(4-pyrrolidin-1-ylpiperidin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(4-methylpiperazin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(4-isopropylpiperazin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(4-cyclopentylpiperazin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(morpholin-4-ylprop-1-enyl)phenyl)-1H-1,2,4-triazole-3,5-diamine; and
1-(6,7-dihydro-5H-benzo[6,7]cyclohepta[1,2-c]pyridazin-3-yl)-N³-(4-(1-methylpiperidin-3-yl-oxy)phenyl)-1H-1,2,4-triazole-3,5-diamine.

In some embodiments in the compound of formula (Ia) as set forth above:
R² is phenyl optionally substituted by one or more substitutents selected from the group consisting of alkyl, halo, haloalkyl, cyano, and optionally substituted heterocyclyl where the optionally substituted heterocyclyl is selected from the group consisting of piperidinyl, piperazinyl, pyrrolidinyl, azepanyl, decahydropyrazino[1,2-a]azepinyl, octahydropyrrolo[3,4-c]pyrrolyl, azabicyclo[3.2.1]octyl, octahydropyrrolo[3,4-b]pyrrolyl, octahydropyrrolo[3,2-c]pyridinyl, 2,7-diazaspiro[4.4]nonanyl and azetidinyl; each independently optionally substituted by one or two substituents selected from the group consisting of —R⁹—OR⁸, —R⁹—N(R⁶)R⁷, —R⁹—C(O)OR⁶, —R⁹—C(O)N(R⁶)R⁷, —R⁹—N(R⁶)C(O)R⁷, —R⁹—N(R⁶)C(O)OR⁷, alkyl, halo, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl; and
R³ is selected from the group consisting of 6,7-dihydro-5H-benzo[6,7]cyclohepta[1,2-d]pyrimidin-4-yl and 6,7-dihydro-5H-benzo[6,7]cyclohepta[1,2-d]pyrimidin-2-yl, each optionally substituted by one or more substituents selected from the group consisting of alkyl, aryl, halo and —R⁹—OR⁸.

In some embodiments the compound of formula (Ia), as set forth above, is selected from the group consisting of:
1-(6,7-dihydro-5H-benzo[6,7]cyclohepta[1,2-d]pyrimidin-4-yl)-N³-(4-(4-(bicyclo[2.2.1]heptan-2-yl)piperazin-1-yl)phenyl)-1H-1,2,4-triazole-3,5-diamine;
1-(6,7-dihydro-5H-benzo[6,7]cyclohepta[1,2-d]pyrimidin-4-yl)-N³-(3-fluoro-4-(4-(diethylamino)piperidin-1-yl)phenyl)-1H-1,2,4-triazole-3,5-diamine;
1-(6,7-dihydro-5H-benzo[6,7]cyclohepta[1,2-d]pyrimidin-2-yl)-N³-(4-(N-methylpiperazin-1-yl)phenyl)-1H-1,2,4-triazole-3,5-diamine;
1-(6,7-dihydro-5H-benzo[6,7]cyclohepta[1,2-d]pyrimidin-2-yl)-N³-(3-fluoro-4-(4-cyclohexylpiperazinyl)phenyl)-1H-1,2,4-triazole-3,5-diamine; and
1-(6,7-dihydro-5H-benzo[6,7]cyclohepta[1,2-d]pyrimidin-2-yl)-N³-(4-(4-(2S)-bicyclo[2.2.1]heptan-2-yl)-piperazinylphenyl)-1H-1,2,4-triazole-3,5-diamine.

In some embodiments in the compound of formula (Ia) as set forth above:
R² is phenyl optionally substituted by one or more substitutents selected from the group consisting of halo, alkyl, heterocyclylalkenyl, —R¹³—OR¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)N(R¹²)₂, and —R¹³—N(R¹²)C(O)R¹²; and
R³ is selected from the group consisting of 6,7-dihydro-5H-benzo[6,7]cyclohepta[1,2-d]pyrimidin-4-yl and 6,7-dihydro-5H-benzo[6,7]cyclohepta[1,2-d]pyrimidin-2-yl, each optionally substituted by one or more substituents selected from the group consisting of alkyl, aryl, halo and —R⁹—OR¹.

In some embodiments the compound of formula (Ia), as set forth above, is selected from the group consisting of:
1-(6,7-dihydro-5H-benzo[6,7]cyclohepta[1,2-d]pyrimidin-2-yl)-N³-(3-fluoro-4-(2-(pyrrolidin-1-yl)ethoxy)phenyl)-1H-1,2,4-triazole-3,5-diamine; and
1-(6,7-dihydro-5H-benzo[6,7]cyclohepta[1,2-d]pyrimidin-4-yl)-N³-(4-(2-(pyrrolidin-1-yl)ethoxy)phenyl)-1H-1,2,4-triazole-3,5-diamine.

In some embodiments in the compound of formula (Ia) as set forth above:
R² is phenyl optionally substituted by one or more substitutents selected from the group consisting of alkyl, halo, haloalkyl, cyano, and optionally substituted heterocyclyl where the optionally substituted heterocyclyl is selected from the group consisting of piperidinyl, piperazinyl, pyrrolidinyl, azepanyl, decahydropyrazino[1,2-a]azepinyl, octahydropyrrolo[3,4-c]pyrrolyl, azabicyclo[3.2.1]octyl, octahydropyrrolo[3,4-b]pyrrolyl, octahydropyrrolo[3,2-c]pyridinyl, 2,7-diazaspiro[4.4]nonanyl and azetidinyl; each independently optionally substituted by one or two substituents selected from the group consisting of —R⁹—OR⁸, —R⁹—N(R⁶)R⁷, —R⁹—C(O)OR⁶, —R⁹—C(O)N(R⁶)R⁷, —R⁹—N(R⁶)C(O)R⁷, —R⁹—N(R⁸)C(O)OR⁷, alkyl, halo, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl; and
R³ is selected from the group consisting of 6,7-dihydro-5H-benzo[2,3]azepino[4,5-c]pyridazin-3-yl, (Z)-dibenzo[b,][1,4]thiazepin-11-yl, 6,7-dihydro-5H-benzo[2,3]oxepino[4,5-c]pyridazin-3-yl, and 6,7-dihydro-5H-benzo[2,3]thiepino[4,5-c]pyridazin-3-yl, each optionally substituted by one or more substituents selected from the group consisting of alkyl, aryl, halo and —R⁹—OR⁸.

In some embodiments the compound of formula (Ia), as set forth above, is selected from the group consisting of:
1-(7-methyl-6,7-dihydro-5H-benzo[2,3]azepino[4,5-c]pyridazin-3-yl)-N³-(4-(N-methylpiperazin-1-yl)phenyl)-1H-1,2,4-triazole-3,5-diamine;
1-(7-methyl-6,7-dihydro-5H-benzo[2,3]azepino[4,5-c]pyridazin-3-yl)-N³-(3-fluoro-4-(4-cyclohexylpiperazinyl)phenyl)-1H-1,2,4-triazole-3,5-diamine;
1-((Z)-dibenzo[b,f][1,4]thiazepin-11-yl)-N³-(4-(4-N-methylpiperazinyl)phenyl)-1H-1,2,4-triazole-3,5-diamine;
1-((Z)-dibenzo[b,f][1,4]thiazepin-11-yl)-N³-(3-fluoro-4-(4-diethylaminopiperidin-1-yl)phenyl)-1H-1,2,4-triazole-3,5-diamine;
1-(6,7-dihydro-5H-benzo[2,3]oxepino[4,5-c]pyridazin-3-yl)-N³-(4-(4-pyrrolidin-1-ylpiperidinyl)phenyl)-1H-1,2,4-triazole-3,5-diamine;
1-(6,7-dihydro-5H-benzo[2,3]oxepino[4,5-c]pyridazin-3-yl)-N³-(3-fluoro-4-(4-pyrrolidin-1-ylpiperidinyl)phenyl)-1H-1,2,4-triazole-3,5-diamine;
1-(6,7-dihydro-5H-benzo[2,3]thiepino[4,5-c]pyridazin-3-yl)-N³-(3-fluoro-4-(4-pyrrolidin-1-ylpiperidinyl)phenyl)-1H-1,2,4-triazole-3,5-diamine;
1-(6,7-dihydro-5H-benzo[2,3]thiepino[4,5-c]pyridazin-3-yl)-N³-(4-(4-pyrrolidin-1-ylpiperidinyl)phenyl)-1H-1,2,4-triazole-3,5-diamine;
1-(6,7-dihydro-5H-benzo[2,3]thiepino[4,5-c]pyridazin-3-yl)-N³-(3-fluoro-4-(4-(pyrrolidinylmethyl)piperidinyl)phenyl)-1H-1,2,4-triazole-3,5-diamine;
1-(6,7-dihydro-5H-benzo[2,3]thiepino[4,5-c]pyridazin-3-yl)-N³-(3-fluoro-4-((4aR,8aS)-decahydroisoquinolin-2-yl)phenyl)-1H-1,2,4-triazole-3,5-diamine; and
1-(6,7-dihydro-5H-benzo[2,3]thiepino[4,5-c]pyridazin-3-yl)-N³-(3-fluoro-4-(octahydro-1H-pyrido[1,2-a]pyrazin-2-yl)phenyl)-1H-1,2,4-triazole-3,5-diamine.

In some embodiments in the compound of formula (Ia) as set forth above:
R² is phenyl optionally substituted by one or more substitutents selected from the group consisting of halo, alkyl, heterocyclylalkenyl, —R¹³—OR¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)N(R¹²)₂, and —R¹³—N(R¹²)C(O)R¹²; and
R³ is selected from the group consisting of 6,7-dihydro-5H-benzo[2,3]azepino[4,5-c]pyridazin-3-yl, (Z)-dibenzo[b,f][1,4]thiazepin-11-yl, 6,7-dihydro-5H-benzo[2,3]oxepino[4,5-c]pyridazin-3-yl, and 6,7-dihydro-5H-benzo[2,3]thiepino[4,5-c]pyridazin-3-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁶, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶) C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments the compound of formula (Ia), as set forth above, is selected from the group consisting of:
1-(7-methyl-6,7-dihydro-5H-benzo[2,3]azepino[4,5-c]pyridazin-3-yl)-N³-(3-fluoro-4-(2-(pyrrolidin-1-yl)ethoxy)phenyl)-1H-1,2,4-triazole-3,5-diamine; and
1-((Z)-dibenzo[b,f][1,4]thiazepin-11-yl)-N³-(4-(2-(pyrrolidin-1-yl)ethoxy)phenyl)-1H-1,2,4-triazole-3,5-diamine.

In some embodiments in the compound of formula (Ia) as set forth above:
R² is phenyl optionally substituted by a substitutent selected from the group consisting of optionally substituted heterocyclylalkyl, optionally substituted heteroaryl and optionally substituted heteroarylalkyl;
R³ is selected from the group consisting of 6,7-dihydro-5H-benzo[6,7]cyclohepta[1,2-c]pyridazin-3-yl and 6,7-dihydro-5H-benzo[2,3]thiepino[4,5-c]pyridazin-3-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁵)C(O)OR¹², —R⁹—N(R⁶) C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2);
each R⁶ and R⁷ is independently selected from the group consisting of hydrogen, alkyl, alkenyl, alkynyl, haloalkyl, hydroxyalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, alkenyl, alkynyl, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl;
each R⁹ is independently selected from the group consisting of a direct bond and an optionally substituted straight or branched alkylene chain;
each R¹⁰ is an optionally substituted straight or branched alkylene chain; and R¹² is independently selected from the group consisting of hydrogen, alkyl, haloalkyl, alkenyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl.

In some embodiments the compound of formula (Ia), as set forth above, is selected from the group consisting of:
1-(6,7-dihydro-5H-benzo[6,7]cyclohepta[1,2-c]pyridazin-3-yl)-N³-(4-((4-methylpiperazin-1-yl)methyl)phenyl)-1H-1,2,4-triazole-3,5-diamine;
1-(6,7-dihydro-5H-benzo[6,7]cyclohepta[1,2-c]pyridazin-3-yl)-N³-(4-((5-fluoroindolin-2-on-3-yl)methyl)phenyl)-1H-1,2,4-triazole-3,5-diamine;
1-(6,7-dihydro-5H-benzo[6,7]cyclohepta[1,2-c]pyridazin-3-yl)-N³-(4-((4-pyrrolidin-1-ylpiperidinyl)methyl)phenyl)-1H-1,2,4-triazole-3,5-diamine;
1-(6,7-dihydro-5H-benzo[6,7]cyclohepta[1,2-c]pyridazin-3-yl)-N³-(4-((4-cyclopentylpiperazinyl)methyl)phenyl)-1H-1,2,4-triazole-3,5-diamine;
1-(6,7-dihydro-5H-benzo[6,7]cyclohepta[1,2-c]pyridazin-3-yl)-N³-(4-((4-isopropylpiperazinyl)methyl)phenyl)-1H-1,2,4-triazole-3,5-diamine; and
1-(6,7-dihydro-5H-benzo[2,3]thiepino[4,5-c]pyridazin-3-yl)-N³-(3-fluoro-4-(isoindolin-2-yl)phenyl)-1H-1,2,4-triazole-3,5-diamine.

In some embodiments in the compound of formula (Ia) as set forth above:
R¹, R⁴ and R⁵ are each independently hydrogen;
R² is 6,7,8,9-tetrahydro-5H-benzo[7]annulene-2-yl optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl, optionally substituted heteroarylalkenyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)R¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms selected from the group consisting of 6,7-dihydro-5H-benzo[6,7]cyclohepta[1,2-c]pyridazin-3-yl, 6,7-dihydro-5H-pyrido[2′,3′:6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C (O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2); and each R⁶, each R⁷, each R⁸, each R⁹, each R¹², each R¹³ and each R¹⁴ are as described above for compounds of formula (Ia).

In some embodiments the compound of formula (Ia), as set forth above, is selected from the group consisting of:
1-(6,7-dihydro-5H-benzo[6,7]cyclohepta[1,2-c]pyridazin-3-yl)-N³-(7-(pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((bicyclo[2.2.1]heptan-2-yl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((bicyclo[2.2.1]heptan-2-yl)(methyl)amino) 6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-azetidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-(R)-pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-diethylamino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-cyclopentylamino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-(S)-pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-(2-(S)-methyloxycarbonyl)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-(2-(S)-carboxy)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(8-diethylaminoethyl-9 hydroxy-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(3-(S)-fluoropyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(2-(S)-methylpyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(3-(R)-hydroxypyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(2-(R)-methylpyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(3-(S)-hydroxypyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(3-(R)-fluoropyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-oxo-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-cyclohexylamino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-cyclopropylamino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-hydroxy-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-methylpiperazin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(tetrahydrofuran-2-ylmethyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-cyclobutylamino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(cyclopropylmethyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(2-(diethylamino)ethyl)methylamino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-pyrrolidin-1-ylpiperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(piperidin-1-ylmethyl)piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(2-(dimethylamino)ethyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(carboxymethyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(t-butoxycarbonylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(acetamido)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((2R)-2-(methoxycarbonyl)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4,4-difluoropiperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((methoxycarbonylmethyl)(methyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((2R)-2-(carboxy)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(ethoxycarbonyl)piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(carboxy)piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((carboxymethyl)(methyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(ethoxycarbonylmethyl)piperazin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(carboxymethyl)piperazin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(pyrrolidin-1-yl)-6,7,8,9-tetrahydro-5H-benzo[7]annulene-1-yl)-1H-1,2,4-triazole-3,5-diamine;
1-(6,7-dihydro-5H-benzo[6,7]cyclohepta[1,2-c]pyridazin-3-yl)-N³-((7S)-7-amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7s)-7-(di(cyclopropylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((2-methylpropyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((propyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(dipropylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(diethylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(cyclohexylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(cyclopentylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((1-cyclopentylethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(2-propylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((3,3-dimethylbut-2-yl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((cyclohexylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(cyclohexylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((5-chlorothien-2-yl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((2-carboxyphenyl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((3-bromophenyl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(dimethylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(cyclobutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(3-pentylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((2,2-dimethylpropyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(cyclopentylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((cyclopentylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(bicyclo[2.2.1]hept-2-en-5-ylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((bicyclo[2.2.1]hept-2-en-5-ylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(3-methylbutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(3-methylbutyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(2-ethylbutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(but-2-enylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(butyl(but-2-enyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N⁵-((7S)-7-(t-butoxycarbonylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-amino-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(dimethylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(diethylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(dipropylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(di(cyclopropylmethyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(di(3-methylbutyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(cyclobutylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(cyclohexylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-((methylethyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(cyclopentylamino)-6,7,8,9-tetrahydro-5H-benzo[7]annulene-2-yl)-1H-1,2,4-triazole-3,5-diamine; and
1-(6,7-dihydro-5H-pyrido[2′,3′:6,7]cyclohepta[1,2-c]pyridazin-3-yl)-N³-((7S)-7-(2-butylamino)-6,7,8,9-tetrahydro-5H-benzo[7]annulene-2-yl)-1H-1,2,4-triazole-3,5-diamine.

In some embodiments in the compound of formula (Ia) as set forth above:
R¹, R⁴ and R⁵ are each independently hydrogen;
R² is heteroaryl optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkenyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O) N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²) S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2);
R³ is a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N (R⁶)R⁷, —R⁹—C(O)R⁸, —R⁹—C(O)OR⁸, —R⁹—C(O)N(R⁶)R⁷, —R⁹—N(R⁸)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2); and each R⁶, each R⁷, each R⁸, each R⁹, each R¹², each R¹³ and each R¹⁴ are as described above for compounds of formula (Ia); and each R⁶, each R⁷, each R⁸, each R⁹, each R¹⁰, each R¹¹, each R¹², each R¹³ and each R¹⁴ are as described above for compounds of formula (Ia).

In some embodiments in the compound of formula (Ia) as set forth above:
R² is heteroaryl selected from the group consisting of pyridinyl, pyrimidinyl, 4,5-dihydro-1H-benzo[b]azepin-2(3H)-on-8-yl, benzo[d]imidazolyl, 6,7,8,9-tetrahydro-5H-pyrido[3,2-d]azepin-3-yl, 6,7,8,9-tetrahydro-5H-pyrido[3,2-c]azepin-3-yl, 5,6,7,8-tetrahydro-1,6-naphthyridin-3-yl, 5,6,7,8-tetrahydroquinolin-3-yl, 1,2,3,4-tetrahydroisoquinolin-7-yl, 2,3,4,5-tetrahydrobenzo[b]oxepin-7-yl, 3,4-dihydro-2H-benzo[b][1,4]dioxepin-7-yl, benzo[d]oxazol-5-yl, 3,4-dihydro-2H-benzo[b][1,4]oxazin-7-yl, benzo[b]thiophenyl, and 6,7,8,9-tetrahydro-5H-cyclohepta[b]pyridin-3-yl, each optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl, optionally substituted heteroarylalkenyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms selected from the group consisting of 6,7-dihydro-5H-benzo[6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C (O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2). Another embodiment is the use where, in the compound of formula (Ia) as set forth above: R² is selected from the group consisting of pyridinyl and pyrimidinyl, each optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl, optionally substituted heteroarylalkenyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2).

In some embodiments the compound of formula (Ia), as set forth above, is selected from the group consisting of:
1-(6,7-dihydro-5H-benzo[6,7]cyclohepta[1,2-c]pyridazin-3-yl)-N³-(6-(4-(bicyclo[2.2.1]heptan-2-yl)piperazin-1-yl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(4-cyclopentyl-1,4-diazepan-1-yl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(4-methylpiperazin-1-yl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(4-(4-methylpiperazin-1-yl)piperidin-1-yl)pyridine-3-yl)-1H-1,2,4-triazole-3,5-diamine;
1-(6,7-dihydro-5H-benzo[6,7]cyclohepta[1,2-c]pyridazin-3-yl)-N³-(pyridin-3-yl)-1H-1,2,4-triazole-3,5-diamine;
1-(6,7-dihydro-5H-benzo[6,7]cyclohepta[1,2-c]pyridazin-3-yl)-N³-(6-(6-aminopyridin-3-yl)pyridine-3-yl)-1H-1,2,4-triazole-3,5-diamine;
1-(6,7-dihydro-5H-benzo[6,7]cyclohepta[1,2-c]pyridazin-3-yl)-N³-(6-(3-aminophenyl)pyridine-3-yl)-1H-1,2,4-triazole-3,5-diamine;
1-(6,7-dihydro-5H-benzo[6,7]cyclohepta[1,2-c]pyridazin-3-yl)-N³-(6-(3-cyanophenyl)pyridine-3-yl)-1H-1,2,4-triazole-3,5-diamine;
1-(6,7-dihydro-5H-benzo[6,7]cyclohepta[1,2-c]pyridazin-3-yl)-N³-(6-(benzo[d][1,3]dioxole-6-yl)pyridine-3-yl)-1H-1,2,4-triazole-3,5-diamine;
1-(6,7-dihydro-5H-benzo[6,7]cyclohepta[1,2-c]pyridazin-3-yl)-N³-(6-(3-methylsulfonamidylphenyl)pyridine-3-yl)-1H-1,2,4-triazole-3,5-diamine;
1-(6,7-dihydro-5H-benzo[6,7]cyclohepta[1,2-c]pyridazin-3-yl)-N³-(6-(2-diethylaminomethyl)pyrrolidin-1-ylpyridin-3-yl)-1H-1,2,4-triazole-3,5-diamine;
1-(6,7-dihydro-5H-benzo[6,7]cyclohepta[1,2-c]pyridazin-3-yl)-N³-(6-(3-diethylaminopyrrolidin-1-yl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(3-(4-(N-methylpiperazin-4-yl)piperidin-1-yl)-(E)-propenyl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(4-(pyrrolidin-1-yl)piperidin-1-yl)-5-methylpyridin-3-yl)-1H-1,2,4-triazole-3,5-diamine;
1-(6,7-dihydro-5H-benzo[6,7]cyclohepta[1,2-c]pyridazin-3-yl)-N³-(6-(3-piperidin-1-yl-(E)-propenyl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(4-(bicyclo[2.2.1]heptan-2-yl)-1,4-diazepan-1-yl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(3-(4-(pyrrolidin-1-yl)piperidin-1-yl)-(E)-propenyl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(3-piperidin-1-yl)-propanylpyridin-3-yl)-1H-1,2,4-triazole-3,5-diamine;
1-(6,7-dihydro-5H-benzo[6,7]cyclohepta[1,2-c]pyridazin-3-yl)-N³-(6-(3-(4-(piperidin-1-yl)piperidin-1-yl)-(E)-propenyl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(3-(4-dimethylaminopiperidin-1-yl)-(E)-propenyl)pyridin-3-yl)-1H-1,2,4-triazole-3,5-diamine;
1-(6,7-dihydro-5H-benzo[6,7]cyclohepta[1,2-c]pyridazin-3-yl)-N³-(2-(4-pyrrolidin-1-ylpiperidin-1-yl)pyrimidin-5-yl)-1H-1,2,4-triazole-3,5-diamine;
1-(6,7-dihydro-5H-benzo[6,7]cyclohepta[1,2-c]pyridazin-3-yl)-N³-(2-(4-(piperidin-1-ylmethyl)piperidin-1-yl)pyrimidin-5-yl)-1H-1,2,4-triazole-3,5-diamine;
1-(6,7-dihydro-5H-benzo[6,7]cyclohepta[1,2-c]pyridazin-3-yl)-N³-(6-((4-piperidin-1-ylpiperidin-1-yl)carbonyl)pyridin-3-yl)-1H-1,2,4-triazole-3,5-diamine;
1-(6,7-dihydro-5H-benzo[6,7]cyclohepta[1,2-c]pyridazin-3-yl)-N³-(2-(4-cyclopropylmethylpiperazin-1-yl)pyridine-5-yl)-1H-1,2,4-triazole-3,5-diamine; and
1-(6,7-dihydro-5H-benzo[6,7]cyclohepta[1,2-c]pyridazin-3-yl)-N³-(2-(3-(S)-methyl-4-cyclopropylmethylpiperazin-1-yl)pyridine-5-yl)-1H-1,2,4-triazole-3,5-diamine.

In some embodiments in the compound of formula (Ia) as set forth above:
R¹, R⁴ and R⁵ are each independently hydrogen;
R² is selected from the group consisting of 4,5-dihydro-1H-benzo[b]azepin-2(3H)-on-8-yl, benzo[d]imidazolyl, 6,7,8,9-tetrahydro-5H-pyrido[3,2-d]azepin-3-yl, 6,7,8,9-tetrahydro-5H-pyrido[3,2-c]azepin-3-yl, 5,6,7,8-tetrahydro-1,6-naphthyridin-3-yl, 5,6,7,8-tetrahydroquinolin-3-yl, 1,2,3,4-tetrahydroisoquinolin-7-yl, 2,3,4,5-tetrahydrobenzo[b]oxepin-7-yl, 3,4-dihydro-2H-benzo[b][1,4]dioxepin-7-yl, benzo[d]oxazol-5-yl, 3,4-dihydro-2H-benzo[b][1,4]oxazin-7-yl, benzo[b]thiophenyl, and 6,7,8,9-tetrahydro-5H-cyclohepta[b]pyridin-3-yl, each optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl, optionally substituted heteroarylalkenyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms selected from the group consisting of 6,7-dihydro-5H-benzo[6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C (O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments the compound of formula (Ia), as set forth above, is selected from the group consisting of:
1-(6,7-dihydro-5H-benzo[6,7]cyclohepta[1,2-c]pyridazin-3-yl)-N³-(4,5-dihydro-1H-benzo[b]azepin-2(3H)-on-8-yl)-1H-1,2,4-triazole-3,5-diamine;
1-(6,7-dihydro-5H-benzo[6,7]cyclohepta[1,2-c]pyridazin-3-yl)-N³-(2-(dimethylaminomethyl)-1H-benzo[d]imidazol-5-yl)-1H-1,2,4-triazole-3,5-diamine;
1-(6,7-dihydro-5H-benzo[6,7]cyclohepta[1,2-c]pyridazin-3-yl)-N³-(7-cyclopentyl-6,7,8,9-tetrahydro-5H-pyrido[3,2-d]azepin-3-yl)-1H-1,2,4-triazole-3,5-diamine;
1-(6,7-dihydro-5H-benzo[6,7]cyclohepta[1,2-c]pyridazin-3-yl)-N³-(6-methyl-5,6,7,8-tetrahydro-1,6-naphthyridin-3-yl)-1H-1,2,4-triazole-3,5-diamine;
1-(6,7-dihydro-5H-benzo[6,7]cyclohepta[1,2-c]pyridazin-3-yl)-N³-(6-(4-(4-methylpiperazin-1-yl)piperidin-1-yl)pyridine-3-yl)-1H-1,2,4-triazole-3,5-diamine;
1-(6,7-dihydro-5H-benzo[6,7]cyclohepta[1,2-c]pyridazin-3-yl)-N³-(6-(4-methylpiperazin-1-yl)carbonyl-5,6,7,8-tetrahydroquinolin-3-yl)-1H-1,2,4-triazole-3,5-diamine, compound #31, 1H-1,2,4-triazole-3,5-diamine;
1-(6,7-dihydro-5H-benzo[6,7]cyclohepta[1,2-c]pyridazin-3-yl)-N³-(2,3,4,5-tetrahydrobenzo[b]oxepin-7-yl)-1H-1,2,4-triazole-3,5-diamine;
1-(6,7-dihydro-5H-benzo[6,7]cyclohepta[1,2-c]pyridazin-3-yl)-N³-(3,4-dihydro-2H-benzo[b][1,4]dioxepin-7-yl)-1H-1,2,4-triazole-3,5-diamine;
1-(6,7-dihydro-5H-benzo[2,3]oxepino[4,5-c]pyridazin-3-yl)-N³-(2-(pyrrolidin-1-ylmethyl)benzo[d]oxazol-5-yl)-1H-1,2,4-triazole-3,5-diamine;
1-(6,7-dihydro-5H-benzo[6,7]cyclohepta[1,2-c]pyridazin-3-yl)-N³-(4-(2-dimethylaminoethyl)-(3,4-dihydro-2H-benzo[b][1,4]oxazin-7-yl))-1H-1,2,4-triazole-3,5-diamine;
1-(6,7-dihydro-5H-benzo[2,3]oxepino[4,5-c]pyridazin-3-yl)-N³-(4-(2-dimethylaminoethyl)-(3,4-dihydro-2H-benzo[b][1,4]oxazin-7-yl))-1H-1,2,4-triazole-3,5-diamine;
1-(6,7-dihydro-5H-benzo[2,3]thiepino[4,5-c]pyridazin-3-yl)-N³-(2-(1-(4-(2-(dimethylamino)ethyl)piperazin-1-yl)oxomethyl)benzo[b]thiophen-5-yl)-1H-1,2,4-triazole-3,5-diamine;
1-(6,7-dihydro-5H-benzo[6,7]cyclohepta[1,2-c]pyridazin-3-yl)-N³-(6-cyclopentyl-6,7,8,9-tetrahydro-5H-pyrido[3,2-c]azepin-3-yl)-1H-1,2,4-triazole-3,5-diamine;
1-(6,7-dihydro-5H-benzo[6,7]cyclohepta[1,2-c]pyridazin-3-yl)-N³-((7-pyrrolidin-1-yl)-6,7,8,9-tetrahydro-5H-cyclohepta[b]pyridine-3-yl)-1H-1,2,4-triazole-3,5-diamine;
1-(6,7-dihydro-5H-benzo[6,7]cyclohepta[1,2-c]pyridazin-3-yl)-N³ (2-cyclopentyl-1,2,3,4-tetrahydroisoquinolin-7-yl)-1H-1,2,4-triazole-3,5-diamine;
1-(6,7-dihydro-5H-benzo[6,7]cyclohepta[1,2-c]pyridazin-3-yl)-N³-(6-(pyrrolidin-1-yl)-5,6,7,8-tetrahydroquinolin-3-yl)-1H-1,2,4-triazole-3,5-diamine;
1-(6,7-dihydro-5H-benzo[6,7]cyclohepta[1,2-c]pyridazin-3-yl)-N³-(6-cyclopentyl-5,6,7,8-tetrahydro-1,6-naphthyridine-3-yl)-1H-1,2,4-triazole-3,5-diamine;
1-(6,7-dihydro-5H-benzo[6,7]cyclohepta[1,2-c]pyridazin-3-yl)-N³-((S)-7-(pyrrolidin-1-yl)-6,7,8,9-tetrahydro-5H-cyclohepta[b]pyridine-3-yl)-1H-1,2,4-triazole-3,5-diamine;
1-(6,7-dihydro-5H-benzo[6,7]cyclohepta[1,2-c]pyridazin-3-yl)-N³-(1,2,3,4-tetrahydroisoquinolin-7-yl)-1H-1,2,4-triazole-3,5-diamine;
1-(6,7-dihydro-5H-benzo[6,7]cyclohepta[1,2-c]pyridazin-3-yl)-N³-(2-(1-methylpiperidin-4-yl)-1,2,3,4-tetrahydroisoquinolin-7-yl)-1H-1,2,4-triazole-3,5-diamine; and
1-(6,7-dihydro-5H-benzo[6,7]cyclohepta[1,2-c]pyridazin-3-yl)-N³-(2-(cyclopropylmethyl)-1,2,3,4-tetrahydroisoquinolin-7-yl)-1H-1,2,4-triazole-3,5-diamine.

In some embodiments the compound of formula (Ia), as set forth above, is a compound of formula (Ia1):

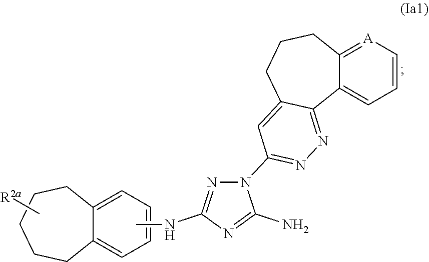

wherein:
A is ═C(H)— or ═N—;
each R^{2a} is independently selected from the group consisting of —N(R^{12a})₂ and —N(R^{12a})C(O)R^{12a},
or R^{2a} is an N-heterocyclyl optionally substituted by one or more substituents selected from the group consisting of halo and —R²¹—C(O)OR²⁰,
each R^{12a} is independently selected from the group consisting of hydrogen, alkyl, alkenyl, optionally substituted aralkyl, optionally substituted cycloalkyl, optionally substituted cycloalkylalkyl, optionally substituted heteroaryl and optionally substituted heteroarylalkyl;
R²⁰ is independently selected from the group consisting of hydrogen, alkyl, alkenyl, optionally substituted aralkyl, optionally substituted cycloalkyl, optionally substituted cycloalkylalkyl, optionally substituted heteroaryl and optionally substituted heteroarylalkyl; and
R²¹ is independently selected from the group consisting of a direct bond or an optionally substituted straight or branched alkylene chain;
as an isolated stereoisomer or mixture thereof, or a pharmaceutically acceptable salt thereof.

In some embodiments the compound of formula (I) is a compound of formula (Ib):

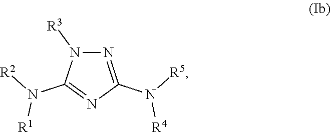

wherein R¹, R², R³, R⁴ and R⁵ are as described above for compounds of formula (I), as an isolated stereoisomer or mixture thereof or as a tautomer or mixture thereof, or a pharmaceutically acceptable salt or N-oxide thereof.

In some embodiments in the compound of formula (Ib) as set forth above, R² and R³ are each independently a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N (R⁶)R⁷, —R⁹—C(O)R⁸, —R⁹—C(O)OR⁸, —R⁹—C(O)N(R⁶)R⁷, —R⁹—N(R⁵)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2); and R¹, R⁴, R⁵, each R⁶, each R⁷, each R⁸, each R⁹, each R¹⁰, each R¹¹ and R¹² are as described above in relation to formula (I).

In some embodiments in the compound of formula (Ib) as set forth above:
R¹, R⁴ and R⁵ are each hydrogen;
each R⁶ and R⁷ is independently selected from the group consisting of hydrogen, alkyl, alkenyl, alkynyl, haloalkyl, hydroxyalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl;
each R⁹ is independently selected from the group consisting of a direct bond and an optionally substituted straight or branched alkylene chain;
each R¹⁰ is an optionally substituted straight or branched alkylene chain; and
each R¹¹ is independently selected from the group consisting of hydrogen, alkyl, cyano, nitro and —OR⁸.

In some embodiments in the compound of formula (Ib) as set forth above:
R² and R³ are each independently a polycyclic heteroaryl containing more than 14 ring atoms selected from the group consisting of 6,7-dihydro-5H-benzo[6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C (O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments the compound of formula (Ib), as set forth above, is 1-(6,7-dihydro-5H-benzo[6,7]cyclohepta[1,2-c]pyridazin-3-yl)-N⁵-(5,7,8,9-tetrahydrospiro[cyclohepta[b]pyridine-6,2′[1,3]dioxolane]-3-yl)-1H-1,2,4-triazole-3,5-diamine.

In some embodiments in the compound of formula (Ib) as set forth above:
R² is selected from the group consisting of aryl and heteroaryl, where the aryl and the heteroaryl are each independently optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkenyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁸)R⁷, —R⁹—O—R¹⁰—C(NR¹¹)N(R¹¹)H, —R⁹—OC(O)—R⁸, —R⁹—N (R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments in the compound of formula (Ib) as set forth above:
R¹, R⁴ and R⁵ are each independently hydrogen;
each R⁶ and R⁷ is independently selected from the group consisting of hydrogen, alkyl, alkenyl, alkynyl, haloalkyl, hydroxyalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, alkenyl, alkynyl, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl; each R⁹ is independently selected from the group consisting of a direct bond and an optionally substituted straight or branched alkylene chain;
each R¹⁰ is an optionally substituted straight or branched alkylene chain;
each R¹¹ is independently selected from the group consisting of hydrogen, alkyl, cyano, nitro and —OR⁸;
each R¹² is independently selected from the group consisting of hydrogen, alkyl, alkenyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R^{12's}, together with the common nitrogen to which they are both attached, form an optionally substituted N-heterocyclyl or an optionally substituted N-heteroaryl;
each R¹³ is independently selected from the group consisting of a direct bond and an optionally substituted straight or branched alkylene chain; and
each R¹⁴ is an optionally substituted straight or branched alkylene chain.

In some embodiments in the compound of formula (Ib) as set forth above:
R² is aryl optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkenyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N (R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments in the compound of formula (Ib) as set forth above:
R¹, R⁴ and R⁵ are each independently hydrogen;
R² is aryl selected from the group consisting of phenyl and 6,7,8,9-tetrahydro-5H-benzo[7]annulene-2-yl, each optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkenyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms selected from the group consisting of 6,7-dihydro-5H-benzo[6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C (O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments in the compound of formula (Ib) as set forth above:
R² is phenyl optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl, optionally substituted heteroarylalkenyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C (O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2).

In some embodiments the compound of formula (Ib), as set forth above, is selected from the group consisting of:
1-(6,7-dihydro-5H-benzo[6,7]cyclohepta[1,2-c]pyridazin-3-yl)-N⁵-(3-fluoro-4-(4-(indolin-2-on-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N⁵-(3-fluoro-4-(4-(morpholin-4-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N⁵-(4-(3,5-dimethylpiperazin-1-yl)phenyl)-1H-1,2,4-triazole-3,5-diamine;
1-(7-methyl-6,7-dihydro-5H-benzo[2,3]azepino[4,5-c]pyridazin-3-yl)-N⁵-(4-(N-methylpiperazin-1-yl)phenyl)-1H-1,2,4-triazole-3,5-diamine;
1-(6,7-dihydro-5H-benzo[6,7]cyclohepta[1,2-c]pyridazin-3-yl)-N⁵-(4-((5-fluoroindolin-2-on-3-yl)methyl)phenyl)-1H-1,2,4-triazole-3,5-diamine;
1-(6,7-dihydro-5H-benzo[6,7]cyclohepta[1,2-c]pyridazin-3-yl)-N⁵-(4-(4-pyrrolidin-1-ylpiperidinyl)phenyl)-1H-1,2,4-triazole-3,5-diamine;
1-(6,7-dihydro-5H-benzo[6,7]cyclohepta[1,2-c]pyridazin-3-yl)-N⁵-(4-((4-pyrrolidin-1-ylpiperidinyl)methyl)phenyl)-1H-1,2,4-triazole-3,5-diamine;
1-(6,7-dihydro-5H-benzo[6,7]cyclohepta[1,2-c]pyridazin-3-yl)-N⁵-(4-((4-cyclopentylpiperazinyl)methyl)phenyl)-1H-1,2,4-triazole-3,5-diamine;
1-(6,7-dihydro-5H-benzo[6,7]cyclohepta[1,2-c]pyridazin-3-yl)-N⁵-(4-((4-isopropylpiperazinyl)methyl)phenyl)-1H-1,2,4-triazole-3,5-diamine;
1-(6,7-dihydro-5H-benzo[6,7]cyclohepta[1,2-c]pyridazin-3-yl)-N⁵-(3-fluoro-4-(4-N-methylpiperid-4-ylpiperazinyl)phenyl)-1H-1,2,4-triazole-3,5-diamine;
1-(6,7-dihydro-5H-benzo[6,7]cyclohepta[1,2-c]pyridazin-3-yl)-N⁵-(3-fluoro-4-(7-methyl-2,7-diazaspiro[4.4]nonan-2-yl)phenyl)-1H-1,2,4-triazole-3,5-diamine;
1-(6,7-dihydro-5H-benzo[6,7]cyclohepta[1,2-c]pyridazin-3-yl)-N⁵-(3-fluoro-4-(3-pyrrolidin-1-ylazetidinyl)phenyl)-1H-1,2,4-triazole-3,5-diamine;
1-(6,7-dihydro-5H-benzo[6,7]cyclohepta[1,2-c]pyridazin-3-yl)-N⁵-(3-methyl-4-(4-(N-methylpiperazin-4-yl)piperidin-1-yl)phenyl)-1H-1,2,4-triazole-3,5-diamine;
1-(6,7-dihydro-5H-benzo[2,3]thiepino[4,5-c]pyridazin-3-yl)-N-(4-(4-pyrrolidin-1-ylpiperidinyl)phenyl)-1H-1,2,4-triazole-3,5-diamine;
1-(6,7-dihydro-5H-benzo[6,7]cyclohepta[1,2-c]pyridazin-3-yl)-N⁵-(3-fluoro-(4-(3-pyrrolidin-1-yl)pyrrolidin-1-yl)phenyl)-1H-1,2,4-triazole-3,5-diamine;
1-(6,7-dihydro-5H-benzo[6,7]cyclohepta[1,2-c]pyridazin-3-yl)-N⁵-(3-fluoro-4-(4-methylpiperazin-1-yl)phenyl)-1H-1,2,4-triazole-3,5-diamine; and
1-(6,7-dihydro-5H-benzo[6,7]cyclohepta[1,2-c]pyridazin-3-yl)-N⁵-(3-fluoro-4-(4-cyclopropylmethylpiperazin-1-yl)phenyl)-1H-1,2,4-triazole-3,5-diamine.

In some embodiments in the compound of formula (Ib) as set forth above:
R¹, R⁴ and R⁵ are each independently hydrogen;
R² is 6,7,8,9-tetrahydro-5H-benzo[7]annulene-2-yl optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl, optionally substituted heteroarylalkenyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²) —R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms selected from the group consisting of 6,7-dihydro-5H-benzo[6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR³, —R⁹—OC(O)—R⁸, —R⁹—N(R⁶)R⁷, —R⁹—C(O)R⁸, —R⁹—C(O)OR⁸, —R⁹—C (O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments the compound of formula (Ib), as set forth above, is selected from the group consisting of:
1-(6,7-dihydro-5H-benzo[6,7]cyclohepta[1,2-c]pyridazin-3-yl)-N⁵-(7-(pyrrolidin-1-yl)-6,7,8,9-tetrahydro-5H-benzo[7]annulene-1-yl)-1H-1,2,4-triazole-3,5-diamine;
1-(6,7-dihydro-5H-benzo[6,7]cyclohepta[1,2-c]pyridazin-3-yl)-N⁵-((7S)-7-(t-butoxycarbonylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N⁵-(7-((bicyclo[2.2.1]heptan-2-yl)(methyl)amino)-6,7,8,9-tetrahydro-5H-benzo[7]annulene-2-yl)-1H-1,2,4-triazole-3,5-diamine; and
1-(6,7-dihydro-5H-benzo[6,7]cyclohepta[1,2-c]pyridazin-3-yl)-N⁵-(7-(S)-pyrrolidin-1-yl-6,7,8,9-tetrahydro-5H-benzo[7]annulene-2-yl)-1H-1,2,4-triazole-3,5-diamine.

In some embodiments in the compound of formula (Ib) as set forth above:
R¹, R⁴ and R⁵ are each independently hydrogen;
R² is heteroaryl optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkenyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C (O)R⁸, —R⁹—N(R⁵)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments in the compound of formula (Ib) as set forth above:
R² is heteroaryl selected from the group consisting of pyridinyl, pyrimidinyl, 4,5-dihydro-1H-benzo[b]azepin-2(3H)-on-8-yl, benzo[d]imidazolyl, 6,7,8,9-tetrahydro-5H-pyrido[3,2-d]azepin-3-yl, 6,7,8,9-tetrahydro-5H-pyrido[3,2-c]azepin-3-yl, 5,6,7,8-tetrahydro-1,6-naphthyridin-3-yl, 5,6,7,8-tetrahydroquinolin-3-yl, 1,2,3,4-tetrahydroisoquinolin-7-yl, 2,3,4,5-tetrahydrobenzo[b]oxepin-7-yl, 3,4-dihydro-2H-benzo[b][1,4]dioxepin-7-yl, benzo[d]oxazol-5-yl, 3,4-dihydro-2H-benzo[b][1,4]oxazin-7-yl, benzo[b]thiophenyl, and 6,7,8,9-tetrahydro-5H-cyclohepta[b]pyridin-3-yl, each optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl, optionally substituted heteroarylalkenyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms selected from the group consisting of 6,7-dihydro-5H-benzo[6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C (O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments the compound of formula (Ib), as set forth above, is selected from the group consisting of:
1-(6,7-dihydro-5H-benzo[6,7]cyclohepta[1,2-c]pyridazin-3-yl)-N⁵-(6-(4-(pyrrolidin-1-yl)piperidin-1-yl)-5-methylpyridin-3-yl)-1H-1,2,4-triazole-3,5-diamine;
1-(6,7-dihydro-5H-benzo[6,7]cyclohepta[1,2-c]pyridazin-3-yl)-N⁵-(4-(3,5-dimethylpiperazin-1-yl)phenyl)-1H-1,2,4-triazole-3,5-diamine;
1-(6,7-dihydro-5H-benzo[6,7]cyclohepta[1,2-c]pyridazin-3-yl)-N⁵-(1,2,3,4-tetrahydroisoquinolin-7-yl)-1H-1,2,4-triazole-3,5-diamine; and
1-(6,7-dihydro-5H-benzo[6,7]cyclohepta[1,2-c]pyridazin-3-yl)-N⁵-(2-(1-methylpiperidin-4-yl)-1,2,3,4-tetrahydroisoquinolin-7-yl)-1H-1,2,4-triazole-3,5-diamine.

In some embodiments the compound of formula (Ib), as set forth above, is a compound of formula (Ib1):

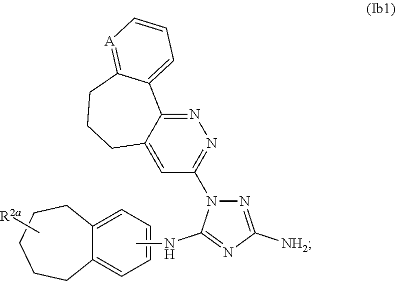

wherein:
A is ═C(H)— or ═N—;
each R^{2a} is independently selected from the group consisting of —N(R^{12a})₂ and —N(R^{12a})C(O)R^{12a},
or R^{2a} is an N-heterocyclyl optionally substituted by one or more substituents selected from the group consisting of halo and —R²¹—C(O)OR²⁰,
each R^{12a} is independently selected from the group consisting of hydrogen, alkyl, alkenyl, optionally substituted aralkyl, optionally substituted cycloalkyl, optionally substituted cycloalkylalkyl, optionally substituted heteroaryl and optionally substituted heteroarylalkyl;
R²⁰ is independently selected from the group consisting of hydrogen, alkyl, alkenyl, optionally substituted aralkyl, optionally substituted cycloalkyl, optionally substituted cycloalkylalkyl, optionally substituted heteroaryl and optionally substituted heteroarylalkyl; and
R²¹ is independently selected from the group consisting of a direct bond or an optionally substituted straight or branched alkylene chain;
as an isolated stereoisomer or mixture thereof, or a pharmaceutically acceptable salt thereof.

### PREFERRED EMBODIMENTS

Preferably, the AXL inhibitor is 1-(6,7-dihydro-5H-benzo[6,7]cyclohepta[1,2-c]pyridazin-3-yl)-N³-((7-(S)-pyrrolidin-1-yl)-6,7,8,9-tetrahydro-5H-benzo[7]annulene-2-yl)-1H-1,2,4-triazole-3,5-diamine.

The most preferred AXL inhibitor is bemcentinib (CAS No. 1037624-75-1; UNII 0ICW2LX8AS). Bemcentinib may be referred to as BGB324 or R428.

### OTHER EMBODIMENTS

In some other embodiments the AXL inhibitor is selected from the group consisting of:
Dubermatinib (CAS No. 1341200-45-0; UNII 14D65TV20J);
Gilteritinib (CAS No. 1254053-43-4; UNII 66D92MGC8M);
Cabozantinib (CAS No. 849217-68-1; UNII 1C39JW444G);
SGI7079 (CAS No. 1239875-86-5);
Merestinib (CAS No. 1206799-15-6; UNII 5OGS5K699E);
Amuvatinib (CAS No. 850879-09-3; UNII SO9S6QZB4R);
Bosutinib (CAS No. 380843-75-4; UNII 5018V4AEZ0);
Glesatinib (CAS No. 936694-12-1; UNII 7Q29OXD98N); and
foretinib (CAS No. 849217-64-7; UNII 81FH7VK1C4).
TP0903 (CAS No. 1341200-45-0).

In some other embodiments the AXL inhibitor is an AXL inhibitor as described in any of the following references: WO2008/083367, WO2010/083465, and WO2012/028332 (the contents of each of which is hereby incorporated by reference).

### Definitions

As used herein, unless specified to the contrary, the following terms have the meaning indicated:
“Amino” refers to the —NH₂ radical.
“Carboxy” refers to the —C(O)OH radical.
“Cyano” refers to the —CN radical.
“Nitro” refers to the —NO₂ radical.
“Oxa” refers to the —O— radical.
“Oxo” refers to the ═O radical.
“Thioxo” refers to the ═S radical.
“Alkyl” refers to a straight or branched hydrocarbon chain radical consisting solely of carbon and hydrogen atoms, containing no unsaturation, having from one to twelve carbon atoms, preferably one to eight carbon atoms or one to six carbon atoms and which is attached to the rest of the molecule by a single bond, for example, methyl, ethyl, n-propyl, 1-methylethyl (iso-propyl), n-butyl, n-pentyl, 1,1-dimethylethyl (t-butyl), 3-methylhexyl, 2-methylhexyl, and the like. For purposes of this disclosure, the term “lower alkyl” refers to an alkyl radical having one to six carbon atoms.

“Optionally substituted alkyl” refers to an alkyl radical, as defined above, which is optionally substituted by one or more substituents selected from the group consisting of halo, cyano, nitro, oxo, thioxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²⁰, —N(R²⁰)C(O)R²⁰, —N(R²⁰)S(O)₂R²⁰, —S(O)ₜOR²⁰ (where t is 1 or 2), —S(O)ₚR²⁰ (where p is 0, 1 or 2), and —S(O)₂N(R²⁰)₂ where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R^{20's}, together with the common nitrogen to which they are both attached, form an optionally substituted N-heterocyclyl or an optionally substituted N-heteroaryl.

“Alkenyl” refers to a straight or branched hydrocarbon chain radical consisting solely of carbon and hydrogen atoms, containing at least one double bond, having from two to twelve carbon atoms, preferably one to eight carbon atoms and which is attached to the rest of the molecule by a single bond, for example, ethenyl, prop-1-enyl, but-1-enyl, pent-1-enyl, and penta-1,4-dienyl.

“Optionally substituted alkenyl” refers to an alkenyl radical, as defined above, which is optionally substituted by one or more substituents selected from the group consisting of halo, cyano, nitro, oxo, thioxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²⁰, —N(R²⁰)C(O)R²⁰, —N(R²⁰)S(O)₂R²⁰, —S(O)ₜOR²⁰ (where t is 1 or 2), —S(O)ₚR²⁰ (where p is 0, 1 or 2), and —S(O)₂N(R²⁰)₂ where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R^{20's}, together with the common nitrogen to which they are both attached, form an optionally substituted N-heterocyclyl or an optionally substituted N-heteroaryl.

“Alkynyl” refers to a straight or branched hydrocarbon chain radical consisting solely of carbon and hydrogen atoms, containing at least one triple bond, optionally containing at least one double bond, having from two to twelve carbon atoms, preferably one to eight carbon atoms and which is attached to the rest of the molecule by a single bond, for example, ethynyl, propynyl, butynyl, pentynyl, and hexynyl.

“Optionally substituted alkynyl” refers to an alkynyl radical, as defined above, which is optionally substituted by one or more substituents selected from the group consisting of halo, cyano, nitro, oxo, thioxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²⁰, —N(R²⁰)C(O)R²⁰, —N(R²⁰)S(O)₂R²⁰, —S(O)ₜOR²⁰ (where t is 1 or 2), —S(O)ₚR²⁰ (where p is 0, 1 or 2), and —S(O)₂N(R²⁰)₂ where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R^{20's}, together with the common nitrogen to which they are both attached, form an optionally substituted N-heterocyclyl or an optionally substituted N-heteroaryl.

“Straight or branched alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, and n-butylene. The alkylene chain is attached to the rest of the molecule through a single bond and to the radical group through a single bond. The points of attachment of the alkylene chain to the rest of the molecule and to the radical group can be through one carbon in the alkylene chain or through any two carbons within the chain.

“Optionally substituted straight or branched alkylene chain” refers to an alkylene chain, as defined above, which is optionally substituted by one or more substituents selected from the group consisting of halo, cyano, nitro, aryl, cycloalkyl, heterocyclyl, heteroaryl, oxo, thioxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²⁰, —N(R²⁰)C(O)R²⁰, —N(R²⁰)S(O)₂R²⁰, —S(O)ₜOR²⁰ (where t is 1 or 2), —S(O)ₚR²⁰ (where p is 0, 1 or 2), and —S(O)₂N(R²⁰)₂ where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R^{20's}, together with the common nitrogen to which they are both attached, form an optionally substituted N-heterocyclyl or an optionally substituted N-heteroaryl.

“Straight or branched alkenylene chain” refers to a straight or branched divalent hydrocarbon chain linking the rest of the molecule to a radical group, consisting solely of carbon and hydrogen, containing at least one double bond and having from two to twelve carbon atoms, for example, ethenylene, propenylene, and n-butenylene. The alkenylene chain is attached to the rest of the molecule through a double bond or a single bond and to the radical group through a double bond or a single bond. The points of attachment of the alkenylene chain to the rest of the molecule and to the radical group can be through one carbon or any two carbons within the chain.

“Optionally substituted straight or branched alkenylene chain” refers to an alkenylene chain, as defined above, which is optionally substituted by one or more substituents selected from the group consisting of halo, cyano, nitro, aryl, cycloalkyl, heterocyclyl, heteroaryl, oxo, thioxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²⁰, —N(R²⁰)C(O)R²⁰, —N(R²⁰)S(O)₂R²⁰, —S(O)ₜOR²⁰ (where t is 1 or 2), —S(O)ₚR²⁰ (where p is 0, 1 or 2), and —S(O)₂N(R²⁰)₂ where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R^{20's}, together with the common nitrogen to which they are both attached, form an optionally substituted N-heterocyclyl or an optionally substituted N-heteroaryl.

“Straight or branched alkynylene chain” refers to a straight or branched divalent hydrocarbon chain linking the rest of the molecule to a radical group, consisting solely of carbon and hydrogen, containing at least one triple bond and having from two to twelve carbon atoms, for example, propynylene, and n-butynylene. The alkynylene chain is attached to the rest of the molecule through a single bond and to the radical group through a double bond or a single bond. The points of attachment of the alkynylene chain to the rest of the molecule and to the radical group can be through one carbon or any two carbons within the chain.

“Optionally substituted straight or branched alkynylene chain” refers to an alkynylene chain, as defined above, which is optionally substituted by one or more substituents selected from the group consisting of alkyl, alkenyl, halo, haloalkenyl, cyano, nitro, aryl, cycloalkyl, heterocyclyl, heteroaryl, oxo, thioxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²⁰, —N(R²⁰)C(O)R²⁰, —N(R²⁰)S(O)₂R²⁰, —S(O)ₜOR²⁰ (where t is 1 or 2), —S(O)ₚR²⁰ (where p is 0, 1 or 2), and —S(O)₂N(R²⁰)₂ where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R^{20's}, together with the common nitrogen to which they are both attached, form an optionally substituted N-heterocyclyl or an optionally substituted N-heteroaryl.

“Aryl” refers to a hydrocarbon ring system radical comprising hydrogen, 6 to 14 carbon atoms and at least one aromatic ring. For purposes of this disclosure, the aryl radical may be a monocyclic, bicyclic, or tricyclic system and which may include spiro ring systems. An aryl radical is commonly, but not necessarily, attached to the parent molecule via an aromatic ring of the aryl radical. For purposes of this disclosure, an “aryl” radical as defined herein can not contain rings having more than 7 members and cannot contain rings wherein two non-adjacent ring atoms thereof are connected through an atom or a group of atoms (i.e., a bridged ring system). Aryl radicals include, but are not limited to, aryl radicals derived from acenaphthylene, anthracene, azulene, benzene, 6,7,8,9-tetrahydro-5H-benzo[7]annulene, fluorene, as-indacene, s-indacene, indane, indene, naphthalene, phenalene, and phenanthrene.

“Optionally substituted aryl” refers to an aryl radical, as defined above, which is optionally substituted by one or more substituents selected from the group consisting of alkyl, alkenyl, alkynyl, halo, haloalkyl, haloalkenyl, haloalkynyl, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkenyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R²¹—OR²⁰, —R²¹—OC(O)—R²⁰, —R²¹—N(R²⁰)₂, —R²¹—C(O)R²⁰, —R²¹—C(O)OR²⁰, —R²¹—C(O)N(R²⁰)₂, —R²¹—O—R²²—C(O)N(R²⁰)₂, —R²¹—N(R²⁰)C(O)OR²⁰, —R²¹—N(R²⁰)C(O)R²⁰, —R²¹—N(R²⁰)S(O)₂R²⁰, —R²¹—C(═NR²⁰)N(R²⁰)₂, —R²¹—S(O)ₜOR²⁰ (where t is 1 or 2), —R²¹—S(O)ₚR²⁰ (where p is 0, 1 or 2), and —R²¹—S(O)₂N(R²⁰)₂, where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R^{20's}, together with the common nitrogen to which they are both attached, form an optionally substituted N-heterocyclyl or an optionally substituted N-heteroaryl, each R²¹ is independently a direct bond or a straight or branched alkylene or alkenylene chain, and R²² is a straight or branched alkylene or alkenylene chain.

“Aralkyl” refers to a radical of the formula —R_{b}—R_{c} where R_{b} is an alkylene chain as defined above and R_{c} is one or more aryl radicals as defined above, for example, benzyl and diphenylmethyl.

“Optionally substituted aralkyl” refers to an aralkyl radical, as defined above, wherein the alkylene chain of the aralkyl radical is an optionally substituted alkylene chain, as defined above, and each aryl radical of the aralkyl radical is an optionally substituted aryl radical, as defined above.

“Aralkenyl” refers to a radical of the formula —R_{d}—R_{c} where R_{d} is an alkenylene chain as defined above and R_{c} is one or more aryl radicals as defined above.

“Optionally substituted aralkenyl” refers to an aralkenyl radical, as defined above, wherein the alkenylene chain of the aralkenyl radical is an optionally substituted alkenylene chain, as defined above, and each aryl radical of the aralkenyl radical is an optionally substituted aryl radical, as defined above.

“Aralkynyl” refers to a radical of the formula —RₑR_{c} where Rₑ is an alkynylene chain as defined above and R_{c} is one or more aryl radicals as defined above.

“Optionally substituted aralkynyl” refers to an aralkynyl radical, as defined above, wherein the alkynylene chain of the aralkynyl radical is an optionally substituted alkynylene chain, as defined above, and each aryl radical of the aralkynyl radical is an optionally substituted aryl radical, as defined above.

“Cycloalkyl” refers to a stable non-aromatic monocyclic or polycyclic hydrocarbon radical consisting solely of carbon and hydrogen atoms, which includes fused, spiro or bridged ring systems, having from three to fifteen carbon atoms, preferably having from three to ten carbon atoms, more preferably from five to seven carbons and which is saturated or unsaturated and attached to the rest of the molecule by a single bond. For purposes of this disclosure, a bridged ring system is a system wherein two non-adjacent ring atoms thereof are connected through an atom or a group of atoms, wherein the atom or the group of atoms are the bridging element. An example of a bridged cycloalkyl (monovalent) radical is norbornanyl (also called bicyclo[2.2.1]heptanyl). For purposes of this disclosure, a non-bridged ring system is a system which does not contain a bridging element, as described above. For purposes of this disclosure, a fused ring system is a system wherein two adjacent ring atoms thereof are connected through an atom or a group of atoms. An example of a fused cycloalkyl (monovalent) radical is decahydronaphthalenyl (also called decalinyl). For purposes of this disclosure, a spiro ring system is a system wherein two rings are joined via a single carbon (quaternary) atom. An example of a spiro cycloalkyl (monovalent) radical is spiro[5.5]undecanyl. Monocyclic cycloalkyl radicals do not include spiro, fused or bridged cycloalkyl radicals, but do include for example, cyclopropyl, cyclobutyl, cyclopentyl, cyclohexyl, cycloheptyl, and cyclooctyl. Polycyclic radicals include fused, spiro or bridged cycloalkyl radicals, for example, C₁₀ radicals such as adamantanyl (bridged) and decalinyl (fused), and C₇ radicals such as bicyclo[3.2.0]heptanyl (fused), norbornanyl and norbornenyl (bridged), as well as substituted polycyclic radicals, for example, substituted C₇ radicals such as 7,7-dimethylbicyclo[2.2.1]heptanyl (bridged).

“Optionally substituted cycloalkyl” refers to a cycloalkyl radical, as defined above, which is optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, cycloalkylalkyl, optionally substituted cycloalkylalkenyl, optionally substituted cycloalkylalkenyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R²¹—OR²⁰, —R²¹—OC(O)—R²⁰, —R²¹—N(R²⁰)₂, —R²¹—C(O)R²⁰, —R²¹—C(O)OR²⁰, —R²¹—C(O)N(R²⁰)₂, —R²¹—N(R²⁰)C(O)OR²⁰, —R²¹—N(R²⁰)C(O)R²⁰, —R²¹—N(R²⁰)S(O)₂R²⁰, —R²¹—C(═NR²⁰)N(R²⁰)₂, —R²¹—S(O)ₜOR²⁰ (where t is 1 or 2), —R²¹—S(O)ₚR²⁰ (where p is 0, 1 or 2), and —R²¹—S(O)₂N(R²⁰)₂, where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R^{20's}, together with the common nitrogen to which they are both attached, form an optionally substituted N-heterocyclyl or an optionally substituted N-heteroaryl, and each R²¹ is independently a direct bond or a straight or branched alkylene or alkenylene chain.

“Cycloalkylalkyl” refers to a radical of the formula —R_{b}R_{g} where R_{b} is an alkylene chain as defined above and R_{g} is a cycloalkyl radical as defined above.

“Optionally substituted cycloalkylalkyl” refers to a cycloalkylalkyl radical, as defined above, wherein the alkylene chain of the cycloalkylalkyl radical is an optionally substituted alkylene chain, as defined above, and the cycloalkyl radical of the cycloalkylalkyl radical is an optionally substituted cycloalkyl radical, as defined above.

“Cycloalkylalkenyl” refers to a radical of the formula —R_{d}R_{g} where R_{d} is an alkenylene chain as defined above and R_{g} is a cycloalkyl radical as defined above.

“Optionally substituted cycloalkylalkenyl” refers to a cycloalkylalkenyl radical, as defined above, wherein the alkenylene chain of the cycloalkylalkenyl radical is an optionally substituted alkenylene chain, as defined above, and the cycloalkyl radical of the cycloalkylalkenyl radical is an optionally substituted cycloalkyl radical as defined above.

“Cycloalkylalkenyl” refers to a radical of the formula —RₑR_{g} where Rₑ is an alkynylene radical as defined above and R_{g} is a cycloalkyl radical as defined above.

“Optionally substituted cycloalkylalkenyl” refers to a cycloalkylalkenyl radical, as defined above, wherein the alkynylene chain of the cycloalkylalkenyl radical is an optionally substituted alkynylene chain, as defined above, and the cycloalkyl radical of the cycloalkylalkenyl radical is an optionally substituted cycloalkyl radical as defined above.

“Halo” refers to bromo, chloro, fluoro or iodo.

“Haloalkyl” refers to an alkyl radical, as defined above, that is substituted by one or more halo radicals, as defined above, for example, trifluoromethyl, difluoromethyl, trichloromethyl, 2,2,2-trifluoroethyl, 1-fluoromethyl-2-fluoroethyl, 3-bromo-2-fluoropropyl, and 1-bromomethyl-2-bromoethyl.

“Haloalkenyl” refers to an alkenyl radical, as defined above, that is substituted by one or more halo radicals, as defined above.

“Haloalkynyl” refers to an alkynyl radical, as defined above, that is substituted by one or more halo radicals, as defined above.

“Heterocyclyl” refers to a stable 3- to 18-membered non-aromatic ring system radical which comprises one to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include spiro or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of a bridged heterocyclyl include, but are not limited to, azabicyclo[2.2.1]heptanyl, diazabicyclo[2.2.1]heptanyl, diazabicyclo[2.2.2]octanyl, diazabicyclo[3.2.1]octanyl, diazabicyclo[3.3.1]nonanyl, diazabicyclo[3.2.2]nonanyl and oxazabicyclo[2.2.1]heptanyl. A “bridged N-heterocyclyl” is a bridged heterocyclyl containing at least one nitrogen, but which optionally contains up to four additional heteroatoms selected from O, N and S. For purposes of this disclosure, a non-bridged ring system is a system wherein no two non-adjacent ring atoms thereof are connected through an atom or a group of atoms. Examples of heterocyclyl radicals include, but are not limited to, dioxolanyl, 1,4-diazepanyl, decahydroisoquinolyl, imidazolinyl, imidazolidinyl, isothiazolidinyl, isoxazolidinyl, morpholinyl, octahydroindolyl, octahydroisoindolyl, octahydro-1H-pyrrolo[3,2-c]pyridinyl, octahydro-1H-pyrrolo[2,3-c]pyridinyl, octahydro-1H-pyrrolo[2,3-b]pyridinyl, octahydro-1H-pyrrolo[3,4-b]pyridinyl, octahydropyrrolo[3,4-c]pyrrolyl, octahydro-1H-pyrido[1,2-a]pyrazinyl, 2-oxopiperazinyl, 2-oxopiperidinyl, 2-oxopyrrolidinyl, oxazolidinyl, 3,7-diazabicyclo[3.3.1]nonan-3-yl, piperidinyl, piperazinyl, 4-piperidonyl, pyrrolidinyl, pyrazolidinyl, quinuclidinyl, thiazolidinyl, tetrahydrofuranyl, thienyl[1,3]dithianyl, trithianyl, tetrahydropyranyl, thiomorpholinyl, thiamorpholinyl, 1-oxo-thiomorpholinyl, 1,1-dioxo-thiomorpholinyl, azetidinyl, octahydropyrrolo[3,4-c]pyrrolyl, octahydropyrrolo[3,4-b]pyrrolyl, decahydropyrazino[1,2-a]azepinyl, azepanyl, azabicyclo[3.2.1]octyl, and 2,7-diazaspiro[4.4]nonanyl.

“Optionally substituted heterocyclyl” refers to a heterocyclyl radical, as defined above, which is optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkenyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R²¹—OR²⁰, —R²¹—OC(O)—R²⁰, —R²¹—N(R²⁰)₂, —R²¹—C (O)R²⁰, —R²¹—C(O)OR²⁰, —R²¹—C(O)N(R²⁰)₂, —R²¹—N(R²⁰)C(O)OR²⁰, —R²¹—N(R²⁰)C(O)R²⁰, —R²¹—N(R²⁰)S(O)₂R²⁰, —R²—C(═NR²⁰)N(R²⁰)₂, —R²¹—S(O)ₜOR²⁰ (where t is 1 or 2), —R²¹—S(O)ₚR²⁰ (where p is 0, 1 or 2), and —R²¹—S(O)₂N(R²⁰)₂, where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R^{20's}, together with the common nitrogen to which they are both attached, form an optionally substituted N-heterocyclyl or an optionally substituted N-heteroaryl, and each R²¹ is independently a direct bond or a straight or branched alkylene or alkenylene chain.

“N-heterocyclyl” refers to a heterocyclyl radical as defined above containing at least one nitrogen and where the point of attachment of the N-heterocyclyl radical to the rest of the molecule may be through a nitrogen atom in the N-heterocyclyl radical or through a carbon in the N-heterocyclyl radical.

“Optionally substituted N-heterocyclyl” refers to an N-heterocyclyl, as defined above, which is optionally substituted by one or more substituents as defined above for optionally substituted heterocyclyl.

“Heterocyclylalkyl” refers to a radical of the formula —R_{b}Rₕ where R_{b} is an alkylene chain as defined above and Rₕ is a heterocyclyl radical as defined above, and when the heterocyclyl is a nitrogen-containing heterocyclyl, the heterocyclyl may be attached to the alkylene chain at the nitrogen atom.

“Optionally substituted heterocyclylalkyl” refers to a heterocyclylalkyl radical, as defined above, wherein the alkylene chain of the heterocyclylalkyl radical is an optionally substituted alkylene chain, as defined above, and the heterocyclyl radical of the heterocyclylalkyl radical is an optionally substituted heterocyclyl radical, as defined above.

“Heterocyclylalkenyl” refers to a radical of the formula —R_{d}Rₕ where R_{d} is an alkenylene chain as defined above and Rₕ is a heterocyclyl radical as defined above, and when the heterocyclyl is a nitrogen-containing heterocyclyl, the heterocyclyl may be attached to the alkenylene chain at the nitrogen atom.

“Optionally substituted heterocyclylalkenyl” refers to a heterocyclylalkenyl radical, as defined above, wherein the alkenylene chain of the heterocyclylalkenyl radical is an optionally substituted alkenylene chain, as defined above, and the heterocyclyl radical of the heterocyclylalkenyl radical is an optionally substituted heterocyclyl radical, as defined above.

“Heterocyclylalkynyl” refers to a radical of the formula —RₑRₕ where Rₑ is an alkynylene chain as defined above and Rₕ is a heterocyclyl radical as defined above, and when the heterocyclyl is a nitrogen-containing heterocyclyl, the heterocyclyl may be attached to the alkynylene chain at the nitrogen atom.

“Optionally substituted heterocyclylalkynyl” refers to a heterocyclylalkynyl radical, as defined above, wherein the alkynylene chain of the heterocyclylalkynyl radical is an optionally substituted alkynylene chain, as defined above, and the heterocyclyl radical of the heterocyclylalkynyl radical is an optionally substituted heterocyclyl radical, as defined above.

“Heteroaryl” refers to a 5- to 14-membered ring system radical comprising hydrogen atoms, one to thirteen carbon atoms, one to six heteroatoms selected from the group consisting of nitrogen, oxygen and sulfur, and at least one aromatic ring. A heteroaryl radical is commonly, but not necessarily, attached to the parent molecule via an aromatic ring of the heteroaryl radical. For purposes of this disclosure, the heteroaryl radical may be a monocyclic, bicyclic or tricyclic ring system, which may include spiro or bridged ring systems; and the nitrogen, carbon or sulfur atoms in the heteroaryl radical may be optionally oxidized and the nitrogen atom may be optionally quaternized. For purposes of this disclosure, the aromatic ring of the heteroaryl radical need not contain a heteroatom, as long as one ring of the heteroaryl radical contains a heteroatom. For example benzo-fused heterocyclyls such as 1,2,3,4-tetrahydroisoquinolin-7-yl are considered a “heteroaryl” for the purposes of this disclosure. Except for the polycyclic heteroaryls containing more than 14 ring atoms, as defined below, a “heteroaryl” radical as defined herein can not contain rings having more than 7 members and cannot contain rings wherein two non-adjacent members thereof are connected through an atom or a group of atoms (i.e., a bridged ring system). Examples of heteroaryl radicals include, but are not limited to, azepinyl, acridinyl, benzimidazolyl, benzindolyl, 1,3-benzodioxolyl, benzofuranyl, benzoxazolyl, benzothiazolyl, benzothiadiazolyl, benzo[b][1,4]dioxepinyl, benzo[b][1,4]oxazinyl, benzo[b]azepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 3,4-dihydro-2H-benzo[b][1,4]dioxepinyl, cyclopenta[4,5]thieno[2,3-d]pyrimidinyl such as 6,7-dihydro-5H-cyclopenta[4,5]thieno[2,3-d]pyrimidinyl, 5,6-dihydrobenzo[h]quinazolinyl, 3,4-dihydro-2H-benzo[b][1,4]thiazinyl, 5,6-dihydrobenzo[h]cinnolinyl, 7′,8′-dihydro-5′H-spiro[[1,3]dioxolane-2,6′-quinoline]-3′-yl, 6,7-dihydro-5H-benzo[6,7]cyclohepta[1,2-c]pyridazinyl, 2,3-dihydro-1H-pyrido[2,3-b][1,4]oxazinyl, 3′,4′-dihydrospiro[cyclobutane-1,2′-pyrido[3,2-b][1,4]oxazinyl, dihydropyridooxazinyl such as 3,4-dihydro-2H-pyrido[3,2-b][1,4]oxazinyl, dihydropyridothiazinyl such as 3,4-dihydro-2H-pyrido[3,2-b][1,4]thiazinyl, dibenzofuranyl, dibenzothiophenyl, furanyl, furanonyl, furo[3,2-c]pyridinyl, furopyrimidinyl, furopyridazinyl, furopyrazinyl, isothiazolyl, imidazolyl, imidazopyrimidinyl, imidazopyridazinyl, imidazopyrazinyl, imidazo[1,2-a]pyridinyl, indazolyl, indolyl, indazolyl, isoindolyl, indolinyl, isoindolinyl, isoquinolinyl (isoquinolyl), indolizinyl, isoxazolyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 3′-oxo-3′,4′-dihydrospiro[cyclobutane-1,2′-pyrido[3,2-b][1,4]oxazine]yl, 7-oxo-5,6,7,8-tetrahydro-1,8-naphthyridinyl, 1-phenyl-1H-pyrrolyl, phenazinyl, phenothiazinyl, phenoxazinyl, phthalazinyl, phenanthridinyl, pteridinyl, purinyl, pyrrolyl, pyrazolyl, pyrazolo[3,4-d]pyrimidinyl, pyridinyl (pyridyl), pyrido[3,2-d]pyrimidinyl, pyrido[3,4-d]pyrimidinyl, pyrazinyl, pyrimidinyl, pyridazinyl (pyridazyl), pyrrolyl, pyrrolopyrimidinyl, pyrrolopyridazinyl, pyrrolopyrazinyl, 2H-pyrido[3,2-b][1,4]oxazinonyl, 1H-pyrido[2,3-b][1,4]oxazinonyl, pyrrolopyridinyl such as 1H-pyrrolo[2,3-b]pyridinyl, quinazolinyl, quinoxalinyl, quinolinyl, quinuclidinyl, tetrahydroquinolinyl, 5,6,7,8-tetrahydroquinazolinyl, 2,3,4,5-tetrahydrobenzo[b]oxepinyl, 6,7,8,9-tetrahydro-5H-cyclohepta[b]pyridinyl, 6,7,8,9-tetrahydro-5H-pyrido[3,2-c]azepinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, 1,2,3,4-tetrahydroisoquinolin-7-yl, triazinyl, thieno[2,3-d]pyrimidinyl, thienopyrimidinyl (e.g., thieno[3,2-d]pyrimidinyl), thieno[2,3-c]pyridinyl, thienopyridazinyl, thienopyrazinyl, and thiophenyl (thienyl).

“Optionally substituted heteroaryl” refers to a heteroaryl radical, as defined above, which is optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkenyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R²¹—OR²⁰, —R²¹—OC(O)—R²⁰, —R²¹—N(R²⁰)₂, —R²¹—C (O)R²⁰, —R²¹—C(O)OR²⁰, —R²¹—C(O)N(R²⁰)₂, —R²¹—N(R²⁰)C(O)OR²⁰, —R²¹—N(R²⁰)C(O)R²⁰, —R²¹—N(R²⁰)S(O)₂R²⁰ 2, —R²¹—C(═NR²⁰)N(R²⁰)₂, —R²¹—S(O)ₜOR²⁰ (where t is 1 or 2), —R²¹—S(O)ₚR²⁰ (where p is 0, 1 or 2), and —R²¹—S(O)₂N(R²⁰)₂, where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R^{20's}, together with the common nitrogen to which they are both attached, form an optionally substituted N-heterocyclyl or an optionally substituted N-heteroaryl, and each R²¹ is independently a direct bond or a straight or branched alkylene or alkenylene chain.

“N-heteroaryl” refers to a heteroaryl radical as defined above containing at least one nitrogen and where the point of attachment of the N-heteroaryl radical to the rest of the molecule may be through a nitrogen atom in the N-heteroaryl radical or through a carbon atom in the N-heteroaryl radical.

“Optionally substituted N-heteroaryl” refers to an N-heteroaryl, as defined above, which is optionally substituted by one or more substituents as defined above for optionally substituted heteroaryl.

“Polycyclic heteroaryl containing more than 14 ring atoms” refers to a 15- to 20-membered ring system radical comprising hydrogen atoms, one to fourteen carbon atoms, one to eight heteroatoms selected from the group consisting of nitrogen, oxygen and sulfur, and at least one aromatic ring. A “polycyclic heteroaryl containing more than 14 ring atoms” radical is commonly, but not necessarily, attached to the parent molecule via an aromatic ring of the “polycyclic heteroaryl containing more than 14 ring atoms” radical. For purposes of this disclosure, the “polycyclic heteroaryl containing more than 14 ring atoms” radical may be a bicyclic, tricyclic or tetracyclic ring system, which may include fused or spiro ring systems; and the nitrogen, carbon or sulfur atoms in the “polycyclic heteroaryl containing more than 14 ring atoms” radical may be optionally oxidized and the nitrogen atom may also be optionally quaternized. For purposes of this disclosure, the aromatic ring of the “polycyclic heteroaryl containing more than 14 ring atoms” radical need not contain a heteroatom, as long as one ring of the “polycyclic heteroaryl containing more than 14 ring atoms” radical contains a heteroatom. Examples of “polycyclic heteroaryl containing more than 14 ring atoms” radicals include, but are not limited to, 6,7-dihydro-5H-benzo[6,7]cyclohepta[1,2-c]pyridazin-3-yl, 6,7-dihydro-5H-pyrido[2′,3′:6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl.

“Optionally substituted polycyclic heteroaryl containing more than 14 ring atoms” is meant to include “polycyclic heteroaryl containing more than 14 ring atoms” radicals, as defined above, which are optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkenyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R²¹—OR²⁰, —R²¹—OC(O)—R²⁰, —R²¹—N(R²⁰)₂, —R²¹—C (O)R²⁰, —R²¹—C(O)OR²⁰, —R²¹—C(O)N(R²⁰)₂, —R²¹—N(R²⁰)C(O)OR²⁰, —R²¹—N(R²⁰)C(O)R²⁰, —R²¹—N(R²⁰)S(O)ₜR²⁰ (where t is 1 or 2), —R²¹—S(O)ₜOR²⁰ (where t is 1 or 2), —R²¹—S(O)ₚR²⁰ (where p is 0, 1 or 2), and —R²¹—S(O)ₜN(R²⁰)₂ (where t is 1 or 2), where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R^{20's}, together with the common nitrogen to which they are both attached, may optionally form an optionally substituted N-heterocyclyl or an optionally substituted N-heteroaryl, and each R²¹ is independently a direct bond or a straight or branched alkylene or alkenylene chain.

“Heteroarylalkyl” refers to a radical of the formula —R_{b}Rᵢ where R_{b} is an alkylene chain as defined above and Rᵢ is a heteroaryl radical as defined above, and when the heteroaryl is a nitrogen-containing heteroaryl, the heteroaryl may be attached to the alkylene chain at the nitrogen atom.

“Optionally substituted heteroarylalkyl” refers to a heteroarylalkyl radical, as defined above, wherein the alkylene chain of the heteroarylalkyl radical is an optionally substituted alkylene chain, as defined above, and the heteroaryl radical of the heteroarylalkyl radical is an optionally substituted heteroaryl radical, as defined above.

“Heteroarylalkenyl” refers to a radical of the formula —R_{d}Rᵢ where R_{d} is an alkenylene chain as defined above and Rᵢ is a heteroaryl radical as defined above, and when the heteroaryl is a nitrogen-containing heteroaryl, the heteroaryl may be attached to the alkenylene chain at the nitrogen atom.

“Optionally substituted heteroarylalkenyl” refers to a heteroarylalkenyl radical, as defined above, wherein the alkenylene chain of the heteroarylalkenyl radical is an optionally substituted alkenylene chain, as defined above, and the heteroaryl radical of the heteroarylalkenyl radical is an optionally substituted heteroaryl radical, as defined above.

“Heteroarylalkynyl” refers to a radical of the formula —RₑRᵢ where Rₑ is an alkynylene chain as defined above and Rᵢ is a heteroaryl radical as defined above, and when the heteroaryl is a nitrogen-containing heteroaryl, the heteroaryl may be attached to the alkynylene chain at the nitrogen atom.

“Optionally substituted heteroarylalkynyl” refers to a heteroarylalkynyl radical, as defined above, wherein the alkynylene chain of the heteroarylalkynyl radical is an optionally substituted alkynylene chain, as defined above, and the heteroaryl radical of the heteroarylalkynyl radical is an optionally substituted heteroaryl radical, as defined above.

“Hydroxyalkyl” refers to an alkyl radical as defined above which is substituted by one or more hydroxy radicals (—OH).

Certain chemical groups named herein may be preceded by a shorthand notation indicating the total number of carbon atoms that are to be found in the indicated chemical group. For example; C₇-C₁₂alkyl describes an alkyl group, as defined below, having a total of 7 to 12 carbon atoms, and C₄-C₁₂ cycloalkylalkyl describes a cycloalkylalkyl group, as defined below, having a total of 4 to 12 carbon atoms. The total number of carbons in the shorthand notation does not include carbons that may exist in substituents of the group described.

The compounds of formula (I), or their pharmaceutically acceptable salts, may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, such as HPLC using a chiral column. When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposable mirror images of one another.

A “tautomer” refers to a proton shift from one atom of a molecule to another atom of the same molecule. The present disclosure includes tautomers of any said compounds.

“Atropisomers” are stereoisomers resulting from hindered rotation about single bonds where the barrier to rotation is high enough to allow for the isolation of the conformers (Eliel, E. L.; Wilen, S. H. Stereochemistry of Organic Compounds; Wiley & Sons: New York, 1994; Chapter 14). Atropisomerism is significant because it introduces an element of chirality in the absence of stereogenic atoms. The disclosure is meant to encompass atropisomers, for example in cases of limited rotation around the single bonds emanating from the core triazole structure, atropisomers are also possible and are also specifically included in the compounds of the disclosure.

The chemical naming protocol and structure diagrams used herein are a modified form of the IUPAC nomenclature system wherein the compounds of formula (I) are named herein as derivatives of the central core structure, i.e., the triazole structure. For complex chemical names employed herein, a substituent group is named before the group to which it attaches. For example, cyclopropylethyl comprises an ethyl backbone with cyclopropyl substituent. In chemical structure diagrams, all bonds are identified, except for some carbon atoms, which are assumed to be bonded to sufficient hydrogen atoms to complete the valency.

For purposes of this disclosure, the depiction of the bond attaching the R³ substituent to the parent triazole moiety in formula (I), as shown below:

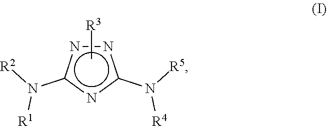

is intended to include only the two regioisomers shown below, i.e., compounds of formula (Ia) and (Ib):

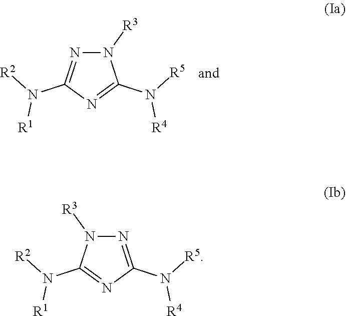

The numbering system of the ring atoms in compounds of formula (Ia) is shown below:

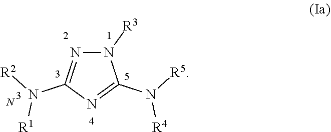

For example, a compound of formula (Ia) wherein R¹, R⁴ and R⁵ are each hydrogen, R² is 4-(2-(pyrrolidin-1-yl)ethoxy)phenyl and R³ is 6,7-dihydro-5H-benzo[6,7]cyclohepta[1,2-c]pyridazin-3-yl; i.e., a compound of the following formula:

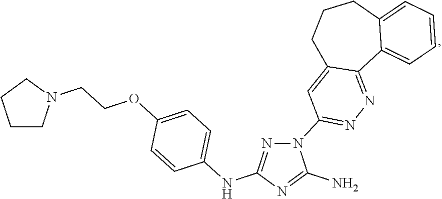 [SMILES: Nc1nc(Nc2ccc(OCCN3CCCC3)cc2)nn1-c1cc2c(nn1)-c1ccccc1CCC2]

is named herein as 1-(6,7-dihydro-5H-benzo[6,7]cyclohepta[1,2-c]pyridazin-3-yl)-N³-(4-(2-(pyrrolidin-1-yl)ethoxy)phenyl)-1H-1,2,4-triazole-3,5-diamine.

The numbering system of the ring atoms in compounds of formula (Ib) is shown below:

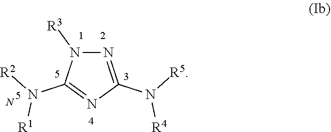

Compounds of formula (Ib) are similarly named herein.

Antibody AXL Inhibitors

In some embodiments the AXL inhibitor is an antibody. Preferably the antibody has AXL inhibitory activity. In some cases the antibody inhibits the binding of AXL to the GAS6 ligand.

In some embodiments, the anti-AXL antibody is an antibody as described in any of the following references: WO/2016/097370, WO/2017/220695, WO/2015/193428, WO/2016/166296, WO/2015/193430, EP2267454, WO/2009/063965, WO/2011/159980, WO/20121175691, WO/2012/175692, WO/2013/064685, WO/2014/068139, WO/2009/062690, and WO/2010/130751 (the contents of each of which is hereby incorporated by reference).

In another embodiment, the anti-AXL antibody is an antibody as described in international patent application WO/2015/193428, the contents of which is hereby incorporated by reference, particularly as shown at pages 82-83.

In another embodiment, the anti-AXL antibody is an antibody as described in international patent application WO/2016/166296, the contents of which is hereby incorporated by reference, particularly the humanized 1H12 antibody disclosed therein.

In another embodiment, the anti-AXL antibody is an antibody as described in international patent application WO/2015/193430, the contents of which is hereby incorporated by reference, particularly as shown at pages 72-73.

In another embodiment, the anti-AXL antibody is an antibody as described in European patent publication EP2267454, the contents of which is hereby incorporated by reference.

In another embodiment, the anti-AXL antibody is an antibody as described in European patent publication WO/2009/063965, the contents of which is hereby incorporated by reference, particularly as shown at pages 31-33.

In another embodiment, the anti-AXL antibody is an antibody as described in US patent publication US 2012/0121587 A1, the contents of which is hereby incorporated by reference, particularly as shown at pages 26-61.

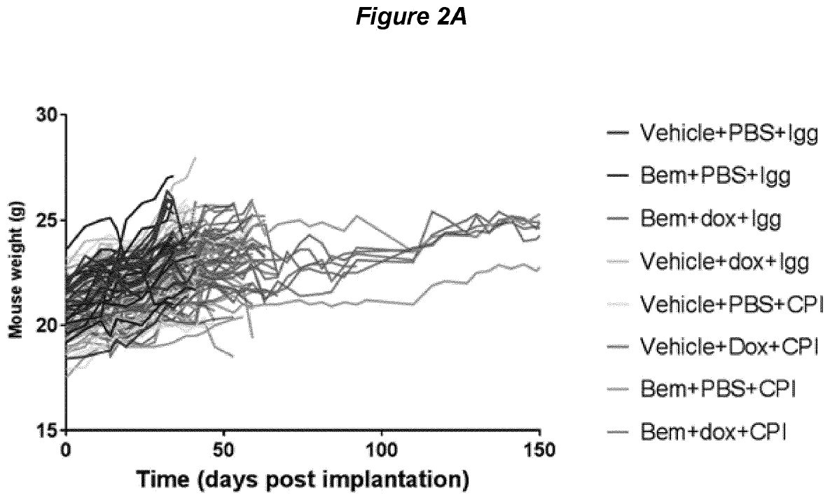

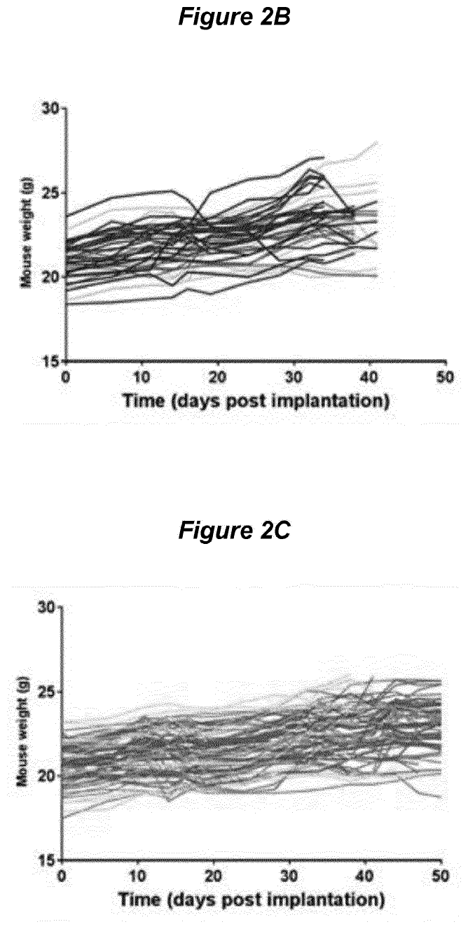

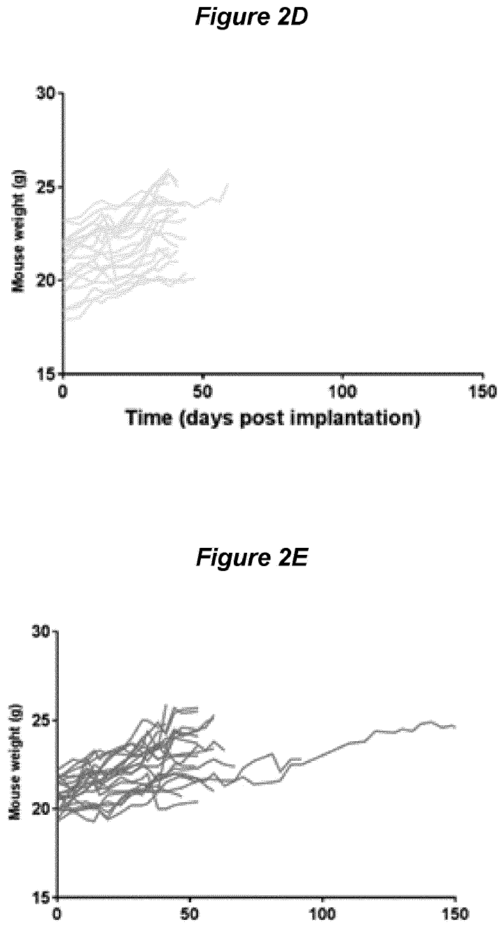

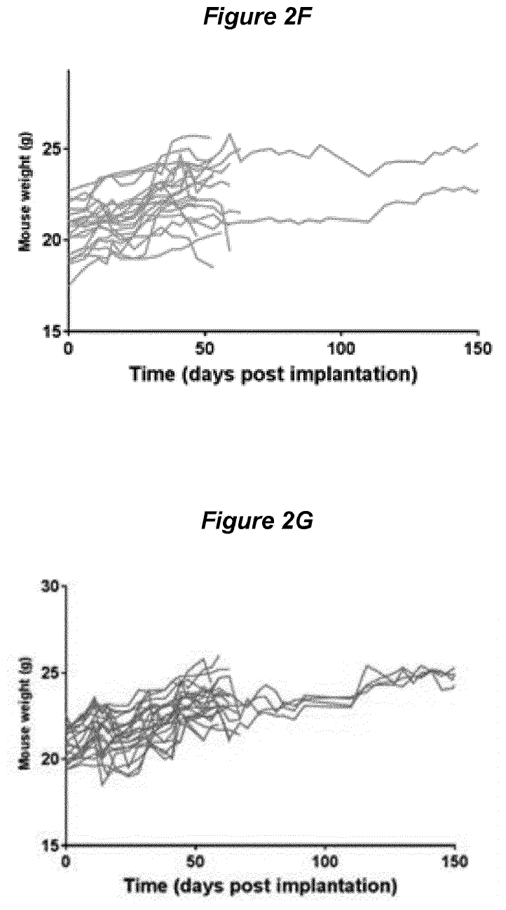

In another embodiment, the anti-AXL antibody is an antibody as described in international patent publication WO/2011/159980, the contents of which is hereby incorporated by reference, particularly the YW327.6S2 antibody as shown in FIG. 2, FIGURE page 6 (of 24).

In another embodiment, the anti-AXL antibody is an antibody as described in international patent publication WO/2012/175691, the contents of which is hereby incorporated by reference, particularly as shown at page 5.

In another embodiment, the anti-AXL antibody is an antibody as described in international patent publication WO/2012/175692, the contents of which is hereby incorporated by reference, particularly as shown at pages 4-5.

In another embodiment, the anti-AXL antibody is an antibody as described in international patent publication WO/2009/062690, the contents of which is hereby incorporated by reference.

In another embodiment, the anti-AXL antibody is an antibody as described in international patent publication WO/2010/130751, the contents of which is hereby incorporated by reference, particularly as shown at pages 1-17 (of 78).

In another embodiment, the anti-AXL antibody is an antibody as described in international patent publication WO/2013/064685, the contents of which is hereby incorporated by reference, particularly the 1613F12 antibody described therein as shown at, for example, Examples 6 to 8.

In another embodiment, the anti-AXL antibody is an antibody as described in international patent publication WO/2014/068139, the contents of which is hereby incorporated by reference, particularly the 110D7, 1003A2, and 1024G11 antibodies described therein as shown at, for example, Examples 6 to 8.

In another embodiment, the anti-AXL antibody is an antibody as described in international patent publication WO/2016/097370, the contents of which is hereby incorporated by reference, particularly the murine 10G5 and 10C9 antibodies described therein as shown at, for example, Examples 6 to 8.

In another embodiment, the anti-AXL antibody is an antibody as described in international patent publication WO/2017/220695, the contents of which is hereby incorporated by reference, particularly the humanized 10G5 antibody described therein as shown at, for example, SEQ ID NO. 1 to 10.

### PREFERRED EMBODIMENTS

Preferably, the anti-AXL antibody is an antibody as described in WO/2016/097370, WO/20171220695, WO/2015/193428, WO/2016/166296, WO/2015/193430, WO/2011/159980, WO12013/064685, or WO/2014/068139 (the contents of each of which is hereby incorporated by reference).

More preferably, the anti-AXL antibody is an antibody as described in WO/2016/097370, WO/2017/220695, WO/2011/159980, WO/2013/064685, or WO/2014/068139 (the contents of each of which is hereby incorporated by reference).

Most preferably the anti-AXL antibody is an antibody as described in WO/2017/220695, particularly the humanized 10G5 antibody described therein as shown at, for example, Examples 6 to 8.

In some embodiments, the anti-AXL antibody comprises the 6 CDRs having the sequences set out herein in SEQ ID Nos. 1 to 6.

In some embodiments, the anti-AXL antibody comprises the 6 CDRs having the sequences set out herein in SEQ ID Nos. 7 to 12.

In some embodiments, the anti-AXL antibody comprises a VH domain having the sequence set out herein in either one of SEQ ID Nos. 13 or 14. In some embodiments the antibody further comprises a VL domain having the sequence set out herein in either one of SEQ ID Nos. 15 or 16.

In some embodiments, the anti-AXL antibody is tilvestamab (BGB149).

Anti-AXL Antibody-Drug Conjugates

In some embodiments the AXL inhibitor is an anti-AXL antibody-drug conjugate (ADC) or immunoconjugate. In some embodiments, the anti-AXL ADC comprises one of the anti-AXL antibodies described above (or a functional fragment thereof). In some embodiments, the anti-AXL ADC may comprise an antibody as described in WO/2016/097370, WO/2017/220695, WO/2015/193428, WO/20161166296, WO/2015/193430, WO/2011/159980, WO/2013/064685, or WO/2014/068139 (the contents of each of which is hereby incorporated by reference). More preferably, the anti-AXL ADC may comprise an antibody as described in WO/2016/097370, WO/20171220695, WO/2011/159980, WO/2013/064685, or WO/2014/068139 (the contents of each of which is hereby incorporated by reference).

In some preferred embodiments the anti-AXL ADC comprises: an antibody as described in WO/2017/220695, particularly the humanized 10G5 antibody described therein as shown at, for example, Examples 6 to 8; the 6 CDRs having the sequences set out herein in SEQ ID Nos. 1 to 6; the 6 CDRs having the sequences set out herein in SEQ ID Nos. 7 to 12; a VH domain having the sequence set out herein in either one of SEQ ID Nos. 13 or 14 and further comprises a VL domain having the sequence set out herein in either one of SEQ ID Nos. 15 or 16; or the anti-AXL antibody tilvestamab (BGB149).

Immune Checkpoint Modulators (ICMs)

In the disclosed methods of treating an AXL-related disease, immune checkpoint inhibitors function to modulate the immune response to the AXL-related disease. This may be achieved in a number of ways, such as increasing the activity of stimulatory pathways and decreasing the activity of inhibitory pathways.

Immune responses to AXL-related diseases such as cancer are known to be able control tumour growth and in some cases lead to elimination of tumours. Therapeutic targeting of tumor immune regulators has resulted in the development of successful immunotherapeutic approaches for cancer treatment—for example agents blocking the activity of negative regulators of T cell immunity, such as a cytotoxic T-lymphocyte antigen 4 (CTLA-4) and programmed death receptor-1 (PD-1).

In some embodiments the immune checkpoint modulator (ICM) may be an immune checkpoint inhibitor (ICI). For example, an agent which acts at T cell co-inhibitory receptors, such as CTLA-4, PD-1, PD-L1, BTLA, TIM-3, VISTA, LAG-3, and TIGIT.

In some embodiments the immune checkpoint modulator (ICM) may be a T cell co-stimulatory agonist. For example, an agonist of a T-cell co-stimulatory receptor such as CD28, ICOS, 4-1BB, OX40, GITR, CD27, TWEAKR, HVEM, and TIM-1.

In some embodiments the immune checkpoint modulator (ICM) may act at dendritic cell co-stimulatory receptors, such as CD40 and 4-1BB.

In some embodiments, the immune checkpoint modulator may be an immune checkpoint modulating antibody. In some embodiments the immune checkpoint modulator may be selected from the group consisting of: anti-CTLA-4 antibodies, anti-PD-1 antibodies, anti-PD-L1 antibodies, anti-4-1 BB antibodies, anti-OX-40 antibodies, anti-GITR antibodies, anti-CD27 antibodies, anti-CD28 antibodies, anti-CD40 antibodies, anti-LAG3 antibodies, anti-ICOS antibodies, anti-TWEAKR antibodies, anti-HVEM antibodies, anti-TIM-1 antibodies, anti-TIM-3 antibodies, anti-VISTA antibodies, and anti-TIGIT antibodies.

In some preferred embodiments the immune checkpoint modulator may be selected from the group consisting of: anti-CTLA-4 antibodies, anti-PD-1 antibodies, anti-PD-L1 antibodies, anti-4-1 BB antibodies, anti-OX-40 antibodies, anti-GITR antibodies, anti-CD27 antibodies, anti-CD40 antibodies, and anti-LAG3 antibodies. In some particularly preferred embodiments the immune checkpoint modulator may be selected from the group consisting of: anti-CTLA-4 antibodies, anti-PD-1 antibodies, and anti-PD-L1 antibodies.

Examples of ICMs suitable for use in the methods described herein include ipilimumab, tremelimumab, pembrolizumab, nivolumab, and urelumab, and those which can be identified by the drug candidate identifiers AMP-514/MEDI0680 (MedImmune/AstraZeneca), MPDL3280A (Genentech/Roche), MEDI4736 (MedImmune/AstraZeneca), MSB0010718C (EMD Serono), BMS-936559 (Bristol-Myers Squibb), PF-05082566 (Pfizer), MEDI6469 (MedImmune/AstraZeneca), MEDI6383 (rOX40L; MedImmune/AstraZeneca), MOXR0916 (Genentech/Roche), TRX518 (Tolerx), CDX-1127 (Celldex), CP-870,893 (Genentech/Roche), and BMS-986016 (Bristol-Myers Squibb) (preferably ipilimumab, tremelimumab, pembrolizumab, and nivolumab).

In some embodiments, the anti-GITR antibody or GITR agonist is selected from MEDI1873, TRX518, GWN323, MK-1248, MK 4166, BMS-986156 and INCAGN1876.

In some embodiments, the anti-OX40 antibody or OX40 agonist is selected from MEDI0562, MEDI6383, MOXR0916, RG7888, OX40mAb24, INCAGN1949, GSK3174998, and PF-04518600.

In some preferred embodiments of the disclosed methods, two or more immune checkpoint modulators may be administered. Results have shown that an improved synergistic effect can be obtained when at least two different immune checkpoint (activity) modulators are employed, especially when such immune checkpoint (activity) modulators act at different cell receptor sub-types. For example, the combination of at least one immune checkpoint inhibitor and at least one T cell co-stimulatory receptor agonist or dendritic cell co-stimulatory receptor agonist.

Preferably, at least one of the two or more immune checkpoint (activity) modulators is an anti-CTLA-4 antibody, an anti-PD-1 antibody, or an anti-PD-L1 antibody. In particular, the combination of an anti-CTLA-4 antibody and an anti-PD-1 antibody has proven to be particularly effective.

In some preferred embodiments the two or more immune checkpoint (activity) modulators may include: (i) an immune checkpoint inhibitor, and (ii) a T cell co-stimulatory receptor agonist or a dendritic cell co-stimulatory receptor agonist. In some embodiments the two or more immune checkpoint (activity) modulators may include: (i) an anti-CTLA-4 antibody; and/or (ii) either an anti-PD-1 antibody or an anti-PD-L1 antibodies.

In some preferred embodiments the anti-CTLA-4 antibody is ipilimumab or tremelimumab.

In some preferred embodiments the anti-PD-1 antibody is pembrolizumab, nivolumab, spartalizumab Camrelizumab, Pidilizumab, or Cemiplimab. Preferably the anti-PD-1 antibody is pembrolizumab or nivolumab.

In some embodiments the anti-PDL1 antibody is Atezolizumab (CAS number 1380723-44-3), Avelumab (CAS number 1537032-82-8), or Durvalumab (CAS number 1428935-60-7).

In some embodiments the two or more immune checkpoint (activity) modulators may be administered concurrently. In other embodiments the two or more immune checkpoint (activity) modulators may be administered separately and/or sequentially in any order.

In some preferred embodiments the two or more immune checkpoint (activity) modulators may be ipilimumab and pembrolizumab.

Chemotherapeutic Agents

In the disclosed methods of treating an AXL-related disease, the chemotherapeutic agent may be any chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy.

In the disclosed methods of treating an AXL-related disease, the chemotherapeutic agents function to cause cell death of cancer cells (e.g. localised tumor cell death), the release of tumour antigens, and a subsequent immune response. Without wishing to be bound by theory, the authors believe that the chemotherapeutic agent induces cell death (apoptosis) and release of tumour antigens, upregulating IFN release and leading to a release of type I IFNs, which in turn activate AXL. Active AXL downregulates the IFN response and inhibits the immune response. AXL inhibition will therefore prevent inhibition of the immune response, and in turn potentiate the effect of the ICMs. Accordingly, the chemotherapeutic agent may be a chemotherapeutic agent which induces immunogenic cell death of cancer cells. The chemotherapeutic agent may be a chemotherapeutic agent which induces a type I interferon response.

Examples of chemotherapeutic agents which may be used in the disclosed methods include: Lenalidomide (REVLIMID®, Celgene), Vorinostat (ZOLINZA®, Merck), Panobinostat (FARYDAK®, Novartis), Mocetinostat (MGCD0103), Everolimus (ZORTRESS®, CERTICAN®, Novartis), Bendamustine (TREAKISYM®, RIBOMUSTIN®, LEVACT®, TREANDA®, Mundipharma International), erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo[4.3.0]nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®), Akti-112, HPPD, and rapamycin.

More examples of chemotherapeutic agents include: oxaliplatin (ELOXATIN®, Sanofi), bortezomib (VELCADE®, Millennium Pharm.), sutent (SUNITINIB®, SU 1248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, II), vandetanib (rINN, ZD6474, ZACTIMA®, AstraZeneca), chlorambucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylmelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g. calicheamicin, calicheamicin gamma1I, calicheamicin omegaI1 (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, nemorubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; antimetabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; eflornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above.

Examples of chemotherapeutic agents used in the treatment of anal cancer include: Gardasil, Gardasil 9, Recombinant Human Papillomavirus (HPV) Nonavalent Vaccine, Recombinant Human Papillomavirus (HPV) Quadrivalent Vaccine.

Examples of chemotherapeutic agents used in the treatment of bladder cancer include: Atezolizumab, Avelumab, Balversa (Erdafitinib), Bavencio (Avelumab), Cisplatin, Doxorubicin Hydrochloride, Durvalumab, Erdafitinib, Imfinzi (Durvalumab), Keytruda (Pembrolizumab), Nivolumab, Opdivo (Nivolumab), Pembrolizumab, Tecentriq (Atezolizumab), Thiotepa, Valrubicin, and Valstar (Valrubicin).

Examples of chemotherapeutic agents used in the treatment of bone cancer include: Cosmegen (Dactinomycin), Dactinomycin, Denosumab, Doxorubicin Hydrochloride, Methotrexate, Trexall (Methotrexate), and Xgeva (Denosumab).

Examples of chemotherapeutic agents used in the treatment of brain tumors include: Afinitor (Everolimus), Afinitor Disperz (Everolimus), Avastin (Bevacizumab), Bevacizumab, BiCNU (Carmustine), Carmustine, Carmustine Implant, Everolimus, Gliadel Wafer (Carmustine Implant), Lomustine, Mvasi (Bevacizumab), Temodar (Temozolomide), and Temozolomide.

Examples of chemotherapeutic agents used in the treatment of breast cancer include: Abemaciclib, Abraxane (Paclitaxel Albumin-stabilized Nanoparticle Formulation), Ado-Trastuzumab Emtansine, Afinitor (Everolimus), Afinitor Disperz (Everolimus), Alpelisib, Anastrozole, Aredia (Pamidronate Disodium), Arimidex (Anastrozole), Aromasin (Exemestane), Atezolizumab, Capecitabine, Cyclophosphamide, Docetaxel, Doxorubicin Hydrochloride, Ellence (Epirubicin Hydrochloride), Enhertu (Fam-Trastuzumab Deruxtecan-nxki), Epirubicin Hydrochloride, Eribulin Mesylate, Everolimus, Exemestane, 5-FU (Fluorouracil Injection), Fam-Trastuzumab Deruxtecan-nxki, Fareston (Toremifene), Faslodex (Fulvestrant), Femara (Letrozole), Fluorouracil Injection, Fulvestrant, Gemcitabine Hydrochloride, Gemzar (Gemcitabine Hydrochloride), Goserelin Acetate, Halaven (Eribulin Mesylate), Herceptin Hylecta (Trastuzumab and Hyaluronidase-oysk), Herceptin (Trastuzumab), Ibrance (Palbociclib), Ixabepilone, Ixempra (Ixabepilone), Kadcyla (Ado-Trastuzumab Emtansine), Kisqali (Ribociclib), Lapatinib Ditosylate, Letrozole, Lynparza (Olaparib), Megestrol Acetate, Methotrexate, Neratinib Maleate, Nerlynx (Neratinib Maleate), Olaparib, Paclitaxel, Paclitaxel Albumin-stabilized Nanoparticle Formulation, Palbociclib, Pamidronate Disodium, Perjeta (Pertuzumab), Pertuzumab, Piqray (Alpelisib), Ribociclib, Talazoparib Tosylate, Talzenna (Talazoparib Tosylate), Tamoxifen Citrate, Taxol (Paclitaxel), Taxotere (Docetaxel), Tecentriq (Atezolizumab), Thiotepa, Toremifene, Trastuzumab, Trastuzumab and Hyaluronidase-oysk, Trexall (Methotrexate), Tykerb (Lapatinib Ditosylate), Verzenio (Abemaciclib), Vinblastine Sulfate, Xeloda (Capecitabine), and Zoladex (Goserelin Acetate).

Examples of chemotherapeutic agents used in the treatment of cervical cancer include: Avastin (Bevacizumab), Bevacizumab, Bleomycin Sulfate, Hycamtin (Topotecan Hydrochloride), Keytruda (Pembrolizumab), Mvasi (Bevacizumab), Pembrolizumab, Topotecan Hydrochloride.

Examples of chemotherapeutic agents used in the treatment of colon and rectal cancer include: Avastin (Bevacizumab), Bevacizumab, Camptosar (Irinotecan Hydrochloride), Capecitabine, Cetuximab, Cyramza (Ramucirumab), Eloxatin (Oxaliplatin), Erbitux (Cetuximab), 5-FU (Fluorouracil Injection), Fluorouracil Injection, Ipilimumab, Irinotecan Hydrochloride, Keytruda (Pembrolizumab), Leucovorin Calcium, Lonsurf (Trifluridine and Tipiracil Hydrochloride), Mvasi (Bevacizumab), Nivolumab, Opdivo (Nivolumab), Oxaliplatin, Panitumumab, Pembrolizumab, Ramucirumab, Regorafenib, Stivarga (Regorafenib), Trifluridine and Tipiracil Hydrochloride, Vectibix (Panitumumab), Xeloda (Capecitabine), Yervoy (Ipilimumab), Zaltrap (Ziv-Aflibercept), Ziv-Aflibercept.

Examples of chemotherapeutic agents used in the treatment of ovarian, fallopian tube, or primary peritoneal cancer include: Alkeran (Melphalan), Avastin (Bevacizumab), Bevacizumab, Carboplatin, Cisplatin, Cyclophosphamide, Doxorubicin Hydrochloride, Doxil (Doxorubicin Hydrochloride Liposome), Doxorubicin Hydrochloride Liposome, Gemcitabine Hydrochloride, Gemzar (Gemcitabine Hydrochloride), Hycamtin (Topotecan Hydrochloride), Lynparza (Olaparib), Melphalan, Niraparib Tosylate Monohydrate, Olaparib, Paclitaxel, Rubraca (Rucaparib Camsylate), Rucaparib Camsylate, Taxol (Paclitaxel), Thiotepa, Topotecan Hydrochloride, Zejula (Niraparib Tosylate Monohydrate).

Examples of chemotherapeutic agents used in the treatment of non small cell lung cancer include: Abraxane (Paclitaxel Albumin-stabilized Nanoparticle Formulation), Afatinib Dimaleate, Afinitor (Everolimus), Afinitor Disperz (Everolimus), Alecensa (Alectinib), Alectinib, Alimta (Pemetrexed Disodium), Alunbrig (Brigatinib), Atezolizumab, Avastin (Bevacizumab), Bevacizumab, Brigatinib, Carboplatin, Ceritinib, Crizotinib, Cyramza (Ramucirumab), Dabrafenib Mesylate, Dacomitinib, Docetaxel, Doxorubicin Hydrochloride, Durvalumab, Entrectinib, Erlotinib Hydrochloride, Everolimus, Gefitinib, Gilotrif (Afatinib Dimaleate), Gemcitabine Hydrochloride, Gemzar (Gemcitabine Hydrochloride), Imfinzi (Durvalumab), Iressa (Gefitinib), Keytruda (Pembrolizumab), Lorbrena (Lorlatinib), Lorlatinib, Mechlorethamine Hydrochloride, Mekinist (Trametinib), Methotrexate, Mustargen (Mechlorethamine Hydrochloride), Mvasi (Bevacizumab), Navelbine (Vinorelbine Tartrate), Necitumumab, Nivolumab, Opdivo (Nivolumab), Osimertinib Mesylate, Paclitaxel, Paclitaxel Albumin-stabilized Nanoparticle Formulation, Paraplat (Carboplatin), Paraplatin (Carboplatin), Pembrolizumab, Pemetrexed Disodium, Portrazza (Necitumumab), Ramucirumab, Rozlytrek (Entrectinib), Tafinlar (Dabrafenib Mesylate), Tagrisso (Osimertinib Mesylate), Tarceva (Erlotinib Hydrochloride), Taxol (Paclitaxel), Taxotere (Docetaxel), Tecentriq (Atezolizumab), Trametinib, Trexall (Methotrexate), Vizimpro (Dacomitinib), Vinorelbine Tartrate, Xalkori (Crizotinib), Zykadia (Ceritinib).

Examples of chemotherapeutic agents used in the treatment of small cell lung cancer include: Afinitor (Everolimus), Atezolizumab, Doxorubicin Hydrochloride, Etopophos (Etoposide Phosphate), Etoposide, Etoposide Phosphate, Everolimus, Hycamtin (Topotecan Hydrochloride), Keytruda (Pembrolizumab), Mechlorethamine Hydrochloride, Methotrexate, Mustargen (Mechlorethamine Hydrochloride), Nivolumab, Opdivo (Nivolumab), Pembrolizumab, Tecentriq (Atezolizumab), Topotecan Hydrochloride, Trexall (Methotrexate).

Examples of chemotherapeutic agents used in the treatment of melanoma include: Aldesleukin, Binimetinib, Braftovi (Encorafenib), Cobimetinib, Cotellic (Cobimetinib), Dabrafenib Mesylate, Dacarbazine, Encorafenib, IL-2 (Aldesleukin), Imlygic (Talimogene Laherparepvec), Interleukin-2 (Aldesleukin), Intron A (Recombinant Interferon Alfa-2b), Ipilimumab, Keytruda (Pembrolizumab), Mekinist (Trametinib), Mektovi (Binimetinib), Nivolumab, Opdivo (Nivolumab), Peginterferon Alfa-2b, PEG-Intron (Peginterferon Alfa-2b), Pembrolizumab, Proleukin (Aldesleukin), Recombinant Interferon Alfa-2b, Sylatron (Peginterferon Alfa-2b), Tafinlar (Dabrafenib Mesylate), Talimogene Laherparepvec, Trametinib, Vemurafenib, Yervoy (Ipilimumab), Zelboraf (Vemurafenib).

Examples of chemotherapeutic agents used in the treatment of mesothelioma include: Alimta (Pemetrexed Disodium), and Pemetrexed Disodium.

Examples of chemotherapeutic agents used in the treatment of AML include: Arsenic Trioxide, Cerubidine (Daunorubicin Hydrochloride), Cyclophosphamide, Cytarabine, Daunorubicin Hydrochloride, Daunorubicin Hydrochloride and Cytarabine Liposome, Daurismo (Glasdegib Maleate), Dexamethasone, Doxorubicin Hydrochloride, Enasidenib Mesylate, Gemtuzumab Ozogamicin, Gilteritinib Fumarate, Glasdegib Maleate, Idamycin PFS (Idarubicin Hydrochloride), Idarubicin Hydrochloride, Idhifa (Enasidenib Mesylate), Ivosidenib, Midostaurin, Mitoxantrone Hydrochloride, Mylotarg (Gemtuzumab Ozogamicin), Rubidomycin (Daunorubicin Hydrochloride), Rydapt (Midostaurin), Tabloid (Thioguanine), Thioguanine, Tibsovo (Ivosidenib), Trisenox (Arsenic Trioxide), Venclexta (Venetoclax), Venetoclax, Vincristine Sulfate, Vyxeos (Daunorubicin Hydrochloride and Cytarabine Liposome), and Xospata (Gilteritinib Fumarate).

Examples of chemotherapeutic agents used in the treatment of pancreatic cancer include: Abraxane (Paclitaxel Albumin-stabilized Nanoparticle Formulation), Afinitor (Everolimus), Erlotinib Hydrochloride, Everolimus, 5-FU (Fluorouracil Injection), Fluorouracil Injection, Gemcitabine Hydrochloride, Gemzar (Gemcitabine Hydrochloride), Irinotecan Hydrochloride Liposome, Lynparza (Olaparib), Mitomycin C, Olaparib, Onivyde (Irinotecan Hydrochloride Liposome), Paclitaxel Albumin-stabilized Nanoparticle Formulation, Sunitinib Malate, Sutent (Sunitinib Malate), and Tarceva (Erlotinib Hydrochloride).

Examples of chemotherapeutic agents used in the treatment of renal cancer include: Afinitor (Everolimus), Afinitor Disperz (Everolimus), Aldesleukin, Avastin (Bevacizumab), Avelumab, Axitinib, Bavencio (Avelumab), Bevacizumab, Cabometyx (Cabozantinib-S-Malate), Cabozantinib-S-Malate, Everolimus, IL-2 (Aldesleukin), Inlyta (Axitinib), Interleukin-2 (Aldesleukin), Ipilimumab, Keytruda (Pembrolizumab), Lenvatinib Mesylate, Lenvima (Lenvatinib Mesylate), Mvasi (Bevacizumab), Nexavar (Sorafenib Tosylate), Nivolumab, Opdivo (Nivolumab), Pazopanib Hydrochloride, Pembrolizumab, Proleukin (Aldesleukin), Sorafenib Tosylate, Sunitinib Malate, Sutent (Sunitinib Malate), Temsirolimus, Torisel (Temsirolimus), Votrient (Pazopanib Hydrochloride), and Yervoy (Ipilimumab).

Example of chemotherapeutic agents used to treat solid tumors anywhere in the body include: Entrectinib, Keytruda (Pembrolizumab), Larotrectinib Sulfate, Rozlytrek (Entrectinib), and Vitrakvi (Larotrectinib Sulfate).

Combination treatments are also included in the definition of “chemotherapeutic agent” used herein. Examples of combination treatments of chemotherapeutic agents include: gemcitabine-cisplatin, MVAC (methotrexate, vinblastine sulfate, doxorubicin hydrochloride, cisplatin), PCV (procarbazine hydrochloride, lomustine, vincristine sulfate), AC (doxorubicin hydrochloride, cyclophosphamide), AC-T (doxorubicin hydrochloride, cyclophosphamide, paclitaxel), CAF (cyclophosphamide, doxorubicin hydrochloride, fluorouracil), CMF (cyclophosphamide, methotrexate, fluorouracil), FEC (fluorouracil, epirubicin hydrochloride, cyclophosphamide), TAC (docetaxel, doxorubicin hydrochloride, cyclophosphamide), CAPOX (capecitabine, oxaliplatin), FOLFIRI (leucovorin calcium, fluorouracil, irinotecan hydrochloride), FOLFIRI-Bevacizumab, FOLFIRI-Cetuximab, FOLFOX (leucovorin calcium, fluorouracil, oxaliplatin), FU-LV (fluorouracil, leucovorin calcium), XELIRI (capecitabine, irinotecan hydrochloride), XELOX (capecitabine, oxaliplatin), TPF (docetaxelm, cisplatin, fluorouracil), ABVD (doxorubicin hydrochloride, bleomycin, vinblastine sulfate, dacarbazine), ABVE (doxorubicin hydrochloride, bleomycin, vincristine sulfate, etoposide phosphate), ABVE-PC (doxorubicin hydrochloride, bleomycin, vincristine sulfate, etoposide phosphate, prednisone, cyclophosphamide), BEACOPP (bleomycin, etoposide phosphate, doxorubicin hydrochloride, cyclophosphamide, vincristine sulfate, procarbazine hydrochloride, prednisone), COPDAC (cyclophosphamide, vincristine sulfate, prednisone, dacarbazine), COPP cyclophosphamide, vincristine sulfate, procarbazine hydrochloride, prednisone), COPP-ABV cyclophosphamide, vincristine sulfate, procarbazine hydrochloride, prednisone, doxorubicin hydrochloride, bleomycin, vinblastine sulfate), ICE (ifosfamide, carboplatin, etoposide phosphate), MOPP (mechlorethamine hydrochloride, vincristine sulfate, procarbazine hydrochloride, prednisone), OEPA (vincristine sulfate, etoposide phosphate, prednisone, doxorubicin hydrochloride), OPPA (vincristine sulfate, procarbazine hydrochloride, prednisone, doxorubicin hydrochloride), STANFORD V (mechlorethamine hydrochloride, doxorubicin hydrochloride, vinblastine sulfate, vincristine sulfate, bleomycin, etoposide phosphate, prednisone), VAMP (vincristine sulfate, doxorubicin hydrochloride, methotrexate, prednisone), hyper-CVAD (cyclophosphamide, vincristine sulfate, doxorubicin hydrochloride, dexamethasone), ADE (cytarabine, daunorubicin hydrochloride, etoposide phosphate), chlorambucil-prednisone, CVP (cyclophosphamide, vincristine sulfate, prednisone), carboplatin-taxol, PAD (bortezomib, doxorubicin hydrochloride, dexamethasone), BuMel (busulfan, melphalan hydrochloride), CEM (carboplatin, etoposide phosphate, melphalan hydrochloride), CHP (doxorubicin, prednisone, cyclophosphamide), CHOP (doxorubicin, prednisone, cyclophosphamide, vincristine), EPOCH (etoposide phosphate, prednisone, vincristine sulfate, cyclophosphamide, doxorubicin hydrochloride) ICE (ifosfamide, carboplatin, etoposide phosphate) R-CHOP (rituximab, doxorubicin, prednisone, cyclophosphamide, vincristine), R-CVP (rituximab, cyclophosphamide, vincristine sulfate, prednisone) R-EPOCH (rituximab, etoposide phosphate, prednisone, vincristine sulfate, cyclophosphamide, doxorubicin hydrochloride), R-ICE (rituximab, ifosfamide, carboplatin, etoposide phosphate), BEP (bleomycin, etoposide phosphate, cisplatin), JEB (carboplatin, etoposide phosphate, bleomycin), PEB (cisplatin, etoposide phosphate, bleomycin) VAC (vincristine sulfate, dactinomycin, cyclophosphamide), VeIP (vinblastine sulfate, ifosfamide, cisplatin), Carboplatin/Doxil, Carboplatin/Gemcitabine, Carboplatin/Topotecan, Taxol/Avastin, FOLFIRINOX (leucovorin calcium, fluorouracil, irinotecan hydrochloride, oxaliplatin), Gemcitabine-cisplatin, gemcitabine oxaliplatin, OFF (oxaliplatin, fluorouracil, leucovorin calcium), CEV (carboplatin, etoposide phosphate, vincristine sulfate), and VIP (etoposide, ifosfamide, cisplatin).

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifene citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as MEK inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H-Ras, such as oblimersen (GENASENSE®, Genta Inc.); (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; topoisomerase 1 inhibitors such as LURTOTECAN®; ABARELIX® rmRH; (ix) anti-angiogenic agents such as bevacizumab (AVASTIN®, Genentech); and pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), ofatumumab (ARZERRA®, GSK), pertuzumab (PERJETA™, OMNITARG™, 2C4, Genentech), trastuzumab (HERCEPTIN®, Genentech), tositumomab (Bexxar, Corixia), MDX-060 (Medarex) and the antibody drug conjugate, gemtuzumab ozogamicin (MYLOTARG®, Wyeth).

Humanized monoclonal antibodies with therapeutic potential as chemotherapeutic agents in the combination treatments of the disclosure include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motavizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

Also included in the definition of “chemotherapeutic agent” are therapeutic immunoconjugates, such as antibody-drug conjugates (ADCs). ADCs are a class of biopharmaceutical drugs designed as targeted therapies, and comprise an antibody (or functional fragment thereof) linked to a payload or drug. The payload may be a cytotoxic drug, for example one or more of the anti-cancer chemotherapeutic agents described above. The antibody portion of the ADC specifically targets an antigen present on a target cell—for example a tumour antigen on tumour cells—delivering the payload to the target cell. The specific targeting of ADCs limits their side effects and gives a wider therapeutic window than other chemotherapeutic agents.

Accordingly, in some embodiments the chemotherapeutic agent may be an antibody-drug conjugate. The antibody-drug conjugate may comprise as its antibody portion one the antibodies disclosed elsewhere herein. The antibody-drug conjugate may comprise as its payload one or more of the anti-cancer chemotherapeutic agents disclosed elsewhere herein. The antibody-drug conjugate may comprise as its payload an anthracycline, such as doxorubicin, or a taxane, such as docetaxel. The antibody drug conjugate may be gemtuzumab ozogamicin, brentuximab vedotin, trastuzumab emtansine, inotuzumab ozogamicin, polatuzumab vedotin, enfortumab vedotin, trastuzumab deruxtecan, sacituzumab govitecan, belantamab mafodotin, or moxetumomab pasudotox.

Certain chemotherapeutic agents are known to influence pathways involved in the immune response. For example, the class of cytotoxic chemotherapeutic agents called anthracyclines are known to induce a Type I Interferon response mimicking immune responses to viruses, and the clinical response to anthracycline therapy correlates with a Type I IFN gene signature (Sistigue et al 2014; Zitvogel et al, 2015). As AXL serves as a key checkpoint for interferon (IFN) signaling, stimulating IFN signaling in the context of AXL-inhibition could lead to enhanced anticancer T cell responses during immune checkpoint inhibition.

Accordingly, in some embodiments the chemotherapeutic agent may be a chemotherapeutic agent which induces an immune response in the subject. In some embodiments the chemotherapeutic agent may be a chemotherapeutic agent which induces immunogenic cell death of cancer cells in the subject. In some embodiments the chemotherapeutic agent may be a chemotherapeutic agent which induces a type I interferon response in the subject.

In some embodiments the chemotherapeutic agent may be a STING (Stimulator of interferon response cGAMP interactor 1; STING1) agonist. In some such embodiments the chemotherapeutic agent may be E7766, GSK3745417, MK-1454, MK-2118, SB11285, ADU-S100, BMS-986301, or DMXAA.

In some preferred embodiments, the chemotherapeutic agent may be an anthracycline. In some such embodiments the chemotherapeutic agent may be selected from the group consisting of: Daunorubicin, Doxorubicin, Epirubicin, Idarubicin, Mitoxantrone, and Valrubicin. In some particularly preferred embodiments, the chemotherapeutic agent may be doxorubicin.

In some preferred embodiments, the chemotherapeutic agent may be a taxane. In some such embodiments, the chemotherapeutic agent may be selected from the group consisting of: docetaxel, paclitaxel, and abraxane. In some preferred embodiments, the chemotherapeutic agent may be docetaxel.

Radiotherapy

In the disclosed methods of treating an AXL-related disease, the terms “radiation therapy” or “radiotherapy” may refer to the medical use of ionizing radiation as part of cancer treatment to control or eradicate malignant cells. Radiotherapy may be used for curative, adjuvant, or palliative treatment. Suitable types of radiotherapy include conventional external beam radiotherapy, stereotactic radiation therapy (e.g., Axesse, Cyberknife, Gamma Knife, Novalis, Primatom, Synergy, X-Knife, TomoTherapy or Trilogy), Intensity-Modulated Radiation Therapy, particle therapy (e.g., proton therapy), brachytherapy, delivery of radioisotopes, intraoperative radiotherapy, Auger therapy, Volumetric modulated arc therapy (VMAT), Virtual simulation, 3-dimensional conformal radiation therapy, and intensity-modulated radiation therapy.

In some embodiments, radiatiotherapy uses high-energy radiation to shrink tumors and kill cancer cells. The radiation may be, for example, X-rays, gamma rays, or charged particles. Modes of cell killing through radiation include DNA damage either directly or by creating free radicals within cells that in turn damage DNA.

Radiation may be delivered by a machine outside the body (external-beam radiation therapy), or may come from radioactive material placed in the body near cancer cells (internal radiation therapy, also called brachy therapy). In one example of systemic radiation therapy, radioactive substances, such as radioactive iodine, are used which travel in the blood to kill cancer cells.

Preferably, the radiotherapy may be administered in a regime designed to minimize any immunosuppressive effects of the radiation. For example, preclinical evidence indicates high radiation doses above 12-18 Gy result in an attenuation of tumor immunogenicity (Vanpouille-Box C., et al., Nat Commun 2017; 8: 15618). In addition, it is known that circulating lymphocytes are particularly radiosensitive (see Yovino S., et al., Cancer Invest 2013; 31: 140-144); this indicates radiotherapy regimes aimed at stimulating an anti-tumour immune response should aim to minimise both (1) the amount of vasculature exposed in each treatment, and (2) the number of exposures in the treatment regime.

Radiation dosages may be fractionated and administered in sequence; for example, on consecutive days until the total desired radiation dose is delivered.

AXL-Related Disease

As referred to herein, an AXL-related disease is one which in which dysfunction of Axl expression or activity is a contributing factor. For example, the AXL-related disease may be one in which overexpression of AXL is a contributing factor. Overexpression of AXL and/or its ligand has been reported in a wide variety of solid tumor types, as well as in other disease states including vascular injury and kidney disease.

In some embodiments of the disclosure the AXL-related disease is a proliferative disease. A proliferative disease in one in which excessive proliferation of cells contributes to the pathogenesis of the disease. Exemplary proliferative diseases include: cancer, atherosclerosis, rheumatoid arthritis, psoriasis, idiopathic pulmonary fibrosis, scleroderma, and cirrhosis of the liver.

In some embodiments of the disclosure the AXL-related disease is a neoplastic disease. A neoplastic disease in one in which abnormal and excessive growth (termed neoplasia) of cells/tissue occurs. Neoplasia is the abnormal growth and proliferation of abnormal cells or abnormal amounts of cells, which can be due to a benign or malignant process. Exemplary neoplastic diseases include: myeloproliferative diseases, myelodysplastic syndromes (MDS), and acute myeloid leukemias (AML).

In some preferred embodiments of the disclosure, the AXL-related disease is cancer.

In some embodiments, the cancer may be one or more of the following cancers: Leukemias such as but not limited to acute myelocytic leukemias (AMLs) such as myeloblastic, promyelocytic, myelomonocytic, monocytic, erythroleukemia leukemias and myelodysplastic syndrome, acute leukemia, acute lymphocytic leukemia, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, metastatic cancers, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer, including, but not limited to, adenocarcinoma, lobular (small cell) carcinoma, intraductal carcinoma, medullary breast cancer, mucinous breast cancer, tubular breast cancer, papillary breast cancer, primary cancers, Paget's disease, and inflammatory breast cancer; adrenal cancer such as but not limited to pheochromocytoma and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipidus; eye cancers such as but not limited to ocular melanoma such as iris melanoma, choroidal melanoma, and ciliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polyploid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; genital cancers such as penile cancer; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypernephroma, fibrosarcoma, transitional cell cancer (renal pelvis and/or ureter); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas. Preferably, the cancer is selected from acute myelocytic leukemia (AML), breast, melanoma, prostate, ovarian, colorectal, lung or glioma cancer; the cancer may be metastatic. Most preferably the cancer is acute myelocytic leukemias (AMLs).

In some embodiments, the cancer may be one or more solid cancer tumors, including, but not limited to, breast, renal, endometrial, ovarian, thyroid, and non-small cell lung carcinoma, melanoma, prostate carcinoma, sarcoma, gastric cancer and uveal melanoma; liquid tumors, including but not limited to, leukemias (particularly myeloid leukemias) and lymphomas; In some embodiments, the cancer may be one or more leukaemias such as but not limited to, acute leukemia, acute lymphocytic leukemia, acute myeloid leukemia, acute myelocytic leukaemias such as myeloblastic, promyelocytic, myelomonocytic, monocytic, erythroleukemia leukaemias and myelodysplastic syndrome, chronic leukaemias such as but not limited to, chronic myelocytic (granulocytic) leukemia, chronic lymphocytic leukemia, hairy cell leukemia; polycythemia vera; In some embodiments, the cancer may be one or more lymphomas such as but not limited to Hodgkin's disease, non-Hodgkin's disease.

In some preferred embodiments, the AXL-related disease may be a solid tumour. In other preferred embodiments, the AXL-related disease may be a cancer selected from the group consisting of: histocytoma, glioma, astrocytoma, osteoma, lung cancer, small-cell lung cancer, non-small-cell lung cancer, gastrointestinal cancer, bowel cancer, colon cancer, breast cancer, ovarian carcinoma, prostate cancer, testicular cancer, liver cancer, kidney cancer, urothelial carcinoma, bladder cancer, pancreas cancer, brain cancer, glioblastoma, sarcoma, osteosarcoma, Kaposi's sarcoma, melanoma, mesothelioma, lymphomas, and leukemias.

In some particularly preferred embodiments, the AXL-related disease may be a cancer selected from the group consisting of: breast cancer, lung cancer, non-small-cell lung cancer, melanoma, mesothelioma, acute myeloid leukemia (AML), myelodysplastic syndrome (MDS), pancreas cancer, kidney cancer, urothelial carcinoma, and glioblastoma. In some most preferred embodiments, the cancer may be breast cancer, melanoma, or lung cancer.

In some preferred embodiments the AXL-related disease may be a cancer or tumor having or expected to have low tumor mutation burden (TMB). Tumors may be classified as high TMB or low TMB based on the prevalence of somatic mutations in their genome. The skilled person, as part of their common general knowledge, is aware of cancers classified as high/low TMB. Similarly, the skilled person is aware of suitable techniques for assessing mutational load of tumours—for example the methods employed in Samstein et al (Nat Genet. 2019 February; 51(2):202-206).

In some preferred embodiments, the AXL-related disease may be a cancer or tumor having or expected to have low numbers of oncogenic driver mutations. The skilled person, as part of their common general knowledge, is aware of oncogenic driver mutations relevant to particular cancers, as well as means for determining these in a subject—for example the methods employed in Grosse et al (Diagn Pathol. 2019 Feb. 11; 14(1):18). For example, the skilled is aware, as part of their common general knowledge that EGFR, KRAS, ALK, RET, ROS1, BRAF, ERBB2, MET and PIK3CA mutational status are oncogenic drivers in lung adenocarcinoma.

Low TMB and oncogenic drivers are known to be associated with poor response to immunotherapy treatment across multiple cancer types (Samstein et al, Nat Genet. 2019 February; 51(2):202-206). These may therefore be particularly sensitive to the triple combination treatments of the invention (via the potentiated IFN response produced by AXL inhibition in combination with cytotoxic chemotherapy reported elsewhere herein). Thus, in some preferred embodiments, the AXL-related disease may be a cancer or tumor that is, or is expected to be, refractory, non-responsive, or otherwise not benefit from immunotherapy treatment alone—for example, treatment with one or more immune checkpoint modulator (ICM).

For example, the AXL-related disease may be a breast cancer, melanoma, or lung cancer having or expected to have low TMB and/or numbers of oncogenic driver mutations. The AXL-related disease may be a breast cancer, melanoma, or lung cancer that is, or is expected to be, refractory to, non-responsive to, or which otherwise does not benefit from immunotherapy treatment.

In some embodiments of the disclosure, the AXL-related disease may be selected from: endometriosis, vascular disease/injury (including but not limited to restenosis, atherosclerosis and thrombosis), psoriasis; visual impairment due to macular degeneration; diabetic retinopathy and retinopathy of prematurity; kidney disease (including but not limited to glomerulonephritis, diabetic nephropathy and renal transplant rejection), rheumatoid arthritis; osteoarthritis, osteoporosis and cataracts.

In some embodiments of the disclosure, the AXL-related disease may be selected from: Immune disorders, cardiovascular disorders, thrombosis, diabetes, immune checkpoint disorders, fibrotic disorders (fibrosis), or proliferative diseases such as cancer, particularly metastatic cancer. Furthermore, Axl is known to play a role in many cancers of epithelial origin.

In some embodiments of the disclosure, the AXL-related disease may be fibrosis (including but not limited to lung fibrosis and liver fibrosis) or a fibrotic disorder. Fibrotic disorders of interest include strabismus, scleroderma, keloid, Nephrogenic systemic fibrosis, pulmonary fibrosis, idiopathic pulmonary fibrosis (IPF), cystic fibrosis (CF), systemic sclerosis, cardiac fibrosis, non-alcoholic steatohepatitis (NASH), other types of liver fibrosis, primary biliary cirrhosis, renal fibrosis, cancer, and atherosclerosis. In these diseases, the chronic development of fibrosis in tissue leads to marked alterations in the architecture of the affected organs and subsequently cause defective organ function.

In some embodiments of the disclosure, the AXL-related disease may be an immune checkpoint disorder. Immune checkpoint disorders of interest include: Chronic viral infections, Melanoma, Colorectal cancer, Breast cancer, Ovarian cancer, Non-small cell lung cancer (NSCLC), Prostate cancer, Renal cell cancer, Pancreatic cancer, Esophagus cancer, Bladder cancer, Myeloma, Kidney cancer, Bladder cancer, Brain tumor, and Lymphoma.

Pattern of Administration

In view of the proposed mechanistic interaction of the combination treatment, the present authors believe that it may not be required to administer the AXL inhibitor, immune checkpoint modulator (ICM), and chemotherapeutic agent and/or radiotherapy to the subject simultaneously in order to achieve the enhanced efficacy discussed herein.

Accordingly, as used herein, “administration in combination” may mean concurrent administration or may mean separate and/or sequential administration in any order. Thus, in some embodiments of the disclosure, the AXL inhibitor, immune checkpoint modulator (ICM), and chemotherapeutic agent may be administered concurrently. In other embodiments the AXL inhibitor, immune checkpoint modulator (ICM), and chemotherapeutic agent may be administered separately and/or sequentially.

In some embodiments of the disclosure, the AXL inhibitor, immune checkpoint modulator (ICM), and radiotherapy may be administered concurrently. In other embodiments the AXL inhibitor, immune checkpoint modulator (ICM), and radiotherapy may be administered separately and/or sequentially. In some embodiments, the AXL inhibitor, immune checkpoint modulator (ICM), and chemotherapeutic agent and/or radiotherapy may be administered concurrently. In other embodiments the AXL inhibitor, immune checkpoint modulator (ICM), and chemotherapeutic agent and/or radiotherapy may be administered separately and/or sequentially.

In some embodiments, the AXL inhibitor may be administered concurrently with the immune checkpoint modulator (ICM) and/or the chemotherapeutic agent. In some embodiments, the AXL inhibitor may be administered concurrently with the immune checkpoint modulator (ICM) and/or the radiotherapy. In some embodiments, the AXL inhibitor may be administered concurrently with the immune checkpoint modulator (ICM) and/or the chemotherapeutic agent and radiotherapy. In some embodiments, the AXL inhibitor may be administered subsequent to administration of the immune checkpoint modulator (ICM) and/or subsequent to administration of the chemotherapeutic agent. In some embodiments, the AXL inhibitor may be administered subsequent to administration of the immune checkpoint modulator (ICM) and/or subsequent to administration of radiotherapy. In some embodiments, the AXL inhibitor may be administered subsequent to administration of the immune checkpoint modulator (ICM) and/or subsequent to administration of the chemotherapeutic agent and radiotherapy.

In some embodiments, the AXL inhibitor may be administered subsequent to administration of the immune checkpoint modulator (ICM) and the chemotherapeutic agent. In some embodiments, the AXL inhibitor may be administered subsequent to administration of the immune checkpoint modulator (ICM) and radiotherapy. In some embodiments, the AXL inhibitor may be administered subsequent to administration of the immune checkpoint modulator (ICM) and the chemotherapeutic agent and radiotherapy. In some other embodiments, the AXL inhibitor may be administered prior to administration of the immune checkpoint modulator (ICM) and/or prior to administration of the chemotherapeutic agent. In some embodiments, the AXL inhibitor may be administered prior to administration of the immune checkpoint modulator (ICM) and/or prior to administration of radiotherapy. In some other embodiments, the AXL inhibitor may be administered prior to administration of the immune checkpoint modulator (ICM) and/or prior to administration of the chemotherapeutic agent and radiotherapy.

In some embodiments, the AXL inhibitor may be administered prior to administration of the immune checkpoint modulator (ICM) and the chemotherapeutic agent. In some embodiments, the AXL inhibitor may be administered prior to administration of the immune checkpoint modulator (ICM) and radiotherapy. In some embodiments, the AXL inhibitor may be administered prior to administration of the immune checkpoint modulator (ICM) and the chemotherapeutic agent and radiotherapy.

In some embodiments, the AXL inhibitor may be administered subsequent to administration of the chemotherapeutic agent, and the immune checkpoint modulator (ICM) may be administered subsequent to administration of the AXL inhibitor. In some embodiments, the AXL inhibitor may be administered prior to administration of the chemotherapeutic agent, and the immune checkpoint modulator (ICM) may be administered prior to administration of the AXL inhibitor. In some embodiments, the AXL inhibitor may be administered subsequent to administration of radiotherapy, and the immune checkpoint modulator (ICM) may be administered subsequent to administration of the AXL inhibitor. In some embodiments, the AXL inhibitor may be administered prior to administration of radiotherapy, and the immune checkpoint modulator (ICM) may be administered prior to administration of the AXL inhibitor.

In some embodiments, the immune checkpoint modulator (ICM) may be administered subsequent to administration of the AXL inhibitor and/or subsequent to administration of the chemotherapeutic agent and/or radiotherapy. In some embodiments, the immune checkpoint modulator (ICM) may be administered subsequent to administration of the AXL inhibitor and the chemotherapeutic agent and/or radiotherapy. In some other embodiments, the immune checkpoint modulator (ICM) may be administered prior to administration of the AXL inhibitor and/or prior to administration of the chemotherapeutic agent and/or radiotherapy. In some embodiments, the immune checkpoint modulator (ICM) may be administered prior to administration of the AXL inhibitor and the chemotherapeutic agent and/or radiotherapy. In some embodiments, the immune checkpoint modulator (ICM) may be administered subsequent to administration of the AXL inhibitor, and the chemotherapeutic agent and/or radiotherapy may be administered subsequent to administration of the immune checkpoint modulator (ICM). In some embodiments, the immune checkpoint modulator (ICM) may be administered prior to administration of the AXL inhibitor, and the chemotherapeutic agent and/or radiotherapy may be administered prior to administration of the immune checkpoint modulator (ICM).

In some embodiments, the chemotherapeutic agent and/radiotherapy may be administered subsequent to administration of the AXL inhibitor and/or subsequent to administration of the immune checkpoint modulator (ICM). In some embodiments, the chemotherapeutic agent and/or radiotherapy may be administered subsequent to administration of the AXL inhibitor and the immune checkpoint modulator (ICM). In some other embodiments, the chemotherapeutic agent and/or radiotherapy may be administered prior to administration of the AXL inhibitor and/or prior to administration of the immune checkpoint modulator (ICM). In some embodiments, the chemotherapeutic agent and/or radiotherapy may be administered prior to administration of the AXL inhibitor and the immune checkpoint modulator (ICM). In some embodiments, the chemotherapeutic agent and/or radiotherapy may be administered subsequent to administration of the AXL inhibitor, and the immune checkpoint modulator (ICM) may be administered subsequent to administration of the chemotherapeutic agent and/or radiotherapy. In some other embodiments, the chemotherapeutic agent and/or radiotherapy may be administered prior to administration of the AXL inhibitor, and the immune checkpoint modulator (ICM) may be administered prior to administration of the chemotherapeutic agent and/or radiotherapy.

In some embodiments of the disclosed methods of treating an AXL-related disease, the method comprises: administering the AXL inhibitor to the subject, when the immune checkpoint modulator (ICM) has been, is, or will be, administered to the subject; and/or administering the AXL inhibitor to the subject, when the chemotherapeutic agent and/or radiotherapy has been, is, or will be, administered to the subject.

In some embodiments of the disclosed methods of treating an AXL-related disease, the method comprises: administering the immune checkpoint modulator (ICM) to the subject, when the AXL inhibitor has been, is, or will be, administered to the subject; and/or administering the immune checkpoint modulator (ICM) to the subject, when the chemotherapeutic agent and/or radiotherapy has been, is, or will be, administered to the subject.

In some embodiments of the disclosed methods of treating an AXL-related disease, the method comprises: administering the chemotherapeutic agent and/or radiotherapy to the subject, when the AXL inhibitor has been, is, or will be, administered to the subject; and/or administering the chemotherapeutic agent and/or radiotherapy to the subject, wherein the immune checkpoint modulator (ICM) has been, is, or will be, administered to the subject.

In embodiments where the AXL inhibitor, immune checkpoint modulator (ICM), and chemotherapeutic agent and/or radiotherapy are not administered concurrently, preferably the AXL inhibitor and ICM are administered to the subject no more than 3 weeks apart, such as no more than 1 week apart, no more than 48 hours apart, or no more than 24 hours apart. In such embodiments, preferably the AXL inhibitor and chemotherapeutic agent and/or radiotherapy are administered to the subject no more than 4 weeks apart, such as no more than 3 weeks apart, no more than 1 week apart, no more than 48 hours apart, or no more than 24 hours apart.

Similarly, in embodiments where only one agent is administered as part of the described method (for example, methods involving administering an AXL inhibitor to a subject wherein an ICM has been or will be administered to the subject, and/or a chemotherapeutic agent and/or radiotherapy has been or will be administered to the subject) the method typically involves administering the AXL inhibitor to the subject no more than 3 weeks before/after the ICM and/or chemotherapeutic agent and/or radiotherapy has been or will be administered—such as no more than 1 week before/after, no more than 48 hours before/after, or no more than 24 hours before/after.

In some embodiments of the disclosed methods of treating an AXL-related disease, the Axl inhibitor may be administered to the subject daily, or every 2, 3, 4, 5, 6, or 7 days. In some embodiments in which the Axl inhibitor is bemcentinib, the Axl inhibitor is preferably administered to the subject daily.

In some embodiments of the disclosed methods of treating an AXL-related disease, the immune checkpoint modulator (ICM) may be administered to the subject every 1, 2, 3, 4, 5, 6, or 7 weeks. In some preferred embodiments the immune checkpoint modulator (ICM) may be administered to the subject every 3 or 4 weeks. In some embodiments in which the immune checkpoint modulator (ICM) is pembrolizumab, the immune checkpoint modulator (ICM) is preferably administered to the subject every 3 weeks. In some embodiments in which the immune checkpoint modulator (ICM) is durvalumab, the immune checkpoint modulator (ICM) is preferably administered to the subject every 4 weeks. In some embodiments in which the immune checkpoint modulator (ICM) is durvalumab and tremelimumab, the immune checkpoint modulator (ICM) is preferably administered to the subject every 4 weeks. In some embodiments in which the immune checkpoint modulator (ICM) is ipilimumab and nivolumab, the immune checkpoint modulator (ICM) is preferably administered to the subject every 2, 3, or 4 weeks.

In some embodiments of the disclosed methods of treating an AXL-related disease, the chemotherapeutic agent may be administered to the subject every 1, 2, 3, 4, 5, 6, or 7 weeks. In some preferred embodiments the chemotherapeutic agent may be administered to the subject every 3 or 4 weeks. In some embodiments in which the chemotherapeutic agent is doxorubicin, the chemotherapeutic agent is preferably administered to the subject every 3 weeks. In some embodiments in which the chemotherapeutic agent is doxorubicin in pegylated liposomal form, the chemotherapeutic agent is preferably administered to the subject every 4 weeks.

In some preferred embodiments of the disclosed methods of treating an AXL-related disease, the Axl inhibitor is administered to the subject daily, the immune checkpoint modulator (ICM) is administered to the subject every 4 weeks, and the chemotherapeutic agent is administered to the subject every 3 weeks.

Methods of Treatment

As outlined above, the present disclosure provides a method of treating an AXL-related disease, the method comprising administering to a subject in need thereof a therapeutically effective amount of an AXL inhibitor. In the disclosed methods of treating an AXL-related disease, the AXL inhibitor may be administered in combination with: an immune checkpoint modulator (ICM); and, a chemotherapeutic agent. In other embodiments, the AXL inhibitor may be administered in combination with: an immune checkpoint modulator (ICM); and, radiotherapy. In yet other embodiments, the AXL inhibitor may be used in combination with: an immune checkpoint modulator (ICM); a chemotherapeutic agent; and, radiotherapy. As used herein, “administration in combination” may mean concurrent administration or may mean separate and/or sequential administration in any order.

Thus, the present disclosure provides a method of treating an AXL-related disease, the method comprising administering to a subject in need thereof a therapeutically effective amount of an AXL inhibitor, wherein the AXL inhibitor is administered in combination with: an immune checkpoint modulator (ICM); and, a chemotherapeutic agent and/or radiotherapy. The disclosure also provides a method of treating an AXL-related disease, the method comprising administering to a subject in need thereof a therapeutically effective amount of an AXL inhibitor in concurrent, separate, or sequential combination with an immune checkpoint modulator (ICM) and a chemotherapeutic agent and/or radiotherapy.

The disclosed methods of treating an AXL-related disease thus include:

A method of treating an AXL-related disease, the method comprising administering to a subject in need thereof a therapeutically effective amount of an AXL inhibitor, wherein the AXL inhibitor is administered in combination with: an immune checkpoint modulator (ICM); and, a chemotherapeutic agent.

A method of treating an AXL-related disease, the method comprising administering to a subject in need thereof a therapeutically effective amount of an AXL inhibitor, wherein the AXL inhibitor is administered in combination with: an immune checkpoint modulator (ICM); and, radiotherapy.

A method of treating an AXL-related disease, the method comprising administering to a subject in need thereof a therapeutically effective amount of an AXL inhibitor, wherein the AXL inhibitor is administered in combination with: an immune checkpoint modulator (ICM); and, a chemotherapeutic agent and radiotherapy.

A method of treating an AXL-related disease, the method comprising administering to a subject in need thereof a therapeutically effective amount of an ICM, wherein the ICM is administered in combination with: an AXL inhibitor; and, a chemotherapeutic agent.

A method of treating an AXL-related disease, the method comprising administering to a subject in need thereof a therapeutically effective amount of an ICM, wherein the ICM is administered in combination with: an AXL inhibitor; and, radiotherapy.

A method of treating an AXL-related disease, the method comprising administering to a subject in need thereof a therapeutically effective amount of an ICM, wherein the ICM is administered in combination with: an AXL inhibitor; and, a chemotherapeutic agent and radiotherapy.

A method of treating an AXL-related disease, the method comprising administering to a subject in need thereof a therapeutically effective amount of a chemotherapeutic agent, wherein the chemotherapeutic agent is administered in combination with: an AXL inhibitor; and, an immune checkpoint modulator (ICM).

A method of treating an AXL-related disease, the method comprising administering to a subject in need thereof a therapeutically effective amount of a chemotherapeutic agent, wherein the chemotherapeutic agent is administered in combination with: an AXL inhibitor; an immune checkpoint modulator (ICM); and, radiotherapy.

A method of treating an AXL-related disease, the method comprising administering to a subject in need thereof a therapeutically effective amount of radiotherapy, wherein the chemotherapeutic agent is administered in combination with: an AXL inhibitor; and, an immune checkpoint modulator (ICM).

A method of treating an AXL-related disease, the method comprising administering to a subject in need thereof a therapeutically effective amount of radiotherapy, wherein the chemotherapeutic agent is administered in combination with: an AXL inhibitor; an immune checkpoint modulator (ICM); and, a chemotherapeutic agent.

A method of treating an AXL-related disease, the method comprising administering to a subject in need thereof a therapeutically effective amount of an AXL inhibitor and an ICM, wherein the AXL inhibitor and ICM are administered in combination with a chemotherapeutic agent.

A method of treating an AXL-related disease, the method comprising administering to a subject in need thereof a therapeutically effective amount of an AXL inhibitor and an ICM, wherein the AXL inhibitor and ICM are administered in combination with radiotherapy.

A method of treating an AXL-related disease, the method comprising administering to a subject in need thereof a therapeutically effective amount of an AXL inhibitor and an ICM, wherein the AXL inhibitor and ICM are administered in combination with a chemotherapeutic agent and radiotherapy.

A method of treating an AXL-related disease, the method comprising administering to a subject in need thereof a therapeutically effective amount of an AXL inhibitor and a chemotherapeutic agent, wherein the AXL inhibitor and chemotherapeutic agent are administered in combination with an immune checkpoint modulator (ICM).

A method of treating an AXL-related disease, the method comprising administering to a subject in need thereof a therapeutically effective amount of an AXL inhibitor and a chemotherapeutic agent, wherein the AXL inhibitor and chemotherapeutic agent are administered in combination with an immune checkpoint modulator (ICM) and radiotherapy.

A method of treating an AXL-related disease, the method comprising administering to a subject in need thereof a therapeutically effective amount of an immune checkpoint modulator (ICM) and a chemotherapeutic agent, wherein the immune checkpoint modulator (ICM) and chemotherapeutic agent are administered in combination with an AXL inhibitor.

A method of treating an AXL-related disease, the method comprising administering to a subject in need thereof a therapeutically effective amount of an immune checkpoint modulator (ICM) and a chemotherapeutic agent, wherein the immune checkpoint modulator (ICM) and chemotherapeutic agent are administered in combination with an AXL inhibitor and radiotherapy.

A method of treating an AXL-related disease, the method comprising administering to a subject in need thereof a therapeutically effective amount of an AXL inhibitor and radiotherapy, wherein the AXL inhibitor and radiotherapy are administered in combination with an immune checkpoint modulator (ICM).

A method of treating an AXL-related disease, the method comprising administering to a subject in need thereof a therapeutically effective amount of an AXL inhibitor and radiotherapy, wherein the AXL inhibitor and radiotherapy are administered in combination with an immune checkpoint modulator (ICM) and a chemotherapeutic agent.

A method of treating an AXL-related disease, the method comprising administering to a subject in need thereof a therapeutically effective amount of an AXL inhibitor, immune checkpoint modulator (ICM) and a chemotherapeutic agent.

A method of treating an AXL-related disease, the method comprising administering to a subject in need thereof a therapeutically effective amount of an AXL inhibitor, immune checkpoint modulator (ICM) and radiotherapy.

A method of treating an AXL-related disease, the method comprising administering to a subject in need thereof a therapeutically effective amount of an AXL inhibitor, immune checkpoint modulator (ICM), a chemotherapeutic agent, and radiotherapy.

In the disclosed methods, “administration in combination” may mean concurrent administration or may mean separate and/or sequential administration in any order.

Also provided are methods of treating an AXL-related disease, the method comprising administering to a subject in need thereof a therapeutically effective amount of an AXL inhibitor, wherein the subject has been or will be administered an immune checkpoint modulator and/or a chemotherapeutic agent and/or radiotherapy.

Also provided are methods of treating an AXL-related disease, the method comprising administering to a subject in need thereof a therapeutically effective amount of an immune checkpoint modulator (ICM), wherein the subject has been or will be administered an AXL inhibitor and/or a chemotherapeutic agent and/or radiotherapy.

Also provided are methods of treating an AXL-related disease, the method comprising administering to a subject in need thereof a therapeutically effective amount of a chemotherapeutic agent and/or radiotherapy, wherein the subject has been or will be administered an AXL inhibitor and/or an immune checkpoint modulator (ICM).

In some embodiments of the methods of the disclosure, the AXL inhibitor and ICM are administered to the subject no more than 4 weeks apart, such as no more than 3 weeks, no more than 1 week apart, no more than 48 hours apart, or no more than 24 hours apart. That is, in some embodiments the AXL inhibitor may be administered to the subject within 4 weeks, within 3 weeks, within 1 week, of the ICM being administered to the subject. For example, in some embodiments the AXL inhibitor may be administered to the subject 4 weeks, 3 weeks, or 1 week after administration of the ICM. In other embodiments, the the AXL inhibitor may be administered to the subject 4 weeks, 3 weeks, or 1 week before administration of the ICM.

In some embodiments of the methods of the disclosure, the AXL inhibitor and chemotherapeutic agent are administered to the subject no more than 4 weeks apart, such as no more than 3 weeks, no more than 1 week apart, no more than 48 hours apart, or no more than 24 hours apart. That is, in some embodiments the AXL inhibitor may be administered to the subject within 4 weeks, within 3 weeks, within 1 week, of the chemotherapeutic agent being administered to the subject. For example, in some embodiments the AXL inhibitor may be administered to the subject 4 weeks, 3 weeks, or 1 week after administration of the chemotherapeutic agent. In other embodiments, the AXL inhibitor may be administered to the subject 4 weeks, 3 weeks, or 1 week before administration of the chemotherapeutic agent.

In some embodiments of the methods of the disclosure, the ICM and chemotherapeutic agent are administered to the subject no more than 4 weeks apart, such as no more than 3 weeks, no more than 1 week apart, no more than 48 hours apart, or no more than 24 hours apart. That is, in some embodiments the ICM may be administered to the subject within 4 weeks, within 3 weeks, within 1 week, of the chemotherapeutic agent being administered to the subject. For example, in some embodiments the ICM may be administered to the subject 4 weeks, 3 weeks, or 1 week after administration of the chemotherapeutic agent. In other embodiments, the ICM may be administered to the subject 4 weeks, 3 weeks, or 1 week before administration of the chemotherapeutic agent.

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

Typically, in the methods of treatment described herein the agents (AXL inhibitors, ICMs, chemotherapeutic agents) are administered in a therapeutically or prophylactically effective amount. The term “therapeutically-effective amount” or “effective amount” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

Similarly, the term “prophylactically-effective amount,” as used herein, pertains to that amount of an active compound, or a material, composition or dosage from comprising an active compound, which is effective for producing some desired prophylactic effect, commensurate with a reasonable benefit/risk ratio, when administered in accordance with a desired treatment regimen.

Typically, the subjects treated are in need of the described treatment.

A “therapeutically effective amount” is an amount sufficient to show benefit to a subject. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors.

The disclosed methods of treatment may involve administration of the “triple combination” of the disclosure alone or in further combination with other treatments, either simultaneously or sequentially dependent upon the condition to be treated. Examples of treatments and therapies include, but are not limited to, chemotherapy (the administration of active agents, including, e.g. drugs, such as chemotherapeutics); surgery; and radiation therapy.

Compositions, Uses, and Kits

In addition to methods of treating an AXL-related disease, the present disclosure provides compositions comprising an AXL inhibitor, immune checkpoint modulator (ICM), and/or chemotherapeutic agent, as well as the use of such compositions in the disclosed methods of treating an Axl-related disease. Also provided are compositions comprising an AXL inhibitor, immune checkpoint modulator (ICM), and/or chemotherapeutic agent for use in a method of treatment according to the present disclosure.

Accordingly, the present disclosure provides an AXL inhibitor, an immune checkpoint modulator (ICM), and a chemotherapeutic agent, for use in a method of treatment according to the present disclosure. Also provided is: an AXL inhibitor for use in a method of treatment according to the present disclosure; an immune checkpoint modulator (ICM) for use in a method of treatment according to the present disclosure; a chemotherapeutic agent for use in a method of treatment according to the present disclosure; an AXL inhibitor and an immune checkpoint modulator (ICM) for use in a method of treatment according to the present disclosure; an AXL inhibitor and a chemotherapeutic agent for use in a method of treatment according to the present disclosure; and, an immune checkpoint modulator (ICM) and a chemotherapeutic agent for use in a method of treatment according to the present disclosure. Also provided is radiotherapy, for use in a method of treatment according to the present disclosure.

Thus, the present disclosure provides an AXL inhibitor, an immune checkpoint modulator (ICM), and a chemotherapeutic agent, for use in a method of treating an AXL-related disease. Also provided is an AXL inhibitor for use in a method of treating an AXL-related disease, the method comprising administering to a subject in need thereof a therapeutically effective amount of an AXL inhibitor, wherein the AXL inhibitor is administered in combination with: an immune checkpoint modulator (ICM); and, a chemotherapeutic agent. Also provided is an immune checkpoint modulator (ICM) for use in a method of treating an AXL-related disease, the method comprising administering to a subject in need thereof a therapeutically effective amount of an ICM, wherein the ICM is administered in combination with: an AXL inhibitor; and, a chemotherapeutic agent. Also provided is a chemotherapeutic agent for use in a method of treating an AXL-related disease, the method comprising administering to a subject in need thereof a therapeutically effective amount of a chemotherapeutic agent, wherein the chemotherapeutic agent is administered in combination with: an AXL inhibitor; and, an immune checkpoint modulator (ICM).

In some embodiments, the disclosure provides an AXL inhibitor and an immune checkpoint modulator (ICM) for use in a method of treating an AXL-related disease, the method comprising administering to a subject in need thereof a therapeutically effective amount of an AXL inhibitor and an ICM, wherein the AXL inhibitor and ICM are administered in combination with a chemotherapeutic agent. In some embodiments, the disclosure provides an AXL inhibitor and a chemotherapeutic agent for use in a method of treating an AXL-related disease, the method comprising administering to a subject in need thereof a therapeutically effective amount of an AXL inhibitor and a chemotherapeutic agent, wherein the AXL inhibitor and chemotherapeutic agent are administered in combination with an immune checkpoint modulator (ICM). In some embodiments, the disclosure provides an immune checkpoint modulator (ICM) and a chemotherapeutic agent for use in a method of treating an AXL-related disease, the method comprising administering to a subject in need thereof a therapeutically effective amount of an immune checkpoint modulator (ICM) and a chemotherapeutic agent, wherein the immune checkpoint modulator (ICM) and chemotherapeutic agent are administered in combination with an AXL inhibitor.

Also provided is the use of an AXL inhibitor, an immune checkpoint modulator (ICM), and a chemotherapeutic agent in the manufacture of a medicament for treating a disorder in a subject, wherein the treatment comprises a method of treatment according to the present disclosure. Also provided is: use of an Axl inhibitor in the manufacture of a medicament for treating a disorder in a subject, wherein the treatment comprises a method of treatment according to the present disclosure; use of an immune checkpoint modulator (ICM) in the manufacture of a medicament for treating a disorder in a subject, wherein the treatment comprises a method of treatment according to the present disclosure; use of a chemotherapeutic agent in the manufacture of a medicament for treating a disorder in a subject, wherein the treatment comprises a method of treatment according to the present disclosure; use of an AXL inhibitor and an immune checkpoint modulator (ICM) in the manufacture of a medicament for treating a disorder in a subject, wherein the treatment comprises a method of treatment according to the present disclosure; use of an AXL inhibitor and a chemotherapeutic agent in the manufacture of a medicament for treating a disorder in a subject, wherein the treatment comprises a method of treatment according to the present disclosure; and, use of an immune checkpoint modulator (ICM) and a chemotherapeutic agent in the manufacture of a medicament for treating a disorder in a subject, wherein the treatment comprises a method of treatment according to the present disclosure.

The present disclosure also provides a kit comprising an AXL inhibitor, an immune checkpoint modulator (ICM), and a chemotherapeutic agent, for use in a method of treating an Axl-related disease as disclosed herein.

Compositions according to the present disclosure are preferably pharmaceutical compositions. Pharmaceutical compositions according to the present disclosure, and for use in accordance with the present disclosure, may comprise, in addition to the active ingredient(s), (i.e. AXL inhibitors, immune checkpoint modulators (ICM), and/or chemotherapeutic agents), a pharmaceutically acceptable excipient, carrier, buffer, stabiliser or other materials well known to those skilled in the art. Such materials should be non-toxic and should not interfere with the efficacy of the active ingredient(s). The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

In some embodiments of the disclosure, the disclosed AXL inhibitor, ICM, chemotherapeutic agent, AXL inhibitor+ICM combination, ICM+chemotherapeutic agent combination, or AXL inhibitor+ICM+chemotherapeutic agent combination, may be comprised in a pharmaceutical composition, optionally further comprising a pharmaceutically acceptable excipient.

The present disclosure also provides such compositions for use in a method of treating an Axl-related disease, and use of such compositions in the manufacture of a medicament for treating a disorder in a subject, wherein the treatment comprises a method of treatment according to the present disclosure.

Subjects

The terms “subject”, “patient” and “individual” are used interchangeably herein. The subject may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutan, gibbon), or a human The subject may be any of its forms of development, for example, a foetus. In preferred embodiments, the subject is a human.

The subject may be a subject who has previously been treated with an immune checkpoint modulator (ICM), and was found to be non-responsive to or to otherwise not benefit from said treatment. The subject may be a subject who is suspected of being non-responsive to or who it is suspected will not benefit from treatment with an immune checkpoint modulator (ICM).

Subject Selection

Also provided by the present disclosure are methods of selecting a subject for treatment with one or more of an AXL inhibitor, an immune checkpoint modulator (ICM), and a chemotherapeutic agent—such methods include:

A method of selecting a subject for treatment with an AXL inhibitor, wherein a subject is selected for treatment if the subject has been, will be, or is being treated with an immune checkpoint modulator (ICM) and/or a chemotherapeutic agent. In some such embodiments, a subject is selected for treatment if the subject has been, will be, or is being treated with an immune checkpoint modulator (ICM) and a chemotherapeutic agent. In other embodiments, a subject is selected for treatment if the subject has been treated with an immune checkpoint modulator (ICM) and a chemotherapeutic agent. In some embodiments, a subject is selected for treatment if the subject is being treated with an immune checkpoint modulator (ICM) and a chemotherapeutic agent. In some embodiments, a subject is selected for treatment if the subject will be treated with an immune checkpoint modulator (ICM) and a chemotherapeutic agent. In other embodiments, a subject is selected for treatment if the subject has been treated with an immune checkpoint modulator (ICM), and is being treated with a chemotherapeutic agent. In some embodiments, a subject is selected for treatment if the subject has been treated with an immune checkpoint modulator (ICM), and will be treated with a chemotherapeutic agent. In other embodiments, a subject is selected for treatment if the subject has been treated with a chemotherapeutic agent, and is being treated with an immune checkpoint modulator (ICM). In some embodiments, a subject is selected for treatment if the subject has been treated with a chemotherapeutic agent, and will be treated with an immune checkpoint modulator (ICM).

A method of selecting a subject for treatment with an immune checkpoint modulator (ICM), wherein a subject is selected for treatment if the subject has been, will be, or is being treated with an AXL inhibitor and/or a chemotherapeutic agent. In some such embodiments, a subject is selected for treatment if the subject has been, will be, or is being treated with an AXL inhibitor and a chemotherapeutic agent. In other embodiments, a subject is selected for treatment if the subject has been treated with an AXL inhibitor and a chemotherapeutic agent. In some embodiments, a subject is selected for treatment if the subject is being treated with an AXL inhibitor and a chemotherapeutic agent. In some embodiments, a subject is selected for treatment if the subject will be treated with an AXL inhibitor and a chemotherapeutic agent. In other embodiments, a subject is selected for treatment if the subject has been treated with an AXL inhibitor, and is being treated with a chemotherapeutic agent. In some embodiments, a subject is selected for treatment if the subject has been treated with an AXL inhibitor, and will be treated with a chemotherapeutic agent. In other embodiments, a subject is selected for treatment if the subject has been treated with a chemotherapeutic agent, and is being treated with an AXL inhibitor. In some embodiments, a subject is selected for treatment if the subject has been treated with a chemotherapeutic agent, and will be treated with an AXL inhibitor.

A method of selecting a subject for treatment with a chemotherapeutic agent, wherein a subject is selected for treatment if the subject has been, will be, or is being treated with an AXL inhibitor and/or an immune checkpoint modulator (ICM). In some such embodiments, a subject is selected for treatment if the subject has been, will be, or is being treated with an AXL inhibitor and an immune checkpoint modulator (ICM). In other embodiments, a subject is selected for treatment if the subject has been treated with an AXL inhibitor and an immune checkpoint modulator (ICM). In some embodiments, a subject is selected for treatment if the subject is being treated with an AXL inhibitor and an immune checkpoint modulator (ICM). In some embodiments, a subject is selected for treatment if the subject will be treated with an AXL inhibitor and an immune checkpoint modulator (ICM). In other embodiments, a subject is selected for treatment if the subject has been treated with an AXL inhibitor, and is being treated with an immune checkpoint modulator (ICM). In some embodiments, a subject is selected for treatment if the subject has been treated with an AXL inhibitor, and will be treated with an immune checkpoint modulator (ICM). In other embodiments, a subject is selected for treatment if the subject has been treated with an immune checkpoint modulator (ICM), and is being treated with an AXL inhibitor. In some embodiments, a subject is selected for treatment if the subject has been treated with an immune checkpoint modulator (ICM), and will be treated with an AXL inhibitor.

A method of selecting a subject for treatment with an AXL inhibitor and an immune checkpoint modulator (ICM), wherein a subject is selected for treatment if the subject has been, will be, or is being treated with a chemotherapeutic agent.

A method of selecting a subject for treatment with an AXL inhibitor and a chemotherapeutic agent, wherein a subject is selected for treatment if the subject has been, will be, or is being treated with an immune checkpoint modulator (ICM).

A method of selecting a subject for treatment with an immune checkpoint modulator (ICM) and a chemotherapeutic agent, wherein a subject is selected for treatment if the subject has been, will be, or is being treated with an AXL inhibitor.

In some such methods, a subject may be selected for treatment if the subject was found to be refractory, non-responsive, or to otherwise not benefit from the recited treatments. For example, a subject may be selected for treatment if the subject was found to be refractory, non-responsive, or to otherwise not benefit from treatment with one or more immune checkpoint modulator (ICM).

The methods of selecting a subject for treatment with one or more of an AXL inhibitor, an immune checkpoint modulator (ICM), and a chemotherapeutic agent also include:

A method of selecting a subject for treatment in a method of treatment as disclosed herein, comprising: identifying subjects having low tumor mutation burden (TMB) and/or low numbers of oncogenic driver mutations; and, selecting thus identified subjects for treatment.

A method of selecting a subject for treatment in a method of treatment as disclosed herein, comprising: selecting a subject for treatment if the subject has been found to be, is suspected of being, or is refractory, non-responsive, or otherwise does not benefit from treatment with immunotherapy. A method of selecting a subject for treatment in a method of treatment as disclosed herein, comprising: selecting a subject for treatment if the subject has been found to be, is suspected of being, or is refractory, non-responsive, or otherwise does not benefit from treatment with an immune checkpoint modulator (ICM).

A method of selecting a subject for treatment in a method of treatment as disclosed herein, comprising: selecting a subject for treatment if the subject has been found to be, is suspected of being, or is refractory, non-responsive, or otherwise does not benefit from treatment with an immune checkpoint modulator (ICM) and a chemotherapeutic agent and/or radiotherapy.

A method of selecting a subject for treatment in a method of treatment as disclosed herein, comprising: selecting a subject for treatment if the subject has been found to be, is suspected of being, or is refractory, non-responsive, or otherwise does not benefit from treatment with an immune checkpoint modulator (ICM) and an AXL inhibitor.

A method of selecting a subject for treatment in a method of treatment as disclosed herein, comprising: selecting a subject for treatment if the subject has been found to be, is suspected of being, or is refractory, non-responsive, or otherwise does not benefit from treatment with an AXL inhibitor.

A method of selecting a subject for treatment in a method of treatment as disclosed herein, comprising: selecting a subject for treatment if the subject has been found to be, is suspected of being, or is refractory, non-responsive, or otherwise does not benefit from treatment with an AXL inhibitor and a chemotherapeutic agent and/or radiotherapy.

A method of selecting a subject for treatment in a method of treatment as disclosed herein, comprising: selecting a subject for treatment if the subject has been found to be, is suspected of being, or is refractory, non-responsive, or otherwise does not benefit from treatment with a chemotherapeutic agent and/or radiotherapy.

The methods of selecting a subject for treatment with one or more of an AXL inhibitor, an immune checkpoint modulator (ICM), and a chemotherapeutic agent also include:

A method of selecting a subject for treatment in a method of treatment as disclosed herein, comprising: identifying subjects having an increased activity or expression of AXL; and, selecting thus identified subjects for treatment.

A method of selecting a subject for treatment in a method of treatment as disclosed herein, comprising: identifying subjects having an AXL-related disease such as cancer, and having increased activity or expression of AXL; and, selecting thus identified subjects for treatment.

In some embodiments, increased activity or expression of AXL may be determined in a sample derived from a subject. In some embodiments, increased activity or expression of AXL is determined relative to a control. The skilled person is readily able to determine suitable controls against which to assess increased activity or expression of AXL—for example, the control may be a level of activity or expression of AXL in healthy subjects, or in subjects known to respond to or benefit from treatment with the combination therapies disclosed herein.

Increased expression or expression of AXL can be determined by any suitable method known in the art—for example, by determining the copy number of the gene encoding AXL relative to a control sample (wherein an increase in the copy number indicates an increased level of expression), or by determining the level of AXL mRNA or protein relative to a control sample.

In some embodiments, the disclosed methods of selecting a subject for treatment further comprise administering to the subject a therapeutically effective amount of an AXL inhibitor, an immune checkpoint modulator (ICM), and/or a chemotherapeutic agent as appropriate. Such methods form part of the disclosed method of treating an AXL-related disease.

Dosage

It will be appreciated by one of skill in the art that appropriate dosages of the AXL inhibitors, immune checkpoint modulators (ICM), chemotherapeutic agents, and compositions comprising these active elements, can vary from subject to subject. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the subject. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

In some cases, the dosage of AXL inhibitor may be determined by the expression of a first marker observed in a sample obtained from the subject. Thus, the level or localisation of expression of the first marker in the sample may be indicative that a higher or lower dose of AXL inhibitor is required. For example, a high expression level of the first marker may indicate that a higher dose of AXL inhibitor would be suitable. In some cases, a high expression level of the first marker may indicate a more aggressive therapy.

In some cases, the dosage of the ICM may be determined by the expression of a second marker observed in a sample obtained from the subject. Thus, the level or localisation of expression of the second marker in the sample may be indicative that a higher or lower dose of ICM is required. For example, a high expression level of the second marker may indicate that a higher dose of ICM would be suitable. In some cases, a high expression level of the second marker may indicate a more aggressive therapy.

In some cases, the dosage of the chemotherapeutic agent may be determined by the expression of a third marker observed in a sample obtained from the subject. Thus, the level or localisation of expression of the third marker in the sample may be indicative that a higher or lower dose of chemotherapeutic agent is required. For example, a high expression level of the third marker may indicate that a higher dose of chemotherapeutic agent would be suitable. In some cases, a high expression level of the third marker may indicate a more aggressive therapy.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of each active compound is in the range of about 100 ng to about 25 mg (more typically about 1 μg to about 10 mg) per kilogram body weight of the subject per day. Where the active compound is a salt, an ester, an amide, a prodrug, or the like, the amount administered is calculated on the basis of the parent compound and so the actual weight to be used is increased proportionately.

In some embodiments, each active compound is administered to a human subject according to the following dosage regime: about 100 mg, 3 times daily. In other embodiments, each active compound is administered to a human subject according to the following dosage regime: about 150 mg, 2 times daily. In other embodiments, each active compound is administered to a human subject according to the following dosage regime: about 200 mg, 2 times daily. In yet other embodiments, each active compound is administered to a human subject according to the following dosage regime: about 50 or about 75 mg, 3 or 4 times daily. In other embodiments, each active compound is administered to a human subject according to the following dosage regime: about 100 or about 125 mg, 2 times daily.

Antibodies

The term “antibody” herein is used in the broadest sense and specifically covers monoclonal antibodies, polyclonal antibodies, dimers, multimers, multispecific antibodies (e.g., bispecific antibodies), intact antibodies (also described as “full-length” antibodies) and antibody fragments, so long as they exhibit the desired biological activity, for example, the ability to bind a first target protein (Miller et al (2003) Jour. of Immunology 170:4854-4861). Antibodies may be murine, human, humanized, chimeric, or derived from other species such as rabbit, goat, sheep, horse or camel.

An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by Complementarity Determining Regions (CDRs) on multiple antibodies. Each antibody that specifically binds to a different epitope has a different structure. Thus, one antigen may have more than one corresponding antibody. An antibody may comprise a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass, or allotype (e.g. human G1 m1, G1m2, G1m3, non-G1 m1 [that, is any allotype other than G1 m1], G1m17, G2m23, G3m21, G3m28, G3m11, G3m5, G3m13, G3m14, G3m10, G3m15, G3m16, G3m6, G3m24, G3m26, G3m27, A2 m1, A2m2, Km1, Km2 and Km3) of immunoglobulin molecule. The immunoglobulins can be derived from any species, including human, murine, or rabbit origin.

“Antibody fragments” comprise a portion of a full length antibody, generally the antigen binding or variable region thereof. Examples of antibody fragments include Fab, Fab′, F(ab′)₂, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al (1975) Nature 256:495, or may be made by recombinant DNA methods (see, U.S. Pat. No. 4,816,567). The monoclonal antibodies may also be isolated from phage antibody libraries using the techniques described in Clackson et al (1991) Nature, 352:624-628; Marks et al (1991) J. Mol. Biol., 222:581-597 or from transgenic mice carrying a fully human immunoglobulin system (Lonberg (2008) Curr. Opinion 20(4):450-459).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies include “primatized” antibodies comprising variable domain antigen-binding sequences derived from a non-human primate (e.g. Old World Monkey or Ape) and human constant region sequences.

An “intact antibody” herein is one comprising VL and VH domains, as well as a light chain constant domain (CL) and heavy chain constant domains, CH1, CH2 and CH3. The constant domains may be native sequence constant domains (e.g. human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors such as B cell receptor and BCR.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

### Embodiments of the Disclosure

Certain preferred embodiments of the disclosure are as follows:

In some preferred embodiments the AXLi is bemcentinib, the ICM is a PD-1/PD-L1 inhibitor (such as Pembrolizumab or Durvalumab) and/or a CTLA-4 inhibitor (such as Ipilimumab or tremelimumab), and the chemotherapeutic agent is an anthracycline (such as doxorubicin).

In some preferred embodiments the AXLi is bemcentinib, the ICM is a PD-1/PD-L1 inhibitor (such as Pembrolizumab or Durvalumab) and/or a CTLA-4 inhibitor (such as Ipilimumab or tremelimumab), and the chemotherapeutic agent is a taxane (such as docetaxel).

In some preferred embodiments the AXL-related disease is cancer, such as breast cancer, lung cancer, non-small-cell lung cancer, melanoma, mesothelioma, acute myeloid leukemia (AML), myelodysplastic syndrome (MDS), pancreas cancer, kidney cancer, urothelial carcinoma, and glioblastoma. In some particularly preferred embodiments the cancer is breast cancer.

In some preferred embodiments: the AXLi is bemcentinib, the ICM is a PD-1/PD-L1 inhibitor (such as Pembrolizumab or Durvalumab) and/or a CTLA-4 inhibitor (such as Ipilimumab or tremelimumab), and the chemotherapeutic agent is an anthracycline (such as doxorubicin); and, the AXL-related disease is cancer (such as breast cancer or melanoma).

In some preferred embodiments the AXLi is bemcentinib, the ICM is a PD-1/PD-L1 inhibitor (such as Pembrolizumab or Durvalumab) and/or a CTLA-4 inhibitor (such as Ipilimumab or tremelimumab), and the chemotherapeutic agent is a taxane (such as docetaxel); and, the AXL-related disease is cancer (such as lung cancer).

In some preferred embodiments: the AXLi is administered prior to administration of the chemotherapeutic agent and prior to administration of the immune checkpoint modulator (ICM); and, the chemotherapeutic agent is administered prior to administration of the immune checkpoint modulator (ICM).

In some preferred embodiments: bemcentinib is administered prior to administration of doxorubicin and prior to administration of PD-1/PD-L1 inhibitor and CTLA-4 inhibitor; and, the doxorubicin is administered prior to administration of PD-1/PD-L1 inhibitor and CTLA-4 inhibitor.

In some preferred embodiments: the AXLi and chemotherapeutic agent are administered to the subject no more than 3 weeks apart, preferably no more than 1 week apart; and, the AXLi and ICM are administered to the subject no more than 3 weeks apart, preferably no more than 1 week apart.

In some preferred embodiments: bemcentenib and doxorubicin are administered to the subject no more than 3 weeks apart, preferably no more than 1 week apart; and, bemcentinib and PD-1/PD-L1 inhibitor and CTLA-4 inhibitor are administered to the subject no more than 3 weeks apart, preferably no more than 1 week apart.

In some preferred embodiments: the AXLi is administered to the subject daily; the ICM is administered to the subject every 3 weeks; and, the chemotherapeutic agent is administered to the subject every 3 weeks.

In some preferred embodiments: bemcentinib administered to the subject daily; PD-1/PD-L1 inhibitor and CTLA-4 inhibitor are administered to the subject every 3 weeks; and, doxorubicin is administered to the subject every 3 weeks.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

SEQUENCES

SEQ ID NO. 1 [10C9 Heavy CDR1]
DYNFTRYYIH
SEQ ID NO. 2 [10C9 Heavy CDR2]
WIYPGTGDSKYNEKFKG
SEQ ID NO. 3 [10C9 Heavy CDR3]
NGNYWYFDV
SEQ ID NO. 4 [10C9 Light CDR1]
RSSKSLLHSNGNTYLY
SEQ ID NO. 5 [10C9 Light CDR2]
RMSNLAS
SEQ ID NO. 6 [10C9 Light CDR3]
MQHREYPFT
SEQ ID NO. 7 [10G5 Heavy CDR1]
GYSFTDFYIN
SEQ ID NO. 8 [10G5 Heavy CDR2]
RIFPGGDNTYYNEKFKG
SEQ ID NO. 9 [10G5 Heavy CDR3]
RGLYYAMDY
SEQ ID NO. 10 [10G5 Light CDR1]
RSSQSLVHSNGIPYLH
SEQ ID NO. 11 [10G5 Light CDR2]
RVSNRFS
SEQ ID NO. 12 [10G5 Light CDR3]
SQGTHVPPT
SEQ ID NO. 13 [hu10G5 VH(GH1)]
EVQLVQSGAGLVQPGGSVRLSCAASGYSFTDFYINWRQAPGKGLEWIARIFPGGDNTYYNEKFKGRFT
LSADTSSSTAYLQLNSLRAEDTAVYYCARRGLYYAMDYWGQGTLVTVSS
SEQ ID NO. 14 [hu10G5 VH(GH2)]
EVQLVESGGGLVQPGGSLRLSCAASGYSFTDFYINWRQAPGKGLEWVARIFPGGDNTYYNEKFKGRF
TLSADTSKSTAYLQMNSLRAEDTAVYYCARRGLYYAMDYWGQGTLVTVSS
SEQ ID NO. 15 [hu10G5 VL(GL1)]
DIQMTQSPSSLSASVGDRVTITCRSSQSLVHSNGIPYLHWYQQKPGKAPKLLIYRVSNRFSGVPSRFSGS
GSGTDFTLTISSLQPEDFATYYCSQGTHVPPTFGQGTKVEIK
SEQ ID NO. 16 [hu10G5 VL(GL2)]
DIQMTQSPSSLSASVGDRVTITCRSSQSLVHSNGIPYLHWYQQKPGKAPKLLIYRVSNRFSGVPSRFSGS
RSGTDFTLTISSLQPEDFATYYCSQGTHVPPTFGQGTKVEIK
SEQ ID NO. 17 [10G5 GH1 Heavy chain]
EVQLVQSGAGLVQPGGSVRLSCAASGYSFTDFYINWRQAPGKGLEWIARIFPGGDNTYYNEKFKGRFT
LSADTSSSTAYLQLNSLRAEDTAVYYCARRGLYYAMDYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVDK
SRWQQGNVFSCSVMHEALHNHYTQKSLSLSPGK
SEQ ID NO. 18 [10G5 GH2 Heavy chain]
EVQLVESGGGLVQPGGSLRLSCAASGYSFTDFYINWWRQAPGKGLEWWARIFPGGDNTYYNEKFKGRF
TLSADTSKSTAYLQMNSLRAEDTAVYYCARRGLYYAMDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKKVEPKSCDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVDK
SRWQQGNVFSCSVMHEALHNHYTQKSLSLSPGK
SEQ ID NO. 19 [10G5 GL1 Light chain]
DIQMTQSPSSLSASVGDRVTITCRSSQSLVHSNGIPYLHWYQQKPGKAPKLLIYRVSNRFSGVPSRFSGS
GSGTDFTLTISSLQPEDFATYYCSQGTHVPPTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
SEQ ID NO. 20 [10G5 GL2 Light chain]
DIQMTQSPSSLSASVGDRVTITCRSSQSLVHSNGIPYLHWYQQKPGKAPKLLIYRVSNRFSGVPSRFSGS
RSGTDFTLTISSLQPEDFATYYCSQGTHVPPTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC

Statements of Disclosure

The following numbered statements, outlining aspects of the present disclosure, are part of the description.
101. A method of treating an AXL-related disease, the method comprising administering to a subject in need thereof a therapeutically effective amount of an AXL inhibitor, wherein the AXL inhibitor is administered in combination with: one or more immune checkpoint modulator (ICM); and, one or more chemotherapeutic agent and/or radiotherapy.

AXL Inhibitor
102. The method of statement 101, wherein the AXL inhibitor is a compound of formula (I) as set out in the description.
103. The method of statement 102, wherein the AXL inhibitor is selected from the group consisting of:
1-(6,7-dihydro-5H-benzo[6,7]cyclohepta[1,2-c]pyridazin-3-yl)-N³-(7-(pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-(S)-pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-(R)-pyrrolidin-1-yl)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-(3-fluoro-4-(4-(pyrrolidin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N⁵-(7-(pyrrolidin-1-yl)-6,7,8,9-tetrahydro-5H-benzo[7]annulene-1-yl)-1H-1,2,4-triazole-3,5-diamine;
1-(6,7-dihydro-5H-benzo[6,7]cyclohepta[1,2-c]pyridazin-3-yl)-N⁵-(7-(S)-pyrrolidin-1-yl-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(t-butoxycarbonylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(acetamido)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((2R)-2-(methoxycarbonyl)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4,4-difluoropiperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((methoxycarbonylmethyl)(methyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((2R)-2-(carboxy)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyrdazin-3-yl)-N³-(7-(4-(ethoxycarbonyl)piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(carboxy)piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((carboxymethyl)(methyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(ethoxycarbonylmethyl)piperazin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(carboxymethyl)piperazin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(pyrrolidin-1-yl)-6,7,8,9-tetrahydro-5H-benzo[7]annulene-1-yl)-1H-1,2,4-triazole-3,5-diamine;
1-(6,7-dihydro-5H-benzo[6,7]cyclohepta[1,2-c]pyridazin-3-yl)-N³-((7S)-7-amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7s)-7-(di(cyclopropylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((2-methylpropyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((propyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(dipropylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(diethylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(cyclohexylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(cyclopentylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((1-cyclopentylethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(2-propylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((3,3-dimethylbut-2-yl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((cyclohexylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(cyclohexylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((5-chlorothien-2-yl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((2-carboxyphenyl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((3-bromophenyl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(dimethylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(cyclobutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(3-pentylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((2,2-dimethylpropyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(cyclopentylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((cyclopentylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(bicyclo[2.2.1]hept-2-en-5-ylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((bicyclo[2.2.1]hept-2-en-5-ylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(3-methylbutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(3-methylbutyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(2-ethylbutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(but-2-enylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(butyl(but-2-enyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N⁵-((7S)-7-(t-butoxycarbonylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-amino-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(dimethylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(diethylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(dipropylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(di(cyclopropylmethyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(di(3-methylbutyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(cyclobutylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(cyclohexylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-((methylethyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(cyclopentylamino)-6,7,8,9-tetrahydro-5H-benzo[7]annulene-2-yl)-1H-1,2,4-triazole-3,5-diamine; and
1-(6,7-dihydro-5H-pyrido[2′,3′:6,7]cyclohepta[1,2-c]pyridazin-3-yl)-N³-((7S)-7-(2-butylamino)-6,7,8,9-tetrahydro-5H-benzo[7]annulene-2-yl)-1H-1,2,4-triazole-3,5-diamine;

or pharmaceutically acceptable salts thereof.
104. The method of statement 102, wherein the AXL inhibitor is 1-(6,7-dihydro-5H-benzo[6,7]cyclohepta[1,2-c]pyridazin-3-yl)-N³-((7-(S)-pyrrolidin-1-yl)-6,7,8,9-tetrahydro-5H-benzo[7]annulene-2-yl)-1H-1,2,4-triazole-3,5-diamine, or a pharmaceutically acceptable salt thereof.
105. The method of statement 102, wherein the AXL inhibitor is bemcentinib (BGB324/R428).
106. The method of statement 101, wherein the AXL inhibitor is selected from the group consisting of: dubermatinib (CAS No. 1341200-45-0; UNII 14D65TV20J); gilteritinib (CAS No. 1254053-43-4; UNII 66D92MGC8M); cabozantinib (CAS No. 849217-68-1; UNII 1C39JW444G); SG17079 (CAS No. 1239875-86-5); merestinib (CAS No. 1206799-15-6; UNII 50GS5K699E); amuvatinib (CAS No. 850879-09-3; UNII SO9S6QZB4R); bosutinib (CAS No. 380843-75-4; UNII 5018V4AEZ0); glesatinib (CAS No. 936694-12-1; UNII 7Q29OXD98N); foretinib (CAS No. 849217-64-7; UNII 81FH7VK1C4); and, TP0903 (CAS No. 1341200-45-0).
107. The method of statement 101, wherein the AXL inhibitor is an AXL inhibitor disclosed in WO2008/083367, WO2010/083465, or WO2012/028332.
108. The method of statement 101, wherein the AXL inhibitor is an anti-AXL antibody or anti-AXL antibody-drug conjugate which comprises an anti-AXL antibody.
109. The method of statement 108, wherein the antibody is an anti-AXL antibody disclosed in WO2015/193428, WO2015/193430, WO2016/097370, or WO2016/166296.
110. The method of statement 108, wherein the antibody is an anti-AXL antibody selected from the group consisting of: the 1613F12 antibody disclosed in WO2013/064685; the 110D7 antibody disclosed in WO2014/068139; the 1003A2 antibody disclosed in WO2014/068139; the 1024G11 antibody disclosed in WO2014/068139; the hu10G5 antibody disclosed in WO2017/220695; and, the YW327.6S2 antibody disclosed in WO2011/159980.
110. The method of statement 108, wherein the antibody comprises the 6 CDRs having the sequences of SEQ ID Nos. 1 to 6.
111. The method of statement 108, wherein the antibody comprises the 6 CDRs having the sequences of SEQ ID Nos. 7 to 12.
112. The method of statement 108, wherein the antibody comprises:
   a VH domain having the sequence of SEQ ID No. 13 and a VL domain having the sequence of SEQ ID NO. 15;
   a VH domain having the sequence of SEQ ID No. 13 and a VL domain having the sequence of SEQ ID NO. 16;
   a VH domain having the sequence of SEQ ID No. 14 and a VL domain having the sequence of SEQ ID NO. 15; or
   a VH domain having the sequence of SEQ ID No. 14 and a VL domain having the sequence of SEQ ID NO. 16.

Immune Checkpoint Modulators
113. The method of any preceding statement, wherein the one or more immune checkpoint modulator includes one or more immune checkpoint inhibitors (ICI).
114. The method of any preceding statement, wherein the one or more immune checkpoint modulator includes one or more immune checkpoint modulating antibody.
115. The method of statement 114, wherein one or more immune checkpoint modulating antibody is selected from the group consisting of: anti-CTLA-4 antibodies, anti-PD-1 antibodies, anti-PD-L1 antibodies, anti-4-1 BB antibodies, anti-OX-40 antibodies, anti-GITR antibodies, anti-CD27 antibodies, anti-CD28 antibodies, anti-CD40 antibodies, anti-LAG3 antibodies, anti-ICOS antibodies, anti-TWEAKR antibodies, anti-HVEM antibodies, anti-TIM-1 antibodies, anti-TIM-3 antibodies, anti-VISTA antibodies, and anti-TIGIT antibodies.
116. The method of statement 114, wherein one or more immune checkpoint modulating antibody is selected from the group consisting of: anti-CTLA-4 antibodies, anti-PD-1 antibodies, anti-PD-L1 antibodies, anti-4-1 BB antibodies, anti-OX-40 antibodies, anti-GITR antibodies, anti-CD27 antibodies, anti-CD40 antibodies, and anti-LAG3 antibodies.
117. The method of statement 114, wherein one or more immune checkpoint modulating antibody is selected from the group consisting of: anti-CTLA-4 antibodies, anti-PD-1 antibodies, and anti-PD-L1 antibodies.
118. The method of any preceding statement, wherein the one or more immune checkpoint modulator includes: one or more T-cell co-stimulatory agonist; and/or one or more dendritic cell co-stimulatory receptor agonist.
119. The method of any preceding statement, wherein the one or more immune checkpoint modulator includes at least two immune checkpoint modulators.
120. The method of any preceding statement, wherein the one or more immune checkpoint modulator includes: (i) an immune checkpoint inhibitor, and (ii) a T cell co-stimulatory receptor agonist or a dendritic cell co-stimulatory receptor agonist.
121. The method of any preceding statement, wherein the one or more immune checkpoint modulator includes: (i) an anti-CTLA-4 antibody; and, (ii) an anti-PD-1 antibody and/or an anti-PD-L1 antibody.
122. The method of statement 121, wherein the anti-CTLA-4 antibody is ipilimumab or tremelimumab.
123. The method of statement 121, wherein the anti-PD-1 antibody is pembrolizumab or nivolumab.
124. The method of statement 121, wherein the anti-PD-L1 antibody is atezolizumab (CAS number 1380723-44-3), avelumab (CAS number 1537032-82-8), or durvalumab (CAS number 1428935-60-7).
125. The method of any preceding statement, wherein the one or more immune checkpoint modulator includes, or is: ipilimumab and pembrolizumab; tremelimumab and durvalumab; or ipilimumab and nivolumab.
126. The method of any one of statements 119 to 125, wherein the at least two immune checkpoint modulators are administered concurrently.
127. The method of any one of statements 119 to 125, wherein the at least two immune checkpoint modulators are administered separately and/or sequentially.

Chemotherapeutic Agent
128. The method of any preceding statement, wherein the chemotherapeutic agent is a chemotherapeutic agent which induces immunogenic cell death of cancer cells.
129. The method of any preceding statement, wherein the chemotherapeutic agent is a chemotherapeutic agent which induces an immune response in the subject.
130. The method of statement 129, wherein the chemotherapeutic agent is a chemotherapeutic agent which induces a type I interferon response in the subject.
131. The method of any preceding statement, wherein the chemotherapeutic agent is an anthracycline or a taxane.
132. The method of statement 131, wherein the anthracycline is doxorubicin, daunorubicin, epirubicin, idarubicin, mitoxantrone, or valrubicin, preferably doxorubicin.
133. The method of statement 131, wherein the taxane is docetaxel, paclitaxel, or abraxane, preferably docetaxel.

AXL-Related Disease
134. The method of any preceding statement, wherein the AXLI-related disease is a proliferative disease.
135. The method of any preceding statement, wherein the AXL-related disease is a neoplastic disease.
136. The method of any preceding statement, wherein the AXL-related disease is a solid tumour.
137. The method of any preceding statement, wherein the AXL-related disease is cancer.
138. The method of statement 137, wherein the cancer is selected from the group consisting of: histocytoma, glioma, astrocytoma, osteoma, lung cancer, small-cell lung cancer, non-small-cell lung cancer, gastrointestinal cancer, bowel cancer, colon cancer, breast cancer, ovarian carcinoma, prostate cancer, testicular cancer, liver cancer, kidney cancer, urothelial carcinoma, bladder cancer, pancreas cancer, brain cancer, glioblastoma, sarcoma, osteosarcoma, Kaposi's sarcoma, melanoma, mesothelioma, lymphomas, and leukemias.
139. The method of statement 137, wherein the cancer is selected from the group consisting of: breast cancer, lung cancer, non-small-cell lung cancer, melanoma, mesothelioma, acute myeloid leukemia (AML), myelodysplastic syndrome (MDS), pancreas cancer, kidney cancer, urothelial carcinoma, and glioblastoma.
140. The method of statement 137, wherein the cancer:
   i) is breast cancer, melanoma, or lung cancer;
   ii) is a cancer or tumor having or expected to have low tumor mutation burden (TMB) and/or low numbers of oncogenic driver mutations; and/or
   iii) is, or is expected to be, refractory, non-responsive, or otherwise not benefit from treatment with one or more immune checkpoint modulator (ICM).

Administration Schedule Features
141. The method of any preceding statement, wherein the AXL inhibitor is administered concurrently with the one or more immune checkpoint modulator (ICM) and/or the one or more chemotherapeutic agent.
142. The method of any preceding statement, wherein the AXL inhibitor is administered separately and/or sequentially to the one or more immune checkpoint modulator (ICM) and/or the one or more chemotherapeutic agent.
143. The method of any preceding statement, wherein the AXL inhibitor is administered subsequent to administration of the one or more immune checkpoint modulator (ICM) and/or subsequent to administration of the one or more chemotherapeutic.
144. The method of any one of statements 101-140, wherein the one or more chemotherapeutic agent is administered subsequent to administration of the AXL inhibitor and/or subsequent to administration of the one or more immune checkpoint modulator (ICM).
145. The method of any one of statements 101-140, wherein the one or more immune checkpoint modulator (ICM) is administered subsequent to administration of the AXL inhibitor and/or subsequent to administration of the one or more chemotherapeutic agent.
146. The method of any one of statements 101-140, wherein:
   the one or more chemotherapeutic agent is administered subsequent to administration of the AXL inhibitor; and
   the one or more immune checkpoint modulator (ICM) is administered subsequent to administration of the one or more chemotherapeutic agent.
147. The method of any one of statements 101-140, wherein the method comprises:
   i) administering the AXL inhibitor to the subject, wherein the immune checkpoint modulator (ICM) has been, is, or will be, administered to the subject; and/or
   ii) administering the AXL inhibitor to the subject, wherein the chemotherapeutic agent has been, is, or will be, administered to the subject.
148. The method of any one of statements 101-140, wherein the method comprises:
   i) administering the immune checkpoint modulator (ICM) to the subject, wherein the AXL inhibitor has been, is, or will be, administered to the subject; and/or
   ii) administering the immune checkpoint modulator (ICM) to the subject, wherein the chemotherapeutic agent has been, is, or will be, administered to the subject.
149. The method of any one of statements 101-140, wherein the method comprises:
   i) administering the chemotherapeutic agent to the subject, wherein the AXL inhibitor has been, is, or will be, administered to the subject; and/or
   ii) administering the chemotherapeutic agent to the subject, wherein the immune checkpoint modulator (ICM) has been, is, or will be, administered to the subject.
150. The method of any preceding statement, wherein the method comprises administering the AXL inhibitor, immune checkpoint modulator, and/or chemotherapeutic agent alone or in further combination with other treatments.
201. An AXL inhibitor, an immune checkpoint modulator (ICM), and a chemotherapeutic agent, for use in a method of treating an AXL-related disease according to any one of statements 101-150.
202. An AXL inhibitor for use in a method of treating an AXL-related disease according to any one of statements 101-150, the method comprising administering to a subject in need thereof a therapeutically effective amount of an AXL inhibitor, wherein the AXL inhibitor is administered in combination with: an immune checkpoint modulator (ICM); and, a chemotherapeutic agent.
203. An immune checkpoint modulator (ICM) for use in a method of treating an AXL-related disease according to any one of statements 101-150, the method comprising administering to a subject in need thereof a therapeutically effective amount of an ICM, wherein the ICM is administered in combination with: an AXL inhibitor; and, a chemotherapeutic agent.
204. A chemotherapeutic agent for use in a method of treating an AXL-related disease according to any one of statements 101-150, the method comprising administering to a subject in need thereof a therapeutically effective amount of a chemotherapeutic agent, wherein the chemotherapeutic agent is administered in combination with: an AXL inhibitor; and, an immune checkpoint modulator (ICM).
205. An AXL inhibitor and an immune checkpoint modulator (ICM) for use in a method of treating an AXL-related disease according to any one of statements 101-150, the method comprising administering to a subject in need thereof a therapeutically effective amount of an AXL inhibitor and an ICM, wherein the AXL inhibitor and ICM are administered in combination with a chemotherapeutic agent.
206. An AXL inhibitor and a chemotherapeutic agent for use in a method of treating an AXL-related disease according to any one of statements 101-150, the method comprising administering to a subject in need thereof a therapeutically effective amount of an AXL inhibitor and a chemotherapeutic agent, wherein the AXL inhibitor and chemotherapeutic agent are administered in combination with an immune checkpoint modulator (ICM).
207. An immune checkpoint modulator (ICM) and a chemotherapeutic agent for use in a method of treating an AXL-related disease according to any one of statements 101-150, the method comprising administering to a subject in need thereof a therapeutically effective amount of an immune checkpoint modulator (ICM) and a chemotherapeutic agent, wherein the immune checkpoint modulator (ICM) and chemotherapeutic agent are administered in combination with an AXL inhibitor.
301. Use of an AXL inhibitor, an immune checkpoint modulator (ICM), and a chemotherapeutic agent in the manufacture of a medicament for treating a disorder in a subject, wherein the treatment comprises a method of treating an AXL-related disease according to any one of statements 101-150.
302. Use of an AXL inhibitor in the manufacture of a medicament for treating a disorder in a subject, wherein the treatment comprises a method of treating an AXL-related disease according to any one of statements 101-150.
303. Use of an immune checkpoint modulator (ICM) in the manufacture of a medicament for treating a disorder in a subject, wherein the treatment comprises a method of treating an AXL-related disease according to any one of statements 101-150.
304. Use of a chemotherapeutic agent in the manufacture of a medicament for treating a disorder in a subject, wherein the treatment comprises a method of treating an AXL-related disease according to any one of statements 101-150.
305. Use of an AXL inhibitor and an immune checkpoint modulator (ICM) in the manufacture of a medicament for treating a disorder in a subject, wherein the treatment comprises a method of treating an AXL-related disease according to any one of statements 101-150.
306. Use of an AXL inhibitor and a chemotherapeutic agent in the manufacture of a medicament for treating a disorder in a subject, wherein the treatment comprises a method of treating an AXL-related disease according to any one of statements 101-150.
307. Use of an immune checkpoint modulator (ICM) and a chemotherapeutic agent in the manufacture of a medicament for treating a disorder in a subject, wherein the treatment comprises a method of treating an AXL-related disease according to any one of statements 101-150.
401. A kit comprising an AXL inhibitor, an immune checkpoint modulator (ICM), and a chemotherapeutic agent, for use in a method of treating an Axl-related disease according to any one of statements 101-150.
402. A kit comprising an AXL inhibitor and an immune checkpoint modulator (ICM), for use in a method of treating an Axl-related disease according to any one of statements 101-150.
403. A kit comprising an AXL inhibitor and a chemotherapeutic agent, for use in a method of treating an Axl-related disease according to any one of statements 101-150.
404. A kit comprising an immune checkpoint modulator (ICM) and a chemotherapeutic agent, for use in a method of treating an Axl-related disease according to any one of statements 101-150.
501. A pharmaceutical composition comprising: an AXL inhibitor; one or more immune checkpoint modulator (ICM); one or more chemotherapeutic agent; and, a pharmaceutically acceptable excipient.
502. A method of treating an AXL-related disease, the method comprising administering to a subject in need thereof a therapeutically effective amount of an AXL inhibitor in concurrent, separate, or sequential combination with one or more immune checkpoint modulator (ICM) and one or more chemotherapeutic agent.
503. A method of treating an AXL-related disease, the method comprising administering to a subject in need thereof a therapeutically effective amount of an AXL inhibitor, wherein the subject has been, will be, or is being treated with an immune checkpoint modulator (ICM) and/or one or more chemotherapeutic agent.
504. A method of treating an AXL-related disease, the method comprising administering to a subject in need thereof a therapeutically effective amount of one or more immune checkpoint modulator (ICM), wherein the subject has been, will be, or is being treated with an AXL inhibitor and/or one or more chemotherapeutic agent.
505. A method of treating an AXL-related disease, the method comprising administering to a subject in need thereof a therapeutically effective amount of one or more chemotherapeutic agent, wherein the subject has been, will be, or is being treated with an AXL inhibitor and/or one or more immune checkpoint modulator (ICM).
506. A method of selecting a subject for treatment with an AXL inhibitor, wherein a subject is selected for treatment if the subject has been, will be, or is being treated with one or more chemotherapeutic agent and/or one or more immune checkpoint modulator (ICM).
507. The method of statement 506, wherein the subject is selected for treatment if the subject has been treated with one or more immune checkpoint modulator (ICM), and one or more chemotherapeutic agent.
508. The method of statement 506, wherein the subject is selected for treatment if the subject is being treated with one or more immune checkpoint modulator (ICM), and has been, or will be, treated with one or more chemotherapeutic agent.
509. The method of any one of statements 506-508, further comprising administering to the subject a therapeutically effective amount of an AXL inhibitor.
510. A method of selecting a subject for treatment with one or more immune checkpoint modulator (ICM), wherein a subject is selected for treatment if the subject has been, will be, or is being treated with an AXL inhibitor and/or one or more chemotherapeutic agent.
511. The method of statement 510, wherein the subject is selected for treatment if the subject has been treated with an AXL inhibitor and one or more chemotherapeutic agent.
512. The method of statement 510, wherein the subject is selected for treatment if the subject is being treated with an AXL inhibitor, and has been or will be treated with one or more chemotherapeutic agent.
513. The method of any one of statements 510-512, further comprising administering to the subject a therapeutically effective amount of one or more immune checkpoint modulator (ICM).
514. A method of selecting a subject for treatment with one or more chemotherapeutic agent,
   wherein a subject is selected for treatment if the subject has been, will be, or is being treated with an AXL inhibitor and/or one or more immune checkpoint modulator (ICM).
515. The method of statement 514, wherein the subject is selected for treatment if the subject has been treated with an AXL inhibitor and one or more immune checkpoint modulator (ICM).
516. The method of statement 514, wherein the subject is selected for treatment if the subject is being treated with an AXL inhibitor, and has been or will be treated with one or more immune checkpoint modulator (ICM).
517. The method of any one of statements 514-516, further comprising administering to the subject a therapeutically effective amount of one or more chemotherapeutic agent.
518. A method of selecting a subject for treatment with an AXL inhibitor and one or more immune checkpoint modulator (ICM),
   wherein a subject is selected for treatment if the subject has been, will be, or is being treated with one or more chemotherapeutic agent.
519. The method of statement 518, further comprising administering to the subject a therapeutically effective amount of an AXL inhibitor and one or more immune checkpoint modulator (ICM).
520. A method of selecting a subject for treatment with an AXL inhibitor and one or more chemotherapeutic agent,
   wherein a subject is selected for treatment if the subject has been, will be, or is being treated with one or more immune checkpoint modulator (ICM).
521. The method of statement 520, further comprising administering to the subject a therapeutically effective amount of an AXL inhibitor and one or more chemotherapeutic agent.
522. A method of selecting a subject for treatment with one or more immune checkpoint modulator (ICM) and one or more chemotherapeutic agent,
   wherein a subject is selected for treatment if the subject has been, will be, or is being treated with an AXL inhibitor.
523. The method of statement 522, further comprising administering to the subject a therapeutically effective amount of one or more immune checkpoint modulator (ICM) and one or more chemotherapeutic agent.

### EXAMPLES

In the following examples the terms “Bemcentinib” and “Bern”, and the terms “doxorubicin” and “dox” are used interchangeably. Similarly, the terms “immune checkpoint inhibitors”, “checkpoint inhibitors”, “CPI”, “immune checkpoint blockade”, and “ICB” are used variously to refer to refer to the immunotherapy (anti-CTLA-4+anti-PD-1) component of the triple combination treatments.

### Example 1

Materials & Methods

Cell Harvest

Exponentially growing 4T1 mammary carcinoma cells were harvested and re-suspended at 4×10⁶ per ml in the mixture of serum-free RPMI medium and Matrigel (1:1). Only cells with over 95% viability have been used for in vivo study.

Tumor Injection Procedure

Each animal was weighed before cell implantation. Mice were anesthetized by inhalation of Sevoflurane. Anesthesia was induced at 8% Sevoflurane and maintained at 4%. Every mouse was placed on a heating pad and appropriately marked. Under a suitable depth of unconsciousness, animals were shaved, and the skin and surrounding region washed with Chlorhexidine (1 mg/ml) with use of sterile gauze. Injection was done orthotopically with one tumor per mouse with 0.05 ml of approximately 2×10⁵ 4T1 cells in serum-free RPMI medium/Matrigel (1:1).

Randomization

Treatment groups are indicated in Table 1. 2 randomizations were performed on 150 mice in 30 cages, with 5 animals per cage. The first randomization was performed on day 11 post implantation. The average of tumor volume of animals in each cage was determined. The average tumor volume in the cages was from 3.8 mm³ to 19.7 mm³. All animals in each cage were assigned to AXL inhibitor (bemcentinib) or vehicle treatment. The first randomization was conducted with Latin square method based on the average tumor volume of all animals in each cage. 15 cages were treated with bemcentinib and 15 animals treated with vehicle. Once the average tumor size reached 70 mm³ for vehicle treated animals and 76 mm³ for bemcentinib treated animals (tumor volume=L×W×W/2) the second randomization was performed prior to injection of chemotherapeutic agent (doxorubicin) on day 16. Randomization 2 was conducted with Latin square method based on average tumor volume of all animals in the bemcentinib group and in the vehicle group from randomization 1. The tumor volume was 7-153 mm³ for the vehicle group and 12-171 mm³ for the bemcentinib group.

TABLE 1
experimental groups

| | | | | | Dose | | | |
|---|---|---|---|---|---|---|---|---|
| | | | Dose | Concentration | volume | | | Euthanasia |
| Group | No. | Treatment | (mg/kg) | (mg/ml) | (ml/kg) | Dosing schedule | Route | (Day) |
| A | 11 + 4 | PBS | na | na | na | Day 16 | | 4 day 19 |
| | | IgG | 20 | 1 | 10 | Day19, 21, 23, 25* | IP | 34-41 |
| | | Vehicle | na | na | 10 | Bid from Day 11 | PO | |
| B | 10 + 4 | PBS | na | na | na | Day 16 | IT | 34-41 |
| | | IgG | 20 | 1 | 10 | Day19, 21, 23, 25* | IP | |
| | | Bemcentinib | 50 | 5 | 10 | Bid from day 11 | PO | |
| C | 10 | Doxorubicin | ~1 | 0.5 | na | Day 16* | IT | 38-41 |
| | | IgG | 20 | 1 | 10 | Day19, 21, 23, 25* | IP | |
| | | Bemcentinib | 50 | 5 | 10 | Bid from day 11 | PO | |
| D | 10 | Doxorubicin | ~1 | 0.5 | na | Day 16* | IT | 38-41 |
| | | IgG | 20 | 1 | 10 | Day19, 21, 23, 25* | IP | |
| | | Vehicle | na | na | 10 | Bid from Day 11 | PO | |
| E | 20 | PBS | na | na | na | Day 16 | IT | 41-59 |
| | | anti-CTLA-4 + | 10 + 10 | 2 + 2 | 5 + 5 | Day19, 21, 23, 25* | IP | |
| | | anti-PD-1 | | | | | | |
| | | Vehicle | na | na | 10 | Bid from Day 11 | PO | |
| F | 22 + 4 | Doxorubicin | ~1 | 0.5 | na | Day 16* | IT | 4 day 19 |
| | | anti-CTLA-4 + | 10 + 10 | 2 + 2 | 5 + 5 | Day19, 21, 23, 25* | IP | 41-152 |
| | | anti-PD-1 | | | | | | |
| | | Vehicle | na | na | 10 | Bid from Day 11 | PO | |
| G | 20 | PBS | na | na | na | Day 16 | IT | 52-152 |
| | | anti-CTLA-4 + | 10 + 10 | 2 + 2 | 5 + 5 | Day 19, 21, 23 and | IP | |
| | | anti-PD-1 | | | | 25* | | |
| | | Bemcentinib | 50 | 5 | 10 | Bid from Day 11 | PO | |
| H | 22 + 4 | Doxorubicin | ~1 | na | na | Day 16* | IT | 4 day 19 |
| | | anti-CTLA-4 + | 10 + 10 | 2 +2 | 5 + 5 | Day 19, 21, 23 and | IP | 53-152 |
| | | anti-PD-1 | | | | 25* | | |
| | | Bemcentinib | 50 | 5 | 10 | Bid from Day 11 | PO | |

*Time from tumor cells implantation (Day 0)
bid = bis in die; twice (two times) a day

Treatment Schedule

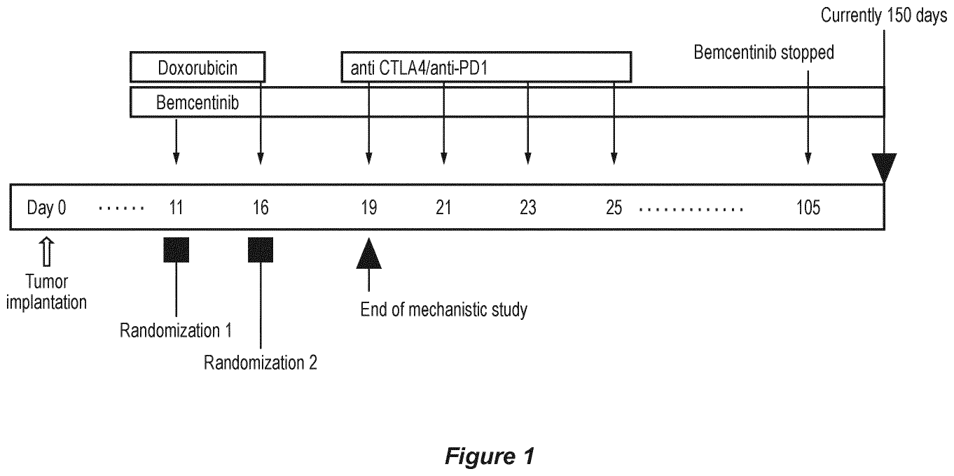

AXL inhibitor bemcentinib was given Bid (twice a day) to groups as described in Table 1, starting on the day of randomization 1 as shown in FIG. 1. Bemcentinib was given as a loading dose starting day 11 after tumor implantation and lasting for 3 days prior to treatment with the chemotherapeutic agent doxorubicin. Doxorubicin was administered as a single injection to the appropriate groups according to Table 1, on day 16 after tumor cell implantation, 3 days after randomization. Immune checkpoint modulator (anti-CTLA4/anti-PD1; or IgG control) was given to groups as described in Table 1 starting 19 days after implantation, as shown in FIG. 1.

Tumor Growth Measurements

Tumor growth was measured with a digital hand held caliper at least twice weekly (once tumor emerges), prior to the first dosing, and then on the day of euthanasia.

Body Weights

Animals were weighed prior to tumor cell injection, prior to dosing, two-to-three times weekly with tumor growth measurements, and prior to euthanasia.

Intra Tumor (IT) Dosing Procedure

When tumors reached an average of 7-170 mm³, each animal received Doxorubicin (Dox) at a concentration of 0.5 mg/ml (groups C,D,F and H) in a total volume of 50 ul×TV/120 as indicated in Table 1. Groups A, B, E, and G did not receive Doxorubicin. Total drug dose was calculated based on each animal's individual tumor size. For IT drug injections, animals were anesthetized as described above, and drug was slowly injected into the tumor at a rate of approximately 5 ul/second.

Intra Peritoneal (IP) Dosing Procedure

On the appropriate days, each animal received a specific amount of IgG (group A,B,C,D) or a combination of the immune check inhibitors (CPI) anti-mCTRL-4+anti-mPD-1 (groups E-H) as indicated in Table 1. Dosing volume was 10 ml/kg by 30-gauge needle. Dosing schedule was on days 19, 21, 23 and 25 after tumor implantation.

Oral Dosing (PO) Procedure

On the appropriate days, each animal in Groups B, C, G, and H received a specific amount of vehicle (0.5% (w/w) hydroxypropyl methylcellulose/0.1% (w/w) Tween 80). After 3 days of a loading dose (100 mg/kg) from day 11, each animal in Group A, D, E, and F received a specific amount of Bemcentinib (Bern) dosing solution at the dose of 50 mg/kg as indicated in Table 1. Dosing schedule was twice daily on a 5 day-2 day off schedule until 105 days post implantation. Dosing volume was 10 ml/kg by oral gavage.

Clinical Observations

Animals were observed once daily for general appearance.

Mortality/Morbidity

All animals were examined once daily for mortality or morbidity. An animal was sacrificed if the animal lost >20% of the body weight or was judged as moribund. Mice bearing tumors that reached a volume exceeding 1000 mm³ were euthanized. Euthanized animals and any animals found dead prior to rigor mortis were necropsied.

Euthanasia

Animals were anesthetized with Sevoflurane and euthanized by cervical dislocation. Euthanasia was conducted by following the Institutional SOP accordingly.

Tissue Collection

Spleen

Spleens were weighed and inspected for macroscopic metastasis and cut in half along the long axis. Half of the spleen was snap frozen, with the other half fixed in 4% formalin for 24 hours at room temperature and stored in 70% ethanol at 4° C. until embedding in paraffin.

Liver

Livers were inspected for macroscopic metastasis and fixed in 4% formalin for 24 hours at room temperature and stored in 70% ethanol at 4° C. until embedding in paraffin.

Lungs

Lungs were inspected for macroscopic metastasis and fixed in 4% formalin for 24 hours at room temperature and stored in 70% ethanol at 4° C. until embedding in paraffin.

Tumor Collection

Tumors were weighed and cut in half along the long axis, one half was snap frozen in liquid nitrogen in 2 cryotubes per tumor, while the other half was fixed in 4% formalin for 24 hours at room temperature and stored in 70% ethanol at 4° C. until embedding in paraffin.

Analysis of Tumor Tissue (Mechanistic Study)

RNA was extracted from snap frozen tumors using the RNEasy Microarray Tissue Mini kit from Qiagen following the manufacturer's instructions. As tumors contained Doxorubicin and powderizing them could expose personnel to harmful substances, the RNA was extracted in a closed system using the GentleMACS dissociator and M Tubes. Purity and concentration of RNA was measured using Nanodrop. cDNA was synthesized from RNA using the RT2 First Strand kit (Qiagen) according to the manufacturer's instructions. cDNA was mixed with RT2 SYBR Green Mastermix (Qiagen) and loaded into RT² Profiler™ PCR Array Mouse Type I Interferon Response, to analyze the expression of the 84 genes listed in Table 2. The plates were analysed using a Light Cycler 480 (Roche) and data were analysed using the Qiagen Data Analysis Centre and Graph Pad (Prism).

16 animals were used in the mechanistic study and were sacrificed day 19 post implantation, 3 days after Doxorubicin injection. These animals did not receive immune checkpoint modulator treatment (with checkpoint inhibitors; CPI). The 4 animals from group A (no treatment) were used as controls. The fold changes of 4 animals of group B (Bemcentinib), group F (Doxorubicin) and group H (Bemcentinib+Doxorubicin) are expressed as compared to the control animals (group A).

TABLE 2
Type I IFN-related Genes analysed

| Position | RefSeq Number | Symbol | Description |
|---|---|---|---|
| A01 | NM_019655 | Adar | Adenosine deaminase, RNA-specific |
| A02 | NM_013863 | Bag3 | Bcl2-associated athanogene 3 |
| A03 | NM_198095 | Bst2 | Bone marrow stromal cell antigen 2 |
| A04 | NM_009807 | Casp1 | Caspase 1 |
| A05 | NM_007616 | Cav1 | Caveolin 1, caveolae protein |
| A06 | NM_011333 | Ccl2 | Chemokine (C-C motif) ligand 2 |
| A07 | NM_013652 | Ccl4 | Chemokine (C-C motif) ligand 4 |
| A08 | NM_013653 | Ccl5 | Chemokine (C-C motif) ligand 5 |
| A09 | NM_001033122 | Cd69 | CD69 antigen |
| A10 | NM_011617 | Cd70 | CD70 antigen |
| A11 | NM_009855 | Cd80 | CD80 antigen |
| A12 | NM_019388 | Cd86 | CD86 antigen |
| B01 | NM_009875 | Cdkn1b | Cyclin-dependent kinase inhibitor 1B |
| B02 | NM_007575 | Ciita | Class II transactivator |
| B03 | NM_007768 | Crp | C-reactive protein, pentraxin-related |
| B04 | NM_021274 | Cxcl10 | Chemokine (C-X-C motif) ligand 10 |
| B05 | NM_172689 | Ddx58 | DEAD (Asp-Glu-Ala-Asp) box polypetide 58 |
| B06 | NM_011163 | Eif2ak2 | Eukaryotic translation initiation factor 2-alpha kinase 2 |
| B07 | NM_010259 | Gbp2b | Guanylate binding protein 1 |
| B08 | NM_008199 | H2-B | Histocompatibility 2, blastocyst |
| B09 | NM_010380 | H2-D1 | Histocompatibility 2, D region locus 1 |
| B10 | NM_001001892 | H2-K1 | Histocompatibility 2, K1, K region |
| B11 | NM_013544 | H2-M10.1 | Histocompatibility 2, M region locus 10.1 |
| B12 | NM_013819 | H2-M3 | Histocompatibility 2, M region locus 3 |
| C01 | NM_010395 | H2-T10 | Histocompatibility 2, T region locus 10 |
| C02 | NM_008329 | Ifi204 | Interferon activated gene 204 |
| C03 | NM_023065 | Ifi30 | Interferon gamma inducible protein 30 |
| C04 | NM_027835 | Ifih1 | Interferon induced with helicase C domain 1 |
| C05 | NM_008331 | Ifit1 | Interferon induced protein with tetratricopeptide repeats 1 |
| C06 | NM_008332 | Ifit2 | Interferon induced protein with tetratricopeptide repeats 2 |
| C07 | NM_010501 | Ifit3 | Interferon induced protein with tetratricopeptide repeats 3 |
| C08 | NM_026820 | Ifitm1 | Interferon induced transmembrane protein 1 |
| C09 | NM_030694 | Ifitm2 | Interferon induced transmembrane protein 2 |
| C10 | NM_025378 | Ifitm3 | Interferon induced transmembrane protein 3 |
| C11 | NM_010503 | Ifna2 | Interferon alpha 2 |
| C12 | NM_010504 | Ifna4 | Interferon alpha 4 |
| D01 | NM_010508 | Ifnar1 | Interferon (alpha and beta) receptor 1 |
| D02 | NM_010509 | Ifnar2 | Interferon (alpha and beta) receptor 2 |
| D03 | NM_010510 | Ifnb1 | Interferon beta 1, fibroblast |
| D04 | NM_177348 | Ifne | Interferon epsilon |
| D05 | NM_197889 | Ifnz | Interferon zeta |
| D06 | NM_010548 | Il10 | Interleukin 10 |
| D07 | NM_008357 | Il15 | Interleukin 15 |
| D08 | NM_001314054 | Il6 | Interleukin 6 |
| D09 | NM_008390 | Irf1 | Interferon regulatory factor 1 |
| D10 | NM_008391 | Irf2 | Interferon regulatory factor 2 |
| D11 | NM_016849 | Irf3 | Interferon regulatory factor 3 |
| D12 | NM_012057 | Irf5 | Interferon regulatory factor 5 |
| E01 | NM_016850 | Irf7 | Interferon regulatory factor 7 |
| E02 | NM_008394 | Irf9 | Interferon regulatory factor 9 |
| E03 | NM_015783 | Isg15 | ISG15 ubiquitin-like modifier |
| E04 | NM_020583 | Isg20 | Interferon-stimulated protein |
| E05 | NM_146145 | Jak1 | Janus kinase 1 |
| E06 | NM_008413 | Jak2 | Janus kinase 2 |
| E07 | NM_010762 | Mal | Myelin and lymphocyte protein, T cell differentiation protein |
| E08 | NM_008591 | Met | Met proto-oncogene |
| E09 | NM_010846 | Mx1 | Myxovirus (influenza virus) resistance 1 |
| E10 | NM_013606 | Mx2 | Myxovirus (influenza virus) resistance 2 |
| E11 | NM_010851 | Myd88 | Myeloid differentiation primary response gene 88 |
| E12 | NM_019401 | Nmi | N-myc (and STAT) interactor |
| F01 | NM_001313921 | Nos2 | Nitric oxide synthase 2, inducible |
| F02 | NM_145211 | Oas1a | 2′-5′ oligoadenylate synthetase 1A |
| F03 | NM_003507 | Oas1b | 2′-5′ oligoadenylate synthetase 1B |
| F04 | NM_145227 | Oas2 | 2′-5′ oligoadenylate synthetase 2 |
| F05 | NM_008884 | Pml | Promyelocytic leukemia |
| F06 | NM_008860 | Prkcz | Protein kinase C, zeta |
| F07 | NM_011190 | Psme2 | Proteasome (prosome, macropain) 28 subunit, beta |
| F08 | NM_011364 | Sh2d1a | SH2 domain protein 1A |
| F09 | NM_001033306 | Shb | Src homology 2 domain-containing transforming protein B |
| F10 | NM_009896 | Socs1 | Suppressor of cytokine signalling 1 |
| F11 | NM_009283 | Stat1 | Signal transducer and activator of transcription 1 |
| F12 | NM_019963 | Stat2 | Signal transducer and activator of transcription 2 |
| G01 | NM_011486 | Stat3 | Signal transducer and activator of transcription 3 |
| G02 | NM_013683 | Tap1 | Transporter 1, ATP-binding cassette, sub-family B (MDR/TAP) |
| G03 | NM_174989 | Ticam1 | Toll-like receptor adaptor molecule 1 |
| G04 | NM_011593 | Timp1 | Tissue inhibitor of metalloproteinase 1 |
| G05 | NM_126166 | Tlr3 | Toll-like receptor 3 |
| G06 | NM_133211 | Tlr7 | Toll-like receptor 7 |
| G07 | NM_133212 | Tlr8 | Toll-like receptor 8 |
| G08 | NM_031178 | Tlr9 | Toll-like receptor 9 |
| G09 | NM_009425 | Tnfsf10 | Tumor necrosis factor (ligand) superfamily, member 10 |
| G10 | NM_011632 | Traf3 | Tnf receptor-associated factor 3 |
| G11 | NM_018793 | Tyk2 | Tyrosine kinase 2 |
| G12 | NM_009505 | Vegfa | Vascular endothelial growth factor A |
| H01 | NM_007393 | Actb | Actin, beta |
| H02 | NM_009735 | B2m | Beta-2 microglobulin |
| H03 | NM_008084 | Gapdh | Glyceraldehyde-3-phophate dehydrogenase |
| H04 | NM_010368 | Gusb | Glucoronidase, beta |
| H05 | NM_008302 | Hsp90ab1 | Heat shock protein 90 alpha (cytosolic), class B member 1 |
| H06 | SA_00106 | MGDC | Mouse Genomic DNA Contamination |
| H07 | SA_00104 | RTC | Reverse Transcription Control |
| H08 | SA_00104 | RTC | Reverse Transcription Control |
| H09 | SA_00104 | RTC | Reverse Transcription Control |
| H10 | SA_00103 | PPC | Positive PCR Control |
| H11 | SA_00103 | PPC | Positive PCR Control |
| H12 | SA_00103 | PPC | Positive PCR Control |

Statistics

Survival curves were made with Graph Pad Prism software also used to perform mantel cox log rank tests. Fold change of the genes analysed were calculated using the ΔΔ Ct method.

Results

150 BALB/c mice were injected orthotopically with 4T1 tumor cells. After 11 days the tumors displayed a volume between 4 mm³ to 20 mm³. 150 animals in 30 cages were used for randomization 1. Average tumor volume from all animals in each cage was calculated. Based on the latin square method 15 cages were assigned to bemcentinib treatment and 15 cages to vehicle treatment. When the average tumor volume was above 70 mm³ for vehicle treated animals and 76 mm³ for the bemcentinib treated animals randomization 2 was performed prior to Doxorubicin injection on day 16. Some animals were lost in doxorubicin injection or excluded because of the absence of tumor; 9 animals were excluded or sacrificed. After doxorubicin injection 141 animals were placed in the different groups: 15 in group A, 14 in group B, 10 in group C, 10 in group D, 20 in group E, 26 in group F, 20 in group G, and 26 in group H (see Table 1).

Upon initiation of immune checkpoint modulator treatment (with checkpoint inhibitors (CPI) anti-CTLA4/anti-PD1) volume was 128 mm³ on average and the maximum tumor volume was 233 mm³. No animals were lost during CPI injection. On day 19, 16 animals were sacrificed for the mechanistic part of the study in order to perform Type I IFN expression analysis. 125 animals were used in the survival study: 11 in group A, 10 in group B, 10 in group C, 10 in group D, 20 in group E, 22 in group F, 20 in group G and 22 in group H (see Table 1).

Combination Therapies are Tolerated by Mice

The weight of mice in the different groups is presented in FIG. 2. Most animals show an increase of body weight during the experiment (FIG. 2A, 2B, 2C), though some animals also display a decrease of body weight before sacrifice, probably due to the systemic disease (FIG. 2A, B, C). The treatments appear to be tolerated by the mice. Some of the animals treated with Bemcentinib (group G and H) show a decrease of weight when the treatment begins at day 11 post tumor implantation (FIG. 2F, 2G). Most of the mice gain weight again some days later (around day 15). The animals seem to need some time to adapt to the Bemcentinb. A similar pattern of weight change is also observed for some vehicle treated animal (FIG. 2D, 2E) so this decrease of body weight may be due to the oral gavage procedure.

Triple Combination Treatment with AXL Inhibitor, Immune Checkpoint Modulator, and Cytotoxic Chemotherapy Delays Tumor Growth

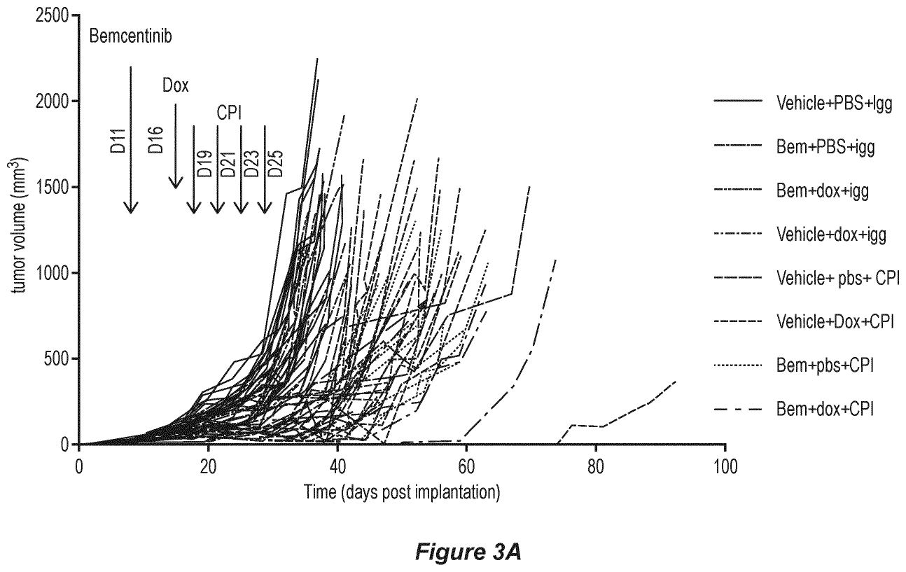

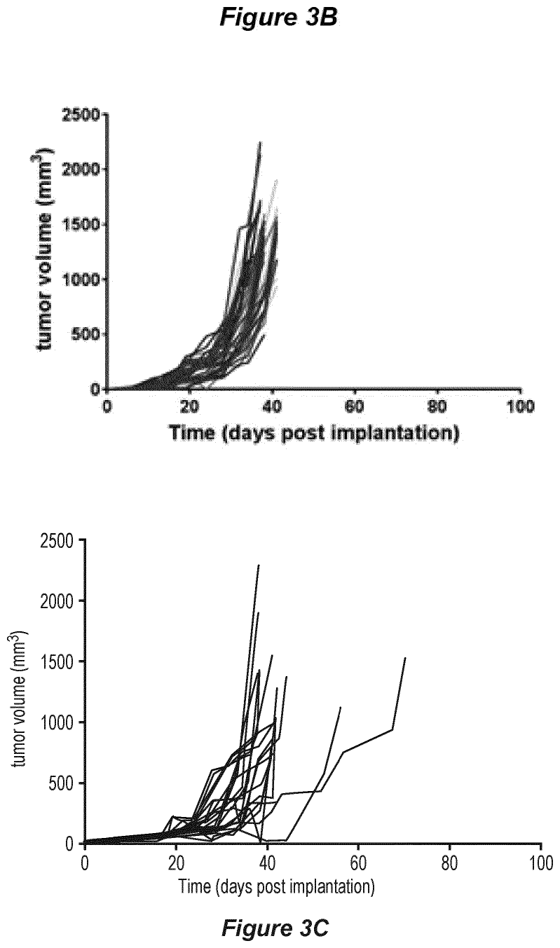

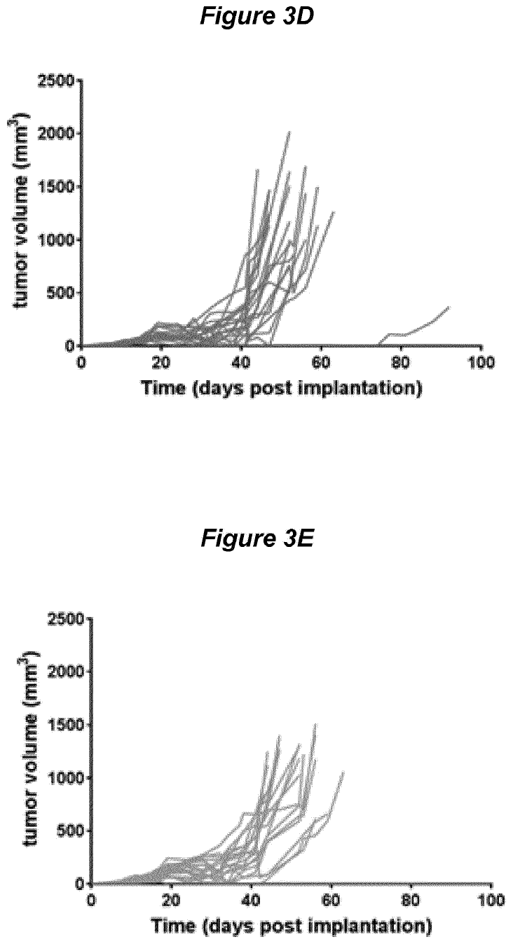

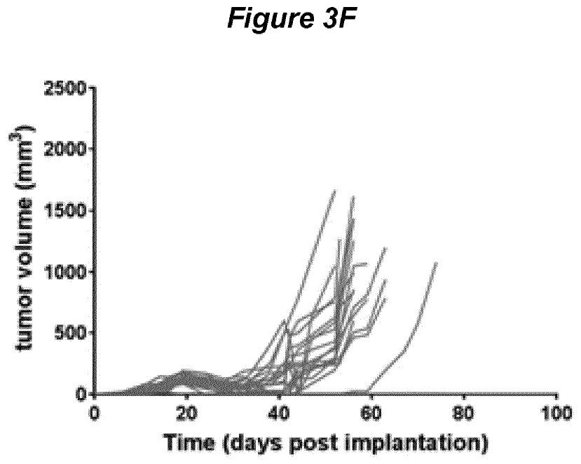

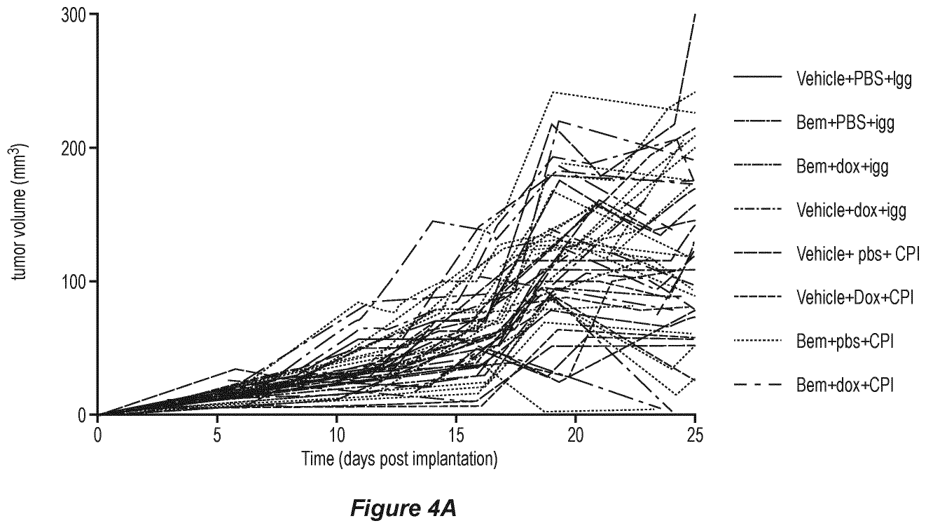

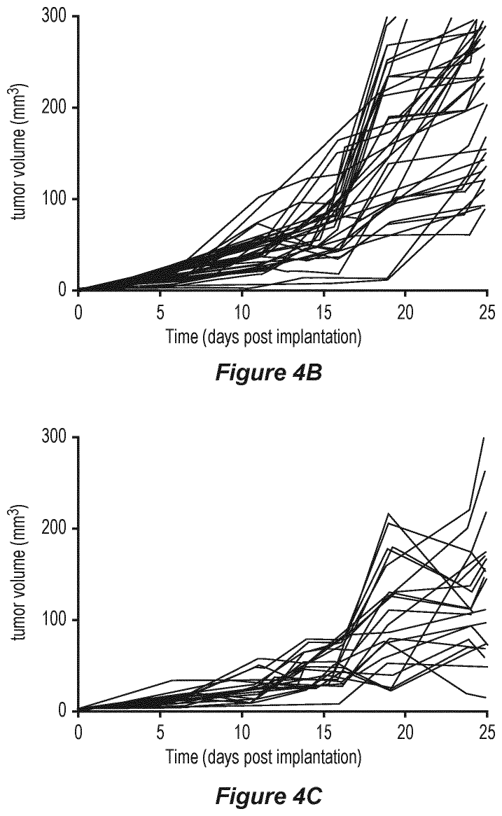

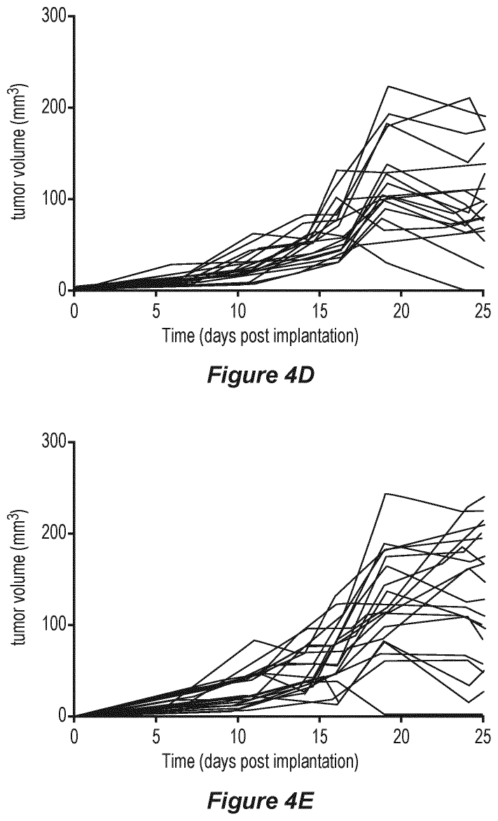

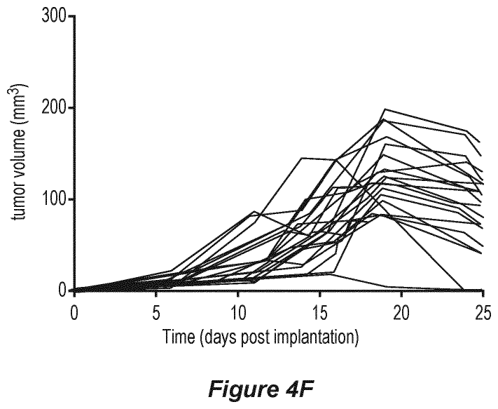

When pre-treatment with Bemcentinib was initiated the tumor volume was between 4 and 20 mm³, 100 mm³ on average. The tumor volume was between 7 mm³ and 170 mm³ (72 mm³ on average) when Doxorubicin was injected. The first CPI injection was initiated when tumor volume was between 14 and 292 mm³, 128 mm³ on average. The tumor growth curves are presented in FIGS. 3 and 4: FIG. 3 shows the tumour growth curves measured across the entire duration of the study; FIG. 4 shows the tumor growth curves for the same groups, but in the first 25 days following implantation only. Tumor growth shows heterogeneity for all groups, with less heterogeneity observed for groups without CPI (A, B, C, D), control, and CPI treatment alone (FIG. 3A, B).

Animals treated with CPI alone display similar growth curves (FIG. 3C). Animals treated with CPI alone (group E) display a small delay of tumor growth compare to all control groups (A,B,C,D) (FIG. 3A, 3C). Contrary to control groups (FIG. 3B) a small phase of tumor decrease or at least a stabilisation phase is observed, this phase is followed by a quick growth (FIG. 3C, 4C).

Animals treated with doxorubicin and CPI (FIG. 3D) display longer growth delay compared to CPI alone. Furthermore for some animals treated with both doxorubicin and CPI the tumor volume decreases after CPI treatment (FIG. 3D, 4D). For some animals the tumor start to grow again around day 25 post implantation (FIG. 4D). For most animals the effects of doxorubicin seem to be temporary. The effect is prolonged for one animal which did not show tumor growth after the initial response.

For animals treated with Bemcentinib and CPI (FIG. 3E) a similar growth delay as for the doxorubicin and CPI group is observed. The same decrease of tumor growth is observed around day 20 post implantation (FIG. 4E). The majority of the tumors start growing again as for the CPI+Dox group. 2 animals show a decrease of tumor growth without any relapse after 150 days.

For the triple combination (Bemcentenib+Doxorubicin+CPI) (FIG. 3F, 4F) a further growth delay is observed for most of the animals. Furthermore all animals display a decrease of tumor growth during the CPI treatment (FIG. 4F). In addition, 4 animals display a complete decrease of the tumor without any relapse after 150 days.

In summary, the growth curves show a small effect of CPI alone, while adding Bemcentinib or doxorubicin to the CPI seem to have the same effect on tumor growth: a decrease followed by a relapse for most of the animals. Some long terms responders were observed in both groups. With the triple combination a delay in tumor growth is observed as compared to all other groups. In addition the highest number of responders is observed for this group.

Triple Combination Treatment with AXL Inhibitor, Immune Checkpoint Modulator, and Cytotoxic Chemotherapy Prolongs Survival

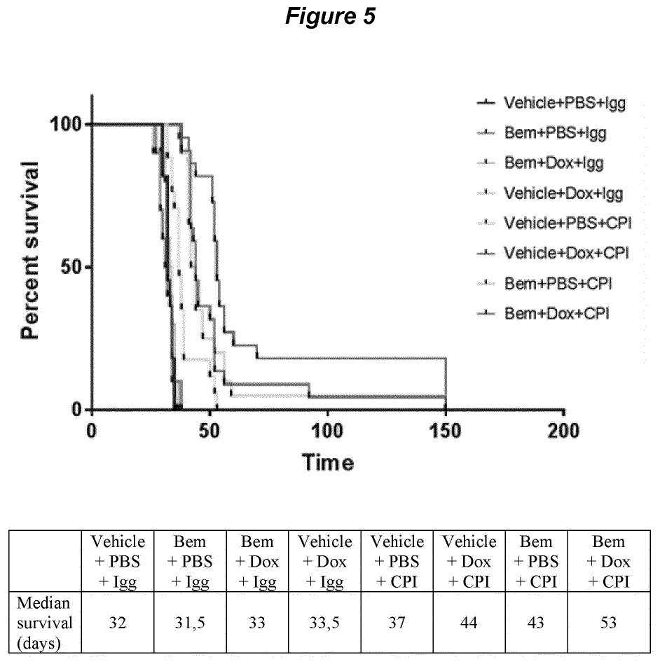

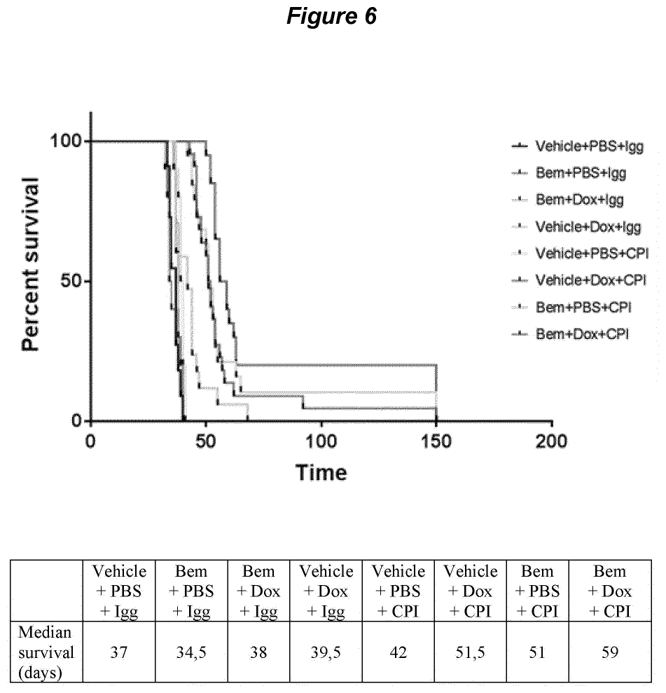

In the present study mice were sacrificed when tumors reached 1000 mm³ and/or severe symptoms of shortage of breath was observed. Transformed survival curves were made based on the day tumors reached 1000 or 500 mm³ (FIGS. 5 and 6 respectively).

At 500 mm³ all groups without CPI treatment display similar Kaplan Meier survival curves with similar median survival 31.5 to 33.5 days (FIG. 5). No significant differences are observed between the groups without CPI (Table 3, LogRank test, Pvalue from 0.5 to 0.8). Without the CPI treatment bemcentinib and doxorubicin do not show any effect on mouse survival.

There is an significant increase of median survival for CPI (37 days) compared to all groups without CPI treatment treated (31.5 to 33.5 days) (FIG. 5; Table 3, Pvalue<0.0004).

Median survival to reach 500 mm³ for CPI plus doxorubicin is 44 days, for CPI plus bemcentinib is 43 days, both of which are significant compared to groups without CPI treatment or CPI treatment alone (Table 3, P value<0.0015). The growth curves of the CPI plus doxorubicin compared to CPI plus Bemcentinib are similar and no significant differences are observed (Table 3 Pvalue=0.6141).

The triple combination treatment mice display a median survival (500 mm³) of 53 days (FIG. 5B). This is significantly different as compared to CPI plus doxorubicin (Table 3, Pvalue=0.011) and to CPI+Bencentinib (Table 3, Pvalue=0.0117). The survival curves of triple combination treated animals are also significantly different compared to animals with no CPI treatment or CPI alone (Table 3, Pvalue<0.0001) The median survival at 500 mm³ and survival curves shows that the triple combination prolongs mouse survival compared to all other treatments.

TABLE 3
P values (log rank mantel cox test, GraphPadPrism) from survival curves at 500 mm³ tumor volume.

| | Bem | Bem | Vehicle | Vehicle | Vehicle | Bem | Bem |
|---|---|---|---|---|---|---|---|
| | PBS | Dox | Dox | PBS | Dox | PBS | Dox |
| | Igg | Igg | Igg | CPI | CPI | CPI | CPI |
| Vehicle | 0.7198 | 0.5005 | 0.5570 | <0.0001 | <0.0001 | <0.0001 | <0.0001 |
| PBS | | | | | | | |
| Igg | | | | | | | |
| Bem | | 0.6863 | 0.6723 | 0.0003 | <0.0001 | <0.0001 | <0.0001 |
| PBS | | | | | | | |
| Igg | | | | | | | |
| Bem | | | 0.7717 | 0.0004 | <0.0001 | <0.0001 | <0.0001 |
| Dox | | | | | | | |
| Igg | | | | | | | |
| Vehicle | | | | <0.0001 | <0.0001 | <0.0001 | <0.0001 |
| Dox | | | | | | | |
| Igg | | | | | | | |
| Vehicle | | | | | 0.0008 | 0.0015 | <0.0001 |
| PBS | | | | | | | |
| CPI | | | | | | | |
| Vehicle | | | | | | 0.6141 | 0.0111 |
| Dox | | | | | | | |
| CPI | | | | | | | |
| Bem | | | | | | | 0.0117 |
| PBS | | | | | | | |
| CPI | | | | | | | |

All the groups are compared together one by one.
Pvalues highlighted in underline are the non-significant Pvalues.
The Triple combination treatment display significant differences with all other treatments.
All groups without CPI treatment are not significantly different.

When considering survival at 1000 mm³ (FIG. 6) or after sacrifice due to severe symptoms, the triple combination treatment again significantly increased the animal survival compared to all other treatments (FIG. 6, Table 4, Pvalue<0.0386).

The CPI+doxorubicin or CPI+Bemcentinib treatments display similar survival curves (FIG. 6, Table 4) and no significant differences (Table 4, Pvalue=0.7403). Both treatments significantly increase the animal survival compared to animals without CPI treatments or CPI alone (FIG. 6, Table 4, Pvalue<0.0026). The CPI treatment increase significantly mouse survival compared to animals without CPI treatment (FIG. 6, table 4, Pvalue<0.0062).

The only difference of survival curves at 1000 mm³ compared to 500 mm³ is observed between animals without CPI treatment. A significant difference is observed for animals treated with Doxorubicin only compared to other groups without CPI treatment (Table 4, Pvalue<0.0371). All other groups display similar growth curves despite the fact that the median survival is 34.5 days for animals treated with Bemcentinib alone (FIG. 6) compare to 37-38.5 days for other group without CPI treatment.

TABLE 4
P values (log rank mantel cox test, GraphPadPrism) from survival curves at 1000 mm³ tumor volume.

| | Bem | Bem | Vehicle | Vehicle | Vehicle | Bem | Bem |
|---|---|---|---|---|---|---|---|
| | PBS | Dox | Dox | PBS | Dox | PBS | Dox |
| | Igg | Igg | Igg | CPI | CPI | CPI | CPI |
| Vehicle | 0.8534 | 0.1363 | 0.0063 | 0.0002 | <0.0001 | <0.0001 | <0.0001 |
| PBS | | | | | | | |
| Igg | | | | | | | |
| Bem | | 0.3782 | 0.0371 | 0.0005 | <0.0001 | <0.0001 | <0.0001 |
| PBS | | | | | | | |
| Igg | | | | | | | |
| Bem | | | 0.0371 | 0.0062 | <0.0001 | <0.0001 | <0.0001 |
| Dox | | | | | | | |
| Igg | | | | | | | |
| Vehicle | | | | 0.0304 | <0.0001 | <0.0001 | <0.0001 |
| Dox | | | | | | | |
| Igg | | | | | | | |
| Vehicle | | | | | 0.0008 | 0.0026 | <0.0001 |
| PBS | | | | | | | |
| CPI | | | | | | | |
| Vehicle | | | | | | 0.7403 | 0.0052 |
| Dox | | | | | | | |
| CPI | | | | | | | |
| Bem | | | | | | | 0.0386 |
| PBS | | | | | | | |
| CPI | | | | | | | |

All the groups are compared together one by one.
Pvalues highlighted in underline are the non-significant Pvalues.
All groups without CPI treatment are not significantly different except the group treated with dox only.
The Triple combination treatment display significant differences with all other treatments.

Triple Combination Treatment Increases Initial Response and Long Term Response Rate as Compared to Sub-Combination Treatments

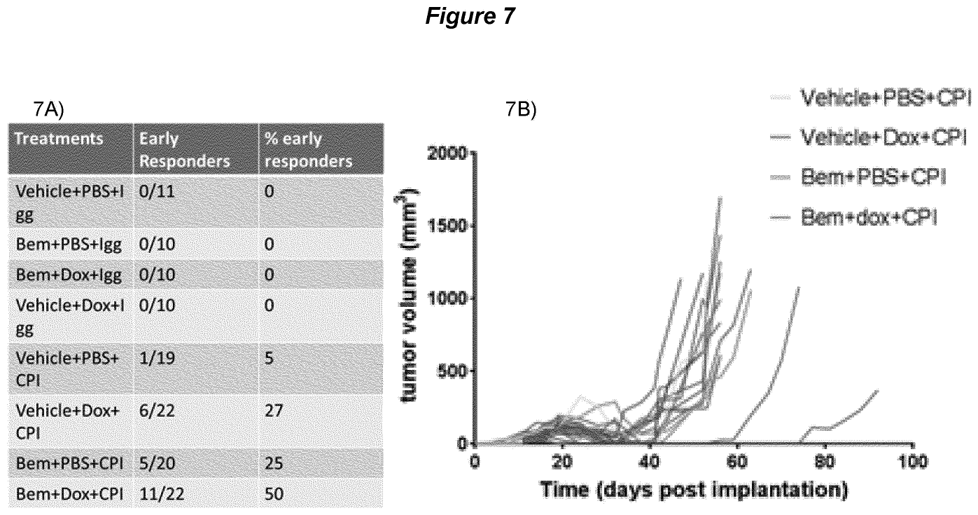

In addition to prolonged survival, an initial response defined as 80% tumor volume reduction (FIG. 7A) is observed for all CPI treated groups, with 5% of initial responders for CPI alone increased to 27 and 25% respectively for CPI+Dox and CPI+Bern, and to 50% for the triple combination.

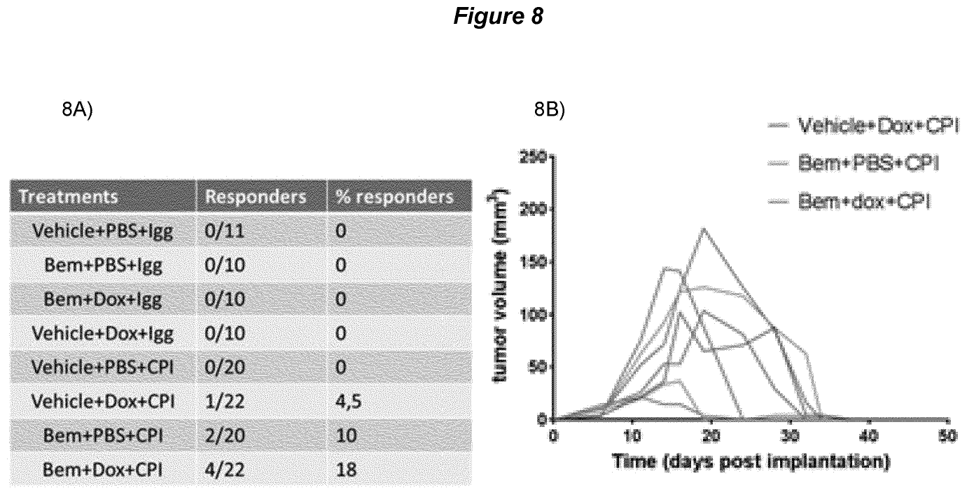

Many of the initial responders had tumor relapse (FIG. 7B) but some long term responders survived in the CPI+Dox, CPI+Bern or triple combination groups, with respectively 4.5%, 10% and 18% of responders (FIG. 8A).

For most of animals the relapse is observed around 40 days post implantation, though for some animals it happens later, at around 60 or 80 days (FIG. 7B). No other relapse has been observed for the long term responders even after the stop of Bemcentinib treatment at day 105 post tumor implantation.

Effects of Combination Therapy on Metastasis, Tumor Weight, and Spleen Weight

Lung, spleen and lung metastasis were examined after animal sacrifice. Only a few animals displayed macro metastasis on liver and spleen and were found in all the different treatment groups. The metastasis will be counted after paraffin embedding. Lung macro metastases were found in many animals in the different groups (Table 5).

TABLE 5
Lung macro metastasis (10+ is noted for animals with massive lung metastasis, A1-A15 is for
the animals in the different groups).

| | A1 | A2 | A3 | A4 | A5 | A6 | A7 | A8 | A9 | A10 | A11 | A12 | A13 | A14 | A15 |
|---|---|---|---|---|---|---|---|---|---|---|---|---|---|---|---|
| Vehicle + | 10+ | 10+ | 3 | 4 | 8 | 7 | 10+ | | | | | | | | |
| PBS + Igg | | | | | | | | | | | | | | | |
| Bem + PBS + | 6 | 5 | 0 | 4 | 10+ | 2 | 10 | 5 | 5 | 10+ | | | | | |
| Igg | | | | | | | | | | | | | | | |
| Bem + Dox + | 3 | 2 | 5 | 10+ | 10+ | 10+ | 5 | 3 | 4 | | | | | | |
| Igg | | | | | | | | | | | | | | | |
| Vehicle + | 10+ | 6 | 3 | 0 | 4 | 0 | 10+ | 4 | 5 | | | | | | |
| Dox + Igg | | | | | | | | | | | | | | | |
| Vehicle + | 4 | 8 | 5 | 10+ | 3 | 3 | 4 | 5 | 4 | 5 | | | | | |
| PBS + CPI | | | | | | | | | | | | | | | |
| Vehicle + | 0 | 0 | 10+ | 3 | 0 | 0 | 0 | 0 | 2 | 6 | 0 | 6 | 10+ | 8 | |
| Dox + CPI | | | | | | | | | | | | | | | |
| Bem + PBS + | 0 | 0 | 6 | 5 | 0 | 10+ | 6 | 0 | 0 | 10+ | 10+ | 0 | 1 | 0 | |
| CPI | | | | | | | | | | | | | | | |
| Bem + Dox + | 0 | 9 | 3 | 5 | 10+ | 7 | 0 | 1 | 0 | 7 | 10+ | 0 | 6 | 2 | 2 |
| CPI | | | | | | | | | | | | | | | |

All animals in the control groups not treated with CPI have lung metastasis. In the CPI treated groups some animals display lung metastasis, some do not. There seems to be no clear effect of the different treatments on metastasis. These results will need to be confirmed by histological observations.

No significant difference in tumor or spleen weight was observed between the different groups (Table 6). There is some variability within the different groups (Tumor and Spleen weight).

TABLE 6
Tumor and spleen weight post sacrifice. The average
for animals of each group is presented.

| | Average tumor | Average spleen |
|---|---|---|
| | weight (g) | weight(g) |
| Vehicle + PBS + Igg | 1.418182 | 1.12 |
| Bem + PBS + Igg | 1.555556 | 0.933333 |
| Bem + Dox + Igg | 1.4 | 1.02 |
| Vehicle + Dox + Igg | 1.51 | 1.16 |
| Vehicle + PBS + CPI | 1.6 | 1.12 |
| Vehicle + Dox + CPI | 1.49375 | 1.193333 |
| Bem + PBS + CPI | 1.307692 | 1.04375 |
| Bem + Dox + CPI | 1.357895 | 1.057143 |

Effects of Combination Therapy on Expression of 84 Type I IFN Related Genes

The survival study results discussed above confirm the potentiating effect of Doxorubicin, Bemcentinib and a combination of those 2 treatments on CPI treatment. The highest effect on survival was obtained with a triple combination treatment of AXL inhibitor (bemcentinib) immune checkpoint modulator (anti-CTLA4/anti-PD1 CPI), and cytotoxic chemotherapy (doxorubicin).

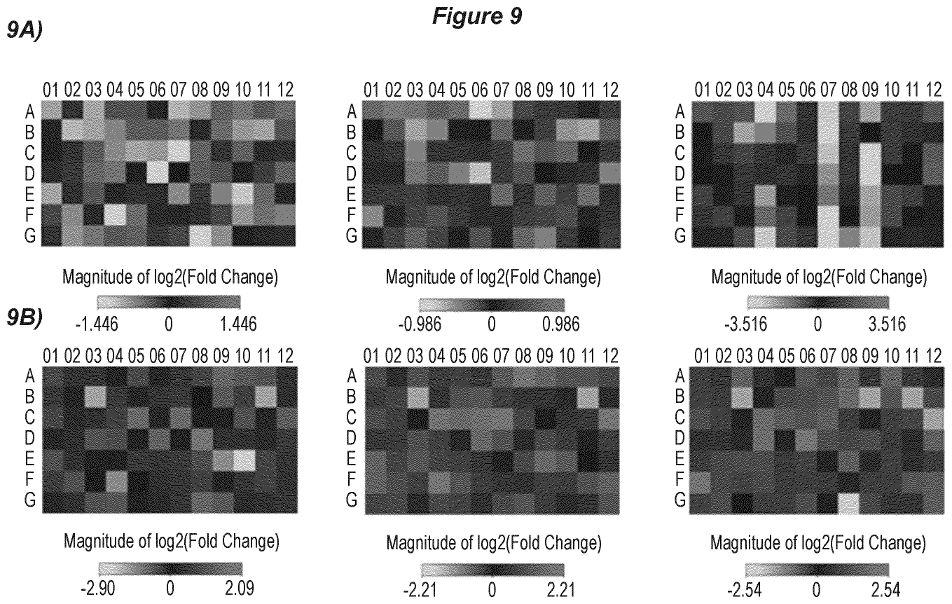

In order to investigate if this effect on survival is due to an increase of IFN type response, a mechanistic study was performed on the day of CPI treatment initiation in order to check IFN type I expression when the CPI treatment was initiated. RT² Profiler™ PCR Array Mouse Type I Interferon Response was performed, Heatmaps of fold changes of treated groups compared to controls are presented in FIG. 9.

No increase of IFN type I one related genes are observed for the 3 treated groups as compared to the control groups, and some genes are even down regulated (FIG. 9A). It is to be noted that animals from control groups have been injected intratumorally with PBS which may have led to an IFN type I response. The expression of IFN type I related genes may also already be high in the controls resulting in no detectable increase in the treated groups.

FIG. 9B illustrates that the array is very dependent on gene expression in the controls. Compared to one of the controls we see an increase of expression of IFN type I related genes in one animal treated with doxorubicin or doxorubicin+bemcentinib (FIG. 9B). Animals treated with bemcentinib show only a few upregulated genes (FIG. 9B).

### Experimental Conclusions

The results presented here confirm a potentiating effect on immune checkpoint modulator treatment (anti-CTLA4/anti-PD1 CPI) of cytotoxic chemotherapy (doxorubicin) or AXL inhibitors (bemcentinib). A higher potentiating effect is observed with cytotoxic chemotherapy (Doxorubicin) and AXL inhibitor (bemcentinib) used together. In the present study this effect was not only observed as a significant delay on tumor growth but as an effect on long term responders (more than 150 without tumor relapse).

In the results described herein, CPI treatment alone had a small effect on tumor growth. A small growth delay was observed compared to all control groups, not treated with CPI. The results described herein also confirmed the potentiating effect of Bemcentinib on CPI treatment. With a delay of tumor growth and an increase of the median survival compared to control animals and CPI treatment alone. 10% of long term responders (no relapse after 150 days post implantation) were also observed. These results shows a similar effect of doxorubicin and Bemcentinib on mice survival when used in combination with CPI. No significant difference is observed in term of mouse survival curves or tumor growth but only 4.5% long term responders were observed for doxorubicin compared to 10% for bemcentinib. Interestingly, when considering animals showing an initial response the percentages were similar, 27 and 25% respectively, so more tumor relapse was observed for doxorubicin treated animals.

The triple combination treatment significantly increased mouse survival compared to all other treatments, as well as increased the number of initial responders: 50% compared to 27% (Dox+CPI), 25% (Bern+CPI) and 5% (CPI alone). More interestingly an increase of long term responders was also observed with 18% for the triple combination treatment compared to 4.5% (Dox+CPI) and 10% (Bern+CPI).

These results demonstrate that adding standard cytotoxic chemotherapy to treatment regimes including AXL inhibitors in combination with immune checkpoint modulators increases the therapeutic benefit of the treatment.

The mechanisms of the effects of the triple combination therapy remain to be explained, as no link between treatment efficacy and IFN type I response was shown is this study. Further investigation will be needed. It is possible that the controls already had a strong IFN response, which might explain why we did not detect any further increase in the treated group. This hypothesis is supported by the analysis of some samples with one of the controls. Here an upregulation was detected. Control animals have received PBS injection intratumorally so this may have induced an interferon response.

Further mechanistic studies should include animals with no intra tumor injection. It might also be that the mice showing no increase of IFN type 1 related genes would have become non responders and those showing increase of IFN type 1 related genes would have become responders. It may also be that the IFN type I status before CPI initiation is not the most important for treatment efficacy as we observed a tumor volume decrease for all CPI treated group and then started to grow back, in a time different for each group.

### Example 2

In the results reported in Example 1, investigation of the molecular mechanism underlying the effects of the triple combination found no significant difference of expression of type I interferon (IFN) related genes at the timepoints at which samples were taken from the different treatment groups.

To further investigate whether an enhanced tumoral IFN response could be detected at earlier timepoints, a new molecular mechanism focused study was performed. To avoid missing the IFN response, 3 timepoints were selected (24, 48 and 72 hours post doxorubicin injection) for tumor sampling. To verify if the doxorubicin dose influenced the response, 3 dose levels were evaluated (1, 3 and 6 mg/kg). In addition, DMXAA (a Sting agonist) was included to directly activate IFN response.

Other potential mechanisms to explain the effects of combined treatments reported in Example 1 were also assessed. EMT related genes were assessed by RT² PCR plates, the concentration of circulating cytokines was assessed by Luminex, and global gene expression changes were assessed by RNA sequencing.

Materials & Methods

Cell Harvest

Exponentially growing 4T1 mammary carcinoma cells were harvested and re-suspended at 4×10⁶ per ml in the mixture of serum-free RPMI medium and Matrigel (1:1). The cell suspension showed over 95% viability.

Tumor Injection Procedure

Each animal was weighed before cell implantation. Mice were anesthetized by inhalation of Sevoflurane. Anesthesia was induced at 8% Sevoflurane and maintained at 4%. Every mouse was placed on a heating pad and animal was appropriately marked. Under a suitable depth of unconsciousness, animals were shaved, and skin and surrounding region was washed with Chlorhexidine (1 mg/ml) with use of sterile gauze. Injection was done orthotopically with one tumor per mouse with 0.05 ml of approximately 2×10⁵ 4T1 cells in serum-free RPMI medium/Matrigel (1:1).

TABLE 7
experimental groups; time indicates days post implantation

| | | | | Time of sacrifice |
|---|---|---|---|---|
| | | Oral | No. of | (hours after dox |
| | IT treatment | dosing | animals | injection) |
| A (no IT) | No (IT) | Vehicle | 9* | 24, 48, |
| B (no ITbem) | No (IT) | Bem | 12 | 72 hours* |
| C (Control) | PBS (IT) | Vehicle | 12 | |
| D (Bem) | PBS (IT) | Bem | 12 | |
| E (Dox1) | Dox (1 mg/kg) | Vehicle | 12 | |
| F (Dox1bem) | Dox (1 mg/kg) | Bem | 12 | |
| G (Dox3) | Dox (3 mg/kg) | Vehicle | 12 | |
| H (Dox3bem) | Dox (3 mg/kg) | Bem | 12 | |
| I (Dox6) | Dox (6 mg/kg) | Vehicle | 12 | |
| J (Dox6bem) | Dox (6 mg/kg) | Bem | 12 | |
| K (Sting) | Sting | Vehicle | 12 | |
| L (Stingbem) | Sting | Bem | 12 | |

4 animals per group were sacrificed at each time point
*3 animals per group were sacrificed at each time point in group A

Treatment Schedule

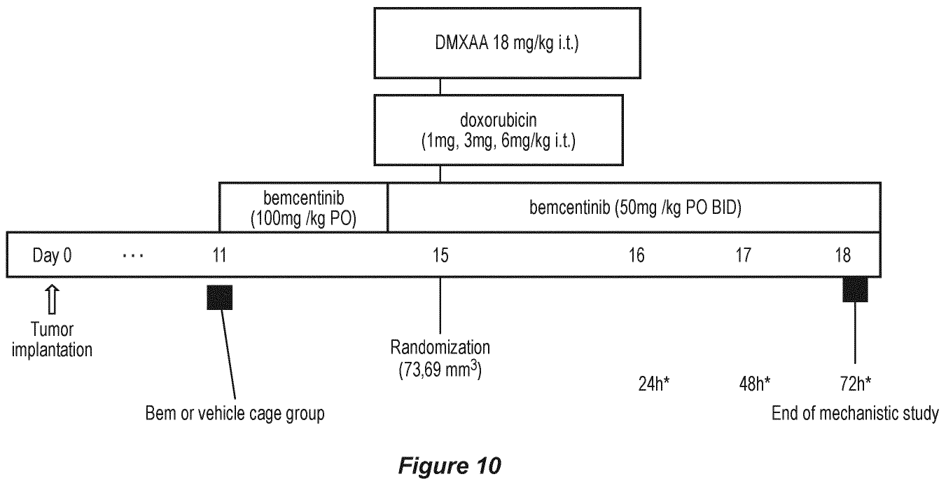

AXL inhibitor bemcentinib was given Bid (twice a day) to groups as described in Table 7, starting on the day of randomization 1 as shown in FIG. 10. Bemcentinib was given as a loading dose for 3 days (11-13 after tumor implantation) and then as a standard dose until sacrifice. Doxorubicin was administered as a single injection to the appropriate groups according to Table 1, on day 15 after tumor cell implantation as described in FIG. 10.

Randomization

Treatment groups are indicated in Table 7. 150 Balb/C mice were implanted and dispatched in 30 cages with 5 animals per cage. 9 mice have been excluded of the study because of the absence of tumor on day 11. On day 11 post implantation half of the cages were placed in the bemcentinib treated group and the other half in the bemcentinib treated group. The average tumor volume when bemcentinib treatment was initiated (day 11) was 54.99 mm³. Once the average tumor size reached 73.69 mm³ (tumor volume=L×W×W/2) the randomization was performed prior to doxorubicin injection on day 15. The randomization was conducted with Latin square method based on average tumor volume of all animals either in the bemcentinib group or in the vehicle treated group. The tumor volumes when bemcentinib treatment was initiated and the day of randomization are recorded in table 8. The intra tumomoral injection of doxorubicin and sting agonists was adjusted to the tumor volume.

TABLE 8
Tumour volumes at randomization

| | Bemcentinib initiation (day 11) | Randomization (day 15) |
|---|---|---|
| A | 45.13 ± 8.13 | 69.29 ± 14.47 |
| B | 47.1 ± 7.16 | 66.92 ± 9.58 |
| C | 73.1 ± 6.07 | 95.12 ± 9.71 |
| D | 40.70 ± 6.68 | 50.35 ± 10.16 |
| E | 62.87 ± 11.96 | 74.09 ± 12.97 |
| F | 46.65 ± 6.82 | 64.51 ± 9.20 |
| G | 64.72 ± 11.33 | 84.26 ± 12.19 |
| H | 53.78 ± 0.97 | 66.61 ± 9.21 |
| I | 71 ± 12.45 | 78.13 ± 12.72 |
| J | 51.36 ± 8.26 | 75.45 ± 11.13 |
| K | 55.82 ± 6.85 | 82.58 ± 11.84 |
| L | 45.33 ± 4.88 | 66.83 ± 9.08 |

Tumor Growth Measurements

Tumor growth was measured with a digital handheld caliper twice weekly from emergence of tumor and until the day of euthanasia

Body Weights

Animals were weighed prior to tumor cell injection, prior to daily dosing and prior to euthanasia. No weight loss was observed during the short mechanistic study except for the group treated with the sting agonist (group K and L).

Intra Tumor (IT) Injection Procedure

The animals were injected either with doxorubicin (E,F,G,H,I,J), or with a Sting agonist (DMXAA(5,6-Dimethylxanthenone-4-acetic Acid, ASA404, Vadimezan) (group K,L). The control groups were injected with PBS (C,D) or not injected (A,B). 141 mice were treated Intratumorally, 2 mice died following the injection of DMXAA. The study was based on 139 balb/C mice.

Doxorubicin Injection

Doxorubicin is provided in a 2 mg/ml solution. As it is technically challenging to inject 50 ul liquid into tumors measuring <100 mm³, the total dosing volume was calculated based on the following formula: V=50 ul×(TV/120). This is done to minimize variability due to leakage during intratumoral injection. For the 1 mg/kg dose (E,F) Doxorubicin was given at a concentration of 0.5 mg/ml, which corresponds to 1 mg/kg for a 25 g mouse with a tumor of 120 mm³, and 1 mg/kg for a 20 g mouse with a tumor of 100 mm³. For 3 mg/kg dose (G,H), a solution of 1.5 mg/ml was used and same volumes was injected. For the 6 mg/kg dose (I,J) the commercial solution of 2 mg/ml will be used and 75 ul was injected for tumor with a volume of 120 mm³.

Sting Agonist Injection

DMXAA (5,6-Dimethylxanthenone-4-acetic Acid, ASA404, Vadimezan) is provided as powder and was reconstituted in PBS (7.5% sodium bicarbonate, at a concentration of 9 mg/ml). The same formula as for doxorubicin was used V=50 ul×(TV/120). 450 ug of DMXAA was injected for a tumor volume of 120 mm³ (K, L).

Oral Dosing (PO) Procedure

On the appropriate days, each animal in Group A, B, E, G, I and K received a specific amount of vehicle (0.5% (w/w) hydroxypropyl methylcellulose/0.1% (w/w) Tween 80). After 3 days of a loading dose (100 mg/kg) from day 11, each animal in Group C, D, F, H, J and L received bemcentinib at the dose of 50 mg/kg as indicated in Table 7. Dosing schedule was twice daily on a 5 day-2 day off schedule until 18 days post implantation. Dosing volume was 10 ml/kg by oral gavage.

Clinical Observations

Animals were observed once daily for general appearance.

Mortality/Morbidity

All animals were examined once daily for mortality or morbidity. An animal was sacrificed if the animal lost >20% of the body weight or was judged as moribund. Mice bearing tumors that reached a volume exceeding 1000 mm³ were euthanized. Euthanized animals and any animals found dead prior to rigor mortis were necropsied.

Euthanasia

Animals were anesthetized with Sevoflurane and euthanized by cervical dislocation. Euthanasia was conducted by following the Institutional SOP accordingly.

Tissue Collection

Spleens were weighed and prepared for Cytof (cut into pieces of 2 mm and frozen in FBS 90%, DMSO 10% and stored at −80C prior to dissociation). Blood was collected by cardiac puncture. Plasma was collected and frozen to test cytokines.

Tumors were weighed and cut in half along the long axis, one half will be snap frozen in liquid nitrogen in 2 cryotubes per tumor, the other half was prepared for Cytof (Cut into pieces of 2 mm and frozen in FBS 90%, DMSO 10% and stored at −80C prior to dissociation). The samples will be dissociated (Macs dissociation) for the groups with IFN type I response.

Analysis of Tumor Tissue

RNA was extracted from 139 mice from the different groups snap frozen tumors using the RNEasy Microarray Tissue Mini kit from Qiagen following the manufacturer's instructions. As tumors contained Doxorubicin and powderizing them could expose personnel to harmful substances, the RNA was extracted in a closed system using the GentleMACS dissociator and M Tubes. Purity and concentration of RNA was measured using Nanodrop. cDNA was synthesized from RNA using the RT² First Strand kit (Qiagen) according to the manufacturer's instructions. cDNA was mixed with RT² SYBR Green Mastermix (Qiagen) and loaded into RT Profiler™ PCR Array Mouse Type I Interferon Response or RT² Profiler™ PCR Array Mouse Epithelial to Mesenchymal Transition (EMT), analyzing the expression of 84 genes listed in Tables 9 and 10. The plates were analyzed using Light Cycler 480 (Roche) and data were analyzed using the Qiagen Data Analysis Centre and Graph Pad (Prism).

The controls without IT injection (A, B) were not analyzed because they are not appropriate controls, they were only included in case of the absence of IFN by comparing treated samples with controls injected with PBS.

Fold change of the genes analyzed were calculated using the ΔΔ Ct method. Quiagen RT² software was used to analyze all the samples from different groups. To be able to generate heatmaps and graph on fold change including error bars, the data have been analyzed manually with Graph Pad (Prism) when the Quiagen RT² software gave significant results between the different groups. For IFN type 1 response 1 mg/kg 48, 72 h and 3 mg/kg 48 h, were analyzed manually. For EMT, 1 mg/kg 48, 72 h, 3 mg/kg 72 h. When the data have been analyzed manually, the results are presented as histograms of mRNA fold changes. For the time points and doses analyzed by the software, the results are presented as tables.

TABLE 9
Type I interferon response (interferon stimulated genes) analysed

| Symbol | Description |
|---|---|
| Adar | Adenosine deaminase, RNA-specific |
| Bag3 | Bcl2-associated athanogene |
| Bst2 | Bone marrow stromal cell antigen 2 |
| Casp1 | Caspase 1 |
| Cav1 | Caveolin 1, caveolae protein |
| Ccl2 | Chemokine (C-C motif) ligand 2 |
| Ccl4 | Chemokine (C-C motif) ligand 4 |
| Cc15 | Chemokine (C-C motif) ligand 5 |
| Cd69 | CD69 antigen |
| Cd70 | CD70 antigen |
| Cd80 | CD80 antigen |
| Cd86 | CD86 antigen |
| Cdkn1b | Cyclin-dependent kinase inhibitor 1B |
| Ciita | Class II transactivator |
| Crp | C-reactive protein, pentraxin-related |
| Cxcl10 | Chemokine (C-X-C motif) ligand 10 |
| Ddx58 | DEAD (Asp-Glu-Ala-Asp) box polypeptide 58 |
| Eif2ak2 | Eukaryotic translation initiation factor 2-alpha kinase 2 |
| Gbp2b | Guanylate binding protein 1 |
| H2-BI | Histocompatibility 2, blastocyst |
| H2-D1 | Histocompatibility 2, D region locus 1 |
| H2-K1 | Histocompatibility 2, K1, K region |
| H2-M10.1 | Histocompatibility 2, M region locus 10.1 |
| H2-M3 | Histocompatibility 2, M region locus 3 |
| H2-T10 | Histocompatibility 2, T region locus 10 |
| Ifi204 | Interferon activated gene 204 |
| Ifi30 | Interferon gamma inducible protein 30 |
| Ifih1 | Interferon induced with helicase C domain 1 |
| Ifit1 | Interferon-induced protein with tetratricopeptide repeats 1 |
| Ifit2 | Interferon-induced protein with tetratricopeptide repeats 2 |
| Ifit3 | Interferon-induced protein with tetratricopeptide repeats 3 |
| Ifitm1 | Interferon induced transmembrane protein 1 |
| Ifitm2 | Interferon induced transmembrane protein 2 |
| Ifitm3 | Interferon induced transmembrane protein 3 |
| Ifna2 | Interferon alpha 2 |
| Ifna4 | Interferon alpha 4 |
| Ifnar1 | Interferon (alpha and beta) receptor 1 |
| Ifnar2 | Interferon (alpha and beta) receptor 2 |
| Ifnb1 | Interferon beta 1, fibroblast |
| Ifne | Interferon epsilon |
| Ifnz | Interferon zeta |
| Il10 | Interleukin 10 |
| Il15 | Interleukin 15 |
| Il6 | Interleukin 6 |
| Irf1 | Interferon regulatory factor 1 |
| Irf2 | Interferon regulatory factor 2 |
| Irf3 | Interferon regulatory factor 3 |
| Irf5 | Interferon regulatory factor 5 |
| Irf7 | Interferon regulatory factor 7 |
| Irf9 | Interferon regulatory factor 9 |
| Isg15 | ISG15 ubiquitin-like modifier |
| Isg20 | Interferon-stimulated protein |
| Jak1 | Janus kinase 1 |
| Jak2 | Janus kinase 2 |
| Mal | Myelin and lymphocyte protein, T-cell differentiation protein |
| Met | Met proto-oncogene |
| Mx1 | Myxovirus (influenza virus) resistance 1 |
| Mx2 | Myxovirus (influenza virus) resistance 2 |
| Myd88 | Myeloid differentiation primary response gene 88 |
| Nmi | N-myc (and STAT) interactor |
| Nos2 | Nitric oxide synthase 2, inducible |
| Oas1a | 2′-5′ oligoadenylate synthetase 1A |
| Oas1b | 2′-5′ oligoadenylate synthetase 1B |
| Oas2 | 2′-5′ oligoadenylate synthetase 2 |
| Pml | Promyelocytic leukemia |
| Prkcz | Protein kinase C, zeta |
| Psme2 | Proteasome (prosome, macropain) 28 subunit, beta |
| Sh2d1a | SH2 domain protein 1A |
| Shb | Src homology 2 domain-containing transforming protein B |
| Socs1 | Suppressor of cytokine signaling 1 |
| Stat1 | Signal transducer and activator of transcription 1 |
| Stat2 | Signal transducer and activator of transcription 2 |
| Stat3 | Signal transducer and activator of transcription 3 |
| Tap1 | Transporter 1, ATP-binding cassette, sub-family B |
| | (MDR/TAP) |
| Ticam1 | Toll-like receptor adaptor molecule 1 |
| Timp1 | Tissue inhibitor of metalloproteinase 1 |
| Tlr3 | Toll-like receptor 3 |
| Tlr7 | Toll-like receptor 7 |
| Tlr8 | Toll-like receptor 8 |
| Tlr9 | Toll-like receptor 9 |
| Tnfsf10 | Tumor necrosis factor (ligand) superfamily, member 10 |
| Traf3 | Tnf receptor-associated factor 3 |
| Tyk2 | Tyrosine kinase 2 |
| Vegfa | Vascular endothelial growth factor A |

TABLE 10
EMT- related genes analysed

| Symbol | Description |
|---|---|
| Ahnak | AHNAK nucleoprotein (desmoyokin) |
| Akt1 | Thymoma viral proto-oncogene 1 |
| Bmp1 | Bone morphogenetic protein 1 |
| Bmp7 | Bone morphogenetic protein 7 |
| Cald1 | Caldesmon 1 |
| Camk2n1 | Calcium/calmodulin-dependent protein kinase II inhibitor 1 |
| Cav2 | Caveolin 2 |
| Cdh1 | Cadherin 1 |
| Cdh2 | Cadherin 2 |
| Col1a2 | Collagen, type I, alpha 2 |
| Col3a1 | Collagen, type III, alpha 1 |
| Col5a2 | Collagen, type V, alpha 2 |
| Ctnnb1 | Catenin (cadherin associated protein), beta 1 |
| Dsc2 | Desmocollin 2 |
| Dsp | Desmoplakin |
| Egfr | Epidermal growth factor receptor |
| Erbb3 | V-erb-b2 erythroblastic leukemia viral oncogene homolog 3 |
| | (avian) |
| Esr1 | Estrogen receptor 1 (alpha) |
| F11r | F11 receptor |
| Fgfbp1 | Fibroblast growth factor binding protein 1 |
| Fn1 | Fibronectin 1 |
| Foxc2 | Forkhead box C2 |
| Fzd7 | Frizzled homolog 7 (Drosophila) |
| Gng11 | Guanine nucleotide binding protein (G protein), gamma 11 |
| Gsc | Goosecoid homeobox |
| Gsk3b | Glycogen synthase kinase 3 beta |
| Igfbp4 | Insulin-like growth factor binding protein 4 |
| Il1rn | Interleukin 1 receptor antagonist |
| Ilk | Integrin linked kinase |
| Itga5 | Integrin alpha 5 (fibronectin receptor alpha) |
| Itgav | Integrin alpha V |
| Itgb1 | Integrin beta 1 (fibronectin receptor beta) |
| Jag1 | Jagged 1 |
| Krt14 | Keratin 14 |
| Krt19 | Keratin 19 |
| Krt7 | Keratin 7 |
| Mitf | Microphthalmia-associated transcription factor |
| Mmp2 | Matrix metallopeptidase 2 |
| Mmp3 | Matrix metallopeptidase 3 |
| Mmp9 | Matrix metallopeptidase 9 |
| Msn | Moesin |
| Mst1r | Macrophage stimulating 1 receptor (c-met-related tyrosine |
| | kinase) |
| Map1b | Microtubule-associated protein 1B |
| Nodal | Nodal |
| Notch1 | Notch gene homolog 1 (Drosophila) |
| Nudt13 | Nudix (nucleoside diphosphate linked moiety X)-type motif |
| | 13 |
| Ocln | Occludin |
| Pdgfrb | Platelet derived growth factor receptor, beta polypeptide |
| Plek2 | Pleckstrin 2 |
| Desi1 | PPPDE peptidase domain containing 2 |
| Ptk2 | PTK2 protein tyrosine kinase 2 |
| Ptp4a1 | Protein tyrosine phosphatase 4a1 |
| Rac1 | RAS-related C3 botulinum substrate 1 |
| Rgs2 | Regulator of G-protein signaling 2 |
| Serpine1 | Serine (or cysteine) peptidase inhibitor, clade E, member 1 |
| Gemin2 | Survival of motor neuron protein interacting protein 1 |
| Smad2 | MAD homolog 2 (Drosophila) |
| Snai1 | Snail homolog 1 (Drosophila) |
| Snai2 | Snail homolog 2 (Drosophila) |
| Snai3 | Snail homolog 3 (Drosophila) |
| Sox10 | SRY-box containing gene 10 |
| Sparc | Secreted acidic cysteine rich glycoprotein |
| Spp1 | Secreted phosphoprotein 1 |
| Stat3 | Signal transducer and activator of transcription 3 |
| Steap1 | Six transmembrane epithelial antigen of the prostate 1 |
| Tcf4 | Transcription factor 4 |
| Tcf711 | Transcription factor 7-like 1 (T-cell specific, HMG box) |
| Tfpi2 | Tissue factor pathway inhibitor 2 |
| Tgfb1 | Transforming growth factor, beta 1 |
| Tgfb2 | Transforming growth factor, beta 2 |
| Tgfb3 | Transforming growth factor, beta 3 |
| Timp1 | Tissue inhibitor of metalloproteinase 1 |
| Tmeff1 | Transmembrane protein with EGF-like and two follistatin- |
| | like domains 1 |
| Tmem132a | Transmembrane protein 132A |
| Tspan13 | Tetraspanin 13 |
| Twist1 | Twist homolog 1 (Drosophila) |
| Vcan | Versican |
| Vim | Vimentin |
| Vps13a | Vacuolar protein sorting 13A (yeast) |
| Wnt11 | Wingless-related MMTV integration site 11 |
| Wnt5a | Wingless-related MMTV integration site 5A |
| Wnt5b | Wingless-related MMTV integration site 5B |
| Zeb1 | Zinc finger E-box binding homeobox 1 |
| Zeb2 | Zinc finger E-box binding homeobox 2 |

Cytokine Analysis (Luminex Assay)

43 mouse cytokines (Table 11) were measured in mouse plasma samples using the Thermo fisher scientific ProcartaPlex™ Multiplex Immunoassay (Luminex). The manufacturer user guide was followed. The samples were analyzed by (MAGPIX) and the results were obtained as concentration in pg/ml of mouse plasma. Plasma from mice from all groups as presented in table 7 was analyzed. At least 3 samples per group, 4 for many, were analyzed. Due to the cost of the experiment, only one time point was performed, 72 hours post doxorubin and 48 hours post DMXAA intratumoral injection.

TABLE 11
Mouse cytokines analysed by ProcartaPlex ™ Multiplex
Immunoassay

| Symbol | Description |
|---|---|
| G-CSF | Granulocyte colony-stimulating factor |
| IL-10 | Interleukin-10 |
| IL-3 | Interleukin-3 |
| CD80 | Cluster of differentiation 80 |
| LIF | Leukemia inhibitory factor |
| IL-1 beta | Interleukin-1 beta |
| IL-2 | Interleukin-2 |
| M-CSF | Macrophage colony stimulating factor |
| CXCL10 (IP-10) | (Interferon gamma-induced protein 10) |
| VEGF-A | Vascular endothelial growth factor-A |
| IL-4 | Interleukin-4 |
| IL-5 | Interleukin-5 |
| IL-6 | Interleukin-6 |
| PD-L1 | Programmed death-ligand 1 |
| IFN alpha | Interferon alpha |
| IL-22 | Interleukin-22 |
| IL-13 | Interleukin-13 |
| IL-27 | Interleukin-27 |
| IL-23 | Interleukin-23 |
| IFN gamma | Interferon gamma |
| IL-12p70 | Interleukin 12p70 |
| GM-CSF | Granulocyte-macrophage colony-stimulating factor |
| CCL5 | Chemokine C-C motif ligand 5 |
| (RANTES) | (Regulated upon Activation, Normal T Cell |
| | Expressed and Presumably Secreted) |
| TNF alpha | Tumor necrosis factor alpha |
| TIM-3 | T-cell immunoglobulin and mucin domain-3 |
| MIP-1 alpha | Macrophage Inflammatory Protein1 alpha |
| CCL7 (MCP-3) | Chemokine C-C motif ligand 2 (Monocyte |
| | Chemotactic Protein 3) |
| CCL2 (MCP-1) | Chemokine C-C motif ligand 7 (Monocyte |
| | chemotactic protein 1) |
| IL-17A | Interleukin-17A |
| PD-L2 | Programmed death-ligand 1 |
| IL-15 | Interleukin-15 |
| MIP-2 alpha | Macrophage Inflammatory Protein2 alpha |
| IL-alpha | Interleukin-alpha |
| CXCL5 (ENA- | C-X-C Motif Chemokine Ligand 5 |
| 87) | (epithelial-derived neutrophil-activating peptide 78) |
| IL-19 | Interleukin-19 |
| CCL11 (Eotaxin) | C-C motif chemokine 11 (eosinophil chemotactic |
| | protein) |
| IL-7 | Interleukin-7 |
| IL-2R | Interleukin-2R |
| IL-7R alpha | Interleukin-7R alpha |
| MIP-1 beta | Macrophage Inflammatory Protein1 beta |
| IL-33 | Interleukin-33 |
| IL-31 | Interleukin-31 |
| IFN beta | interferon beta |

RNAseq Analysis

FASTQ files were generated and quality controlled by the Genomic Core Facility. The trimmed samples were aligned to the reference genome using STAR. Gene expression quantification was achieved using feature Counts. Following QC, 8 and 7 samples from in vitro and in vivo studies respectively were identified as outliers and were excluded from subsequent analysis. RUVseq was used to remove hidden batch effect and/or other unwanted variations.

Read counts were analyzed for differential expression using the R package DESeq2. DESeq2 uses the raw read counts, applies an internal normalization method, and does estimation of library size, estimation of dispersion, and negative binomial generalized linear model fitting. The resulting differentially expressed gene lists (adjusted p-value cutoff=0.05) were then analyzed using Metascape to find the enriched terms.

Statistics

GraphPadPrism was used to generate the heat maps, graphs with mRNA fold change, and to make the statistics. Multiple TTests, paired were performed on ΔΔ Ct values.

Results

Balb/c mice were injected orthotopically with 4T1 carcinoma cells. Bemcentinib treatment was initiated on the day of randomization-1 (11 days after tumor implantation), administered as a loading dose of 100 mg/kg PO Bid for the first 3 days starting day 11 after tumor implantation, and then at 50 mg/kg Bid for the rest of the study. Doxorubicin or Sting agonist (DMXAA; 5,6-dimethylxanthenone-4-acetic acid) were administered as a single i.t injection to the appropriate groups on day 15 after tumor cell implantation, 4 days after initiation of the bemcentinib treatment.

Doxorubicin treatment was found to have a limited effect on the body weight of mice—all mice in different groups gained weight, from 2 to 5% of initial body weight during the experiment on average in the different groups. Before intratumoral treatment, mice treated with the Sting agonist, DMXAA also showed a weight gain. However, on average, a strong decrease of body weight following Sting agonist injection was observed—around 5% of loss for 1 day. This was observed with or without bemcentinib treatment.

In this short in vivo study doxorubicin had no effect on tumor growth, while tumors injected with Sting agonist displayed a decreased tumor growth. Some mice displaying a tumor size greater than 250 mm³ died a few minutes after Sting agonist injection; an increase of breathing rate was observed in those mice. As the dose was adjusted to tumor volume, MTD may have been reached.

Bemcentinib Potentiates IFN Response at Lower i.t. Doses of Doxorubicin

The expression of type I IFN related genes (ISG) was assessed in tumors collected 24, 48 and 72 hours after i.t. doxorubicin treatment. 3 groups were compared per timepoint: bemcentinib (bern), doxorubicin only (dox) and doxorubicin combined with bemcentinib (doxbern), the fold changes were compared with the control groups injected with PBS, and the fold change was 1 for the control groups.

In the groups treated with 1 mg/kg doxorubicin, heat map analysis showed that 24 hours after injection, a few ISG were upregulated in both doxorubicin and doxorubicin+bemcentinb treated tumors. At the 24 hr timepoint no ISG were significantly upregulated compared to control in either bern or dox treated animals. It is believed that the 24 hr timepoint may be too early to detect a significant IFN response.

At the 48 h timepoint following the 1 mg/kg dose, one ISG was up regulated (3 fold) in the doxorubicin treated group while none were upregulated with bemcentinib only. At this low dose, doxorubicin alone was insufficient to induce an IFN type I response. In contrast, the doxorubicin (1 mg/kg)+bemcentinib combination induced significant upregulation of 16 type I IFN related genes compared to control; one of which displayed a 14 fold change, with the others ranging between 2-5 fold.

These results indicate that AXL inhibition generated a synergistic type I interferon response in combination with the 1 mg/kg dose i.t. doxorubicin treatment.

72 h after doxorubicin injection (1 mg/kg), no ISG were significantly upregulated in the different groups compared to control groups. This shows that the IFN response was transient under these conditions.

With 3 mg/kg doxorubicin one ISG was significantly upregulated compared to control for the doxorubicin group only 24 h after injection. The combined treatment Doxbern displayed 3 ISG upregulated after 24 h. At this higher dose few ISG were significantly upregulated at 24 h. Heatmap analysis of all 84 ISG showed that many were unchanged in the bemcentinib group, and many were upregulated in the doxorubicin or doxbern group. This was confirmed when genes with fold changes higher than 2 in one of the groups were selected—all changes were represented in the heatmap analysis; clearly many ISG were upregulated in both doxorubicin treated groups.

No significant upregulated ISG were detected 48 h after 3 mg/kg doxorubicin injection, in the dox and bern only groups. In the bemcentinib+doxorubicin group a significant upregulation was observed for 14 ISG. A few of these genes displayed an average fold change of around 10. For all other significantly upregulated genes the fold changes were between 3 and 4. Of the non-significant genes with a fold change higher than 2 many had similar values for doxorubicin alone and combination treatment.

These results indicate that AXL inhibition with bemcentinib in combination with 3 mg/kg doxorubicin generated a synergistic and significant IFN response.

At the 72 hr timepoint, no genes were significantly up regulated compared to control, consistent with the results observed for the 1 mg/kg dose.

With 6 mg/kg doxorubicin, 2 ISG genes were significantly upregulated 24 hours after injection in presence of bern, and only 1 ISG with doxorubicin only. Even with the higher dose of doxorubicin, no IFN type 1 response was observed 24 hours after the injection.

48 hours after 6 mg/kg doxorubicin injection, 8 genes were significantly upregulated in both the doxorubicin only group and in the combined treatment group. Some of the upregulated genes differed between these groups. The number of genes upregulated was lower than for the lower doses in presence of bemcentinib. The fold changes were not much higher for many of the genes, between 2 and 6 fold. These results indicate that the higher dose of doxorubicin may limit the effects of bemcentinib treatment observed at the lower doses of doxorubicin.

72 hours after 6 mg/kg doxorubicin injection, 6 genes were significantly upregulated in both the doxorubicin only group and in the combined treatment group. Compared to the other doses the Interferon type response was maintained at 72 hours. Few differences were observed with or without bemcentinib. Some of the overexpressed genes were similar in both groups and the fold changes were higher in the dox bern group. Some displayed a similar fold change. Contrary to what was observed for lower doses, the IFN type I response was maintained 72 hours after doxorubicin injection.

These results indicate that the potentiating effect of bemcentinib on doxorubicin induced type I response is limited at higher doxorubicin doses.

The results outlined above show that bemcentinib was needed to obtain a broad IFN type I response after doxorubicin i.t. The response was observed after 48 h and lost after 72 hours; this transient effect may be necessary to avoid long term IFN response-associated pro-tumor effects. The response was similar with 1 and 3 mg/kg i.t. doxorubicin, while little effect of bemcentinib was measured at the 6 mg/kg dose level.

Combination Bemcentinib and Doxorubicin has a Significant Effect on EMT Related Genes as Compared to Control and Single Treatments

EMT related gene expression in tumors was assessed by the RT² Profiler™ PCR Array for the 1 and 3 mg/kg doses at 48 and 72 hr timepoints. The highest dose and 24 hr timepoint samples were excluded based on the results outlined above.

48 hours after doxorubicin 1 mg/kg i.t. treatment, some EMT genes were upregulated in the different groups, while more genes seemed to be upregulated in the combination treatment compared to mono-treatments. Heatmap analysis also clearly showed many (twenty) downregulated genes in the bemcentinib+doxorubicin treatment group as compared to only one for bemcentinib and doxorubicin alone for the 1 mg/kg dose at the 72 hr timepoint. Ten of the twenty downregulated genes are known to normally be upregulated in EMT process—indicating a decrease of EMT in the combined treatment compared to control, that does not occur with the single treatments. Because these conditions were used in the efficacy studies in Example 1, the decrease of EMT in the combined treatment may explain the survival differences between the groups.

In the 3 mg/kg doxorubicin i.t. treated tumors, more downregulated EMT genes were observed with both the combination and doxorubicin alone as compared to bemcentinib monotherapy. 72 hours after doxorubicin injection, 1 EMT related gene was significantly downregulated in presence of bemcentinib compared to none with doxorubicin or bemcentinib only. Contrary to what was observed with the lower dose of doxorubicin, no significant effect on EMT related gene expression was observed.

These results indicate that the doxorubicin i.t. protocol is associated with an interferon response then a decrease of EMT and confirm that the combined treatment has a significant effect on EMT related genes as compared to control and single treatments.

Effects of Sting Agonist Treatment on IFN Response

RT² Profiler™ PCR Array Mouse Type I Interferon Response was performed on tumors collected at 24 and 48 h following intratumor injection of Sting agonist DMXAA. The 72 h time point was omitted as earlier studies (Dhakal, unpublished) have found that the IFN stimulation occurs within 24 h in vitro.

24 hours after DMXAA i.t. treatment, 8 ISG were significantly upregulated compared to control. A few genes had a fold change around 5, while 2 genes displayed a fold change above 5. DMXAA alone was sufficient to trigger a IFN response.

In the presence of bemcentinib and DMXAA, 15 genes were significantly upregulated compared to control. Bemcentinib addition generated a broader interferon response than DMXAA alone. For all but one ISG, all genes significantly upregulated with DMXAA were also upregulated with the combined treatment and displayed a higher fold change than with DMXAA alone. Only 2 ISG were significantly upregulated in the DMXXA group and not in the combined treatment. 3 ISG were upregulated in the bemcentinib only group (albeit at only around 2 fold). Bemcentinib alone did not trigger an IFN response.

These results show that 24 hours after injection bemcentinib potentiated the IFN type 1 response observed with DMXAA alone.

48 hours after DMXAA injection, 4 ISG were still upregulated in tumors from the combined treatment group, compared to 1 in the DMXAA only group. For many other ISG, an upregulation was observed but these were not significant as for the observations after 24 hours.

Together, these results show that DMXAA induced an IFN response at 24 h that attenuated by 48 h whereas in presence of bemcentinib, the IFN response was potentiated at 24 hours and maintained for some ISG at 48 hours.

Transcriptional Analysis of Tumors Collected 72 Hours after Doxorubicin i.t. Treatment

RNA sequence analysis (RNAseq) was performed on tumor samples from mice sacrificed 72 hours after doxorubicin i.t. treatment (corresponding to group E and F at the 72 hr timepoint in Example 1).

Many downregulated genes were observed in the bemcentinib group compared to control, with few genes upregulated in the bemcentinib group. Only 2 GO terms were significantly upregulated in the bemcentinib group compared to 20 downregulated GO terms in the bemcentinib group. All the terms downregulated in bemcentinib group were related with interferon response, and immune response. Surprisingly the IFN response seemed to be downregulated in bemcentinib treated animals compared to control.

Very few genes were significantly upregulated in the doxorubicin treated group compared to control—gene ontology analysis showed only 1 term with significant upregulation. No Interferon response was observed between the 2 groups.

Almost no genes were found to be upregulated in the doxbern group as compared to control, but many downregulated genes were observed. 20 terms were significantly downregulated in doxbern compared to control, though none of them seemed to be related to IFN response.

Comparison of the doxorubicin+bemcentinib group against bemcentinib alone identified many genes that were differentially expressed. Gene ontology showed that the interferon and immune response was upregulated in the doxbern compared to bern only group.

Comparison of the bemcentinib group against doxorubicin identified many genes that were differentially expressed. Gene ontology showed that the interferon and immune response is upregulated with similar results as when doxbern was compared to bern.

These results show that when compared with bemcentinib, both doxorubicin and doxorubicin combined with bemcentinib display an Interferon type 1 response.

Effects of Doxorubicin and DMXAA Treatment on Plasma Cytokines

A panel 43 of cytokines was measured in plasma samples from tumor-bearing mice 72 hours after doxorubicin i.t. treatment (3 dose levels 1 mg/kg, 3 mg/kg and 6 mg/kg) and 48 hours after DMXAA i.t. treatment, in control, single treatment (doxorubicin, or DMXAA, or bemcentinib), and combination (doxorubicin or DMXAA+bemcentinib) groups.

For 1 mg/kg doxorubicin i.t., 72 h post injection, out of 43 measured cytokines, 7 displayed a significantly different concentration between the control and other groups. 4 displayed a significant decrease of concentration in both bemcentib and bemcentinib in addition to doxorubicin—systemic effects which appear to be attributable to bemcentinib treatment. 2 of the 4 are immune checkpoint proteins, for which a reduced plasma concentration may be associated with a higher immune response. Only one cytokine was significantly upregulated in the Doxbern group compared with control.

For 3 mg/kg doxorubicin 72 h post injection. 5 cytokines had significantly different concentrations in the different groups. For 6 mg/kg doxorubicin, 72 h post injection. 3 cytokines displayed different concentrations in the different groups. For DMXAA, 48 h post injection. 8 cytokines had different concentration in the treated groups compared with controls.

The results show 3 major cytokines were consistently modified by the combination treatments—including downregulation of two immune checkpoint receptor ligands which play a role in negative regulation of the adaptive immune response, and upregulation of a chemoattractant cytokine.

### Experimental Conclusions

The results presented here confirm that at a low i.t. dose of doxorubicin (1 mg/kg), bemcentinib was necessary to generate an IFN response. Changes in interferon stimulated genes (ISG) were detected 24 hours before the beginning of ICB treatment in efficacy study in Example 1 (e.g. at 48 hours and not when ICB was started at 72 hours). The 3 mg/kg doxorubicin dose led to a similar IFN response. With the highest dose, 6 mg/kg the IFN response was observed with and without bemcentinib, thus obscuring the effect. The potentiating effects on IFN response observed at 48 hours was not maintained at 72 hours—consistent with the results in the mechanistic study of Example 1.

In addition, a down regulation of some EMT related genes was observed in the combined treatment with the 1 mg/kg dose of doxorubicin, 72 hours after injection. A decrease of EMT may explain the better survival in the combination group reported in Example 1.

RNAseq analysis of samples showed an upregulation of genes associated to interferon and immune response, between the bemcentinib compared to combined groups, correlating with survival differences between these groups. Changes in some plasma cytokines were observed upon treatment.

The results of this study demonstrate that the doxorubicin 1 mg/kg i.t. dose in combination with bemcentinib induced a significant IFN response 48 hours after injection, accompanied by a downregulation of EMT genes at 72 hours after tumor injection. These effects were not observed with single treatments.

In addition, the results of this study highlighted good results for DMXAA in combination with bemcentinb. The interferon response observed was as broad as for the low doses of doxorubicin, but with higher fold changes for many of the genes.

In conclusion, the present mechanism focused study found that in combination with low dose of doxorubicin (1 mg/kg), bemcentinib is necessary to obtain an interferon type one response. This response happened 24 hours before the beginning of treatment in the efficacy study in Example 1 (at 48 hours and not when ICB was started at 72 hours).

We can conclude that in vivo in the syngeneic 4T1 model, bemcentinib leads to an IFN response, a downregulation of EMT related genes, as well as few effects on circulating cytokines. Taken together this may explain the potentiating effect of bemcentinib on immune checkpoint inhibitor treatment in combination with doxorubicin observed in Example 1.

### Example 3

Materials & Methods

Cell Harvest

Yale university mouse melanoma cells 1.7 (Yumm 1.7). BrafV600E/wt (knock in conditionally activated Braf allele) Cdkn2a−/− Pten−/− (conditionally inactivated). Cultured in DMEM F12 (10% FBS, 1% NEAA, 1% pen-strep).

Animals

80 C57BIL/6 males (7 weeks old, 20-30 g) from Janvier were used.

Tumor Injection Procedure

Each animal was weighed before cell implantation. Mice were anesthetized by inhalation of Sevoflurane. Anesthesia was induced at 8% Sevoflurane and maintained at 4%. Every mouse was placed on a heating pad and animal was appropriately marked. Under a suitable depth of unconsciousness, animals were shaved, and skin and surrounding region was washed with Chlorhexidine (1 mg/ml) with use of sterile gauze. Injection was done in the right rear flank with one tumor per mouse with 0.1 ml of approximately 5×10⁵ Yumm 1.7 cells in PBS.

Experimental Groups.

TABLE 12
Experimental groups; time indicates days post implantation

| | | | IP | No. of |
|---|---|---|---|---|
| | IT treatment | Oral dosing | treatment | animals |
| A | PBS (IT) | Vehicle | Igg | 7 |
| B | PBS (IT) | Vehicle | ICB | 7 |
| C | PBS (IT) | Bem* | Igg | 5 |
| D | Dox (3 mg/kg) | Vehicle | Igg | 5 |
| E | Dox (3 mg/kg) | Bem* | Igg | 7 |
| F | Dox (3 mg/kg) | Vehicle | ICB | 7 |
| G | PBS (IT) | Bem* | ICB | 7 |
| H | Dox (3 mg/kg) | Bem* | ICB | 7 |

*Bemcentinib was given Bid as a loading dose (100 mg/kg) for 3 days (day 20-13) then at the dose indicated in table1 (50 mg/kg). animals per group were sacrificed at each time point

Treatment Schedule

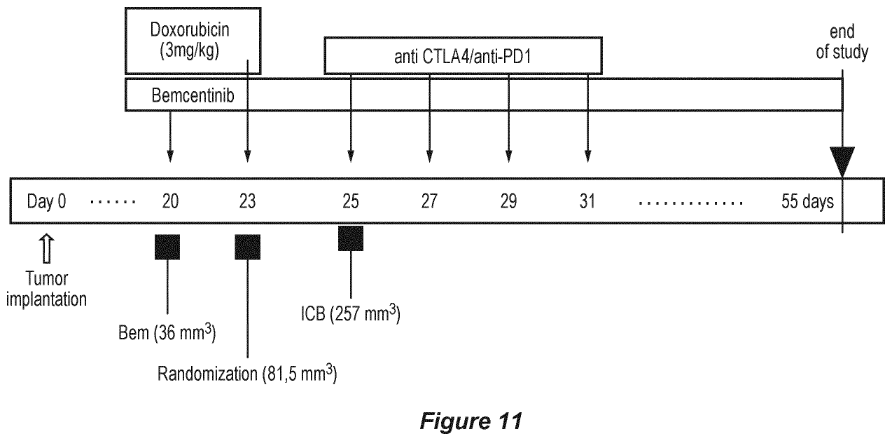

Bemcentinib was administered Bid to groups as described in Table 12 (oral dosing column), starting on the day of randomization 1 as described in FIG. 11. Bemcentinib was given as a loading dose starting day 20 after tumor implantation and lasting for 3 days prior to doxorubicin treatment. Doxorubicin was administered as a single injection to the appropriate groups according to Table 12 (IT treatment column), on day 23 after tumor cell implantation. Anti-CTLA4/anti-PD1 (ICB) or IgG was given to groups as described in Table 12 (IP treatment column) starting 25 days after implantation, as described in FIG. 11.

Randomization

Treatment groups are indicated in Table 12. 80 mice were implanted in 16 cages with 5 animals per cage. 20 days post tumor implantation, the cages were divided in 2 groups based on average tumor size in each cage, half of the cages were treated with bemcentinib, half with the vehicle. A loading dose (100 mg/kg) of bemcentinib was used for 3 days. The average tumor volume of animals in each cage was determined to have a similar repartition of tumor sizes in both groups. 8 cages were treated with bemcentinib and 8 cages treated with vehicle. 3 days later, 23 days post tumor implantation many of the mice displayed a tumor size around 80 mm³, but 24 mice had no or non-measurable tumors. In addition, 4 mice had a tumor volume >250 mm³ and were excluded of the study.

52 mice were used in the study. Once the average tumor size reached 72 mm³ (tumor volume=L×W×W/2) the randomization was performed prior to doxorubicin injection on day 23, and the 52 mice were dispatched in the different groups based on table 1 (A-H). (FIG. 11) The randomization was conducted with Latin square method based on average tumor volume of all animals either in the bemcentinib group or in the vehicle treated group.

The tumor volumes for the different groups at first day of each treatment are recorded in table 13. Similar tumor volumes are obtained for the different groups except group D, doxorubicin alone, which display a much higher tumor volume on average for both time points.

TABLE 13
Tumour volumes at drug treatment initiation

| | Group | Tumour volume | Tumour volume | Tumour volume |
|---|---|---|---|---|
| | name | day 20 (Bem) | day 23 (Dox) | day 25 (ICB) |
| A | CTR | 32.86 | 112.47 | 283.89 |
| B | ICB | 25.42 | 94.01 | 259.74 |
| C | Bem | 36.30 | 74.68 | 243.33 |
| D | Dox | 76.48 | 129.54 | 391.20 |
| E | Dox Bem | 31.56 | 94.06 | 231.74 |
| F | Dox ICB | 26.08 | 91.36 | 253.89 |
| G | Bem ICB | 30.70 | 84.94 | 177.24 |
| H | Dox Bem | 44.04 | 98.48 | 251.18 |
| | ICB | | | |

Tumor Growth Measurements

Tumor growth was measured with a digital handheld caliper twice weekly from emergence of tumor and until the day of euthanasia.

Body Weights

Animals have been weighed prior to tumor cell injection, prior to daily dosing and prior to euthanasia.

Intratumor Injection (i.t) Procedure

When tumors reached an average of 72 mm³, each animal received doxorubicin (3 mg/kg) at a concentration of 1.5 mg/ml (groups D, E, F, G and H) in a total volume of 50 ul×Tumor Volume (mm³)/120 as indicated in Table 12. In the study described in Example 1 the doxorubicin dose was adapted to the tumor volume, to be able to inject smaller tumors. 50 uL is the volume injected for tumors of 120 mm³. Groups A, B, C and G did not receive doxorubicin but received PBS IT. Total drug dose was calculated based on each animal's individual tumor size. For IT drug injections, animals were anesthetized, and drug was slowly injected into the tumor at a rate of approximately 5 ul/second.

Intraperitoneal (IP) Dosing Procedure

On the appropriate days, each animal received a specific amount of IgG (group A, C, D, E) or a combination of the immune check inhibitors anti-mCTRL-4+anti-mPD-1 (groups B, F, G, H) as indicated in Table 12. Dosing volume was 10 ml/kg by 30-gauge needle. Dosing schedule was on days 25, 27, 29 and 31 after tumor implantation (FIG. 11).

Oral Dosing (PO) Procedure

On the appropriate days, each animal in Group A, B, D, F received a specific amount of vehicle (0.5% (w/w) hydroxypropyl methylcellulose/0.1% (w/w) Tween 80). After 3 days of a loading dose (100 mg/kg) from day 11, each animal in Group C, E, G, H received bemcentinib at the dose of 50 mg/kg as indicated in Table 12. Dosing schedule was twice daily on a 5 day-2 day off schedule until 105 days post implantation. Dosing volume was 10 ml/kg by oral gavage.

Clinical Observations

Animals were observed once daily for general appearance.

Mortality/Morbidity

All animals were examined once daily for mortality or morbidity. An animal was sacrificed if the animal lost >20% of the body weight or was judged as moribund. Mice bearing tumors that reach a volume exceeding 1000 mm³ was euthanized. Euthanized animals and any animals found dead prior to rigor mortis was necropsied.

Euthanasia

Animals were anesthetized with Sevoflurane and euthanized by cervical dislocation. Euthanasia was conducted by following the Institutional SOP accordingly.

Tissue Collection

Spleen

Spleens were weighed and prepared for Cytof (cut into pieces of 2 mm and frozen in FBS 90%, DMSO 10% and stored at −80° C. prior to dissociation).

Blood/Plasma

Blood was collected by cardiac puncture. Plasma was collected and frozen to test cytokines.

Tumor Collection

Tumors were weighed and cut in 3 along the long axis, one third was snap frozen in liquid nitrogen in 2 cryotubes per tumor, another third was prepared for Cytof (Cut into pieces of 2 mm and frozen in FBS 90%, DMSO 10% and stored at −80C prior to dissociation). The last piece was fixed in 4% formalin for 24 hours at room temperature and stored in 70% ethanol at 4° C. until embedding in paraffin.

Analysis of Tumor Tissue (Mechanistic Study)

RNA was extracted from snap frozen tumors using the RNEasy Microarray Tissue Mini kit from Qiagen following the manufacturer's instructions. As tumors contained Doxorubicin and powderizing them could expose personnel to harmful substances, the RNA was extracted in a closed system using the GentleMACS dissociator and M Tubes. Purity and concentration of RNA was measured using Nanodrop. cDNA was synthesized from RNA using the RT² First Strand kit (Qiagen) according to the manufacturer's instructions. cDNA was mixed with SYBR Green Mastermix (Qiagen) and loaded into PCR plates with different probes against interferon related genes (listed in table 14). The plates were analyzed using Light Cycler 480 (Roche) and data were analyzed using Graph Pad (Prism).

Tumors from 4 animals treated either with Vehicle (group A), bemcentinib (Group B), Doxorubicin (group F) or bemcentinib+Doxorubicin (group H) were analyzed. Data are presented as fold changes compared to Vehicle treated mice. 35 IFN related genes (Table 14) were tested by RT PCR. Fold change was obtained compared to the control group (A). PDL1 expression was also tested.

TABLE 14
Genes analysed by RT-PCR

| Symbol | Full name | |
|---|---|---|
| TLR3 | Toll-like receptor 3 | IFN related gene |
| TLR9 | Toll-like receptor 9 | IFN related gene |
| IRF7 | Interferon regulatory factor 7 | IFN related gene |
| CCL5 | Chemokine (C-C motif) ligand 5 | IFN related gene |
| STAT1 | Signal transducer and activator of transcription 1 | IFN related gene |
| STAT2 | Signal transducer and activator of transcription 2 | IFN related gene |
| MX1 | Myxovirus (influenza virus) resistance 1 | IFN related gene |
| TNF | Tumor necrosis factor | IFN related gene |
| CXCL10 | Chemokine (C-X-C motif) ligand 10 | IFN related gene |
| ISG15 | ISG15 ubiquitin-like modifier | IFN related gene |
| APOL9B | Apolipoprotein L 9b | IFN related gene |
| IFIT1 | Interferon-induced protein with tetratricopeptide repeats 1 | IFN related gene |
| H2M3 | Histocompatibility 2, M region locus 3 | IFN related gene |
| DDX60 | DEAD (Asp-Glu-Ala-Asp) box polypeptide 60 | IFN related gene |
| RSAD2 | radical SAM domain-containing 2 | IFN related gene |
| OAS1b | 2′-5′ oligoadenylate synthetase 1B | IFN related gene |
| IRF1 | Interferon regulatory factor 1 | IFN related gene |
| IRF9 | Interferon regulatory factor 9 | IFN related gene |
| MX2 | Myxovirus (influenza virus) resistance 2 | IFN related gene |
| IFIT2 | Interferon-induced protein with tetratricopeptide repeats 2 | IFN related gene |
| STAT3 | Signal transducer and activator of transcription 3 | IFN related gene |
| STING | stimulator of interferon gene | IFN related gene |
| JAK1 | Janus kinase 1 | IFN related gene |
| NOS2 | Nitric oxide synthase 2, inducible | IFN related gene |
| SOCS1 | Suppressor of cytokine signaling 1 | IFN related gene |
| IFNAR2 | Interferon (alpha and beta) receptor 2 | IFN related gene |
| SOCS3 | Suppressor of cytokine signaling 3 | IFN related gene |
| IFNAR1 | Interferon (alpha and beta) receptor 1 | IFN related gene |
| TLR7 | Toll-like receptor 7 | IFN related gene |
| STAT6 | Signal transducer and activator of transcription 3 | IFN related gene |
| IRF3 | Interferon regulatory factor 3 | IFN related gene |
| IFNa4 | Interferon alpha 4 | IFN related gene |
| IFNB1 | Interferon beta 1 | IFN related gene |
| IFNa2 | Interferon alpha 2 | IFN related gene |
| TBK1 | TANK-binding kinase 1 | IFN related gene |

Statistics

The tumor volume of 500 or 1000 mm³ was used as an endpoint for the survival analysis. The Kaplan-Meier survival plots were generated using the software program PRISM (GraphPad) and the survival curves were compared using a log-rank (Mantel-Cox) test. FIGURES and Median survival were generated/calculated using software PRISM (GraphPad). Differences between the groups were considered significant when P<0.05.

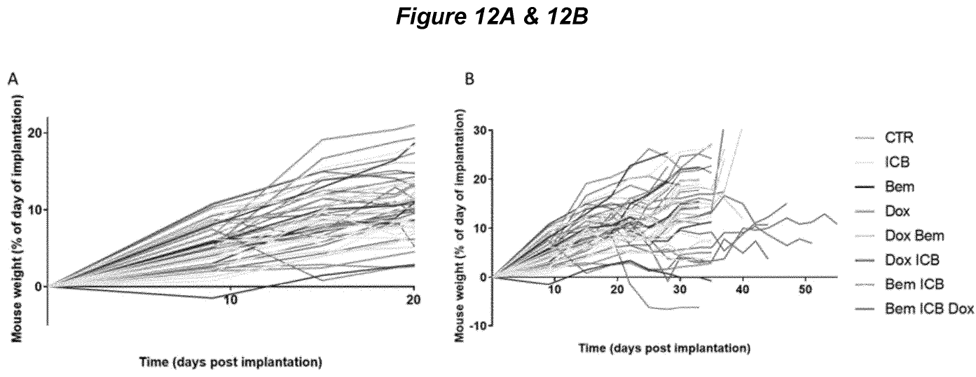

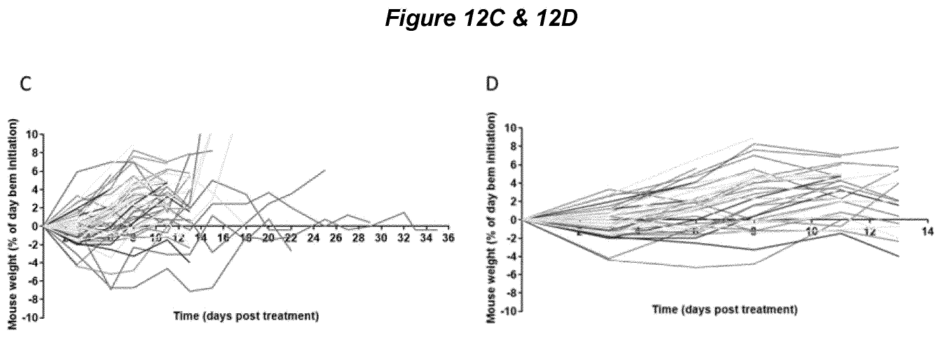

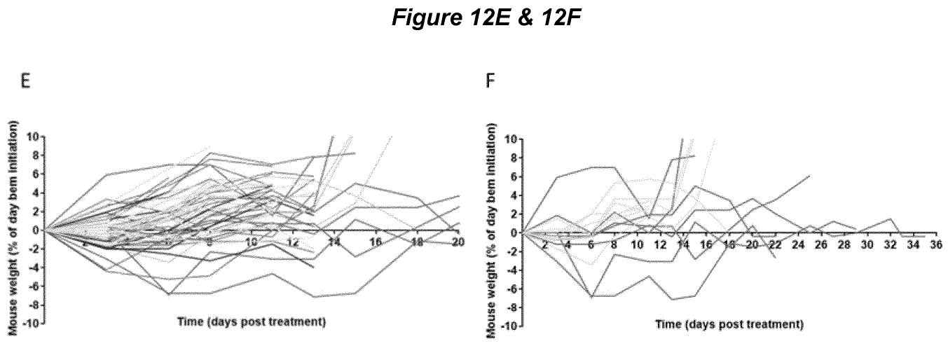

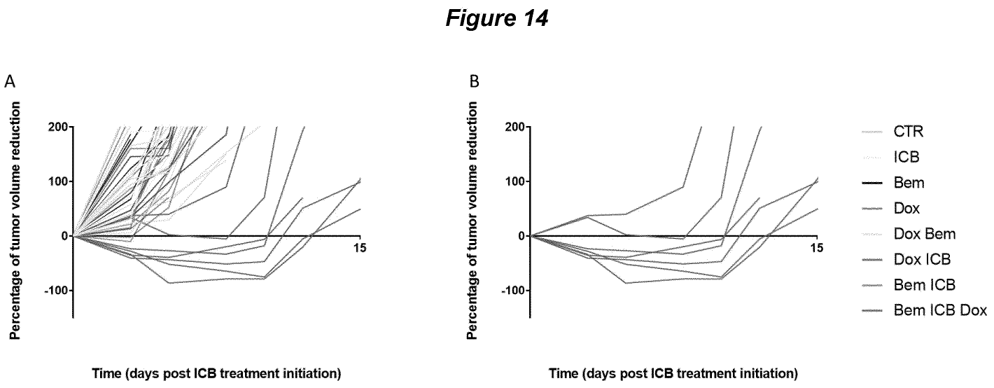

Percentage of mouse weight (FIG. 12A,B) was expressed with the weight of mouse at the day of tumor cells implantation or with the weight on the day of intratumoral treatment (FIG. 12C-F). Percentage of tumor volume (FIG. 14) was calculated relative to the tumor volume measured the day of CPI treatment initiation. A decrease of tumor volume is expressed as negative value and tumor growth with a positive value. Fold change of the genes analyzed were calculated using the ΔΔ Ct method.

Results

Combination Therapies are Tolerated by Mice

The weight of mice measured for up to 55 days post implantation are presented in FIG. 12. Before treatments, all animals showed an increase of body weight (FIG. 12A). After treatment, when percentage weight change was assessed as compared to the day of implantation; only one mouse from the bern+ICB group displayed a weight loss (FIG. 12B). All treatments were well be tolerated.

The mice gained some weight before treatment; therefore, the percentages of weight changes were also calculated compared to the first day of ICB treatment. We observed a small weight gain for many of the animals for the different groups except the triple combination group (Dox bern ICB) (FIG. 12C-F). Only 2 animals from the other groups displayed a weight loss >2%; one from dox+ICB the other from bern+ICB (FIG. 12D). Animals from the triple combination group showed body weight fluctuation; 2 displayed a weight loss (around 6% of body weight at bemcentinib initiation) following treatment, all other animals displayed some weight fluctuation during the treatment (FIG. 12F). This weight loss was probably due to the tumor shrinkage. Some tumors displayed a high volume at the beginning of the bemcentinib treatment, the decrease of this volume may explain the decrease of body weight. The body weight increase may be explained by the tumor relapse. No animals in other group displayed any changes in tumor volume, therefore there was less changes in body weight.

Animals from the triple combination group lived longer than animals from other groups; at the end of experiment the gavage seemed to be more stressful for the animals, due to the tumor at the rear flank, and may also explain the variations of body weight. None of the animals displayed a bodyweight decrease superior to 10% during the experiment (FIG. 12) therefore no animals had to be sacrificed because of a weight loss superior to 20% of body weight.

Triple Combination Treatment with AXL Inhibitor, Immune Checkpoint Modulator, and Cytotoxic Chemotherapy Delays Tumor Growth

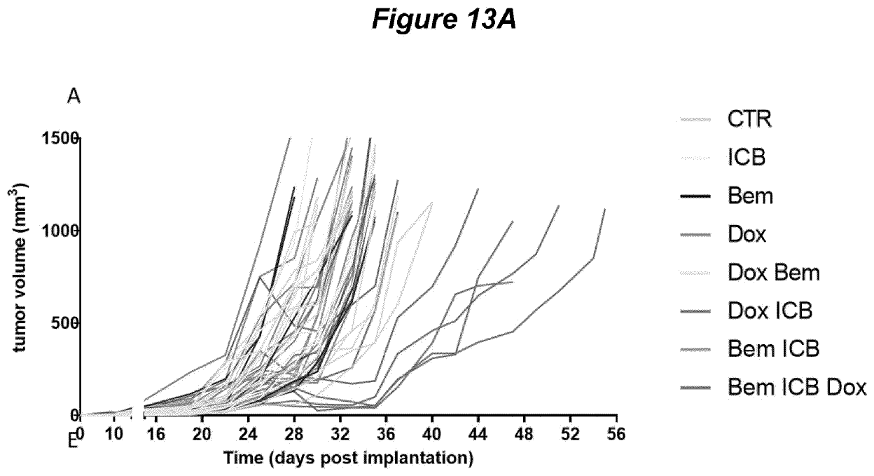

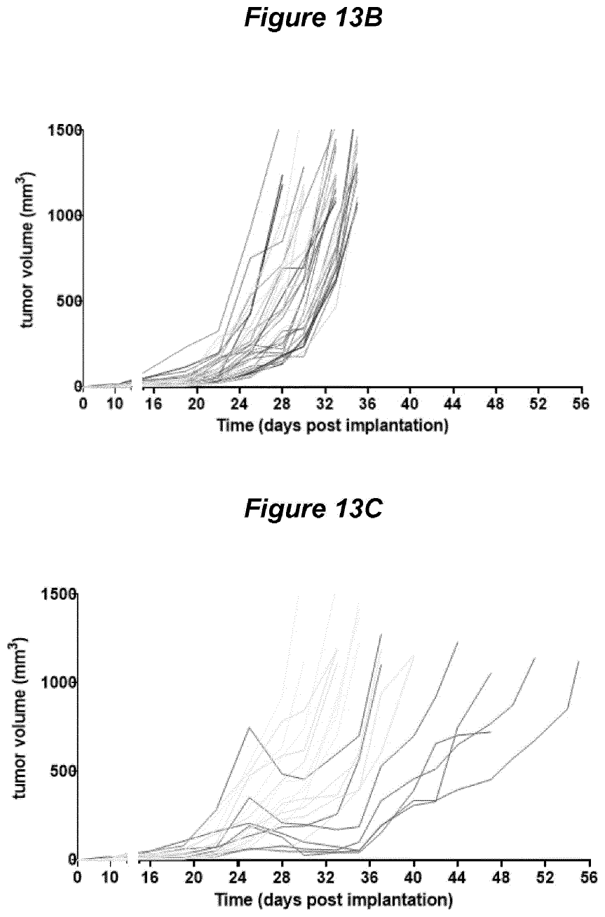

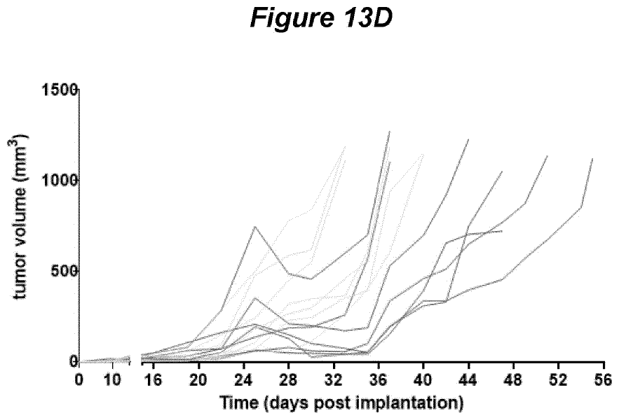

Tumor growth curves are presented in FIG. 13. Heterogeneity in tumor growth was observed for in all groups. The control group, ICB, Dox, Dox ICB and bern ICB groups displayed similar growth curves (FIG. 13A, B). Animals treated with Dox bern (group E) displayed a small delay of tumor growth for few animals compared to all groups (A, B, C, D, F, G) but the triple combination Dox bern ICB group (H) (FIG. 13A, C).

Only the Dox bern ICB (triple combination) group, displayed a tumor growth delay for almost all animals compared to all other groups (FIG. 13A, C, D). In addition, some animals of the Dox bern ICB group displayed reduced tumor volume (FIG. 13D). This decrease is followed by a stable phase for some animals then the tumor volume started increasing again (FIG. 13D). Tumor shrinkage was not observed for any of the other groups.

Tumor growth was slightly delayed for some animals of the dox bern group, but only the dox bern ICB triple combination treatment showed durable response. In the dox bern ICB group, a stable phase was observed (day 25-34) but at day 36 post implantation, all animals relapsed (FIG. 13D). This is not unexpected as dox was only given as a single i.t dose.

These results demonstrate that with the dox bern ICB triple combination, a delay of tumor growth was observed as compared to all other groups.

The delay in tumor growth observed (FIG. 13) was reflected by a tumor volume reduction or growth compared to tumor volume at the time of ICB treatment initiation (FIG. 14). From all other groups than the triple combination dox bern ICB group, only one animal displayed a decrease of tumor volume; this animal was from bern ICB group and displayed a decrease of 10% lasting for 3 days post ICB initiation (FIG. 14A).

In the dox bern ICB triple combination group, 5 animals out of 7 displayed a decrease of tumor growth from −33 to −86% (FIG. 14B). All tumors started growing again around 12 days post treatment initiation, and around day 15 reached a tumor volume superior of day of ICB treatment initiation (FIG. 14B). The dox bern ICB combination induced an initial decrease of tumor growth, not observed in any other group, although all tumors subsequently relapsed.

Triple Combination Treatment with AXL Inhibitor, Immune Checkpoint Modulator, and Cytotoxic Chemotherapy Prolongs Survival

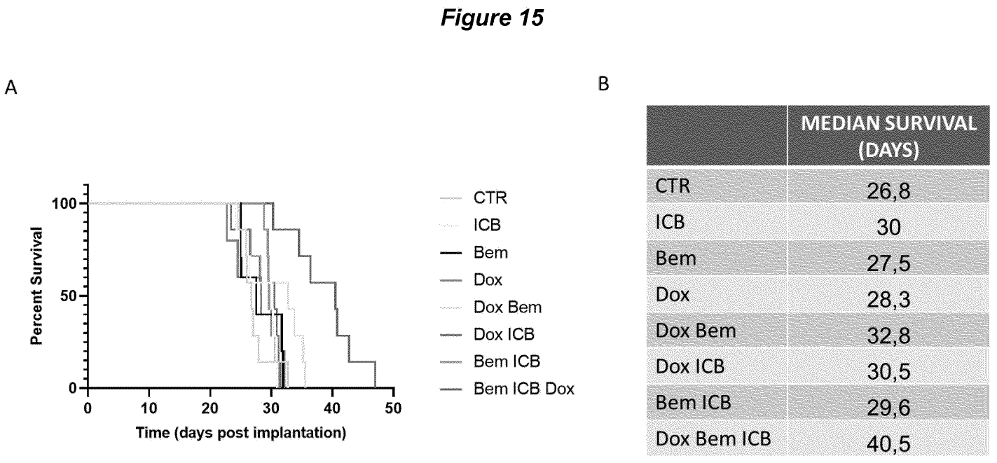

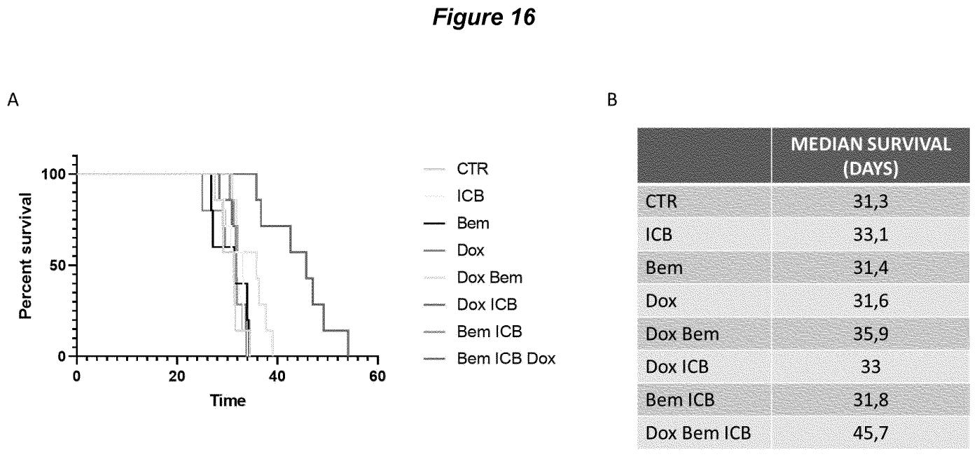

Transformed survival curves were made based on the day tumors reached 500 or 1000 mm³ (FIGS. 15 and 16 respectively).

At 500 mm³, all groups but the dox bern ICB triple combination group and dox bern group displayed similar Kaplan Meier survival curves with similar median survival of 26.8 to 32.8 days (FIG. 15A, 15B). No significant differences were observed between the groups that did not receive CPI (Table 15, LogRank test, P values from 0.2 to 0.8). A significant increase of median survival was observed for Dox bern group (32.8 days) compared to control group (26.8 days) (FIG. 15B; Table 15, P=0.049).

The dox bern ICB triple combination treatment displayed a Kaplan Meir curve with a median survival of 40.5 days (FIG. 15B). This was significantly different compared to all other groups (FIG. 15B, Table 15; P values from 0,0004 to 0.0064).

In conclusion, the median survival at 500 mm³ and survival curves based on this end point showed that the dox bern ICB combination prolongs mouse survival compared to all other treatments.

TABLE 15
P values (log rank mantel cox test, GraphPadPrism) from survival curves at 500 mm³ tumor volume.

| | Dox Bem | | | | | | |
|---|---|---|---|---|---|---|---|
| | ICB | Bem ICB | Dox ICB | Dox Bem | Dox | Bem | ICB |
| CTR | 0.0004* | 0.2060 | 0.5354 | 0.0487* | 0.8832 | 0.8065 | 0.8065 |
| ICB | 0.0009* | 0.7739 | 0.2474 | 0.0929 | 0.4272 | 0.3952 | |
| Bem | 0.0022* | 0.6173 | 0.4449 | 0.1389 | 0.7279 | | |
| Dox | 0.0022* | 0.6173 | 0.4272 | 0.0931 | | | |
| Dox Bem | 0.0064* | 0.6146 | 0.0929 | | | | |
| Dox ICB | 0.0018* | 0.6316 | | | | | |
| Bem ICB | 0.0008* | | | | | | |

Statistical analysis of transformed survival curves.
Survival were set to days of reaching tumor volume of 500 mm³ and statistical analysis was performed by Log-rank (Mantel Cox).
All the groups were compared together one by one.
P-values indicated by * are significant.

At 1000 mm³, all groups but the dox bern ICB combination group and dox bern group displayed similar Kaplan Meier survival curves with similar median survival of 31.3 to 45.7 days (FIG. 16A, 16B). No significant differences are observed between the groups that did not receive ICB (Table 16, LogRank test, P values from 0.39 to 0.94).

An increase of median survival was observed for Dox bern group (35.9 days) compared to all groups (FIG. 16B, 31.3-33.1 days) except the triple combination group (FIG. 16B, 45.7 days). A significant increase of survival was observed for dox bern compare to control (P=0.0084), dox ICB (Pv=0.0751) and bern ICB (0.0391). (FIG. 16A, Table 16).

The triple combination treatment displayed a Kaplan Meir curve with a median survival of 45.7 days (FIG. 16B). This was significantly different compared to all other groups (FIG. 16B, Table 16, P values from 0,0001 to 0.0064).

In conclusion, the median survival at 1000 mm³ and survival curves based on this end point showed that the triple combination prolongs the mouse survival compared to all other treatments. The Dox bern treatment also slightly prolong survival compared to some of the treatments.

To be noted is that in the absence of dox bern, ICB had no effect on tumor growth or survival.

TABLE 16
P values (log rank mantel cox test, GraphPadPrism) from survival curves at 1000 mm³ tumor volume.

| | Dox Bem | | | | | | |
|---|---|---|---|---|---|---|---|
| | ICB | Bem ICB | Dox ICB | Dox Bem | Dox | Bem | ICB |
| CTR | 0.0001* | 0.4834 | 0.506 | 0.0084* | 0.7921 | 0.9415 | 0.6457 |
| ICB | 0.0002* | 0.5199 | 0.9227 | 0.0584 | 0.6447 | 0.7406 | |
| Bem | 0.0003* | 0.5187 | 0.6712 | 0.0522 | 0.6682 | | |
| Dox | 0.0003* | 0.4776 | 0.6258 | 0.0522 | | | |
| Dox Bem | 0.0064* | 0.0391* | 0.0751* | | | | |
| Dox ICB | 0.0001* | 0.3925 | | | | | |
| Bem ICB | 0.0001* | | | | | | |

Statistical analysis of transformed survival curves.
Survival were set to days of reaching tumor volume of 1000 mm³ and statistical analysis was performed by Log-rank (Mantel Cox).
All the groups were compared together one by one.
P-values indicated by * are significant.

Effects of Combination Therapy on Metastasis, Tumor Weight, and Spleen Weight

Different organs were examined for macrometastasis. No metastasis was found in any of the animals from the different treatment groups. Because of that, no animals were sacrificed due to symptoms, and the whole data of the study were based on the tumor volume measured with digital caliper.

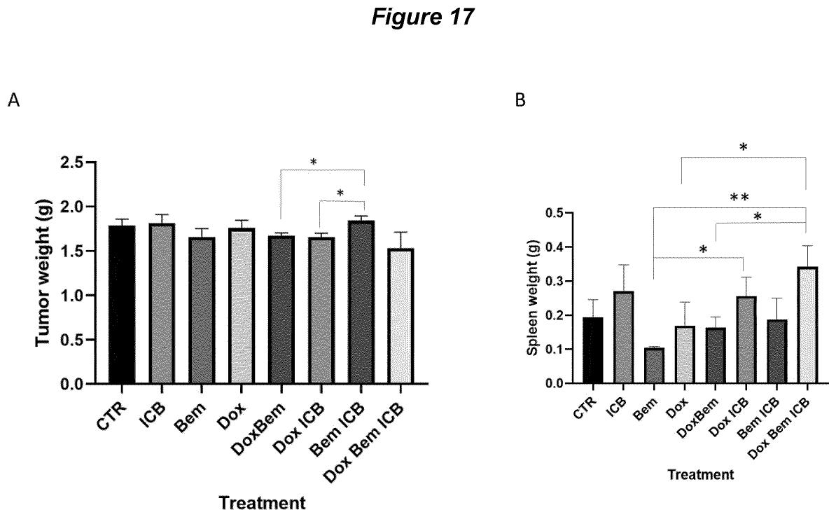

The tumor weights were similar for all groups with no significant difference (weight from 1.53 g to 1.81 g; TTEST, P value from 0.15 to 0.97) between all the groups but bern ICB (FIG. 17A). The mice from bern ICB had the highest tumor weight 1.83 g which was significantly higher than mice from dox bern (1.67 g) and bern ICB (1.66 g) with a P value of 0.0018. The significant difference (Table 16, Log Rank testPvalue=0.0391) in mouse survival between bern ICB and dox bern group may be explained by the fact that animals with lighter tumors in this group were sacrificed.

Notably, the dox bern ICB triple combination group displayed the smallest tumor weight (1.53 g), mostly because 2 animals displayed a much lower weight due to the presence of copious liquid accumulation around the tumor. This weight different was not significant (P value from 0.11 to 0.57) because of the variability and because many of animals from this group had similar tumor weight as observed in other groups. This did not affect the results because in the worst case those tumors were harvested too early, and we were still able to see a significant difference on mice survival. The animals were sacrificed when the tumor volume exceeded 1000 mm³; for most treatment groups, tumors displayed a similar weight had similar volumes.

Splenomegaly is an indication of systemic disease and is observed in some syngeneic mouse cancer models such as 4T1 which display metastasis in many organs (see Example 1). Normal C57bl6 mouse spleen weight is around or below 0.1 g (based on monitoring of mice from transgenic colonies). In this study the spleen weights were between 0.1 and 0.7 g. In 4T1 carcinoma bearing mice, spleens can weigh 2 g and no animals had normal spleens (see Example 1). The increase of tumor weight in the Yumm1.7 model was limited, probably because the tumor cells did not effectively metastasize. The highest spleen weight was observed for the dox bern ICB triple combination group (FIG. 17B, 0.34 g), and was significant to some of the groups bern (0.10 g), Dox (0.17) (Pvalue<0.05) and Dox bern (0.16 g, Pvalue<0.01). (FIG. 17B). To be noted that the difference was not significant compared to control (FIG. 17B).

Some differences of average tumor weights were observed between the different groups, and the variability was important with many animals displaying normal spleen in all the groups. The modification of spleen size may indicate a change of immune cells in the spleen, therefore an increase in the triple combination group may be a sign that the immune environment is modified.

Effects of Combination Therapy on Expression of Type I IFN Related Genes

The current study showed in a model refractory of ICB, bemcentinib and doxorubicin was able to increase mouse survival and delay tumor growth. A molecular mechanism focused study will be performed later; here samples were collected after sacrifice, meaning many days after treatment for some of the groups. The expression of 35 ISG as well, immune checkpoint related genes EMT related genes, Axl and Gas6 were analyzed by qRTPCR (Table 14).

In all of groups (A-G) other than the dox bern ICB triple combination group, a maximum of 4 ISG were found to be significantly upregulated per group as compared to control. No gene was significantly upregulated in the dox group compared to control. 2 genes were upregulated in the ICB treated groups compared to control. 3 genes were upregulated in DoxICB and bernICB groups compared to control. In bern and Dox bern groups group 4 genes were significantly upregulated. In all these groups the IFN response was limited to a few ISG.

Conversely, out of the 35 IFN related genes tested, 19 were found to be significantly upregulated in the dox bern ICB triple combination group as compared to control. The fold changes were between 2 and 4 for all but 3 of these genes (which had higher fold changes). Notably, the 3 ISGs with the highest fold changes were also upregulated in the other treated groups. A broad IFN response was observed with the dox bern ICB triple combination group several days post-treatment.

Axl and Gas6 as well as the other EMT related genes tested were not changed compared to control in any of the groups.

These results show 13 ISG were significantly upregulated in the dox bern ICB triple combination treatment group but not with other treatments, as compared to control. This may be linked to the increased survival observed with the triple combination treatment.

### Experimental Conclusions

The results presented here confirm that the triple combination treatment including immune checkpoint modulator treatment (anti-CTLA4/anti-PD1 CPI), cytotoxic chemotherapy (doxorubicin) and AXL inhibitor (bemcentinib). significantly increased mouse survival compared to all other treatments in an immune refractory melanoma tumor model (YUMM1.7).

ICB treatment, including Anti CTLA4, Anti PD1 antibodies have previously been reported to have no effect in the Yumm1.7 model. This was confirmed by the present study, in which the ICB treated animals displayed a similar median survival as the control group: 33.1 days vs 31.2 days post implantation. In addition all double combination treatments with ICB displayed the same mouse survival as the control group, contrary to what was observed in the 4T1 model (Example 1).

The triple combination treatment displayed a delay of tumor growth and an increase of mice survival compared to all other groups: an increase of medial survival of 7.7 to 13.7 days compared the other treatment combinations. This increase can be explained by the tumor shrinkage followed by a stable phase. In the 4T1 model (Example 1) the increase of median survival of triple combination compared to control was 25 days compared to control and 15 days compared to ICB alone. Furthermore, some long-term responders have been shown in the Example 1 study. In the Yumm1.7 model, no long-term response was observed because all tumors relapsed. A more modest effect is observed in the Yumm1.7 model, most probably because it is refractory to ICB.

It is established that in mouse models the tumor size at treatment initiation can influence the outcome of the specific treatment efficacy. In the present study, very few differences in average tumor size were observed between the different groups when the different treatments were initiated except for the Dox and bern ICB treated group (Table 13). Average tumor volumes for all other groups were between 231.74 and 283.89 mm³ when ICB treatment was initiated compared to 177.24 mm³ for the bern ICB group and 391.2 mm³ for the Dox group. Despite having a smaller tumor volume, no effect on survival compared to control was observed for the bern ICB group so this difference in tumor volume had no effect on the results. For the Dox group, the average tumor volume was higher that all other group at the initiation of doxorubicin treatment (Table 13). No effect on survival was observed for these animals compared to control but we cannot exclude that with smaller tumor would allow to see an effect on mouse survival.

The main in vivo effect on survival is observed for the triple combination group displaying similar tumor volumes than other groups, therefore we concluded that the results we observed for tumor growth inhibition and median survival was not due to difference in tumor volumes at start of ICB treatment.

In summary, administration of bemcentinib associated with type I interferon stimulation by doxorubicin sensitized the syngeneic mouse melanoma model yumm1.7 to immune checkpoint blockade. However, the response was temporary, and the tumors relapsed. The triple combination treatment also induced an interferon response that was maintained until the end of the experiment. This interferon type I response induced in the triple combination may have allowed the tumor response to treatment but may also have caused the tumor relapse.

Potentiating the ICB by bemcentinib associated with interferon stimulation intratumorally has thus been shown in two different models: 4T1 breast cancer model (Example 1) and Yumm1.7 melanoma model (present Example).

### Example 4

Example 1 demonstrates the potentiating effect of cytotoxic chemotherapy (doxorubicin) or AXL inhibitors (bemcentinib) on immune checkpoint modulator treatment (anti-CTLA4/anti-PD1 CPI) in vivo in the syngeneic 4T1 model, with a higher potentiating effect observed when cytotoxic chemotherapy (Doxorubicin) and AXL inhibitor (bemcentinib) are used together (in a triple combination treatment).

Example 2 is a mechanism focused study showing that in the syngeneic 4T1 model, bemcentinib leads to an IFN response, a downregulation of EMT related genes, as well as few effects on circulating cytokines in combination with low dose of doxorubicin (1 mg/kg). Bemcentinib was necessary for the interferon type one response, which may explain the potentiating effect of bemcentinib on immune checkpoint inhibitor treatment in combination with doxorubicin observed in Example 1.

Example 3 reports the ability of cytotoxic chemotherapy (doxorubicin) and AXL inhibition (bemcentinib) to sensitize the syngeneic mouse melanoma model Yumm 1.7 to immune checkpoint modulator treatment (anti-CTLA4/anti-PD1 CPI). Example 3 also demonstrates that triple combination treatment induces an interferon response in these animals.

In this example, an in vitro evaluation of the effect of bemcentinib on the IFN response on different cell lines (4T1, Yumm1.7, LL2) was undertaken. The results form experiments with cells were performed reported previously (cf SR-JL5-002-009). The objectives of this study were: 1. Determine the effect of bemcentinib on the IFN response in 4T1 cells IFN stimulation by IFNB, DMXAA and doxorubicin; 2. Determine the effect of bemcentinib in the IFN response in Yumm1.7 cells following interferon stimulation by IFNB, DMXAA and doxorubicin; and 3. Determine if the potentiation of IFN response by bemcentinib can be applied to Lewis lung carcinoma model using a chemotherapeutic currently used in the clinic.

Materials & Methods

Cell Culture

4T1 cells (ATCC, CRL-2539) were cultured in RPMI-1640 (Sigma, Cat. #R8758) supplemented with 10% fetal bovine serum (FBS), L-glutamine (4 mM), streptomycin (5 μg/ml) and penicillin (5 U/ml). Yale university mouse melanoma cells 1.7 (Yumm 1.7). BrafV600E/wt (knock in conditionally activated Braf allele) Cdkn2a−/− Pten−/− (conditionally inactivated) were cultured in DMEM F12 (10% FBS, 1% NEAA, 1% pen-strep). Lewis lung carcinoma cells LL/2 (LLC1) (ATCC, CRL-1642™) were cultured in Dulbecco's Modified Eagle's Medium ATCC® 30-2002™ (4 mM L-glutamine, 4500 mg/L glucose, 1 mM sodium pyruvate, and 1500 mg/L sodium bicarbonate).

Gene Expression Analysis

RNA Extraction

RNA was extracted from snap frozen tumors using the RNEasy Microarray Tissue Mini kit from Qiagen following the manufacturer's instructions. As tumors contained Doxorubicin and powderizing them could expose personnel to harmful substances, the RNA was extracted in a closed system using the GentleMACS dissociator and M Tubes. Purity and concentration of RNA was measured using Nanodrop. RT² Profiler™ PCR Array. cDNA was synthesized from RNA using the RT² First Strand kit (Qiagen) according to the manufacturer's instructions. cDNA was mixed with RT² SYBR Green Mastermix (Qiagen) and loaded into RT² Profiler™ PCR Array Mouse Type I Interferon Response or RT² Profiler™ PCR Array Mouse Epithelial to Mesenchymal Transition (EMT), analyzing the expression of the genes listed in Table 17. The plates were analyzed using Light Cycler 480 (Roche) and data were analyzed using the Qiagen Data Analysis Centre and Graph Pad (Prism).

Fold change of the genes analyzed were calculated using the ΔΔ Ct method. Quiagen RT² software was used to analyze all the samples from different groups. To be able to generate heatmaps and graph on fold change including error bars, the data have been analyzed manually with Graph Pad (Prism) when the Quiagen RT² software gave significant results between the different groups. For IFN type 1 response 1 mg/kg 48, 72 H and 3 mg/kg 48H, were analyzed manually. For EMT, 1 mg/kg 48, 72 H, 3 mg/kg 72H. When the data have been analyzed manually, the results are presented as histograms of mRNA fold changes. For the time points and doses analyzed by the software, the results are presented as tables.

TABLE 17
IFN related genes tested by RT-PCR

| Number | Symbol | Full name | |
|---|---|---|---|
| 1 | TLR3 | Toll-like receptor 3 | IFN related gene |
| 2 | TLR9 | Toll-like receptor 9 | IFN related gene |
| 3 | IRF7 | Interferon regulatory factor 7 | IFN related gene |
| 4 | CCL5 | Chemokine (C-C motif) ligand 5 | IFN related gene |
| 5 | STAT1 | Signal transducer and activator of transcription 1 | IFN related gene |
| 6 | STAT2 | Signal transducer and activator of transcription 2 | IFN related gene |
| 7 | MX1 | Myxovirus (influenza virus) resistance 1 | IFN related gene |
| 8 | TNF | Tumor necrosis factor | IFN related gene |
| 9 | CXCL10 | Chemokine (C-X-C motif) ligand 10 | IFN related gene |
| 10 | ISG15 | ISG15 ubiquitin-like modifier | IFN related gene |
| 11 | APOL9B | Apolipoprotein L 9b | IFN related gene |
| 12 | IFIT1 | Interferon-induced protein with tetratricopeptide repeats 1 | IFN related gene |
| 13 | H2M3 | Histocompatibility 2, M region locus 3 | IFN related gene |
| 14 | DDX60 | DEAD (Asp-Glu-Ala-Asp) box polypeptide 60 | IFN related gene |
| 15 | RSAD2 | radical SAM domain-containing 2 | IFN related gene |
| 16 | OAS1b | 2′-5′ oligoadenylate synthetase 1B | IFN related gene |
| 17 | IRF1 | Interferon regulatory factor 1 | IFN related gene |
| 18 | IRF9 | Interferon regulatory factor 9 | IFN related gene |
| 19 | MX2 | Myxovirus (influenza virus) resistance 2 | IFN related gene |
| 20 | IFIT2 | Interferon-induced protein with tetratricopeptide repeats 2 | IFN related gene |
| 21 | STAT3 | Signal transducer and activator of transcription 3 | IFN related gene |
| 22 | STING | stimulator of interferon gene | IFN related gene |
| 23 | JAK1 | Janus kinase 1 | IFN related gene |
| 24 | NOS2 | Nitric oxide synthase 2, inducible | IFN related gene |
| 25 | SOCS1 | Suppressor of cytokine signaling 1 | IFN related gene |
| 26 | IFNAR2 | Interferon (alpha and beta) receptor 2 | IFN related gene |
| 27 | SOCS3 | Suppressor of cytokine signaling 3 | IFN related gene |
| 28 | IFNAR1 | Interferon (alpha and beta) receptor 1 | IFN related gene |
| 29 | TLR7 | Toll-like receptor 7 | IFN related gene |
| 30 | STAT6 | Signal transducer and activator of transcription 3 | IFN related gene |
| 31 | IRF3 | Interferon regulatory factor 3 | IFN related gene |
| 32 | IFNa4 | Interferon alpha 4 | IFN related gene |
| 33 | IFNB1 | Interferon beta 1 | IFN related gene |
| 34 | IFNa2 | Interferon alpha 2 | IFN related gene |
| 35 | TBK1 | TANK-binding kinase 1 | IFN related gene |
| 36 | GBP2B | Guanylate binding protein 1 | IFN related gene |
| 37 | H2K1 | Histocompatibility 2, K1, K region | IFN related gene |
| 38 | OAS2 | 2′-5′ oligoadenylate synthetase 2 | IFN related gene |
| 39 | IFNg | interferon gamma | IFN related gene |
| 40 | CD274 (PDL1) | Programmed death-ligand 1 | Immune checkpoint ligand |
| 41 | PDL2 | Programmed death-ligand 1 | Immune checkpoint ligand |
| 42 | CTLA4 | Cytotoxic T-Lymphocyte Associated Protein 4 | Immune checkpoint |
| 43 | TIM3 | T-cell immunoglobulin and mucin-domain containing-3 | Immune checkpoint |
| 44 | MITF | Melanocyte Inducing Transcription Factor | other |
| 45 | FOXP3 | forkhead box P3 | other |
| 46 | VIM | vimentin | EMT related gene |
| 47 | CDH2 | cadherine2 | EMT related gene |
| | CDH1 | cadherine1 | EMT related gene |
| | GAS6 | Growth Arrest Specific 6 | other |
| | AXL | AXL receptor tyrosine kinase | other |
| | ACE2 | Angiotensin-converting enzyme 2 | other |

RNA Sequencing

RNA was extracted from snap frozen tumors using the RNEasy Microarray Tissue Mini kit from Qiagen following the manufacturer's instructions. The RNA was extracted in a closed system using the GentleMACS dissociator and M Tubes. Purity and concentration of RNA was measured using Nanodrop. 3 RNA samples from 4T1 cells were sequenced from control group, bemcentinib only, IFNb only, bemcentinib/IFNb and Gas6, Gas6/IFNb, GAS6/bemcentinib, Gas6/IFNb/bemcentinib. FASTQ files were generated, and quality controlled by the Genomic Core Facility. The trimmed samples were aligned to the reference genome using STAR. Gene expression quantification was achieved using feature Counts. Following QC, 8 samples from were identified as outliers and were excluded from subsequent analysis. RUVseq was used to remove hidden batch effect and/or other unwanted variations. Read counts were analyzed for differential expression using the R package DESeq2. DESeq2 uses the raw read counts, applies an internal normalization method, and does estimation of library size, estimation of dispersion, and negative binomial generalized linear model fitting. The resulting differentially expressed gene lists (adjusted p-value cutoff=0.05) were then analyzed using Metascape to find the enriched terms.

Results

Effects of Bemcentinib on IFN Response in 4T1 Adenocarcinoma Cells

The effect of bemcentinib on IFN type I response was measured aftertreatment with IFNB, doxorubicin, or the Sting agonist DMXAA. The IFN response was assessed by transcriptional analysis using qRTPCR or RNA sequencing.

Bemcentinib Treatment was Found to Increase the IFN Type I Response Induced by IFNB in 4T1 Cells in Vitro.

ISG (N=24) were measured in 4T1 cell extracts by qRTPCR after treatment with IFNB for 48 hours (Table 17, genes 1-35). The experiment was performed in the presence of supplemental Gas6 in the medium. IFNB increased the expression of 10 ISG with a fold change above 2. Out of these 10 ISG, 9 were significantly upregulated in the presence of bemcentinib and IFNB stimulation compared to IFNB stimulation alone. Hence, bemcentinib potentiated the IFNB mediated response.

To confirm the data observed by qRTPCR, RNA sequencing was performed on 4T1 cells stimulated with IFNB the same samples. The experiment was conducted without or with supplemental Gas6. IFN related genes were upregulated in the IFNB treated group as compared to control. Gene Ontology analysis showed that the number of genes upregulated in Bemcentinib treated cells compared to control is limited as compared to IFNB stimulation. A limited IFN type I response is induced by bemcentinib alone. Many more upregulated genes were observed in the bemcentinib+IFNB sample as compared to IFNb alone. This suggests that the IFN response is increased by bemcentinib.

The results show that bemcentinib potentiates the IFN response after stimulation by IFNB-mediated response and increases a type 2 interferon response.

When compared to Gas6 treated 4T1 cells, the IFNB treated cells displayed a significant overexpression of genes in the response to interferon beta and gamma GO terms. When bemcentinib treated 4T1 cells were compared to Gas6 treated cells, response to interferon gamma was significantly upregulated. The same results were obtained by comparing bemcentinib/Gas6 with Gas6 treated cells.

Benmcentinib/IFNB with or without supplemental Gas6 displayed a significant upregulation of genes in the gene terms response to IFN beta and gamma when compared to Gas6 treated cells. No significantly upregulated genes were observed between the control and Gas6 treated cells. When compared to IFNB+Gas 6 compared to Gas6, response to interferon beta and gamma gene were upregulated. When compared directly very few genes were significantly upregulated between bemcentinib and bemcentinib/IFNb in presence of Gas6. The GO terms related to IFN responses were not found, contrary to what was observed without Gas6. In the presence of Gas6, an increase of interferon response by bemcentinib+IFNb treatment was not observed as compared to IFNb treatment alone.

Bemcentinib potentiated the IFN type I response induced by IFNb stimulation of 4T1 cells in vitro with an increase of mRNA fold change of few IFN related genes shown by qRTPCR in presence of Gas6. RNA sequencing confirmed this finding in absence of supplemental Gas6 with more IFN related genes overexpressed. In addition, the direct comparison showed an increase of type 2 IFN response in presence of bemcentinib+IFNB compared to IFNB alone but only in the absence of Gas6.

Bemcentinb Increased the IFN Response Induced by Doxorubicin in 4T1 Cells In Vitro

4T1 cells were treated with doxorubicin in the presence or absence of bemcentinib. The type I IFN related gene expression was tested by qPCR. Out of the 42 ISG measured by qRTPCR, 9 displayed a significant mRNA fold change upregulation when 4T1 cells were treated with doxorubicin compared to control.

When bemcentinib was added to doxorubicin, 13 ISG displayed a significant mRNA fold change increase compare to control. Bemcentinib in combination with doxorubicin also increased the fold change of 11 ISG compared to doxorubicin alone (with an increase of fold change respectively). The differences in fold change between doxorubicin alone and doxorubicin+bemcentinib were not significant, probably because of the variability between samples. Bemcentinib increased the IFN response in 4T1 cells by increasing the number and magnitude of ISG expression.

Bemcentinb Treatment Did not Increase the IFN Type I Response Induced by Sting Agonist DMXAA in 4T1 Cells In Vitro

No significant changes in IFN response we detected after DMXAA stimulation with or without bemcentinib.

Effects of Bemcentinib on IFN Response in Yumm 1.7 Cells

Bemcentinib Increased the IFN Response Induced by IFNB in Yumm 1.7 Cells In Vitro

IFN related gene expression was measured in Yumm1.7 cells treated with IFNB in the presence or absence of bemcentinib. The response observed without bemcentinib was very strong with fold changes above 100 for many ISG. Bemcentinib appeared to increase the IFNB mediated response, although not reaching significance in this experiment.

Bemcentinib Enhanced the IFN Response Induced by Doxorubicin in Yumm 1.7 Cells In Vitro

The Yumm1.7 melanoma cell line, was treated with doxorubicin during 24 hours in presence or absence of bemcentinib. Out of 21 ISG tested by qRTPCR, 9 were significantly upregulated compared to control after doxorubicin treatment. In presence of bemcentinib+doxorubicin, 13 were upregulated. 12 of these 13 genes were significantly upregulated in the combined group compared to doxorubicin alone. Many more ISG showed >2 fold changes with bemcentinib+doxorubicin than with doxorubicin alone. OAS2 increased from 50 to 175 fold, RSAD2 from 20 to 120 fold, IFIT1 from 20 to 70 fold (FIG. 11). With the combined treatment 8 genes displayed a >30-fold change in Yumm1.7 cells.

Bemcentinib Did not Increase the IFN Response Induced by Sting Agonist DMXAA in Yumm 1.7 Cells in Vitro

Out of 13 ISG tested by qRTPCR after DMXAA treatment of Yumm1.7 cells, 4 were upregulated with DMXAA alone compared to only 2 for DMXAA+bemcentinib treated cells. Hence, bemcentinib did not potentiate the IFN response induced by DMXAA treatment.

Evaluation of the Effect of Chemotherapeutics on IFN Response in Lewis Lung Carcinoma (LL2) Cells

Chemotherapeutic agents commonly used to treat lung cancer patients were evaluated for effects on the IFN response. Cisplatin, navelbine and docetaxel were tested in vitro in combination with bemcentinib. Sting agonist DMXAA, doxorubin and IFNB were also evaluated for comparison.

The IC50 of the different drugs were calculated by resazurin viability assay. Bemcentinib addition decreased the IC50 of all tested drugs except the Sting agonist DMXAA (Table 18). The following concentrations were chosen based on the IC50: 5 nM for docetaxel, 1 nM for navelbine, 25 ug/ml for DMXAA, 5 uM for cisplatin. The cells were treated for 48 hours.

TABLE 18
Resazurin viability assay of LL2 cells treated with different
chemotherapeutic in the resence or absence of bemcentinib

| | IC50 without bem (nM) | IC50 with bem (nM) |
|---|---|---|
| Doxorubicin | 177.8 | 70.3 |
| Docetaxel | 14.12 | 7.26 |
| Navelbine | 10.05 | 0.7525 |
| Cisplatin | 6190 | 4649 |
| Sting | No effect | No effect |

Limited Effect of Bemcentinb on IFN Response Induced by IFNB, Doxorubicin or Sting Agonists in LL2 Cells

The Lewis lung carcinoma cells (LL2) were treated 48 hours with IFNB, DMXAA (Sting agonist) and doxorubicin. ISG were measured by qRTPCR. IFNB alone induced a IFN response in LL2 cells with a significant upregulation of 6 ISG and 7 ISG in the presence of bemcentinib. The INFB-mediated response was not potentiated by bemcentinib in LL2 cells. Doxorubicin (50 nM) induced a limited IFN response in LL2 cells compared to control. Three ISG were significantly upregulated compared to control for the combined treatment. Interestingly, Sting and IFNb1 were upregulated.

We cannot conclude if bemcentinib increase the IFN response as the number of ISG tested was limited. It is possible a higher dose of doxorubin would induce a higher IFN type I response. The sting agonist DMXAA was used to treat LL2 cells for 48 hours. A broad IFN response was observed with 20 upregulated ISG. Some of the genes display a 75-fold upregulation with DMXAA alone compared to control. In DMXAA+bemcentinib, 19 ISG were significantly upregulated compared to control, and only 1 displayed a significantly higher fold change compare to DMXAA alone. We can conclude that the IFN response obtained with DMXAA treatment was not increased by bemcentinib.

The Interferon Type I Response Observed in LL2 Cells Treated with Cisplatin or Navelbine was not Increased by Bemcentinib Treatment.

LL2 cells were with cisplatin 5 uM for 48 hours; 11 ISGs were upregulated both with and without bemcentinib compared to control. The Axl expression was also upregulated compared to control both with and without bemcentinib. The fold change increases ranged between 5 and 10 for most of the ISG. The IFN response induced by cisplatin was moderate compared to Sting agonist or IFNB effects. Bemcentinib did not further increase the IFN response observed with cisplatin alone. Lower concentrations of cisplatin were tested, but none of them induced IFN type I response.

The cells were treated with the vinca alkaloid navelbine (1 nM) for 48 hours; a limited IFN response was observed. Four ISG were upregulated for both navelbine with and without bemcentinib. Other ISG were tested but none were changed in either group. The IFN response induced by navelbine was limited and not increased by bemcentinib.

Bemcentinib Increased the IFN Response in Combination with Docetaxel in LL2 Cells In Vitro

The LL2 cells were treated with 5 nM of docetaxel with and without bemcentinib. While only 2 ISG were significantly upregulated with docetaxel alone, 16 ISG were upregulated in cells treated with docetaxel+bemcentinib compared to control. Furthermore, 12 ISG were significantly upregulated in cells treated with docetaxel+bemcentinib, compared to docetaxel alone. In the combined treatment 3 ISG displayed a 50× fold change and 4 ISG displayed a 10× fold change. Other upregulated ISG displayed fold changes between 2 and 8. Hence bemcentinib synergistically enhances the IFN in combination with docetaxel. A higher dose (15 nM) of docetaxel was tested, the effect of bemcentinib was more limited. Hence dose is likely important for an optimal IFN response.

### Experimental Conclusions

Axl kinase inhibition with bemcentinib enhances the anthracyline induced type 1 IFN response in vitro. In particular, bemecentinib treatment synergized with the type 1 IFN stimulation by doxorubicin in inducing a number of ISGs in 4T1 cells.

In an oncogene-driven cell model (Yumm1.7), we found that found that bemcentinib in combination with doxorubicin or IFNB strongly increases the type 1 IFN response. Remarkable fold differences were observed in the mRNA levels of ISGs that are also a hallmark feature of antiviral immune response.

Bemcentinib increased the IFN response in both 4T1 and Yumm1.7 cells with direct stimulation by IFNB or after doxorubicin treatment. Interestingly, bemcentinib did not potentiate the IFN response initiated by DMXAA. This is important to determine exactly how AXL regulates IFNR signaling. The effect of doxorubicin on IFN response has been confirmed in vivo (see Examples 1-3) with a correlation between an increase of ISG (interferon stimulating genes) and an increase of mouse survival.

Bemcentinib significantly increased the IFN type I response in Lewis lung carcinoma cell lines (LL2) treated with docetaxel. The ISG expression enhancement was similar that measured for 4T1 cells treated with doxorubicin and bemcentinib. In LL2 cells a remarkable synergy was detected between bemcentinib and docetaxel. A complete signature of ISG was observed including when bemcentinib is added to docetaxel 5 nM, while only a few ISGs were overexpressed with docetaxel 5 nM alone. Based on these results the combination of bemcentinib with docetaxel+ICB is an optimal chemo-immunotherapeutic combination.

These results support the beneficial effects of a triple combination of AXLi (bemcentinib), chemotherapy (doxorubicin, docetaxel) and immunotherapy (anti-CTLA4/anti-PD1).

### REFERENCES

A number of publications are cited above in order to more fully describe and disclose the invention and the state of the art to which the invention pertains. Full citations for these references are provided below. The entirety of each of these references is incorporated herein.
Rothlin C V, Ghosh S, Zuniga E I, Oldstone M B, Lemke G. TAM receptors are pleiotropic inhibitors of the innate immune response. Cell. 2007; 131(6):1124-36.
Huang M T, Liu W L, Lu C W, Huang J J, Chuang H L, Huang Y T, et al. Feedback regulation of IFN-alpha/beta signaling by Axl receptor tyrosine kinase modulates HBV immunity. Eur J Immunol. 2015; 45(6):1696-705.
Chen J, Yang Y F, Yang Y, Zou P, Chen J, He Y, et al. AXL promotes Zika virus infection in astrocytes by antagonizing type I interferon signalling. Nature microbiology. 2018; 3(3):302-9.
Galluzzi L, Buque A, Kepp O, Zitvogel L, Kroemer G. Immunological Effects of Conventional Chemotherapy and Targeted Anticancer Agents. Cancer cell. 2015; 28(6):690-714.
Yan Y, Kumar A B, Finnes H, Markovic S N, Park S, Dronca R S, et al. Combining Immune Checkpoint Inhibitors With Conventional Cancer Therapy. Front Immunol. 2018; 9:1739.
Emens L A, Adams S, Loi S, Schneeweiss A, Rugo H S, Winer E P, et al. IMpassion130: a Phase III randomized trial of atezolizumab with nab-paclitaxel for first-line treatment of patients with metastatic triple-negative breast cancer (mTNBC). Journal of Clinical Oncology. 2016; 34 (15_suppl):TPS1104-TPS.
Gandhi L, Rodriguez-Abreu D, Gadgeel S, Esteban E, Felip E, De Angelis F, et al. Pembrolizumab plus Chemotherapy in Metastatic Non-Small-Cell Lung Cancer. The New England journal of medicine. 2018; 378(22):2078-92.
Sistigu A, Yamazaki T, Vacchelli E, Chaba K, Enot D P, Adam J, et al. Cancer cell-autonomous contribution of type I interferon signaling to the efficacy of chemotherapy. Nat Med. 2014; 20(11):1301-9.
Zitvogel L, Galluzzi L, Kepp O, Smyth M J, Kroemer G. Type I interferons in anticancer immunity. Nature reviews Immunology. 2015; 15(7):405-14.

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1.-25. (canceled)

26. A method of treating an AXL-related disease, the method comprising administering to a subject in need thereof a therapeutically effective amount of an AXL inhibitor, wherein the AXL inhibitor is administered in combination with: an immune checkpoint modulator (ICM), a chemotherapeutic agent, radiotherapy, or any combination thereof;
wherein the AXL inhibitor is selected from the group consisting of:
1-(6,7-dihydro-5H-benzo[6,7]cyclohepta[1,2-c]pyridazin-3-yl)-N³-(7-(pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-(S)-pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-(R)-pyrrolidin-1-yl)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-(3-fluoro-4-(4-(pyrrolidin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N⁵-(7-(pyrrolidin-1-yl)-6,7,8,9-tetrahydro-5H-benzo[7]annulene-1-yl)-1H-1,2,4-triazole-3,5-diamine;
1-(6,7-dihydro-5H-benzo[6,7]cyclohepta[1,2-c]pyridazin-3-yl)-N⁵-(7-(S)-pyrrolidin-1-yl-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(t-butoxycarbonylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(acetamido)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((2R)-2-(methoxycarbonyl)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4,4-difluoropiperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((methoxycarbonylmethyl)(methyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((2R)-2-(carboxy)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(ethoxycarbonyl)piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(carboxy)piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((carboxymethyl)(methyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(ethoxycarbonylmethyl)piperazin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(carboxymethyl)piperazin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(pyrrolidin-1-yl)-6,7,8,9-tetrahydro-5H-benzo[7]annulene-1-yl)-1H-1,2,4-triazole-3,5-diamine;
1-(6,7-dihydro-5H-benzo[6,7]cyclohepta[1,2-c]pyridazin-3-yl)-N³-((7S)-7-amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7s)-7-(di(cyclopropylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((2-methylpropyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((propyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(dipropylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(diethylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(cyclohexylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(cyclopentylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((1-cyclopentylethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(2-propylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((3,3-dimethylbut-2-yl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((cyclohexylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(cyclohexylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((5-chlorothien-2-yl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((2-carboxyphenyl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((3-bromophenyl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(dimethylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(cyclobutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(3-pentylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((2,2-dimethylpropyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(cyclopentylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((cyclopentylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(bicyclo[2.2.1]hept-2-en-5-ylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((bicyclo[2.2.1]hept-2-en-5-ylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(3-methylbutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(3-methylbutyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(2-ethylbutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(but-2-enylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(butyl(but-2-enyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N⁵-((7S)-7-(t-butoxycarbonylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-amino-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(dimethylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(diethylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(dipropylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(di(cyclopropylmethyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(di(3-methylbutyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(cyclobutylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(cyclohexylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-((methylethyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(cyclopentylamino)-6,7,8,9-tetrahydro-5H-benzo[7]annulene-2-yl)-1H-1,2,4-triazole-3,5-diamine; and
1-(6,7-dihydro-5H-pyrido[2′,3′:6,7]cyclohepta[1,2-c]pyridazin-3-yl)-N³-((7S)-7-(2-butylamino)-6,7,8,9-tetrahydro-5H-benzo[7]annulene-2-yl)-1H-1,2,4-triazole-3,5-diamine, or pharmaceutically acceptable salts thereof, or
an anti-AXL antibody.

27. The method of claim 26, wherein the AXL-related disease is a proliferative disease, a solid tumor, or cancer.

28. The method of claim 26, wherein the AXL inhibitor is bemcentinib (BGB324/R428).

29. The method of claim 26, wherein the anti-AXL antibody:
i) comprises the 6 CDRs having the sequences of SEQ ID NOs: 1 to 6;
ii) comprises the 6 CDRs having the sequences of SEQ ID NOs: 7 to 12;
iii) comprises a VH domain having the sequence of SEQ ID NO: 13 and a VL domain having the sequence of SEQ ID NO: 15;
iv) comprises a VH domain having the sequence of SEQ ID NO: 13 and a VL domain having the sequence of SEQ ID NO: 16;
v) comprises a VH domain having the sequence of SEQ ID NO: 14 and a VL domain having the sequence of SEQ ID NO: 15; or
vi) comprises a VH domain having the sequence of SEQ ID NO: 14 and a VL domain having the sequence of SEQ ID NO: 16.

30. The method of claim 26, wherein the Axl inhibitor is administered in combination with one or more immune checkpoint inhibitors (ICI).

31. The method of claim 26, wherein the immune checkpoint modulator is an antibody selected from the group consisting of anti-CTLA-4, anti-PD-1, anti-PD-L1, anti-4-1BB, anti-OX-40, anti-GITR, anti-CD27, anti-CD28, anti-CD40, anti-LAG3, anti-ICOS, anti-TWEAKR, anti-HVEM, anti-TIM-1, anti-TIM-3, anti-VISTA, and anti-TIGIT.

32. The method of claim 31, wherein the anti-CTLA-4 antibody is ipilimumab or tremelimumab.

33. The method of claim 31, wherein the anti-PD-1 antibody is pembrolizumab or nivolumab.

34. The method of claim 31, wherein the anti-PD-L1 antibody is atezolizumab (CAS number 1380723-44-3), avelumab (CAS number 1537032-82-8), or durvalumab (CAS number 1428935-60-7).

35. The method of claim 26, wherein the chemotherapeutic agent induces immunogenic cell death of cancer cells.

36. The method of claim 26, wherein the chemotherapeutic agent induces an immune response in the subject.

37. The method of claim 36, wherein the immune response is a type I interferon response in the subject.

38. The method of claim 26, wherein the chemotherapeutic agent is an anthracycline.

39. The method of claim 38, wherein the anthracycline is selected from the group consisting of doxorubicin, daunorubicin, epirubicin, idarubicin, mitoxantrone, and valrubicin.

40. A method of treating an AXL-related disease, the method comprising administering to a subject in need thereof a therapeutically effective amount of the AXL inhibitor bemcentinib (BGB324/R428), wherein the AXL inhibitor is administered in combination with an anti-PD-1 antibody and a chemotherapeutic agent.

41. The method of claim 40, wherein the anti-PD-1 antibody is pembrolizumab.

42. The method of claim 40, wherein the chemotherapeutic agent is selected from the group consisting of carboplatin and pemetrexed disodium.

43. The method of claim 40, wherein the chemotherapeutic agent is carboplatin and pemetrexed disodium.

44. The method of claim 40, wherein the AXL-related disease is non-small cell lung cancer.

45. A method of treating an AXL-related disease, the method comprising administering to a subject in need thereof a therapeutically effective amount of the AXL inhibitor bemcentinib (BGB324/R428), wherein the AXL inhibitor is administered in combination with pembrolizumab, carboplatin, and pemetrexed disodium; and wherein the AXL-related disease is non-small cell lung cancer.


SEQUENCE LISTING (20 sequences)

SEQ ID NO: 1 - protein, 10 aa, Artificial Sequence
  10C9 Heavy CDR1
DYNFTRYYIH

SEQ ID NO: 2 - protein, 17 aa, Artificial Sequence
  10C9 Heavy CDR2
WIYPGTGDSKYNEKFKG

SEQ ID NO: 3 - protein, 9 aa, Artificial Sequence
  10C9 Heavy CDR3
NGNYWYFDV

SEQ ID NO: 4 - protein, 16 aa, Artificial Sequence
  10C9 Light CDR1
RSSKSLLHSNGNTYLY

SEQ ID NO: 5 - protein, 7 aa, Artificial Sequence
  10C9 Light CDR2
RMSNLAS

SEQ ID NO: 6 - protein, 9 aa, Artificial Sequence
  10C9 Light CDR3
MQHREYPFT

SEQ ID NO: 7 - protein, 10 aa, Artificial Sequence
  10G5 Heavy CDR1
GYSFTDFYIN

SEQ ID NO: 8 - protein, 17 aa, Artificial Sequence
  10G5 Heavy CDR2
RIFPGGDNTYYNEKFKG

SEQ ID NO: 9 - protein, 9 aa, Artificial Sequence
  10G5 Heavy CDR3
RGLYYAMDY

SEQ ID NO: 10 - protein, 16 aa, Artificial Sequence
  10G5 Light CDR1
RSSQSLVHSNGIPYLH

SEQ ID NO: 11 - protein, 7 aa, Artificial Sequence
  10G5 Light CDR2
RVSNRFS

SEQ ID NO: 12 - protein, 9 aa, Artificial Sequence
  10G5 Light CDR3
SQGTHVPPT

SEQ ID NO: 13 - protein, 118 aa, Artificial Sequence
  hu10G5 VH(GH1)
EVQLVQSGAGLVQPGGSVRLSCAASGYSFTDFYINWVRQAPGKGLEWIARIFPGGDNTYYNEKFKGRFTLSADTSSSTAYLQLNSLRAEDTAVYYCARRGLYYAMDYWGQGTLVTVSS

SEQ ID NO: 14 - protein, 118 aa, Artificial Sequence
  hu10G5 VH(GH2)
EVQLVESGGGLVQPGGSLRLSCAASGYSFTDFYINWVRQAPGKGLEWVARIFPGGDNTYYNEKFKGRFTLSADTSKSTAYLQMNSLRAEDTAVYYCARRGLYYAMDYWGQGTLVTVSS

SEQ ID NO: 15 - protein, 112 aa, Artificial Sequence
  hu10G5 VL(GL1)
DIQMTQSPSSLSASVGDRVTITCRSSQSLVHSNGIPYLHWYQQKPGKAPKLLIYRVSNRFSGVPSRFSGSGSGTDFTLTISSLQPEDFATYYCSQGTHVPPTFGQGTKVEIK

SEQ ID NO: 16 - protein, 112 aa, Artificial Sequence
  hu10G5 VL(GL2)
DIQMTQSPSSLSASVGDRVTITCRSSQSLVHSNGIPYLHWYQQKPGKAPKLLIYRVSNRFSGVPSRFSGSRSGTDFTLTISSLQPEDFATYYCSQGTHVPPTFGQGTKVEIK

SEQ ID NO: 17 - protein, 448 aa, Artificial Sequence
  10G5 GH1 Heavy chain
EVQLVQSGAGLVQPGGSVRLSCAASGYSFTDFYINWVRQAPGKGLEWIARIFPGGDNTYYNEKFKGRFTLSADTSSSTAYLQLNSLRAEDTAVYYCARRGLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 448 aa, Artificial Sequence
  10G5 GH2 Heavy chain
EVQLVESGGGLVQPGGSLRLSCAASGYSFTDFYINWVRQAPGKGLEWVARIFPGGDNTYYNEKFKGRFTLSADTSKSTAYLQMNSLRAEDTAVYYCARRGLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 219 aa, Artificial Sequence
  10G5 GL1 Light chain
DIQMTQSPSSLSASVGDRVTITCRSSQSLVHSNGIPYLHWYQQKPGKAPKLLIYRVSNRFSGVPSRFSGSGSGTDFTLTISSLQPEDFATYYCSQGTHVPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 219 aa, Artificial Sequence
  10G5 GL2 Light chain
DIQMTQSPSSLSASVGDRVTITCRSSQSLVHSNGIPYLHWYQQKPGKAPKLLIYRVSNRFSGVPSRFSGSRSGTDFTLTISSLQPEDFATYYCSQGTHVPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC